SEQUENCE LISTING (44 sequences)

SEQ ID NO: 1 - DNA, 1701 bp, synthetic construct
  source 1..1701: mol_type = other DNA
atgaagactatcattgctttgagctacattctatgtctggttttcgctcaaaaaattcctggaaatgacaatagcacggcaacgctgtgccttgggcaccatgcagtaccaaacggaacgatagtgaaaacaatcactaacgatagaatcgaagtgactaacgctactgagttagtgcaaaatagctcaatcggtgagatatgcgatagcccacaccaaatattggacggagagaattgtacactgatagacgcactgttaggcgatccacaatgcgacggattccaaaataaaaaatgggacctattcgttgagcgatctaaggcatactctaattgttatccatacgacgtacccgattacgctagtcttaggtcactagttgcgtcaagcggaacactcgaattcaaaaacgaatcattcaattggacaggcgttacgcaaaacggaacatctagcgcatgcattagggggtcaagctcaagcttttttagtagactgaattggttgacacatctgaattatacttatcccgctcttaacgttactatgcctaataaagagcaattcgataaactgtatatatggggcgtacaccatcccggaactgacaaagaccagatattcctatacgctcaatctagcggacggataaccgttagtactaaacgatcacagcaagccgttataccgaatatcggatctagacctagaattagagacataccgtcacgaatatcaatctattggactatcgttaagccaggcgatatactgttgattaatagtacaggtaatctaatcgcacctagggggtatttcaaaattagatccggtaagtctagtattatgagatccgacgctccaatcggtaagtgtaaaagcgaatgcataacacctaatgggtcaataccgaacgataagccattccaaaatgtgaataggattacatacggagcatgccctagatacgttaagcatagtacacttaaactcgcaaccggaatgcgaaacgtacccgaaaaacagactagggggatattcggagctatagccggatttatcgaaaacggatgggagggaatggtagacggatggtacggatttagacaccaaaatagcgaaggtagggggcaagccgcagaccttaaaagtacacaagccgcaatcgatcagattaacggaaagcttaataggttgatcggtaagactaacgaaaaatttcaccaaatcgaaaaagagtttagcgaagtcgagggtagggtgcaagaccttgagaaatacgttgaggatactaaaatcgatctatggtcatacaatgccgaactgttggttgcgcttgagaatcagcatacaatcgatctaaccgatagcgaaatgaataaattgttcgaaaaaactaaaaaacaattgagagagaatgccgaagacatgggtaacggatgttttaagatatatcacaaatgcgataacgcttgtatcggatcaattagaaacgaaacatacgatcataacgtatatagggacgaagcactgaataatagattccaaattaagggcgtagagttgaaatccggatacaaagattggatattgtggatatcctttgccatatcatgttttttgctttgtgttgctttgttggggttcatcatgtgggcctgccaaaagggcaacattagatgcaacatttgcatttga

SEQ ID NO: 2 - DNA, 1410 bp, synthetic construct
  source 1..1410: mol_type = other DNA
atgaatccaaatcaaaagataataacgattggctctgtttctctcaccatttccacaatatgcttcttcatgcaaattgccatcctgataactactgtaacattgcatttcaagcaatacgaattcaactcaccccctaacaatcaggttatgttgtgcgaacctactataatcgaacgtaatataaccgagatagtgtatctgactaataccacaatcgaaaaagagatatgccctaaacccgctgagtataggaattggtctaaaccgcaatgcggtattaccggtttcgcaccattttcgaaagataactccattaggttgtccgccggaggcgatatatgggtgacacgcgaaccatatgtgagttgcgatcccgataaatgttatcaattcgcactcggacaggggactacacttaataacgttcatagcaataataccgttagggataggacaccttatagaacactattgatgaacgaattgggagtgccattccatctaggtacgaaacaggtgtgtatcgcatggtcctctagctcatgtcatgacggtaaggcatggttgcacgtatgcataaccggagacgataaaaacgctaccgctagtttcatatataacggacggttagtcgatagcgtagtgagttggtctaaagacatacttagaacacaggaatccgaatgcgtatgcattaacggtacatgtacagtcgttatgactgacggaaatgctacgggaaaggccgatactaagatactgtttatcgaagagggaaagatagtgcatactagtaagctatccggatccgctcaacatgtcgaagagtgctcatgttatcctagatatcccggagtgagatgcgtttgtagggataattggaaagggtcaaatagacctatcgttgacataaacataaaagatcattcaatcgttagctcatacgtttgttccggactggtaggcgatacacctagaaaaaacgatagctcaagctcaagccattgtcttaaccctaataacgaagagggggggcatggcgttaaagggtgggcattcgacgacggtaacgacgtatggatgggacggacaattaacgaaactagtagattggggtatgagacattcaaagtcgttgagggatggtctaaccctaaatccaaattgcaaattaatcggcaagtgatagtcgatagaggcgatagatccggatattccggtatttttagcgttgagggaaagtcatgtattaataggtgtttttatgtcgaattgattagggggcgaaaagaggaaaccgaagtgctttggacctcaaacagtattgttgtgttttgtggcacctcaggtacatatggaacaggctcatggcctgatggggcggacctcaatctcatgcatatataa

SEQ ID NO: 3 - DNA, 1701 bp, synthetic construct
  source 1..1701: mol_type = other DNA
atgaaggcaatactagtagttctgctatatacatttgcaaccgcaaatgcagacacattatgtataggttatcatgcgaacaattcaacagacactgtagacacagtactagaaaagaatgtaacagtaacacactctgttaacctattggaggataagcataacggtaagctatgcaaactgagaggcgtagcaccattgcatctaggtaagtgtaatatagccggatggattctagggaatcccgaatgcgaatcactatcaaccgctagctcatggtcatacatagtcgaaacaccatcaagcgataacggtacatgttatcccggagactttatcgattacgaagagcttagagagcaattgtctagcgtaagctcattcgaaagattcgaaatttttccgaaaactagctcatggcctaatcacgatagtaataaaggcgtaactgccgcatgcccacacgccggagctaaatcattctataagaatctgatttggttagtgaaaaaagggaattcatatccgaaactatctaaatcatacattaacgataagggtaaggaggtactagtgttgtgggggatacaccatccatcaactagcgccgatcagcaatcattgtatcagaacgcagacgcatacgtattcgtagggtctagtagatactctaaaaaatttaaacccgaaatcgcaattagaccgaaagtgagagaccaagagggtagaatgaattactattggacactagtcgaaccaggcgataagattacattcgaagcgacagggaatctagtcgtaccgagatacgcattcgcaatggagagaaacgccggatccggaattattattagcgatacacccgtacacgattgcaatactacatgtcagacaccaaaaggcgcaattaatactagtctgccatttcagaatatacacccaattacaatcggtaagtgtccaaaatacgttaagtcaactaagttgagactcgcaacagggttgagaaatataccgtcaattcaatctagggggttgttcggagcaatcgcagggtttatcgaaggggggtggacaggtatggttgacggatggtacggataccatcatcaaaacgaacagggatccggatacgcagccgatctgaaaagtacacagaacgctatagacgaaattacgaataaagtgaatagcgtaatcgaaaaaatgaatacgcaatttacagccgtaggtaaggagtttaatcatctcgaaaaaaggattgagaatctgaataaaaaagtcgacgacggattcttagacatttggacttataacgccgaactgttagtgttactcgaaaacgaaagaacactagactatcacgattcaaacgttaagaatctatacgaaaaagtgagatcgcaattgaaaaataacgctaaagagatagggaatgggtgtttcgaattctatcataaatgcgataatacatgtatggaatccgttaaaaacggaacatacgattaccctaagtatagcgaagaggctaaactgaatagggaagagatagacggagtgaaactcgaatcaactaggatttatcagatactcgcaatttatagtacggttgccagttcattggtactggtagtctccctgggggcaatcagtttctggatgtgctctaatgggtctctacagtgtagaatatgtatttaa

SEQ ID NO: 4 - DNA, 1410 bp, synthetic construct
  source 1..1410: mol_type = other DNA
atgaatccaaaccaaaagataataaccattggttcggtctgtatgacaattggaatggctaacttaatattacaaattggaaacataatctcaatatggattagccactcaatccaattggggaatcagaatcaaatcgaaacatgcaatcaatccgtaattacatacgagaataatacttgggtgaatcagacatacgttaacatatcgaatactaatttcgctgccggacaatccgtcgtgagtgtgaaactagccggtaatagtagtctatgtcccgttagcggatgggctatatactctaaagacaatagcgttagaatcggatctaaaggcgacgtattcgttatacgcgaaccattcataagttgtagtccattagagtgtaggactttttttctgacacagggcgcactattgaacgataagcattctaacggtacaatcaaagataggtcaccatatagaacactaatgtcatgtccgataggcgaagtgcctagtccatacaatagtagattcgaatccgtcgcttggtccgctagcgcatgccatgacggtattaattggttgacaatcgggattagcggacccgataacggcgcagtcgccgtacttaagtataacggtataattaccgatactattaagagttggcgaaataatatattgcgaacacaggaatccgaatgcgcatgcgttaacggatcatgttttaccgttatgactgacggaccatctaacgggcaagcgtcatataagatttttagaatcgaaaaaggtaagatagtgaaatccgtcgaaatgaacgctcctaattatcattacgaagagtgctcatgttatcccgattctagcgaaattacatgcgtatgtagagacaattggcacggatctaatagaccttgggtgtcattcaatcagaatctagagtatcaaatcgggtatatatgctcagggatattcggagacaatcctagacctaacgataagacagggtcatgcggaccagtgagttctaacggcgctaacggcgttaaggggtttagtttcaaatacggtaacggcgtatggatagggagaactaagtcaatctctagtagaaacggattcgaaatgatatgggaccctaacggatggaccggaaccgataataatttttcgattaaacaggatatcgtagggattaacgaatggtcagggtatagcggatcattcgtacagcatccagagttaaccggactcgattgcatacgaccatgtttttgggtcgaactgattagggggagaccgaaagagaatactatatggactagcgggagcagcatatccttttgtggtgtaaacagtgacactgtgggttggtcttggccagacggtgctgagttgccatttaccattgacaagtaa

SEQ ID NO: 5 - DNA, 1701 bp, synthetic construct
  source 1..1701: mol_type = other DNA
atgaaggcaatactagtagttctgctatatacatttgcaaccgcaaatgcagacacattatgtataggttatcatgcgaacaattcaacagacactgtagacacagtactagaaaagaatgtaacagtaacacactctgttaacctattggaggataagcataacagtaagctatgcaaactgagaggcgtagcaccattgcatctaggtaagtgtaatatagccggatggattctagggaatcccgaatgcgaatcactatcaaccgctagctcatggtcatacatagtcgaaacaccatcaagcgataacggtacatgttatcccggagactttatcgattacgaagagcttagagagcaattgtctagcgtaagctcattcgaaagattcgaaatttttccgaaaactagctcatggcctaatcacgatagtagtaaaggcgtaactgccgcatgcccacacgccggagctaaatcattctataagaatctgatttggttagtgaaaaaagagaattcatatccgaaactatctaaatcatacattaacgataagggtaaggaggtactagtgttgtgggggatacaccatccatcaactagcgccgatcagcaatcattgtatcagaacgcagacgcatacgtattcgtagggtctagtagatactctaaaaaatttaaacccgaaatcgcaattagaccgaaagtgagaggccaagagggtagaatgaattactattggacactagtcgaaccaggcgataagattacattcgaagcgacagggaatctagtcgtaccgagatacgcattcgcaatggagagaaacgccggatccggaattattattagcgatacacccgtacacgattgcaatactacgtgtcagacaccaaaaggcgcaattaatactagtctgccatttcagaatatacacccaattacaatcggtaagtgtccaaaatacgttaagtcaactaagttgagactcgcaacagggttgagaaatacaccgtcaattcaatctagggggttgttcggagcaatcgcagggtttatcgaaggggggtggacaggtatggttgacggatggtacggataccatcatcaaaacgaacagggatccggatacgcagccgaactgaaaagtacacagaacgctatagacgaaattacgaataaagtgaatagcgtaatcgaaaaaatgaatacgcaatttacagccgtaggtaaggagtttaatcatctcgaaaaaaggattgagaatctgaataaaaaagtcgacgacggattcttagacatttggacttataacgccgaaatgttagtgttactcgaaaacgaaagaacactagactatcacgattcaaacgttaagaatctatacgaaaaagtgagatcgcaattgaaaaataacgctaaagagatagggaatgggtgtttcgaattctatcataaatgcgataatacatgtatggaatccgttaaaaacggaacatacgattaccctaagtatagcgaagaggctaaactgaatagggaagagatagacggagtggaacttgaatcaactaggatttatcagatactcgcaatttatagtacggttgccagttcattggtactggtagtctccctgggggcaatcagtttctggatgtgctctaatgggtctctacagtgtagaatatgtatttaa

SEQ ID NO: 6 - DNA, 1410 bp, synthetic construct
  source 1..1410: mol_type = other DNA
atgaatccaaaccaaaagataataaccattggttcggtctgtatgacaattggaatggctaacttaatattacaaattggaaacataatctcaatatggattagccactcaatccaattggggaatcagaatcaaatcgaaacatgcaatcaatccgtaattacatacgagaataatacttgggtgaatcagacatacgttaacatatcgaatactaatttcactgccggacaatccgtcgtgagtgtgaaactagccggtaatagtagtctatgtcccgttagcggatgggctatatactctaaagacaatagcgttagaatcggatctaaaggcgacgtattcgttatacgcgaaccattcataagttgtagtccattagagtgtaggactttttttctgacacagggcgcactattgaacgataagcattctaacggtacaatcaaagataggtcaccatatagaacactaatgtcatgtccgataggcgaagtgcctagtccatacaatagtagattcgaatccgtcgcttggtccgctagcgcatgccatgacggtattaattggttgacaatcgggattagcggacccgataacggcgcagtcgccgtacttaagtataacggtataattaccgatactattaagagttggcgaaataatatattgcgaacacaggaatccgaatgcgcatgcgttaacggatcatgttttaccgttatgactgacggaccatctaacgggcaagcgtcatataagatttttagaatcgaaaaaggtaagatagtgaaatccgtcgaaatgaacgctcctaattatcattacgaagagtgctcatgttatcccgattctagcgaaattacatgcgtatgtagagacaattggcacggatctaatagaccttgggtgtcattcaatcagaatctagagtatcaaatcgggtatatatgctcagggatattcggagacaatcctagacctaacgataagacagggtcatgcggaccagtgagttctaacggcgctaacggcgttaaggggtttagtttcaaatacggtaacggcgtatggatagggagaactaagtcaatctctagtagaaacggattcgaaatgatatgggaccctaacggatggaccggaaccgataataatttttcgattaaacaggatatcgtagggattaacgaatggtcagggtatagcggatcattcgtacagcatccagagttaaccggactcgattgcatacgaccatgtttttgggtcgaactgattagggggagaccgaaagagaatactatatggactagcgggagcagcatatccttttgtggtgtaaacagtgacactgtgggttggtcttggccagacggtgctgagttgccatttaccattgacaagtaa

SEQ ID NO: 7 - DNA, 1701 bp, synthetic construct
  source 1..1701: mol_type = other DNA
atgaagactatcattgctttgagctacattctatgtctggttttcgctcaaaaacttcctggaaatgacaatagcacggcaacgctgtgccttgggcaccatgcagtaccgaacggaactatcgttaagactattactaacgatagaatcgaagtgactaacgctaccgaattggtgcaaaattcttctatcggcgaaatttgcgatagcccacaccaaatacttgacggagagaattgtacactgatagacgcactgttaggcgatccacaatgcgacggattccaaaataaaaaatgggacctattcgttgagcgatctaaagcatactctaattgttatccatacgacgtacccgattacgctagtcttagatcactggttgcgtcaagcggaacactcgaatttaataacgaatcattcaattggaacggcgtaacgcaaaacggaacaagtagcgcatgtattagacggtctaataatagcttcttcagcagactgaattggttgacacatctgaatttcaaatatcccgctcttaacgttactatgcctaataacgaacaattcgataaactgtatatatggggcgtacaccatcccggaactgacaaagaccaaatctttctatacgctcaacctagcggtaggattaccgtttctactaaacgatcacagcaagccgttataccgaatatcggatcaagacctagaatacggaatataccgtcacgaattagcatatattggacaatcgttaaaccaggagacatactgttaatcaatagtacagggaatctaatcgcaccaagggggtatttcaaaattagatccggtaagagttcaattatgagatccgacgcaccaatcggcaagtgtaaatccgaatgcataacacctaacggatcaatcccaaacgataaaccgtttcagaatgtgaataggattacatacggcgcctgccctagatacgttaagcaatctacacttaaactagctaccggtatgcgaaacgtacccgaaaaacagactagggggatattcggagcgatagccggattcatagagaatgggtgggagggaatggtagacggatggtacggattcagacaccaaaatagcgaaggtagggggcaagccgcagaccttaaatcaacgcaagccgcaatcgatcagattaacggcaagttgaataggttgatcggcaagactaacgaaaaatttcaccaaatcgaaaaagagtttagcgaagtggagggtaggatacaggatctcgaaaaatacgttgaggacactaaaatcgatctatggtcatacaatgccgaactgttagtcgcacttgagaatcagcatacaatcgatctaaccgatagcgaaatgaataagttgttcgaaaaaactaaaaaacaattgagagagaacgctgaggatatggggaatgggtgttttaagatataccataaatgcgataacgcttgcatagggtcaattagaaacggaacatacgatcacgacgtatatagagacgaagcactgaataatagattccaaatcaaaggcgtagagcttaaaagcggatataaggattggatattgtggatatcctttgccatatcatgttttttgctttgtgttgctttgttggggttcatcatgtgggcctgccaaaagggcaacattaggtgcaacatttgcatttga

SEQ ID NO: 8 - DNA, 1410 bp, synthetic construct
  source 1..1410: mol_type = other DNA
atgaatccaaatcaaaagataataacgattggctctgtttctctcaccatttccacaatatgcttcttcatgcaaattgccatcttgataactactgtaacattgcatttcaaacaatacgagttcaatagcccccctaacaatcaggttatgttgtgcgaacctactataatcgaaagaaatattaccgaaatcgtttatctgactaacactactatcgaaaaagaaatatgccctaaacccgctgagtataggaattggtcaaaaccgcaatgcggaataaccggattcgctccatttagtaaggataactcaattaggttgtccgccggaggagatatatgggttacacgcgaaccatatgtgagttgcgatcccgataagtgttatcaattcgcactaggtcagggtacgacacttaataacgtacattccaataataccgtacacgatagaaccccttataggacactattgatgaacgaactcggagtgccattccatctagggactaagcaagtgtgtatcgcatggtcaagctctagttgccatgacggaaaggcatggttgcatgtgtgtattaccggtgacgataaaaacgctaccgcaagctttatatataacgggaggcttgtcgatagcgtagtgagttggtctaaagagatacttagaacacaggaatccgaatgcgtatgcataaacggtacatgtacagtcgttatgaccgacggttccgcttccggaaaagccgatactaagatactgtttatcgaagaggggaaaatagtgcatacaagtacacttagcggctccgctcaacatgtcgaagagtgttcatgttatcctcggtatcccggagtgagatgcgtttgtagggataattggaaagggtcaaatagacctatagtcgatattaacataaaggaccattcaatcgttagctcatacgtttgttccggactggtaggcgatacacctagaaaaaacgatagctctagctcaagtcattgcttagaccctaataacgaagagggagggcatggcgttaagggatgggcattcgacgacggaaacgacgtttggatgggtagaacgattaacgaaacatcacgattggggtatgagacattcaaagtgatagagggatggtctaaccctaaatcgaaattgcaaattaataggcaggtgatagtcgatagaggcgatagatccggatattccggtattttttccgttgagggcaaatcatgtattaatcggtgtttttatgtcgaactgattaggggtaggaaagaggaaaccgaagtgttgtggacttcaaacagtattgttgtgttttgtggcacctcaggtacatatggaacaggctcatggcctgatggggcggacctcaatctcatgcctatataa

SEQ ID NO: 9 - DNA, 1027 bp, synthetic construct
  source 1..1027: mol_type = other DNA
agcaaaagcaggtagatatttaaagatgagtcttctaaccgaggtcgaaacgtacgttctttctatcatcccgtcaggccccctcaaagccgagatcgcgcagagactggaaagtgtctttgcaggaaagaacacagatcttgaggctctcatggaatggctaaagacaagaccaatcttgtcacctctgactaagggaattttaggatttgtgttcacgctcaccgtgcccagtgagcgaggactgcagcgtagacgctttgtccaaaatgccctaaatgggaatggggacccgaacaacatggatagagcagttaaactatacaagaagctcaaaagagaaataacgttccatggggccaaggaggtgtcactaagctattcaactggtgcacttgccagttgcatgggcctcatatacaacaggatgggaacagtgaccacagaagctgcttttggtctagtgtgtgccacttgtgaacagattgctgattcacagcatcggtctcacagacagatggctactaccaccaatccactaatcaggcatgaaaacagaatggtgctggctagcactacggcaaaggctatggaacagatggctggatcgagtgaacaggcagcggaggccatggaggttgctaatcagactaggcagatggtacatgcaatgagaactattgggactcatcctagctccagtgctggtctgaaagatgaccttcttgaaaatttgcaggcctaccagaagcgaatgggagtgcagatgcagcgattcaagtgatcctctcgtcattgcagcaaatatcattgggatcttgcacctgatattgtggattactgatcgtctttttttcaaatgtatttatcgtcgctttaaatacggtttgaaaagagggccttctacggaaggagtgcctgagtccatgagggaagaatatcaacaggaacagcagagtgctgtggatgttgacgatggtcattttgtcaacatagagctagagtaaaaaactaccttgtttctact

SEQ ID NO: 10 - DNA, 2341 bp, synthetic construct
  source 1..2341: mol_type = other DNA
agcgaaagcaggtcaaatatattcaatatggagagaataaaagaactgagagatctaatgtcgcagtcccgcactcgcgagatactcactaagaccactgtggaccatatggccataatcaaaaagtacacatcaggaaggcaagagaagaaccccgcactcagaatgaagtggatgatggcaatgagatacccaattacagcagacaagagaataatggacatgattccagagaggaatgaacaaggacaaaccctctggagcaaaacaaacgatgctggatcagaccgagtgatggtatcacctctggccgtaacatggtggaataggaatggcccaacaacaagtacagttcattaccctaaggtatataaaacttatttcgaaaaggtcgaaaggttgaaacatggtaccttcggccctgtccacttcagaaatcaagttaaaataaggaggagagttgatacaaaccctggccatgcagatctcagtgccaaggaggcacaggatgtgattatggaagttgttttcccaaatgaagtgggggcaagaatactgacatcagagtcacagctggcaataacaaaagagaagaaagaagagctccaggattgtaaaattgctcccttgatggtggcgtacatgctagaaagagaattggtccgtaaaacaaggtttctcccagtagccggcggaacaggcagtgtttatattgaagtgttgcacttaacccaagggacgtgctgggagcagatgtacactccaggaggagaagtgagaaatgatgatgttgaccaaagtttgattatcgctgctagaaacatagtaagaagagcagcagtgtcagcagacccattagcatctctcttggaaatgtgccacagcacacagattggaggagtaaggatggtggacatccttagacagaatccaactgaggaacaagccgtagacatatgcaaggcagcaatagggttgaggattagctcatctttcagttttggtgggttcactttcaaaaggacaagcggatcatcagtcaagaaagaagaagaagtgctaacgggcaacctccaaacactgaaaataagagtacatgaagggtatgaagaattcacaatggttgggagaagagcaacagctattctcagaaaggcaaccaggagattgatccagttgatagtaagcgggagagacgagcagtcaattgctgaggcaataattgtggccatggtattctcacaggaggattgcatgatcaaggcagttaggggcgatctgaactttgtcaatagggcaaaccagcgactgaaccccatgcaccaactcttgaggcatttccaaaaagatgcaaaagtgcttttccagaactggggaattgaatccatcgacaatgtgatgggaatgatcggaatactgcccgacatgaccccaagcacggagatgtcgctgagagggataagagtcagcaaaatgggagtagatgaatactccagcacggagagagtggtagtgagtattgaccgatttttaagggttagagatcaaagagggaacgtactattgtctcccgaagaagtcagtgaaacgcaaggaactgagaagttgacaataacttattcgtcatcaatgatgtgggagatcaatggccctgagtcagtgctagtcaacacttatcaatggataatcaggaactgggaaattgtgaaaattcaatggtcacaagatcccacaatgttatacaacaaaatggaatttgaaccatttcagtctcttgtccctaaggcaaccagaagccggtacagtggattcgtaaggacactgttccagcaaatgcgggatgtgcttgggacatttgacactgtccaaataataaaacttctcccctttgctgctgccccaccagaacagagtaggatgcaattttcctcattgactgtgaatgtgagaggatcagggttgaggatactggtaagaggcaattctccagtattcaattacaacaaggcaaccaaacgacttacagttcttggaaaggatgcaggtgcattgactgaagatccagatgaaggcacatctggggtggagtctgctgtcctgagaggatttctcattttgggcaaagaagacaagagatatggcccagcattaagcatcaatgaactgagcaatcttgcaaaaggagagaaggctaatgtgctaattgggcaaggggacgtagtgttggtaatgaaacgaaaacgggactctagcatacttactgacagccagacagcgaccaaaagaattcggatggccatcaattagtgtcgaattgtttaaaaacgaccttgtttctact

SEQ ID NO: 11 - DNA, 2341 bp, synthetic construct
  source 1..2341: mol_type = other DNA
agcgaaagcaggcaaaccatttgaatggatgtcaatccgactctacttttcctaaaaattccagcgcaaaatgccataagcaccacattcccttatactggagatcctccatacagccatggaacaggaacaggatacaccatggacacagtaaacagaacacaccaatactcagaaaagggaaagtggacgacaaacacagagactggtgcaccccagctcaacccgattgatggaccactacctgaggataatgaaccaagtgggtatgcacaaacagactgtgttctagaggctatggctttccttgaagaatcccacccaggaatatttgagaattcatgccttgaaacaatggaagttgttcaacaaacaagggtagataaactaactcaaggtcgccagacttatgattggacattaaacagaaatcaaccggcagcaactgcattggccaacaccatagaagtctttagatcgaatggcctaacagctaatgagtcaggaaggctaatagatttcttaaaggatgtaatggaatcaatgaacaaagaggaaatagagataacaacccactttcaaagaaaaaggagagtaagagacaacatgaccaagaagatggtcacgcaaagaacaatagggaagaaaaaacaaagactgaataagagaggctatctaataagagcactgacattaaatacgatgaccaaagatgcagagagaggcaagttaaaaagaagggctatcgcaacacctgggatgcagattagaggtttcgtatactttgttgaaactttagctaggagcatttgcgaaaagcttgaacagtctgggctcccagtagggggcaatgaaaagaaggccaaactggcaaatgttgtgagaaagatgatgactaattcacaagacacagagatttctttcacaatcactggggacaacactaagtggaatgaaaatcaaaatcctcgaatgttcctggcgatgattacatatatcaccagaaatcaacccgagtggttcagaaacatcctgagcatggcacccataatgttctcaaacaaaatggcaagactagggaaagggtacatgttcgagagtaaaagaatgaagattcgaacacaaataccagcagaaatgctagcaagcattgacctgaagtacttcaatgaatcaacaaagaagaaaattgagaaaataaggcctcttctaatagatggcacagcatcactgagtcctgggatgatgatgggcatgttcaacatgctaagtacggtcttgggagtctcgatactgaatcttggacaaaagaaatacaccaagacaatatactggtgggatgggctccaatcatccgacgattttgctctcatagtgaatgcaccaaaccatgagggaatacaagcaggagtggacagattctacaggacctgcaagttagtgggaatcaacatgagcaaaaagaagtcctatataaataagacagggacatttgaattcacaagctttttttatcgctatggatttgtggctaattttagcatggagctacccagctttggagtgtctggagtaaatgaatcagctgacatgagtattggagtaacagtgataaagaacaacatgataaacaatgaccttggacctgcaacggcccagatggctcttcaattgttcatcaaagactacagatacacatataggtgccataggggagacacacaaattcagacgagaagatcatttgagttaaagaagctgtgggatcaaacccaatcaaaagtagggctattagtatcagatggaggaccaaacttatacaatatacggaatcttcacattcctgaagtctgcttaaaatgggagctaatggatgatgattatcggggaagactttgtaatcccctgaatccctttgtcagtcataaagagattgattctgtaaacaatgctgtggtaatgccagcccatggtccagccaaaagcatggaatatgatgccgttgcaactacacattcctggattcccaagaggaatcgttctattctcaacacaagccaaaggggaattcttgaggatgaacagatgtaccagaagtgctgcaatctattcgagaaatttttccctagcagttcatataggagaccggttggaatttctagcatggtggaggccatggtgtctagggcccggattgatgccagggtcgacttcgagtctggacggatcaagaaagaagagttctctgagatcatgaagatctgttccaccattgaagaactcagacggcaaaaataatgaatttaacttgtccttcatgaaaaaatgccttgtttctact

SEQ ID NO: 12 - DNA, 2233 bp, synthetic construct
  source 1..2233: mol_type = other DNA
agcgaaagcaggtactgatccaaaatggaagactttgtgcgacaatgcttcaatccaatgatcgtcgagcttgcggaaaaggcaatgaaagaatatggggaagatccgaaaatcgaaactaacaagtttgctgcaatatgcacacatttggaagtttgtttcatgtattcggatttccatttcatcgacgaacggggtgaatcaataattgtagaatctggtgacccgaatgcactattgaagcaccgatttgagataattgaaggaagagaccgaatcatggcctggacagtggtgaacagtatatgtaacacaacaggggtagagaagcctaaatttcttcctgatttgtatgattacaaagagaaccggttcattgaaattggagtaacacggagggaagtccacatatattacctagagaaagccaacaaaataaaatctgagaagacacacattcacatcttttcattcactggagaggagatggccaccaaagcggactacacccttgacgaagagagcagggcaagaatcaaaactaggcttttcactataagacaagaaatggccagtaggagtctatgggattcctttcgtcagtccgaaagaggcgaagagacaattgaagaaaaatttgagattacaggaactatgcgcaagcttgccgaccaaagtctcccaccgaacttccccagccttgaaaactttagagcctatgtagatggattcgagccgaacggctgcattgagggcaagctttcccaaatgtcaaaagaagtgaacgccaaaattgaaccattcttgaggacgacaccacgccccctcagattgcctgatgggcctctttgccatcagcggtcaaagttcctgctgatggatgctctgaaattaagtattgaagacccgagtcacgagggggagggaataccactatatgatgcaatcaaatgcatgaagacattctttggctggaaagagcctaacatagtcaaaccacatgagaaaggcataaatcccaattacctcatggcttggaagcaggtgctagcagagctacaggacattgaaaatgaagagaagatcccaaggacaaagaacatgaagagaacaagccaattgaagtgggcactcggtgaaaatatggcaccagaaaaagtagactttgatgactgcaaagatgttggagaccttaaacagtatgacagtgatgagccagagcccagatctctagcaagctgggtccaaaatgaattcaataaggcatgtgaattgactgattcaagctggatagaacttgatgaaataggagaagatgttgccccgattgaacatatcgcaagcatgaggaggaactattttacagcagaagtgtcccactgcagggctactgaatacataatgaagggagtgtacataaatacggccttgctcaatgcatcctgtgcagccatggatgactttcagctgatcccaatgataagcaaatgtaggaccaaagaaggaagacggaaaacaaacctgtatgggttcattataaaaggaaggtctcatttgagaaatgatactgatgtggtgaactttgtaagtatggagttctcactcactgacccgagactggagccacacaaatgggaaaaatactgtgttcttgaaataggagacatgctcttgaggactgcgataggccaagtgtcgaggcccatgttcctatatgtgagaaccaatggaacctccaagatcaagatgaaatggggcatggaaatgaggcgctgccttcttcagtctcttcagcagattgagagcatgattgaggccgagtcttctgtcaaagagaaagacatgaccaaggaattctttgaaaacaaatcggaaacatggccaatcggagagtcacccaggggagtggaggaaggctctattgggaaagtgtgcaggaccttactggcaaaatctgtattcaacagtctatatgcgtctccacaacttgaggggttttcggctgaatctagaaaattgcttctcattgttcaggcacttagggacaacctggaacctggaaccttcgatcttggggggctatatgaagcaatcgaggagtgcctgattaatgatccctgggttttgcttaatgcatcttggttcaactccttcctcacacatgcactgaagtagttgtggcaatgctactatttgctatccatactgtccaaaaaagtaccttgtttctact

SEQ ID NO: 13 - DNA, 890 bp, synthetic construct
  source 1..890: mol_type = other DNA
agcaaaagcagggtgacaaaaacataatggactccaacaccatgtcaagctttcaggtagactgtttcctttggcatatccgcaagcgatttgcagacaatggattgggtgatgccccattccttgatcggctccgccgagatcaaaagtccttaaaaggaagaggcaacacccttggcctcgatatcgaaacagccactcttgttgggaaacaaatcgtggaatggatcttgaaagaggaatccagcgagacacttagaatgacaattgcatctgtacctacttcgcggtacctttctgacatgaccctcgaggaaatgtcacgagactggttcatgctcatgcctaggcaaaagataataggccctctttgcgtgcgattggaccaggcgatcatggaaaagaacatagtactgaaagcgaacttcagtgtaatctttaaccgattagagaccttgatactactaagggctttcactgaggagggagcaatagttggagaaatttcaccattaccttctcttccaggacatacttatgaggatgtcaaaaatgcagttggggtcctcatcggaggacttgaatggaatggtaacacggttcgagtctctgaaaatatacagagattcgcttggagaaactgtgatgagaatgggagaccttcactacctccagagcagaaatgaaaagtggcgagagcaattgggacagaaatttgaggaaataaggtggttaattgaagaaatgcggcacagattgaaagcgacagagaatagtttcgaacaaataacatttatgcaagccttacaactactgcttgaagtagaacaagagataagagctttctcgtttcagcttatttaatgataaaaaacacccttgtttctact

SEQ ID NO: 14 - DNA, 1565 bp, synthetic construct
  source 1..1565: mol_type = other DNA
agcaaaagcagggtagataatcactcaatgagtgacatcgaagccatggcgtctcaaggcaccaaacgatcatatgaacaaatggagactggtggggagcgccaggatgccacagaaatcagagcatctgtcggaagaatgattggtggaatcgggagattctacatccaaatgtgcactgaactcaaactcagtgattatgatggacgactaatccagaatagcataacaatagagaggatggtgctttctgcttttgatgagagaagaaataaatacctagaagagcatcccagtgctgggaaggaccctaagaaaacaggaggacccatatatagaagagtagacggaaagtggatgagagaactcatcctttatgacaaagaagaaataaggagagtttggcgccaagcaaacaatggcgaagatgcaacagcaggtcttactcatatcatgatttggcattccaacctgaatgatgccacatatcagagaacaagagcgcttgttcgcaccggaatggatcccagaatgtgctctctaatgcaaggttcaacacttcccagaaggtctggtgccgcaggtgctgcggtgaaaggagttggaacaatagcaatggagttaatcagaatgatcaaacgtggaatcaatgaccgaaatttctggaggggtgaaaatggacgaaggacaagggttgcttatgaaagaatgtgcaatatcctcaaaggaaaatttcaaacagctgcccagagggcaatgatggatcaagtaagagaaagtcgaaacccaggaaacgctgagattgaagacctcattttcctggcacggtcagcactcattctgaggggatcagttgcacataaatcctgcctgcctgcttgtgtgtatgggcttgcagtagcaagtgggcatgactttgaaagggaagggtactcactggtcgggatagacccattcaaattactccaaaacagccaagtggtcagcctgatgagaccaaatgaaaacccagctcacaagagtcaattggtgtggatggcatgccactctgctgcatttgaagatttaagagtatcaagtttcataagaggaaagaaagtgattccaagaggaaagctttccacaagaggggtccagattgcttcaaatgagaatgtggaaaccatggactccaataccctggaactgagaagcagatactgggccataaggaccaggagtggaggaaataccaatcaacaaaaggcatccgcaggccagatcagtgtgcagcctacattctcagtgcagcggaatctcccttttgaaagagcaaccgttatggcagcattcagcgggaacaatgaaggacggacatccgacatgcgaacagaagttataagaatgatggaaagtgcaaagccagaagatttgtccttccaggggcggggagtcttcgagctctcggacgaaaaggcaacgaacccgatcgtgccttcctttgacatgagtaatgaagggtcttatttcttcggagacaatgcagaggagtatgacagttgaggaaaaatacccttgtttctact

SEQ ID NO: 15 - DNA, 1777 bp, synthetic construct
  source 1..1777: mol_type = other DNA
agcaaaagcaggggaaaataaaagcaacaaaaatgaaggcaatactagtagttctgctatatacatttgcaaccgcaaatgcagacacattatgtataggttatcatgcgaacaattcaacagacactgtagacacagtactagaaaagaatgtaacagtaacacactctgttaacctattggaggataagcataacggtaagctatgcaaactgagaggcgtagcaccattgcatctaggtaagtgtaatatagccggatggattctagggaatcccgaatgcgaatcactatcaaccgctagctcatggtcatacatagtcgaaacaccatcaagcgataacggtacatgttatcccggagactttatcgattacgaagagcttagagagcaattgtctagcgtaagctcattcgaaagattcgaaatttttccgaaaactagctcatggcctaatcacgatagtaataaaggcgtaactgccgcatgcccacacgccggagctaaatcattctataagaatctgatttggttagtgaaaaaagggaattcatatccgaaactatctaaatcatacattaacgataagggtaaggaggtactagtgttgtgggggatacaccatccatcaactagcgccgatcagcaatcattgtatcagaacgcagacgcatacgtattcgtagggtctagtagatactctaaaaaatttaaacccgaaatcgcaattagaccgaaagtgagagaccaagagggtagaatgaattactattggacactagtcgaaccaggcgataagattacattcgaagcgacagggaatctagtcgtaccgagatacgcattcgcaatggagagaaacgccggatccggaattattattagcgatacacccgtacacgattgcaatactacatgtcagacaccaaaaggcgcaattaatactagtctgccatttcagaatatacacccaattacaatcggtaagtgtccaaaatacgttaagtcaactaagttgagactcgcaacagggttgagaaatataccgtcaattcaatctagggggttgttcggagcaatcgcagggtttatcgaaggggggtggacaggtatggttgacggatggtacggataccatcatcaaaacgaacagggatccggatacgcagccgatctgaaaagtacacagaacgytatagacgaaattacgaataaagtgaatagcgtaatcgaaaaaatgaatacgcaatttacagccgtaggtaaggagtttaatcatctcgaaaaaaggattgagaatctgaataaaaaagtcgacgacggattcttagacatttggacttataacgccgaactgttagtgttactcgaaaacgaaagaacactagactatcacgattcaaacgttaagaatctatacgaaaaagtgagatcgcaattgaaaaataacgctaaagagatagggaatgggtgtttcgaattctatcataaatgcgataatacatgtatggaatccgttaaaaacggaacatacgattaccctaagtatagcgaagaggctaaactgaatagggaagagatagacggagtgaaactcgaatcaactaggatttatcagatactcgcaatttatagtacggttgccagttcattggtactggtagtctccctgggggcaatcagtttctggatgtgctctaatgggtctctacagtgtagaatatgtatttaacattaggatttcagaagcatgagaaaaacacccttgtttctact

SEQ ID NO: 16 - DNA, 1777 bp, synthetic construct
  source 1..1777: mol_type = other DNA
agcaaaagcaggggaaaataaaagcaacaaaaatgaaggcaatactagtagttctgctatatacatttgcaaccgcaaatgcagacacattatgtataggttatcatgcgaacaattcaacagacactgtagacacagtactagaaaagaatgtaacagtaacacactctgttaacctattggaggataagcataacggtaagctatgcaaactgagaggcgtagcaccattgcatctaggtaagtgtaatatagccggatggattctagggaatcccgaatgcgaatcactatcaaccgctagctcatggtcatacatagtcgaaacaccatcaagcgataacggtacatgttatcccggagactttatcgattacgaagagcttagagagcaattgtctagcgtaagctcattcgaaagattcgaaatttttccgaaaactagctcatggcctaatcacgatagtaataaaggcgtaactgccgcatgcccacacgccggagctaaatcattctataagaatctgatttggttagtgaaaaaagggaattcatatccgaaactatctaaatcatacattaacgataagggtaaggaggtactagtgttgtgggggatacaccatccatcaactagcgccgatcagcaatcattgtatcagaacgcagacgcatacgtattcgtagggtctagtagatactctaaaaaatttaaacccgaaatcgcaattagaccgaaagtgagagaccaagagggtagaatgaattactattggacactagtcgaaccaggcgataagattacattcgaagcgacagggaatctagtcgtaccgagatacgcattcgcaatggagagaaacgccggatccggaattattattagcgatacacccgtacacgattgcaatactacatgtcagacaccaaaaggcgcaattaatactagtctgccatttcagaatatacacccaattacaatcggtaagtgtccaaaatacgttaagtcaactaagttgagactcgcaacagggttgagaaatataccgtcaattcaatctagggggttgttcggagcaatcgcagggtttatcgaaggggggtggacaggtatggttgacggatggtacggataccatcatcaaaacgaacagggatccggatacgcagccgawctgaaaagtacacagaacgytatagacgaaattacgaataaagtgaatagcgtaatcgaaaaaatgaatacgcaatttacagccgtaggtaaggagtttaatcatctcgaaaaaaggattgagaatctgaataaaaaagtcgacgacggattcttagacatttggacttataacgccgaactgttagtgttactcgaaaacgaaagaacactagactatcacgattcaaacgttaagaatctatacgaaaaagtgagatcgcaattgaaaaataacgctaaagagatagggaatgggtgtttcgaattctatcataaatgcgataatacatgtatggaatccgttaaaaacggaacatacgattaccctaagtatagcgaagaggctaaactgaatagggaagagatagacggagtgaaactcgaatcaactaggatttatcagatactcgcaatttatagtacggttgccagttcattggtactggtagtctccctgggggcaatcagtttctggatgtgctctaatgggtctctacagtgtagaatatgtatttaacattaggatttcagaagcatgagaaaaacacccttgtttctact

SEQ ID NO: 17 - DNA, 1777 bp, synthetic construct
  source 1..1777: mol_type = other DNA
agcaaaagcaggggaaaataaaagcaacaaaaatgaaggcaatactagtagttctgctatatacatttgcaaccgcaaatgcagacacattatgtataggttatcatgcgaacaattcaacagacactgtagacacaatactagaaaagaatgtaacagtaacacactctgttaacctattggaggataagcataacggtaagctatgcaaactgagaggcgtagcaccattgcatctaggtaagtgtaatatagccggatggattctagggaatcccgaatgcgaatcactatcaaccgctagctcatggtcatacatagtcgaaacaccatcaagcgataacggtacatgttatcccggagactttatcgattacgaagagcttagagagcaattgtctagcgtaagctcattcgaaagattcgaaatttttccgaaaactagctcatggcctgatcacgatagtaataaaggcgtaactgccgcatgcccacacgccggagctaaatcattctatgagaatctggtttggttagtgaaaaaagggaattcatatccgaaactatctaaatcatacattaacgataagggtaaggaggtactagtgttgtgggggatacaccatccatcaactagcgccgatcagcaatcattgtatcagaacgcagacgcatacgtattcgtagggtctagtagatactctaaaaaatttaaacccgaaatcgcaattagaccgaaagtgagaggccaagagggtagaatgaattactattggacactagtcgaaccaggcgataagattacattcgaagcgacagggaatctagtcgtaccgagatacgcattcgcaatggagagaaacgccggatccggaattattattagcgatacacccgtacacgattgcaatactacatgtcagacaccagaaggcgcaattaatactagtctgccatttcagaatatacacccaattacaatcggtaagtgtccaaaatacgttaagtcaactaagttgagactcgcaacagggttgagaaatataccgtcaattcaatctagggggttgttcggagcaatcgcagggtttatcgaaggggggtggacaggtatggttgacggatggtacggataccatcatcaaaacgaacatggatccggatacgcagccgaactgaaaagtacacagaacgctatagacgaaattacgaataaagtgaatagcgtaatcgaaaaaatgaatacgcaatttacagccgtaggtaaggagtttaatcatctcgaaaaaaggattgagaatctgaataaaaaagtcgacgacggattcttagacatttggacttataacgccgaactgttagtgttactcgaaaacgaaagaacactagactatcacgattcaaacgttaagaatctatacgaaaaagtgagatcgcaattgaaaaataacgctaaagagatagggaatgggtgtttcgaattctatcataaatgcgataatacatgtatggaatccgttaaaaacggaacatacgattaccctaagtatagcgaagaggctaaactgaatagagaagagatagacggagtggaactcgagtcaactaggatttatcagatactcgcaatttatagtacggttgccagttcattggtactggtagtctccctgggggcaatcagtttctggatgtgctctaatgggtctctacagtgtagaatatgtatttaacattaggatttcagaagcatgagaaaaacacccttgtttctact

SEQ ID NO: 18 - DNA, 3235 bp, synthetic construct
  source 1..3235: mol_type = other DNA
agcaaaagcaggggaaaataaaagcaacaaaaatgaaggcaatactagtagttctgctatatacatttgcaaccgcaaatgcagacacattatgtataggttatcatgcgaacaattcaacagacactgtagacacaatactagaaaagaatgtaacagtaacacactctgttaacctattggaggataagcataacggtaagctatgcaaactgagaggcgtagcaccattgcatctaggtaagtgtaatatagccggatggattctagggaatcccgaatgcgaatcactatcaaccgctagctcatggtcatacatagtcgaaacaccatcaagcgataacggtacatgttatcccggagactttatcgattacgaagagcttagagagcaattgtctagcgtaagctcattcgaaagattcgaaatttttccgaaaactagctcatggcctgatcacgatagtaataaaggcgtaactgccgcatgcccacacgccggagctaaatcattctatgagaatctggtttggttagtgaaaaaagggaattcatatccgaaactatctaaatcatacattaacgataagggtaaggaggtactagtgttgtgggggatacaccatccatcaactagcgccgatcagcaatcattgtatcagaacgcagacgcatacgtattcgtagggtctagtagatactctaaaaaatttaaacccgaaatcgcaattagaccgaaagtgagaggccaagagggtagaatgaattactattggacactagtcgaaccaggcgataagattacattcgaagcgacagggaatctagtcgtaccgagatacgcattcgcaatggagagaaacgccggatccggaattattattagcgatacacccgtacacgattgcaatactacatgtcagacaccagaaggcgcaattaatactagtctgccatttcagaatatacacccaattacaatcggtaagtgtccaaaatacgttaagtcaactaagttgagactcgcaacagggttgagaaatataccgtcaattcaatctagggggttgttcggagcaatcgcagggtttatcgaaggggggtggacaggtatggttgacggatggtacggataccatcatcaaaacgaacatggatccggatacgcagccgaactgaaaagtacacagaacgctatagacgaaattacgaataaagtgaatagcgtaatcgaaaaaatgaatacgcaatttacagccgtaggtaaggagtttaatcatctcgaaaaaaggattgagaatctgaataaaaaagtcgacgacggattcttagacatttggacttataacgccgaactgttagtgttactcgaaaacgaaagaacactagactatcacgattcaaacgttaagaatctatacgaaaaagtgagatcgcaattgaaaaataacgctaaagagatagggaatgggtgtttcgaattctatcataaatgcgataatacatgtatggaatccgttaaaaacggaacatacgattaccctaagtatagcgaagaggctaaactgaatagagaagagatagacggagtggaactcgagtcaactaggatttatcagatactcgcaatttatagtacggttgccagttcattggtactggtagtctccctgggggcaatcagtttctggatgtgctctaatgggtctctacagtgtagaatatgtatttaacattaggatttcagaagcatgagaaaaacacccttgtttctactagcaaaagcaggggtttaaaatgaatccaaaccaaaagataataaccattggttcggtctgtgtgacaattggaatggctaacttaatattacaaattggaaacataatctcaatatggattagccactcaatccaattggggaatcagaatcaaatcgaaacatgcaatcaatccgtaattacatacgagaataatacttgggtgaatcagacatacgttaacatatcgaatactaatttcactgccggaccatccgtcgtgagtgtgaaactagccggtaatagtagtctatgtcccgttagcggatgggctatatactctaaagacaatagcgttagaatcggatctaaaggcgacgtattcgttatacgcgaaccattcataagttgtagtccattagagtgtaggactttttttctgacacagggcgaactattgaacgataagcattctaacggtacaatcaaagataggtcaccatatagaacactaatgtcatgtccgataggcgaagtgcctagtccatacaatagtaaattcgaatccgtcgcttggtccgctagcgcatgccatgacggtattaattggttgacaatcgggattagcggacccgataacggcgcagtcgccgtacttaagtataacggtataattaccgatactattaagagttggcgaaataatatattgcgaacacaggaatccgaatgcgcatgcgttaacggatcatgttttaccgttatgactgacggaccatctaacgggcaagcgtcatataagatttttagaatcgaaaaaggtaagatagtgaaatccgtcgaaatgaacgctcctaattatcattacgaagagtgctcatgttatcccgattctagcgaaattacatgcgtatgtagagacaattggcacggatctaatagaccttgggtgtcattcaatcagaatctagagtatcaaatcgggtatatatgctcagggatattcggagacaatcctagacctaacgataagacagggtcatgcggaccagtgagttctaacggcgctaacggcgttaaggggtttagtttcaaatacggtaacggcgtatggatagggagaactaagtcaatctctagtagaaacggattcgaaatgatatgggaccctaacggatggaccggaaccgataataatttttcgattaaacaggatatcgtagggattaacgaatggtcagggtatagcggatcattcgtacagcatccagagttaaccggactcgattgcatacgaccatgtttttgggtcgaactgattagggggagaccgaaagagaatactatatggactagcgggagcagcatatccttttgtggtgtaaacagtgacactgtgggttggtcttggccagacggtgctgagttgccatttaccattgacaagtaatttgttcaaaaaactccttgtttctact

SEQ ID NO: 19 - DNA, 1683 bp, synthetic construct
  source 1..1683: mol_type = other DNA
atgtataagatagtgctcgtactcgcactattaggcgcagtgcacggactcgacaaaatttgcctagggcatcacgcagtgcctaacggaactatcgttaagacacttactaacgaaaaagaggaagtgactaacgctaccgaaacagtcgaatcaaaatcactcgacaaattgtgtatgaaaagtcggaattataaagacctaggcaattgccatccgatagggatggtgatagggactcccgcttgcgatctgcatctgacagggacatgggatacacttatcgaacgggacaatagtatagcgtattgttatccaggcgctacagtgaacgaagaggcacttagacaaaaaattatggaatccggcgaaatcgataagattagtaccggattcacatacgaatcctctattaatcccgcaggaacaactaaggcttgtatgcgaaacggtaagaattcgttttacgctgaactgaaatggcttgtgagtaaggacaaaggtaggaatttcccacaaactactaatacttataggaataccgattcaaccgaacatctgattatatgggggatacaccatccaagttcgacacaagagaaaaacgatctatacggaacgcaatcccttagcattagcgtagggtctagtacttatcagaataatttcgtaccggtagtgggcgctagaccgcaagtgaacggacaatccggtagaatcgatttccattgggctatggtgcaaccaggcgataacataacttttagccataacggcggactgatagcgcctagtagagtgagtaagcttaagggaagggggttggggatacaatccggcgctagcgtagacaacgattgcgaatcaaaatgcttttggaaaggggggtcaattaatactaaattgccatttcagaatctgtcacctagaacagtgggacaatgccctaaatacgttaataagaaaagtctgttactcgcaaccggtatgcgaaacgtaccagaggtagtgcaaggtagggggctattcggagcgatagcgggatttatcgaaaacggatgggagggtatggtcgacggatggtacgggtttagacaccaaaacgcacagggaaccggacaggcagcagactataaatcgacacaagccgctatagaccaaattaccggtaagcttaacagactgatcgaaaagactaataccgaattcgaatcaatcgaatccgaatttagcgaaatcgaacaccaaatcggaaacgtaattaattggacaaaagactcaattaccgatatatggacatatcaagccgaactgttagtcgctatggagaatcagcatacaatcgatatggccgatagcgaaatgcttaacctttacgaaagggtgagaaaacagcttagacaaaacgctgaagaggacggtaaggggtgtttcgaaatataccataaatgcgacgataattgtatggagtctatacggaataacacatacgaccatacgcaatatagagaggaagcactactgaatagacttaacattaatccggttaagctatctagcggatataaagacgtgatattgtggttctcattcggagcgtcatgtttcgtattgctcgcagtgattatgggactcgtattcttttgccttaaaaacggtaatatgagatgcacaatttgcata

SEQ ID NO: 20 - DNA, 1410 bp, synthetic construct
  source 1..1410: mol_type = other DNA
atgaatcctaaccaaaagctattcgcactaagcggagtcgccatagccctatcaatactgaatctgttaatcggaatatcgaacgttgggttgaacgttagtttgcaccttaaggggtcatccgaccaagacaaaaattggacatgtactagcgttacgcaaacaaatacgactttgatcgaaaatacatacgttaacaatacgacagtgataaataaagagaccggaactactaagcaaaactatctgatgctgaataagtcactatgtaaggtcgagggatgggtggtagtcgctaaagacaacgcaataaggttcggcgaaagcgaacagataatcgtgacacgcgaaccatacgttagttgcgatccgttagggtgtaagacatacgcattacaccaagggactacgatacggaataaacactctaacggaacgatacacgacagaaccgcatttagggggttgatatcgacacctctcggatcacctcccgtagtgagtaatagcgatttcttatgcgtggggtggtcaagtactagttgtcacgacggaatcggacgtatgacaatatgcgtacaggggaataacaataacgcaaccgcaacagtgtattacgataggagactgactacaacaattaagacttgggccggtaagatactgagaacacaggaaagcgaatgcgtttgccataacggtacatgcgtagtgattatgacagacggatccgcaagttcgcaagcccatacgaaagtgctatattttcacaaagggctcgtaatcaaagaggaagcccttaagggatccgctagacatatcgaagagtgtagttgttacggacacaatagtaaggttacatgcgtatgtagggacaattggcaaggcgcaaatagaccagtgatagagatagacatgaacgctatggagcatacgagtcagtatctatgtaccggagtgttaaccgacactagtagacctagcgataagagtatgggcgattgcaataatccgataaccggatcacccggagcaccaggcgttaaggggttcgggtttctcgatagcgataatacatggttaggtaggacaatctcacctaggtcaagatccggattcgaaatgctcaaaatccctaacgccggaacagaccctaatagtaggattaccgaacgacaagagatagtcgacaataacaattggtcagggtatagcggatctttcatagactattgggacgaatcaagcgtatgttataacccatgtttctatgtcgaactgattagggggagacccgaagaggccaaatatgtgtggtggactagtaatagtctcgtagccctatgcggatcaccgataagcgtagggtcagggtcattcccagacggagcccaaatccaatattttagt

SEQ ID NO: 21 - DNA, 1698 bp, synthetic construct
  source 1..1698: mol_type = other DNA
atgaaagcgattctagtcgtactgctatatacattcgctaccgctaacgccgatacactatgcatagggtatcacgctaataatagtacagacacagtagacacagtactcgaaaaaaacgttacggttacacattccgttaatctgttagaggataagcataacggtaagctatgtaaactgagaggcgtagcaccattgcatttgggtaagtgtaatatagccggatggatactaggtaatcccgaatgcgaatcactatcaactgcaagttcatggtcttatatagtcgaaactagttcaagcgataacggtacatgttatcccggagactttatcgattacgaagagttgagagagcaattgtctagcgtaagctcattcgaaagattcgaaatttttccgaaaactagttcatggcctaatcacgattcaaataagggggtaacagccgcatgcccacacgcaggcgctaagtcattctataaaaatctgatatggctagtgaaaaaagggaattcttatccgaaactatcaaaatcatatattaacgataagggtaaggaggtactcgtattgtgggggatacaccatccatcaactagcgcagaccaacaatctctgtatcagaatgccgacgcatacgtattcgtagggactagtaggtactctaaaaaatttaaacccgaaatcgctattagaccgaaagtgagagaccaggagggaagaatgaattactattggacactagtcgaaccaggcgataagattacattcgaagcgacagggaatctagtggtaccgagatacgcattcgcaatggagagaaacgccggatccggaattattattagcgatactcccgtacacgattgcaatacaacatgtcagacaccaaaaggggcaattaatactagcctaccatttcagaatatacacccaattacaatcggtaagtgtccaaaatacgttaagtctacgaaacttagattggcaacagggttgagaaacgtaccatcaatacagtctagagggttgttcggagcaatcgccggattcatagagggggggtggaccggtatggtcgacggatggtacggataccatcatcaaaacgaacaggggtccggatacgcagccgatctgaaatcaacacagaacgcaatcgacgaaattacgaataaagtgaatagcgtaatcgaaaaaatgaatactcagtttacagccgtaggtaaggaatttaatcatctcgaaaaaagaattgagaatctgaataaaaaggtagacgacgggtttctagacatttggacatataatgccgaactgttagtgttactcgaaaacgaaagaacattagactatcacgattctaacgttaagaatctatacgaaaaagtgagatcgcaattgaagaataacgcaaaagagatagggaatgggtgtttcgaattctaccataaatgcgataatacatgtatggaatccgtaaaaaacggtacatacgattatccgaaatatagcgaagaagcaaaactgaatagggaagagattgacggagttaagttggagtcaactaggatttaccagatactcgcaatttactctacagtcgcatcaagtctagtgttagtcgttagcttaggcgcaattagtttttggatgtgttcaaacggatcactgcaatgtaggatttgcata

SEQ ID NO: 22 - DNA, 1407 bp, synthetic construct
  source 1..1407: mol_type = other DNA
atgaatcctaaccaaaaaattataacaatcggatccgtttgtatgacaatcggtatggctaacctaatactgcaaatcggtaatattatatcgatttggatctcacatagtatacaattgggtaatcagaatcagatagagacatgcaatcaatccgttattacatacgaaaataatacttgggttaatcagacatacgttaacatatcgaatactaatttcgctgccggacaatccgtcgttagcgttaagttagccggtaatagttcactatgccccgttagcgggtgggctatatactctaaagacaattcgattagaatcggatctaagggcgacgtattcgtaatacgcgaaccattcataagttgtagtccattagagtgtagaactttttttctaacacaaggcgctctattgaacgataagcatagtaacggtacaattaaggatagatcaccttatagaacattgatgtcatgtcctatcggcgaagtgcctagtccatacaatagtagattcgaatccgtcgcatggtccgctagcgcatgtcacgacgggattaattggttgactatagggattagcggacccgataacggcgcagtcgctgtgcttaagtataacggtattattaccgacactataaagagttggcgaaataacatactgagaacacaggaatccgaatgcgcatgcgtaaacggttcatgttttaccgtaatgactgacggacctagcgacggacaagcgtcatataagatttttagaatcgaaaaaggtaagatagtgaaatctgtcgagatgaacgctccgaattatcattacgaagagtgtagttgttatcccgattctagcgaaattacatgcgtatgtagggacaattggcacgggtctaatcgaccatgggtgtcattcaatcagaacttagagtatcagatagggtatatatgctcagggatattcggcgataatcctagaccgaacgataaaaccggatcatgcggaccagtgtcatctaacggcgctaacggagtgaaagggtttagtttcaaatacggtaacggcgtatggatcggacgaactaagtctatatctagtaggaacggattcgaaatgatatgggacccaaacgggtggaccggtaccgataataacttttcaatcaaacaggacatagtcggaattaacgaatggtccgggtatagcggatcattcgtgcaacatccagagttaaccggactcgattgcataagaccatgtttttgggtcgaattgattagggggagaccaaaagagaatactatatggactagcggatctagtattagcttttgcggagtgaatagcgataccgtagggtggtcatggccagacggagccgaactaccatttacaatcgataag

SEQ ID NO: 23 - DNA, 1698 bp, synthetic construct
  source 1..1698: mol_type = other DNA
atgaaagtgaaactgttaatactgttgtgcacttttaccgctacatacgccgatacaatttgcatagggtatcacgctaataatagtaccgatacagtcgacactgtgttggaaaagaacgtaaccgttacacactccgttaatctgttagaggattcccataacggtaagttgtgtctgttgaaagggatcgcaccattgcaattgggtaattgtagcgtagccggatggatattggggaatcccgaatgcgaactattgattagtaaagagtcatggtcatatatagtcgagacacctaatcccgaaaacggagcatgctatcccggatatttcgccgattacgaagagcttagagagcaattgtctagcgtaagctcattcgaaagattcgaaatttttccaaaagagtcaagttggcctaatcataccgtaacaggcgtatccgcatcatgtagtcataacggtaagtcaagcttttataagaatctgttatggttaaccggtaaaaacggactgtatccaaatctatctaagtcatacgcaaataataaagagaaagaggtactgattctatggggggtgcatcacccacctaatataggcgatcaaagaacattgtatcataccgaaaacgcatacgtatccgtcgttagctcacactatagtagaaggtttacacccgaaattactaagagacctaaggtaagggatcaggagggtaggattaattattattggactctacttgaaccaggcgatactatcatattcgaagctaacggaaatctaatcgcaccatggtacgcattcgcactatctagggggttcggatccgggattattacttctaacgctccaatggacgaatgcgacgcaaagtgtcagacaccacagggagcgattaatagttccctaccattccaaaacgtacaccccgttacaatcggcgaatgtccgaaatacgttagatccgctaaacttagaatggtgaccggactgagaaatataccatcaatccaatctagggggctattcggagccatagccggatttatcgaaggggggtggacagggatggtcgacggatggtatgggtatcaccaccaaaacgaacagggatccggatacgccgccgatcagaaatccacacaaaacgctattaacggaattacgaataaagtgaatagcgtaatcgaaaaaatgaatacacaatttactgccgtaggtaaggaattcaataagttagagagaaggatggagaatctgaataaaaaagtcgacgacggattcctagacatatggacatataacgccgaactgttagtgttgcttgagaacgaaaggacactagactttcacgattcaaacgttaaaaatctatacgaaaaagtcaaatcccaattgaaaaataacgctaaagagatagggaatgggtgtttcgaattctatcataagtgtaataacgaatgtatggaatccgttaaaaacggaacatacgattatccaaagtatagcgaagagtcaaaactgaatagggaaaaaatcgacggagtcaaacttgactcaatgggggtgtatcagatactcgcaatctatagtacagtcgcatctagcctagtactgttagtgagtctgggagcgataagcttttggatgtgttctaacggatcactgcaatgtaggatatgcatatga

SEQ ID NO: 24 - DNA, 1410 bp, synthetic construct
  source 1..1410: mol_type = other DNA
atgaaccctaatcaaaaaataattacaatcggatccgttagtctgacaatcgctactatatgttttctgactcagatagcgatactcgttacaaccgttacattgcatttcaaacaatacgaatgcaattccccccctaacaatcaggtaatgttgtgcgaacctacaataatcgaacggaatattaccgagatagtgtatctgactaatacgactatcgaaaaagagatatgcccaaaactagccgaatatcggaattggtcaaaaccgcaatgtaacataaccggattcgcaccattttcgaaagacaattcgattaggttgtccgccggaggcgatatttgggttacacgcgaaccttatgtgtcatgcgatcccgataaatgctatcaattcgcactcggacaggggactacccttaataacggacattctaacgataccgtacacgatagaactccatatcgaacattgctaatgaacgagttaggcgtaccattccatttgggcactaaacaggtatgtatcgcatggtctagctctagttgccatgacggtaaggcttggttgcatgtgtgcgttaccggcgacgataagaacgcaaccgctagctttatatataacggtaggttggtcgactcaatcgggtcatggtcaaaaaaaatacttagaacgcaagagtccgaatgcgtatgcataaacggtacatgcaccgtagtgatgaccgacggatccgctagcggtaaggccgatacgaaaatactgtttatcgaagagggtaagatagtgcatacgagtctactatccggatccgctcaacatgtcgaagagtgttcatgttatcctaggtatcccggcgttagatgcgtatgtagggataattggaaagggagtaatagacctatagtcgatattaacgttaaggattattcaatcgtaagtagttatgtgtgtagcggactcgtaggcgatacacctagaaaaaacgatagctctagtagctcacattgcctagaccctaataacgaagagggggggcatggcgttaagggatgggcattcgacgacggtaacgacgtttggatgggtaggactattagcgaaaagcttagatccgggtatgagacattcaaagtgatagagggatggtctaaacctaattcaaaactgcaaattaataggcaagtgatagtcgatagggataatagatccgggtattccggaatttttagcgttgagggtaagtcatgtattaataggtgtttttatgtcgagcttattagggggagaaatcaggaaaccgaagtgttgtggacatccaattcaatcgtcgttttttgcggaactagcggaacatacggtaccggatcatggcccgacggagccgatattaaccttatgcctatataa

SEQ ID NO: 25 - DNA, 1689 bp, synthetic construct
  source 1..1689: mol_type = other DNA
atggcaataatctatctgatactgttgtttacagccgttaggggcgatcagatatgcatagggtatcacgctaataatagtaccgaaaaagtcgatacaatactcgaaagaaacgtaaccgttacacacgctaaagatatactcgaaaagacacataacggtaagctatgcaaacttaacggtataccaccacttgagttaggcgattgctcaatcgcaggatggttgttggggaatcccgaatgcgataggctattgagcgtacccgaatggtcttatattatggaaaaagagaatcctagagacggattgtgttatcccggatcttttaacgattacgaagagcttaaacatctgctatctagcgttaaacatttcgaaaaagtgaaaattctgccaaaagataggtggacacagcatacgactaccggaggatctagggcatgcgccgttagcggtaatccgtcattctttagaaatatggtatggttgacaaaaaaggggtctaattatccagtcgctaagggatcgtataataatacaagcggagagcaaatgttgattatatggggagtgcatcaccctaacgacgaaaccgaacaacggacactgtatcaaaacgtcggaacatacgttagcgtcggtacaccaactctgaataaaagatcgactcccgatatcgcaactagaccaaaagtgaacggacagggggggagaatggagtttagttggacactactcgatatgtgggatacaattaatttcgaatcaaccggtaatctgatcgcacccgaatacgggtttaagattagtaaaagggggtcatccggtattatgaaaaccgaaggtacactagggaattgcgaaactaagtgtcagacaccactaggggctattaatacaacactaccatttcataatgtgcatccattgacaatcggagagtgtcctaagtatgtgaaatccgaaaaactagtgcttgcaaccggactgagaaacgtaccgcaaatcgaatccagagggttgttcggagcaatcgcagggtttatcgaaggggggtggcagggaatggtcgacggatggtatgggtatcatcactctaacgatcagggatccggatacgcagccgataaggagtcaacccaaaaagcattcgacggaattactaataaggtgaatagcgtaatcgaaaaaatgaatacacaattcgaagccgtcggtaaagagttttcgaatctcgaaaggagacttgagaatctgaataaaaaaatggaggacggattcttagacgtatggacatataatgccgaactgttagtccttatggagaacgaacggacactagactttcacgatagtaacgttaagaatctgtatgacaaagtgagaatgcaattgagagacaatgtgaaagagctaggtaacggatgtttcgaattctatcataaatgcgacgacgagtgtatgaatagcgttaaaaacggtacatatgactatcctaagtatgaggaagagtcaaagcttaatagaaacgagattaagggagtgaaactatctagtatgggagtgtatcagatactcgcaatatacgctacagtcgccggatccctatcacttgcgattatgatggccggaattagcttttggatgtgctctaacggatcattgcaatgtaggatttgcatatga

SEQ ID NO: 26 - DNA, 1410 bp, synthetic construct
  source 1..1410: mol_type = other DNA
atgaatcctaaccagaaaattattactatagggtcagtgtcattgactatcgcaaccgtatgctttattatgcaaatagcgatactcgcaactaccgtaacattgcattttaaacaacacgaatgcgatagtcccgctagcaatcaggtaatgccatgcgaacctattataatcgaacggaatattaccgagatagtgtatcttaacaatactactatcgaaaaagagatatgcccagaggccgtcgagtatagaaattggtctaaacctcaatgtcagattaccggattcgcaccattctctaaagacaattcgattagattgtccgccggaggcgatatatgggtgacacgcgaaccttatgtgtcatgcgatcccggtaagtgttatcaattcgcactcggacaggggactacactcgataataaacattctaacggtacgatacacgataggattccacataggacactattgatgaacgagttaggcgtaccgtttcatctaggcactaaacaggtatgcgttgcgtggtctagctcatcatgtcatgacggtaaggcatggttgcatgtgtgcgtaaccggcgacgatagaaacgctaccgctagttttatatacgacggtaggctagtcgattcaatcggatcatggtcacagaatatacttagaacacaggaatccgaatgcgtttgtattaacggtacatgtacagtcgttatgaccgacggatccgcatccggtagggccgatactaggatactgtttataaaagagggcaaaatcgtgcatattagcccacttagcggatccgcacaacatatcgaagagtgtagttgctatcctaggtatcctgacgttagatgtatttgcagagacaattggaaagggtctaatagacccgtaatcgatatcaatatggaggattattcaatcgatagctcttatgtgtgtagcggattagtcggcgatacacctagaaacgacgatagctctagtaattcgaattgtagggaccctaataacgagagaggcaatcccggcgttaaagggtgggcattcgataacggcgacgacgtttggatggggcgaacaattaataaggactctagatccgggtatgagacattcaaagtgataggggggtggtctacacctaactcaaaatctcaagtgaataggcaagtgatagtcgacaataacaattggtcagggtatagcggtatattctcagtcgagggtaagtcatgtattaatagatgtttttacgttgagttgattagggggcgaccacaagagactagagtgtggtggactagtaatagtatagtcgttttttgcggaactagcggtacatacggaaccggatcatggcctgacggagcgaatattaattttatgccaatctaa

SEQ ID NO: 27 - DNA, 1701 bp, synthetic construct
  source 1..1701: mol_type = other DNA
atgaaaacaattatcgcactgtcatacatactgtgtctggtattcgctcaaaaattgcccggtaacgacaattcaaccgctacattgtgcttagggcatcacgccgtaccgaacggaactatcgttaagacaattactaacgaccaaatcgaagtgactaacgctacagagttggtgcaatcctctagtacaggcgaaatatgcgattcaccacaccaaatccttgacggagagaattgtacacttatcgacgcactattaggcgatccacaatgcgacggatttcagaataaaaaatgggatctattcgttgagagatccaaagcttattcaaattgttatccatacgacgtaccggattacgctagccttaggtcactcgttgcgtcaagcggtactctcgaattcaataacgagtcattcaattggactggcgttacgcaaaacggaactagtagcgcatgtaaaagacggtctaataatagcttttttagcagactgaattggttgactcatctgaaattcaaatatcccgcacttaacgttactatgcctaataacgaaaaattcgataagctatatatatggggcgtacaccatcccggaacggataacgatcagatattcttgtacgctcaagctagcggtaggattaccgttagtactaaaagatcccaacaaaccgtaattccgaatatcggatctagacctagggtgagaratataccgtctaggattagcatatattggactatcgttaaacccggagacatactgttgatcaatagtacaggcaatctgatcgcacctagggggtatttcaaaattagatccggtaagtctagcattatgagatccgacgcaccaatcggtaaatgtaatagcgaatgcattacaccaaacggatcaatccctaacgataagccattccaaaacgtaaataggattacatacggcgcatgccctagatacgttaaacagaatacgcttaaacttgcgacaggtatgcgaaacgtacccgaaaaacagactagggggatattcggcgcaatcgccggatttatcgaaaacggatgggagggtatggtcgacggatggtacggatttagacatcaaaatagcgaagggatagggcaagccgccgatctgaaatcaacgcaagccgctattaatcaaattaacggaaaactgaatagattgatcggtaagactaacgaaaaatttcaccaaatcgaaaaagagtttagcgaagttgagggaaggatacaagaccttgagaaatacgttgaggatactaagatcgacctatggtcatataatgccgagttgctagtcgcactcgagaatcagcatacaatcgatctgactgatagcgaaatgaataaattgttcgaaagaacgaaaaaacaattgcgcgaaaacgccgaagacatggggaatgggtgttttaagatataccataaatgcgataacgcatgcatagggtcaatcagaaacggaacatacgatcacgacgtatatagagacgaagcccttaataatagattccaaattaaaggcgttgagcttaaaagcggatacaaagactggatactgtggattagtttcgcaatctcatgctttctattgtgcgttgtgctattggggttcataatgtgggcatgtcagaaagggaatattagatgcaatatttgtatatga

SEQ ID NO: 28 - DNA, 1410 bp, synthetic construct
  source 1..1410: mol_type = other DNA
atgaatcctaaccaaaagattattacaatcggatccgttagccttactatatccacaatttgtttttttatgcaaatagcgatactgataactaccgttacattgcatttcaaacaatacgaattcaattcaccccctaataatcaggttatgttgtgcgaacctactattatcgaacggaatataaccgagatagtgtatctaacgaacactacaatcgaaaaagagatatgccctaagctcgcagagtatagaaattggtcaaaaccccaatgcgatataaccggattcgcaccatttagtaaggataatagtattaggttgtccgccggaggcgatatatgggttacacgcgaaccatacgtgtcatgcgatcccgataaatgctatcaattcgctctcggacagggaacgacattgaataacgtacattcaaacgataccgtacacgataggacaccttatagaacactattgatgaacgaactaggcgtacctttccatctcggaactaaacaggtttgtatcgcttggtctagtagctcatgccatgacggtaaggcatggttgcatgtgtgcgttaccggcgacgataaaaacgcaaccgctagtttcatatataacggtaggttagtcgatagcgtagtgagttggtctaaagacatactgcgaacacaggaatccgagtgcgtatgcataaacggtacatgtaccgtagtgatgaccgacggatccgctagcggtaaggccgatacgaaaatattgttcatagaggagggtaagatagtgcatacaagtacactatccggatccgctcaacatgtcgaagagtgctcatgttatcctagatatcccggcgttagatgcgtatgtagagacaattggaaagggtctaatagaccgatagtcgacattaatattaaaaactattcaatcgttagctcatatgtgtgttccggattagtcggcgatacccctagaaaaaccgatagctctagctcatcccattgtcttgaccctaataacgaagagggggggcatggcgttaagggatgggcattcgacgacggtaacgacgtttggatgggacggacaattagcgaaaaacttagattggggtatgagacttttaaggtaatcgaagggtggtctaatcctaattcgaaactgcaaattaataggcaagtgatagtcgatagggggaataggtccggatatagcggaatcttttccgttgagggtaagtcatgtattaataggtgtttttatgtcgaactgattagggggagaaaagaggaaaccgaagtgttatggactagtaactcaatcgttgtgttttgcggtacatccggtacttatggaaccggatcatggccagacggagccgatataaaccttatgccaatttaa

SEQ ID NO: 29 - DNA, 1704 bp, synthetic construct
  source 1..1704: mol_type = other DNA
atggagaaaatagtgctactactcgcaatcgttagtctggttaagtccgatcagatatgcatagggtatcacgctaacaatagtaccgaacaggtcgacactattatggaaaaaaacgttaccgttacacacgcacaggacatactcgaaaaaacccataacggtaagttatgcgatttagacggagttaagccactgatacttagggattgttcagtcgccggatggttgttagggaatccaatgtgcgacgaattcattaacgtacccgaatggtcatacatagtcgaaaaagcgaatcccgctaacgatctatgttatccagggaattttaacgattacgaagagcttaagcatctactatctagaataaaccatttcgaaaagattcagataataccgaaatcgagttggtccgatcacgaagcgtcaagcggagtgagtagcgcatgcccataccaaggaacaccatcattctttagaaacgtcgtttggttgattaaaaaaaataatacatatccgactattaagagatcatataataatacaaaccaagagaatctactgatactatgggggatacaccatagtaacgacgcagccgaacagattaagctatatcagaatccaactacatacattagcgtagggactagtacacttaatcagagactcgtacctaaaatcgctactagatcgaaggtaaacggacaatccggtagaatggactttttttggactatactgaaacctaacgacgcaattaatttcgaatctaacggaaattttatcgctcccgaatacgcatataagatagtgaaagagggggatagcgcaattatgaaatccgaagtcgaatacggaaattgcaatactaagtgtcagacaccaatcggagcaattaactctagtatgccattccataacatacatccacttacaatcggagaatgccctaaatacgttaagtctaacaaactcgtactcgcaaccggacttaggaatagtccacttagagagagacgaagaaagagagggttgttcggagcaatcgcagggttcatagagggggggtggcagggtatggtcgacggatggtacgggtatcatcattctaacgaacagggatccggatacgcagccgataaagagagtactcagaaagcaatcgacggagtgacgaataaagtgaattcgataatcgataagatgaatacgcaattcgaagccgtaggtagggaattcaataatctcgagagacgaatcgaaaaccttaacaaaaaaatggaagacggattcctagacgtatggacttataacgccgaactgttagtgcttatggagaacgaaagaacccttgactttcacgattctaacgttaagaatctatacgatagagtgagactgcaattgagggataacgctaaagagttagggaacgggtgtttcgaattctatcataaatgcgataacgaatgtatggagtcagtgagaaacggtacatacgactatccgcaatattccgaagaggctagattgaaaagagaggagattagcggagtgaaacttgagtcaatggggacatatcagatattgtcaatatactcaaccgtcgctagtagtctcgcactcgcaattatggtcgccggactgtcactatggatgtgttcaaacggtagtctgcaatgtaggatttgtatataa

SEQ ID NO: 30 - DNA, 1350 bp, synthetic construct
  source 1..1350: mol_type = other DNA
atgaatccgaatcaaaaaattataacaatagggtcaatctgtatggtaatcggtatagtgtcacttatgttacaaatcgggaatattatatctatttgggtgtcacactcaatccaaaccggtaatcaacaccaagacgaacctatacggaatgcgaatttcttaacagagaatgccgtagctagcgttacgttagccggtaatagttcattgtgtcccgttagggggtgggctgtgcatagtaaggataatagtattaggatagggtctaaaggcgacgtattcgtgatacgcgaaccttttatctcttgctcacacttagagtgtagaacattttttctgactcaaggcgcactgttaaacgataaacactctaacggtacagttaaggataggtcaccacataggacattgatgtcatgtcccgtaggcgaagctcctagtccatataatagtagattcgaaagcgttgcatggtccgctagcgcttgtcacgacggaactagttggttgacaatcgggatatccggacccgataatggcgcagtcgcagtgttgaagtataatgggattataaccgatactatcaaatcatggagaaataatatactgagaacacaggagtccgaatgcgcttgcgttaacggatcatgctttaccgttatgactgacggaccatctaacgggcaagctagttataaaattttcaaaatggagaaaggtaaggtagtgaaatccgttgagcttaacgctccaaattatcattacgaagagtgtagttgctatccagacgctggcgaaattacttgcgtatgtagagacaattggcacggatctaatagaccatgggttagctttaatcagaatttagagtatcagatagggtatatatgttccggagtgttcggcgataatcctagacctaacgacggtacagggtcatgcgatccagtgagtccaaacggcgcatacggaattaaagggtttagctttaagtatgggaatggcgtatggatcggtaggactaagtctactaatagtagatccggattcgaaatgatatgggaccctaatgggtggactgagactgatagtagttttagcgtaaaacaggatatagtcgctataaccgattggagcgggtatagcggatcattcgtacagcatcccgaattgactgggttagactgtattagaccatgcttttgggtcgaattgattagggggagaccaaaagagtcaactatatggactagcggatctagtattagtttttgcggagtgaattccgataccgttagttggtcatggccagacggagctgagttgccatttacaatcgataaatag

SEQ ID NO: 31 - DNA, 1659 bp, synthetic construct
  source 1..1659: mol_type = other DNA
atgaatatacagatactcgcattcatagcttgcgtacttaccggagctaaaggcgataagatatgtctagggcatcacgcagtcgcaaacggaacgaaagtgaatacacttacagagagagggatagaggtcgttaacgctacagagacagtcgaaaccgcaaatattaaaaaaatttgtacacaaggaaaacgaccaaccgatctgggacaatgcggactgttagggacactgataggaccaccacaatgcgatcaattccttgagtttagtagcgatctgataatcgaacgaagagagggaactgacgtttgttatcccggtaagttcactaacgaagagagtcttagacagatactgagacggtcagggggaatcggaaaagagtcaatggggtttacgtattctgggattaggactaatggcgcaactagcgcatgtactagaagcggatcatcattctatgccgaaatgaaatggttgttgtcgaattccgataacgctgcattcccacaaatgactaaatcgtatagaaatcctaggaataaacccgcactgataatatggggagtgcatcatagcgaatccgtaagtgaacagactaaattgtacggatcaggtaataaactgattaaagtgagatctagtaagtatcagcaatcgtttacacctaatcccggagctagacgtatcgatttccattggctattgctcgaccctaacgataccgttacattcacattcaatggcgcattcatagcgccagatagggcaagtttttttagaggcgaatcaatcggagtgcaatcagacgcaccacttgactcaagttgcggagggaattgtttccatagcggagggactatagtgagtagtctgccattccaaaatattaatcctagaacagtgggtaagtgtcctagatacgttaaacagaaaagtctgttactcgcaaccggaatgcgtaacgtacccgaaaaacctaaaaaaaggggattgttcggagcgatagccggattcatagagaatggatgggagggactgattaacggatggtacggatttagacaccaaaacgctcagggagagggaaccgcagccgattataaatcgacacaatctgcaatcgatcagattaccggtaagcttaatagattgattggtaagactaatcagcaattcggactgatagacaatgagtttaacgaagtcgagcaacagatagggaatgtgattaattggacacaagacgctatgactgagatttggtcttataatgccgaactgctagtcgctatggagaatcaacacacaatcgatctaaccgatagcgaaatgtcaaaattgtatgagagagtgagaaaacagcttagagagaatgcagaggaagacggaactgggtgtttcgagatattccataaatgcgacgatcactgtatggaatctattagaaataatacatacgatcatacacagtatagaacagagtcacttcaaaatcggatacagatagacccagttaaactatctagcggatataaagacataatactgtggttctcattcggagctagttgttttctgttgctcgcaatcgctatgggacttgtattcatatgtattaaaaacggtaatatgcaatgtacaatttgcatatag

SEQ ID NO: 32 - DNA, 1362 bp, synthetic construct
  source 1..1362: mol_type = other DNA
atgaatccgaatcagaaaatcattactatcggatccgttagcttgacaatcgcaaccgtatgttttcttatgcagattgcgatactcgcaatgaccgttacattgcattttagacaaaacgagtgttctattagcgctaactctcaggtcgtgccatgcgaacctacaaccgaaaaagaggtttgttcaaacgtagtcgattataggtcatggtctaaaccgcaatgtcagattaccggattcgcaccattttcgaaagacaattcgattagactatccgccggaggcgatatttggataactagggaaccatacgtgtcatgcgatacaagtaagtgttatcaattcgcactcggccaagggactacactcgataacaaacactctaacggtacaatacacgataggattagtcataggacactgcttatgaacgagttaggcgtaccattccatctgggaactaaacaggtatgcatagcctggtcatctagttcatgtcacgacggtaaggcatggttgcacgtatgcgtaaccggcgacgatagaaacgctaccgcctcattcatatataacggtatgctagtcgactcaatcgggtcatggtcacaaaatatacttaggacacaggaatccgaatgcgtatgtattaacggatcatgtacagtcgttatgaccgacggatccgctagcggtaaggccgatacacggatactgttcgttaaagagggtaagatagtgcatattagcccacttagcggatccgcccaacatatcgaagagtgttcatgttatcctagatatccgaacgttaggtgcgtttgtagggataattggaaagggtctaatcgacccgttatcgatattaatatggccgattatagtatcgatagttcatacgtttgttccggattagtcggcgatactcctagaaacgaagatagttctagctctagtaattgtagagacccaaacgaagagagagggaatcccggagtgaaagggtgggcattcgatagcggtgacgacgtttggatgggtaggacaattagtagggactctagatccgggtatgagacttttagggtgataggcggatggacaaccgcaaactctaagagtcagactagtagacaggtgatagtcgataataataattggtccgggtatagcgggatttttagcgtcgagcataagtcatgtattaatcggtgtttttatgtcgaattgattagggggcgacctaaagagactagggtgtggtggactagcaattcgatagtcgttttttgcggtactagcggaacatacggaaccggaagttggccagacggagcgaatattaattttatgcctatataa

SEQ ID NO: 33 - DNA, 1704 bp, synthetic construct
  source 1..1704: mol_type = other DNA
atgaatacacagatactcgcattcatagcgtgtatgcttatcggaactaaaggcgataaaatttgcttagggcatcacgcagtcgctaacggaactaaagtgaatacgcttaccgaacgcggaatagaggtcgtgaacgctaccgagacagtcgaaacagtcaatataaaaaaaatttgtacacagggaaaaagaccaaccgatctgggacaatgcggactgttagggacactaatcggaccaccacaatgcgatcaattcctcgaattcgacgctaatctgataatcgaacggagagagggaactgacgtatgctatcccggtaagtttacgaacgaagagtcacttagacagatacttagggggtcaggggggatagacaaagagtctatggggtttacatatagcggaatacggactaacggagctacaagtgcatgtagacgatccggatcatcgttttacgccgaaatgaaatggttgttgtctaatagcgataacgctgcattcccacaaatgactaagtcttataggaatcctagaaataaacccgcactgattatttggggagtgcatcatagtggatcagcaaccgaacagactaagttgtacggatcaggtaataaactgattacagtcggatcgagtaaatatcagcaatcgttcacacctagtcccggagctagaccgcaagtgaacggacaatctggtaggattgactttcattggttgcttctagacccaaacgatacagtgacattcacttttaacggagcatttatcgcacccgatagggctagtttctttaggggagagtcactcggagtgcaatcagacgtaccacttgatagcggatgcgaaggcgattgttttcactcagggggaactatagtgagtagtctgccattccaaaatattaatcctagaaccgtcggtaagtgtcctaggtacgttaaacagactagtctattgctcgcaaccggaatgcgtaacgtacccgaaaatcctaaacaggcatatcagaaacggatgactagggggctattcggagcgattgccggattcatagagaatgggtgggagggactgatagacggatggtacgggttcagacaccaaaacgctcagggagagggaacagccgcagactataagtctacgcaatcggcaatcgatcagattaccggtaagcttaatagactgatagacaaaactaatcagcaattcgaactgatagacaacgaatttagtgagatagagcaacagatagggaatgtgataaattggactagagactcaatgactgaggtatggtcatataacgccgaactgttggtcgcaatggagaatcagcatacaatcgatctagccgatagcgaaatgaataaactttacgaaagggtgcgaaaacaattgcgagagaatgcggaagaggacggaaccggatgtttcgaaattttccataaatgcgacgatcaatgtatggaatcgattaggaataatacatacgatcatacacaatatagaaccgaatcacttcagaataggattcaaatcgatcccgttaagttgagtagcggatataaagacattatactatggttctcattcggagctagttgctttctattgcttgcgatagctatgggattggtgttcatatgcataaaaaacggtaatatgcgatgtacgatttgcatatag

SEQ ID NO: 34 - DNA, 1410 bp, synthetic construct
  source 1..1410: mol_type = other DNA
atgaatcctaatcagaaaataattactataggggtcgttaatactacactatctacaatcgctctactaatcggagtcggtaatctagtctttaatacagtgatacacgaaaagataggcgaccatcagatagtgacacatcctacaattatgacacccgaagtgcctaattgtagcgatacaataattacatataacaataccgttataaacaatattacaacaacaattataaccgaagccgaacgaccattcaaaagtccactacccctatgtccatttagggggttttttccgtttcataaggataacgctatacggttaggcgaaaataaagacgtaatcgttactagggagccatacgttagttgcgataacgataattgttggtcattcgcactcgctcaaggcgcactgttagggactaaacactctaacggaacaattaaagacagaacaccttataggtcactgataagattccctatcggaaccgctcccgtactaggcaattataaagagatatgcatagcatggtcaagttcgtcatgtttcgacggtaaagagtggatgcacgtatgtatgaccggtaacgataacgacgctagcgcacagataatatacggagggcgaatgacagactcaattaagagttggcgtaaagacatactgagaacacaagagtccgaatgccaatgcatagacggaacttgcgtagtcgccgttacagacggacccgcagctaactccgctgaccatagagtgtattggattagggagggaaggataataaagtatgagaacgtgcctaagactaagatacaacatcttgaagagtgttcatgttatgtcgacatagacgtgtattgcatatgtagagacaattggaaagggtctaataggccatggatgagaataaataacgaaactatactcgaaaccggatacgtatgttctaagttccatagcgatacacctagacccgcagacccatctattatgtcatgcgatagcccatctaacgttaacggcggacccggagtcaaagggttcggattcaaagccggtaacgacgtttggttagggagaaccgttagtactagcggtaggtccggattcgaaattataaaggttacagaggggtggataaatagtccgaatcacgttaagtcaattacacaaacacttgtgtctaataacgattggtccggatatagcggatcattcatagtcaaagctaaggattgctttcagccatgtttttacgtcgaactgataagggggagaccgaataaaaacgacgacgttagttggactagtaattcgatagtgacattttgcggattggacaacgaacccggatccggtaattggcctgacggatcgaatatagggtttatgcctaaataa

SEQ ID NO: 35 - DNA, 1710 bp, synthetic construct
  source 1..1710: mol_type = other DNA
agtaagagtagggggtataaaatgaatacacagatactcgtattcgcactcgttgcgtcaataccgacaaacgccgataagatttgcctagggcatcacgcagtgtcaaacggaactaaagtgaatacacttaccgaaaggggcgttgaggtagtgaacgctacagagactgtcgaacggactaacgtacctaggatttgtagtaagggtaaaagaacagtcgacctagggcaatgcggactgttaggcacaattaccggaccaccacaatgcgaccaatttctcgaatttagcgctgatctgattatcgaacggagagagggatccgacgtttgttatcccggtaaattcgttaacgaagaggcactgagacagatacttagagaatccggagggatagacaaagagacaatggggtttacatatagcggaattagaactaacggaactactagcgcatgtaggagatccggatctagcttttacgccgaaatgaaatggttactgtcaaataccgataacgccgcatttccgcaaatgactaagtcatataagaatactaggaaagaccccgcactgataatttgggggatacaccatagcggatcgactaccgaacagacaaagctatacggtagcgggaataaactgataacagtgggatcaagtaattaccaacagtcattcgtaccgagtccaggcgctagaccacaagtgaacggacaatccggacgtatagatttccattggttgatactgaatccgaacgatacagtgacatttagctttaacggcgcattcatagcacccgatagggcatcattccttaggggtaagagtatggggatacaaagcgaagtgcaagtcgacgctaattgcgaaggcgattgttatcatagcggggggactattattagtaatctgccattccaaaatattaatagtagggcagtgggaaagtgtccaaggtacgttaaacaggaatcactgttactcgcaaccggaatgaaaaacgtaccagagatacctaagagacgaagaagggggttgttcggcgctatagccggattcatagagaacggatgggagggactgatagacggatggtacgggttcagacaccaaaacgctcaaggcgaagggacagccgcagactataagagtacacaatccgctatcgatcaaattaccggtaagcttaatagactgatcgaaaaaactaatcaacaattcgaactaatcgataacgaatttacggaagtcgaaagacagattggcaatgtgataaattggactagagactctatgactgaggtttggtcatataacgccgaactgttagtcgcaatggaaaatcagcatacgatagaccttgccgatagcgaaatgaataagctatacgaaagggtgaaacgacaattgagggaaaacgccgaagaggacggaacagggtgtttcgaaatttttcacaaatgcgacgacgattgtatggctagtattaggaataatacatacgaccatagtaagtatagagaggaagcgatacagaataggattcaaatcgatcccgtaaaactgtctagcggatacaaagacgttatactgtggttctcattcggagcgtcatgtttcatactgcttgcaatcgctatggggttagtgttcatatgcgttaaaaacggaaatatgcgatgtactatttgtatttaa

SEQ ID NO: 36 - DNA, 1416 bp, synthetic construct
  source 1..1416: mol_type = other DNA
atgaatccgaaccaaaaattgttcgcattaagcggagtcgcaatcgcactaagcgtactgaatctgttgatagggataagtaacgtagggttgaacgtatcactacatttgaaagagaaagggcctaaacaggaagagaatttgacatgtactacaattaatcagaataatactaccgtagtcgaaaatacatacgttaacaatacaacaattattactaagggaaccgatctgaaaactccaagttatctgttactgaataaatctctatgtaacgttgagggatgggtagtgatcgcaaaggataacgccgttagattcggcgaaagcgaacagattatagtgactagagagccatacgtatcatgcgatccaaccggatgcaaaatgtacgcattacaccaagggacaactattaggaataaacactctaacggtacgatacacgatagaaccgcatttagggggttgattagtacaccactcggtacaccaccaaccgtttcgaatagcgactttatgtgcgtagggtggtctagtactacatgtcacgacggaatcgctagaatgacaatttgcatacaggggaataacgataacgctaccgcaaccgtatattataatagaagactaactactactattaagacatgggctaggaatatactgagaacgcaagaatccgaatgcgtttgtcataacggtacatgcgccgtagtgatgaccgacggatccgctagttcgcaagcatatactaaggtaatgtattttcacaaagggttagtagtgaaagaggaagagttgagggggtccgctagacatattgaggaatgctcatgttacggacataatcaaaaggtgacatgcgtatgtagagacaattggcaaggcgcaaatagacccattatcgaaatcgatatgagtacactcgaacatactagtagatatgtgtgtaccggaatactaaccgatacgagtagacccggcgataagtctagcggagattgctcaaacccaattaccggatcacccggagtgccaggcgttaagggattcggattccttaacggagacaatacatggttagggagaactattagtcctaggagtaggtccggattcgaaatgcttaagatacctaacgccggaaccgacccaaatagtaggattgccgaacgacaagagattgtcgacaataacaattggtccggatatagcggatcattcatagactattggaacgacaatagcgaatgctataacccatgtttttacgttgagttgattaggggtagacccgaagaggcaaaatacgtttggtgggcatctaacagtctaatcgcattatgcggatcaccatttcccgtaggtagcggatcatttcccgacggagcccaaattcaatattttagttaa

SEQ ID NO: 37 - DNA, 1683 bp, synthetic construct
  source 1..1683: mol_type = other DNA
atggagacaattagtctgattactatactattggtcgttacagcgtcaaacgctgacaaaatatgtataggccatcaatccactaattcaaccgaaacagtcgatacactaaccgaaacgaatgtgccagtgacacacgctaaagagctactgcataccgaacataacggaatgctatgcgctactagcctagggcatccactgatactcgatacatgtactatcgagggactcgtatacggtaatcctagttgcgatctactgttaggcggtagggaatggtcatacatagtcgaacgatcatccgccgtaaacggaacatgttatcccggtaatgtcgagaatctcgaagagcttaggacactattctcatccgctagctcataccaacgaatacagatttttcccgatactacatggaatgtgacatataccggaactagtagggcatgttccggatcattctatagatcaatgagatggttgacacaaaaatccggcttttaccctgtgcaagacgcacaatatacgaataataggggtaaatctatactattcgtatggggtatacatcatccacctacttataccgaacagactaatctgtatattagaaacgatacaactacatccgttacaaccgaagacttgaataggacattcaaacccgtaatcggacctagaccactagtgaacggattgcagggtagaatcgattactattggtccgtacttaagccagggcaaacacttagagtgagatctaacggtaatctaatcgcaccatggtacggacacgtacttagcggagggtcacacggtaggatacttaagaccgatctgaaaggggggaattgcgtagtgcaatgccaaaccgaaaaaggcggactgaattcgacactaccattccataatattagcaaatacgcattcggaacatgtcctaagtacgttagggtgaatagtctgaaactcgcagtgggattgagaaacgtacccgctagatcgagtagggggctattcggcgcaatcgcagggtttatcgaaggcggatggccaggactagttgccggatggtacggattccaacatagtaacgatcaaggcgtagggatggccgccgatagggatagcacacaaaaagcaatcgataagattactagtaaggttaataatatagtcgataagatgaataagcaatacgaaattatcgatcacgaatttagcgaagtcgaaactagactgaatatgataaataataagatagacgatcagatacaagacgtatgggcatataacgccgaactgttagtgttgcttgagaatcagaagacactcgacgaacacgacgcaaacgttaataatctgtataataaagtgaaaagagcactagggtctaacgctatggaggacggtaagggatgtttcgaactatatcataaatgcgacgatcaatgcatggagacaattagaaacggtacatataatcggagaaagtatagagaggaatctagactcgaaagacagaaaatcgaaggcgttaaactcgaatccgaaggaacatataagatactgactatttatagtacagtcgctagctcactagtgcttgctatgggattcgccgcattcttgttttgggctatgtcaaacggatcatgtaggtgtaatatttgtatttaa

SEQ ID NO: 38 - DNA, 1404 bp, synthetic construct
  source 1..1404: mol_type = other DNA
atgaatccgaatcagaaaataatcgcattagggtccgtttcgattactatagcgactatatgcctattgatgcaaatcgcaatactcgcaacgactatgacattgcattttaacgaatgcactaatccctctaataatcaggccgttccatgcgaaccaatcataatcgaacggaatattaccgagatagtgcatcttaacaatacgactatcgaaaaagagtcatgccctaaggtagcggaatataaaaattggtctaagcctcaatgtcagattaccggattcgcaccattctctaaagataattcaattaggcttagcgcaggcggagatatatgggtgactagagagccatacgtaagttgcggactcggtaagtgttatcaattcgcattaggccaagggacaacccttaataataagcatagtaacggtactatacacgataggagtccacataggactcttcttatgaacgagttaggcgtaccattccatttagggactaaacaggtttgtatcgcatggtctagtagttcatgtcatgacggtaaggcatggttgcatgtttgcgttaccggcgacgatagaaacgctaccgcttcaatcatatacgacggtatgcttaccgattcaatcggatcatggtctaaaaatatacttagaacccaagagtccgaatgcgtatgtattaacggtacatgtacagtcgttatgacagacggatccgctagcggtagggccgatacaaagatactattcatacgcgaaggtaagatagtgcatatcggaccattgtccggatccgcacaacacgttgaggaatgctcatgttatcctagatatcccgaagtgagatgcgtatgtagagataattggaaagggtcaaatagacccgtactgtatataaacgttgccgattatagcgtcgatagttcatatgtgtgtagcggactagtgggcgatacacctagaaacgacgattcatctagtagttcgaattgtagggatcctaataacgaaagaggcggaccaggcgttaaagggtgggcattcgataacggtaacgacgtttggatggggagaactattaaaaaagattctagatcagggtatgagacattcagagtggtgggggggtggactaccgctaactctaagtctcaaattaatagacaggtgatagtcgatagcgataattggtcagggtattccggtatttttagcgttgagggtaagacatgtattaataggtgtttttatgtcgaattgattagggggcgaccacaagagactagggtttggtggactagtaattcgattatagtgttttgcggaactagcggaacatacggaaccggatcatggccagacggagcgaatataaattttatgtctatataa

SEQ ID NO: 39 - DNA, 2341 bp, synthetic construct
  source 1..2341: mol_type = other DNA
agcgaaagcaggcaaaccatttgaatggatgtcaatccgactctactgttcttaaaagttccagcgcaaaatgccataagcacaacattcccttatactggagatcctccatacagccatggaacagggacagggtacactatggacacagtcaacagaacacaccaatattcagagagggggaagtggacgacaaatacagaaactggggcgccccagctcaacccaattgatggaccactacctgaggataatgaaccaagtggatatgcacaaacagactgtgtcctggaggctatggccttccttgaagaatcccacccaggtatctttgagaactcatgccttgaaacaatggaagccgttcaacagacaagggtggacaaactaacccaaggtcgccagacttatgattggacattaaacaggaatcaaccggcagcaactgcattagccaacaccatagaagtctttagatcgaacggattaacagctaatgaatcaggaaggctaatagatttcctcaaggatgtgatggaatcaatggataaagaggaaatggagataacaacacactttcaaagaaaaaggagagtaagggacaacatgaccaagaaaatggtcacacaaagaacaatagggaagaaaaagcaaagagtgaataagagaggctacctaataagagctttgacattgaacacgatgaccaaagatgcagagagaggcaaattaaaaagaagggctattgcaacacccgggatgcaaattagagggttcgtgtacttcgttgaaactttagctagaagcatttgcgaaaagcttgaacagtctggacttccggttgggggtaatgaaaagaaggccaaactggcaaatgttgtgagaaaaatgatgactaattcacaagacacagagctttctttcacaatcactggggataacactaagtggaatgaaaatcaaaacccccgaatgtttttggcgatgattacatacatcacaaagaatcaacctgagtggttcagaaacatcctgagcatcgcaccaataatgttctcaaacaaaatggcaagactgggaaaaggatacatgttcgagagtaagagaatgaagctccggacacaaatacctgcagaaatgctagcaagcattgacctgaagtatttcaatgaatcaacaaggaagaaaattgagaaaataaggcctcttctaatagatggcacagcatcattgagccctggaatgatgatgggcatgttcaacatgctaagtacagttttaggagtctcgatactgaatcttggacaaaagaaatacaccaagacaacatactggtgggatgggctccaatcctcagatgattttgccctcatagtgaatgcaccaaatcatgagggaatacaagcaggagtggatagattctacaggacctgcaagttagtgggaatcaacatgagcaaaaagaagtcctatataaataaaacagggacatttgaattcactagctttttttatcgatatggatttgtggctaattttagcatggagctgccaagttttggagtgtctggaataaacgagtcagctgacatgagcattggagtaacagtgataaagaacaacatgataaacaatgaccttggaccagcaacagcccaaatggctctccaattgttcatcaaagattacagatacacatatcggtgccatagaggagacacacaaatccaaacgagaagatcattcgagataaagaagctgtgggaccaaacccaatcaaggacaggactattggtatcagatgggggaccaaacttatacaatatccggaatcttcacatccctgaagtctgcttaaagtgggagctgatggatgagaattatcggggaagactttgtaatcccctgaatccctttgtcagccataaagaaattgagtctgtaaacaatgctgtagtaatgccagcccatggtccggccaaaagtatggaatatgatgccgttgcaactacgcactcctggattcccaaaaggaaccgctctattctaaacacaagccaaaggggaattcttgaggatgaacagatgtaccagaagtgctgcaacttgttcgagaaatttttccctagtagttcatataggagaccgattggaatttctagcatggtggaggccatggtgtctagggcccggattgatgccagaattgacttcgagtctggaaggattaagaaggaagagttctctgagatcatgaagatctgttccaccattgaagaactcagacggcaaaaataatgaatttagcttgtccttcatgaaaaaatgccttgtttctact

SEQ ID NO: 40 - DNA, 2341 bp, synthetic construct
  source 1..2341: mol_type = other DNA
agcaaaagcaggtcaattatattcagtatggaaagaataaaagaactacggaatctgatgtcgcagtctcgcactcgcgagatactgacaaaaaccacagtggaccatatggccataattaagaagtacacatcagggagacaggaaaagaacccgtcacttaggatgaaatggatgatggcaatgaaatatccaatcactgctgacaaaagggtaacagaaatggttccggagagaaatgaacaaggacaaactctatggagtaaaatgagtgatgctggatcagatagagtgatggtatcacctttggctgtaacatggtggaataggaatggacccgtgacaagtacggtccattacccaaaagtgtacaaaacttatttcgacaaagtcgaaaggttaaaacatggaacctttggccctgtccattttagaaatcaagtcaagatacgcagaagagtagacataaaccctggtcatgcagacctcagtgccaaagaggcacaagatgtaattatggaagttgtttttcccaatgaagtgggagccagaatactaacatcagaatcacaactaacaataactaaagagaaaaaagaagaactccgagattgcaaaatttctcccttgatggtcgcatacatgttagagagagaacttgtgcgaaaaacaagatttctcccagttgctggcggaacaagcagtatatacattgaagttttacatttgactcagggaacgtgttgggaacaaatgtacactccaggtggaggagtgaggaatgacgatgttgatcaaagcctaattattgcggccaggaacatagtaaggagagccgcagtatcagcagatccactagcatctttattggagatgtgccacagcacgcaaattggcggaacaaggatggtggacattcttagacagaaccctactgaagaacaagctgtggatatatgcaaggctgcaatgggattgagaatcagctcatccttcagctttggtggctttacatttaaaagaacaagcgggtcatcagtcaaaaaagaagaagaggtgcttacaggcaatctccaaacattgagaataagagtacatgaggggtatgaggagttcacaatggtggggaaaagagcaacagctatactaagaaaagcaaccagaagattggttcaactcatagtgagtggaagagacgaacagtcaatagccgaagcaataatcgtggccatggtgttttcacaagaagattgcatgataaaagcagttagaggtgacctgaattttgtcaacagagcaaatcagcggttgaaccccatgcatcagcttttaaggcattttcagaaagatgcgaaagtgctctttcaaaattggggagttgaacacatcgacagtgtgatgggaatggttggagtattaccagatatgactccaagcacagagatgtcaatgagaggaataagagtcagcaaaatgggtgtggatgaatactccagtacagagagggtggtggttagcattgatcggtttttgagagttcgagaccaacgtgggaatgtattattatctcctgaggaggtcagtgaaacacagggaactgagagactgacaataacttattcatcgtcgatgatgtgggagattaacggtcctgagtcggttttggtcaatacctatcaatggatcatcaggaattgggaagctgtcaaaattcaatggtctcagaatcctgcaatgttgtacaacaaaatggaatttgaaccatttcaatctttagtccccaaggccattagaagccaatacagtgggtttgtcagaactctattccaacaaatgagagacgtacttgggacatttgacactgcccagataataaagcttctcccttttgcagctgctccaccgaagcaaagcagaatgcagttctcttcactgactgtgaatgtgaggggatcagggatgagaatacttgtaaggggcaattctcctgtattcaactacaacaagaccactaaaaggctaacaattctcggaaaagatgccggcactttaattgaagacccagatgaaagcacatccggagtggagtccgccgtcttgagagggttcctcattataggtaaggaagacagaagatacggaccagcattaagcatcaatgaactgagtaaccttgcaaaaggggaaaaggctaatgtgctaatcgggcaaggagacgtggtgttggtaatgaaacgaaaacgggactctagcatacttactgacagccagacagcgaccaaaagaattcggatggccatcaattaatactgaatagtttaaaaacgaccttgtttctact

SEQ ID NO: 41 - DNA, 2233 bp, synthetic construct
  source 1..2233: mol_type = other DNA
agcaaaagcaggtactgattcaaaatggaagattttgtgcgacaatgcttcaacccgatgattgtcgaacttgcagaaaaagcaatgaaagagtatggggaggatctgaaaattgaaaccaacaaatttgcagcaatatgcactcacttggaggtgtgtttcatgtattcagatttccatttcatcaatgaacaaggcgaatcaatagtggtagaacttgacgatccaaatgcactgttaaagcacagatttgaaataatcgaggggagagacagaacaatggcctggacagtagtaaacagtatctgcaacactactggagctggaaaaccgaagtttctaccggatttgtatgattacaaagagaacagattcatcgaaattggagtgacaaggagagaagtccacatatattaccttgaaaaggccaataagattaaatctgagaacacacacattcacattttttcattcactggggaggaaatggccacaaaggcagactacactctcgacgaggaaagcagggctaggattaaaaccaggctgtttaccataagacaagaaatggccaacagaggcctctgggattcctttcgtcagtccgaaagaggcgaagaaacaattgaagaaaaatttgagatcacaggaactatgcgcaggcttgccgaccaaagtctcccaccgaacttctcctgccttgagaattttagagcctatgtggatggattcgaaccgaacggctgcattgagggcaagctttctcaaatgtccaaagaagtgaatgcccaaattgaaccttttctgaagacaacaccaagaccaatcaaacttcctaatggacctccttgttatcagcggtccaaattcctcctgatggatgctttgaaattgagcattgaagacccaagtcacgaaggagaagggatcccattatatgatgcgatcaagtgcataaaaacattctttggatggaaagaaccttatatagtcaaaccacacgaaaagggaataaattcaaattacctgctgtcatggaagcaagtactgtcagaattgcaggacattgaaaatgaggagaagattccaagaactaaaaacatgaagaagacgagtcaactgaagtgggctcttggtgaaaacatggcaccagagaaggtagactttgaaaactgcagagacataagcgatttgaagcaatatgatagtgaagaacctgaattaaggtcactttcaagctggatacagagtgagttcaacaaggcctgtgagctaactgattcagtctggatagagctcgatgaaattggagaggacgtagccccaattgagcacattgcaagcatgagaaggaattatttcacagcagaggtgtcccattgtagagctactgaatacataatgaagggggtatacattaacactgccctgctcaatgcatcctgtgcagcaatggacgattttcaactaattcccatgataagcaagtgcagaactaaagagggaaggcgaaaaaccaatttatatggattcatcataaagggaagatctcatttgaggaatgacacagacgtggtaaattttgtgagcatggagttttctctcacagacccgagacttgaaccacataaatgggagaaatactgtgtccttgagataggagatatgttactaagaagtgccataggccaaatttcaaggccgatgttcttgtatgtgaggacaaacggaacatcaaaggtcaaaatgaaatggggaatggagatgagacgttgcctccttcagtcactccagcagatcgagagcatgattgaagccgagtcctcagttaaagagaaagacatgaccaaagagttttttgagaataaatcagaagcatggcccattggagagtcccccaagggagtggaagaaggttccattgggaaagtctgtaggactttattagctaagtcagtattcaatagcctgtatgcatcaccacaattggaaggattttcagcggagtcaagaaaactgctccttgttgttcaggctcttagggacaacctcgaacctgggacctttgatcttggggggctatatgaagcaattgaggagtgcctgattaatgatccctgggttttgctcaatgcgtcttggttcaactccttcctgacacatgcattaaaatagttatggcagtgctactatttgttatccgtactgtccaaaaaagtaccttgtttctact

SEQ ID NO: 42 - DNA, 1566 bp, synthetic construct
  source 1..1566: mol_type = other DNA
agcaaaagcagggttaataatcactcactgagtgacatcaaaatcatggcgtcccaaggcaccaaacggtcttatgaacagatggaaactgatggagatcgccagaatgcaactgagattagggcatccgtcgggaagatgattgatggaattgggagattctacatccaaatgtgcactgaacttaaactcagtgatcatgaagggcggttgatccagaacagcttgacaatagagaaaatggtactctctgcttttgatgaaagaaggaataaatacctggaagaacaccccagcgcggggaaagatcccaagaaaactggggggcccatatacaggagagtcgatgggaaatggatgagggaactcgtcctttatgacaaagaagaaataaggcgaatctggcgccaagccaacaatggtgaggatgctacatctggtctaactcacataatgatttggcattccaatttgaatgatgcaacataccagaggacaagagctcttgttcgaactggaatggatcccagaatgtgctctctgatgcagggctcgactctccctagaaggtccggagctgcaggtgctgcagtcaaaggaatcgggacaatggtgatggaactgatcagaatggtcaaacgggggatcaatgatcgaaatttttggagaggtgagaatgggcggaaaacaagaagtgcttatgagagaatgtgcaacattcttaaaggaaaatttcaaacagctgcacaaagagcaatggtggatcaagttagagaaagtcggaacccaggaaacgctgagatcgaagatctcatatttttagcaagatctgcactgatattgagaggatcagttgctcacaaatcttgcctacctgcctgtgcgtatggacctgcagtatccagtgggtacgacttcgaaaaagagggatattccttggtgggaatagaccctttcaaactacttcaaaatagccaaatatacagcttaatcagacctaacgagaatccagcacacaagagtcagctggtgtggatggcatgccattctgctgcatttgaagatttaagattgttaagcttcatcagagggacaaaagtatctcctcgggggaaactgtcaactagaggagtacaaattgcttcaaatgagaacatggataatatgggatcgagcactcttgaactgagaagcaggtactgggccataaggaccaggagtggaggaaacactaatcaacagagggcctccgcaggccaaaccagtgtgcaacctacgttttctgtacaaagaaacctcccatttgaaaagtcaaccatcatggcagcattcactggaaatacggagggaagaacttcagacatgagggcagaaatcataaggatgatggaaggtgcaaaaccagaagaagtgtcattccgggggaggggagttttcgagctctcagacgagaaggcaacgaacccgatcgtgccctcttttgatatgagtaatgaaggatcttatttcttcggagacaatgcagaagagtacgacaactaaggaaaaaatacccttgtttctact

SEQ ID NO: 43 - DNA, 1027 bp, synthetic construct
  source 1..1027: mol_type = other DNA
agcaaaagcaggtagatattgaaagatgagccttctaaccgaggtcgaaacgtatgttctctctatcgttccatcaggccccctcaaagccgagatcgcgcagagacttgaagatgtctttgctgggaaaaacacagatcttgaggctctcatggaatggctaaagacaagaccaattctgtcacctctgactaaggggattttagggtttgttttcacgctcaccgtgcccagtgagcgaggactgcagcgtagacgctttgtccaaaatgccctcaatgggaatggagacccaaataacatggacaaagcagttaaactgtataggaaacttaagagggagataacgttccatggggccaaagaaatagctctcagttattctgctggtgcacttgccagttgcatgggcctcatatacaataggatgggggctgtaaccactgaagtggcatttggcctagtgtgtgcaacatgtgagcagattgctgattcccagcacaggtctcataggcagatggtggcaacaaccaatccattaataaaacatgagaacagaatggttttggccagcactacagctaaggctatggagcaaatggctggatcaagtgagcaggcagcggaggccatggagattgctagtcaggccaggcagatggtgcaggcaatgagagccattgggactcatcctagttccagtactggtctaagagatgatcttcttgaaaatttgcagacctatcagaaacgaatgggggtgcagatgcaacgattcaagtgacccgcttgttgttgccgcgaatatcattgggatcttgcacttgatattgtggattcttgatcgtctttttttcaaatgcgtctatcgactcttcaaacacggccttaagagaggcccttctacggaaggagtacctgagtctatgagggaagaatatcgaaaggaacagcagaatgctgtggatgctgacgacagtcattttgtcagcatagagttggagtaaaaaactaccttgtttctact

SEQ ID NO: 44 - DNA, 887 bp, synthetic construct
  source 1..887: mol_type = other DNA
agcaaaagcagggtgacaaagacataatggattccaacactgtgtcaagtttccaggtagattgctttctttggcatatccggaaacaagttgtagaccaagaactgagtgatgccccattccttgatcggcttcgccgagatcagaggtccctaaggggaagaggcaatactctcggtctagacatcaaagcagccacccatgttggaaagcaaattgtagaaaagattctgaaagaagaatctgatgaggcacttaaaatgaccatggtctcaacacctgcttcgcgatacataactgacatgactattgaggaattgtcaagaaactggttcatgctaatgcccaagcagaaagtggaaggacctctttgcatcagaatggaccaggcaatcatggagaaaaacatcatgttaaaagcgaatttcaatgtgatttttggccgactagagaccatagtattactaagggctttcaccgaagagggagcaattgttggcgaaatctcaccattgccttcttttccaggacatactattgaggatgtcaaaaatgcaattggggtcctcatcggaggacttgaatggaatgataacacagttcaagtctctaaaaatctacagagattcgcttggagaagcagtaatgagaatgggggacctccacttactccaaaacagaaacgggaaatggcgagaacagctaggtcagaagtttgaagagataagatggctaattgaagaagtgagacacagattaagaacaaccgaaaatagctttgaacaaataacattcatgcaagcattacaactgctgtttgaagtggaacaggagataagaactttctcatttcagcttatttaatgataaaaaacacccttgtttct

(19) United States
(12) Patent Application Publication
(10) Pub. No.: US 2025/0025547 A1
(43) Pub. Date: Jan. 23, 2025

(54) PARENTERALLY ADMINISTRABLE INFLUENZA VACCINE AND USES THEREOF

(71) Applicant: Codagenix Inc., Farmingdale, NY (US)

(72) Inventors: John Robert Coleman, Blauvelt, NY (US); Steffen Mueller, Great Neck, NY (US); Chen Yang, Plainview, NY (US); James W. Harpel, West Palm Beach, FL (US)

(73) Assignee: Codagenix Inc., Farmingdale, NY (US)

(21) Appl. No.: 18/714,291

(22) PCT Filed: Dec. 2, 2022
(86) PCT No.: PCT/US2022/080808

§ 371 Date: May 29, 2024

Related U.S. Application Data
(60) Provisional application No. 63/332,449, filed on Apr. 19, 2022.
(60) Provisional application No. 63/285,398, filed on Dec. 2, 2021.

(51) Int. Cl. A61K 39/145; A61K 39/00; A61P 37/04; C12N 7/00

(52) U.S. Cl. CPC A61K 39/145; A61P 37/04; C12N 7/00; A61K 2039/5254; A61K 2039/54; A61K 2039/575; C12N 2760/16122; C12N 2760/16134; C12N 2760/16162

(57) ABSTRACT

The present invention provides for parenterally administrable live attenuated influenza immune compositions. Methods of using parenterally administrable live attenuated influenza immune compositions to elicit an immune response, and particularly, a protective immune response are also provided.

### CROSS-REFERENCE TO RELATED APPLICATIONS

This application includes a claim of priority under 35 U.S.C. § 119(e) to U.S. provisional patent application No. 63/285,398, filed Dec. 2, 2021, and provisional patent application No. 63/332,449, filed Apr. 19, 2022, the entirety of both which are hereby incorporated by reference.

### REFERENCE TO SEQUENCE LISTING

This application contains a Sequence Listing submitted as an electronic xml file named “Sequence_Listing_064955_000024WOPT”, having a size in bytes of 111,758 bytes, and created on Dec. 1, 2022. The information contained in this electronic xml file is hereby incorporated by reference in its entirety.

### FIELD OF INVENTION

This invention relates to immune compositions and methods of eliciting an immune response and/or prophylaxis against influenza.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Influenza is a human disease that leads every year to >30,000 deaths in the US and several hundred thousand deaths globally. While annual influenza vaccines are available, their efficacy is suboptimal. Additionally, the immunogenicity a live attenuated vaccine given by the intranasal route in some cases may be insufficient to elicit a protective immune response. Thus, there remains a need in the art for influenza vaccines with increased efficacy and methods of eliciting an immunity, and particularly, eliciting a protective immunity against influenza.

### SUMMARY OF THE INVENTION

The following embodiments and aspects thereof are described and illustrated in conjunction with compositions and methods which are meant to be exemplary and illustrative, not limiting in scope.

Various embodiments of the invention provide for a method of eliciting an immune response in a subject in need thereof, comprising: parenterally administering a composition comprising a live attenuated influenza virus in which expression of hemagglutinin (HA) and neuraminidase (NA) is reduced compared to a natural isolate virus, wherein the reduction in expression is a result of recoding the HA protein-encoding sequence and recoding the NA protein-encoding sequence, thereby eliciting the immune response in the subject.

Various embodiments of the invention provide for a method of eliciting an immune response in a subject in need thereof, comprising: parenterally administering a composition comprising a live attenuated influenza virus in which its hemagglutinin (HA) protein encoding sequence and neuraminidase (NA) protein encoding sequence are recoded compared to its natural isolate virus, and wherein the amino acid sequence of HA protein or NA protein of the live attenuated influenza virus remains the same compared to its natural isolate virus, or wherein the amino acid sequence of the HA protein or NA protein of the live attenuated influenza virus comprises up to 20 amino acid substitutions, additions, or deletions compared to the natural isolate virus, thereby eliciting the immune response in the subject.

In various embodiments, the composition can comprise two or more different subtypes of the live attenuated influenza viruses. In various embodiments, the composition can comprise a H1N1 subtype and a H3N2 subtype.

In various embodiments, the composition can comprise about 10³-10⁹ PFU of the live attenuated influenza virus. In various embodiments, the composition can comprise about 10⁷-10⁸ PFU of the live attenuated influenza virus.

In various embodiments, parenterally administering the composition can comprise parenterally administering a single dose. In various embodiments, parenterally administering the composition can comprise parenterally administering a prime dose and parenterally administering at least one boost dose.

In various embodiments, the prime dose can be about 10³-10⁹ PFU of the live attenuated influenza virus. In various embodiments, each of the least one boost dose can be about 10³-10⁹ PFU of the live attenuated influenza virus. In various embodiments, the prime dose can be about 10⁷-10⁸ PFU of the live attenuated influenza virus. In various embodiments, each of the least one boost dose can be about 10⁷-10⁸ PFU of the live attenuated influenza virus.

In various embodiments, parenterally administering the at least one boost dose can occur about 2 weeks or more after parenterally administering the prime dose. In various embodiments, parenterally administering the at least one boost dose can occur about 3-4 weeks after parenterally administering the prime dose.

In various embodiments, the least one boost dose can comprise a first boost dose and a second boost dose, and the second boost dose can be parenterally administered about 2 weeks or more after parenterally administering the first boost dose. In various embodiments, the least one boost dose can comprise a first boost dose and a second boost dose, and the second boost dose can be parenterally administered about 3-4 weeks after parenterally administering the first boost dose.

In various embodiments, parenterally administering can comprise intramuscular injection. In various embodiments, parenterally administering can comprise subcutaneous injection.

In various embodiments, eliciting the immune response can provide protective immunity against an influenza virus of the same subtype. In various embodiments, eliciting the immune response can provide cross protective immunity against a heterologous influenza virus.

In various embodiments, eliciting the immune response can comprise at least a 10-fold increase in hemagglutination inhibition (HAI) titer, neuraminidase inhibition (NAI) titer, or both, in the subject about 14 to 35 days after parenteral administration of the composition as compared to day 0 before parenteral administration with the composition. In various embodiments, eliciting the immune response can comprise a 30-150-fold increase in hemagglutination inhibition (HAI) titer, neuraminidase inhibition (NAI) titer, or both, in the subject about 14 to 35 days after parenteral administration of the composition as compared to day 0 before parenteral administration with the composition.

In various embodiments, the expression of other influenza proteins in attenuated influenza virus are not substantially reduced.

In various embodiments, one or both of the HA protein-encoding sequence and the NA protein-encoding sequence on the attenuated influenza virus can be recoded by lowering the codon-pair bias of the protein-encoding sequence as compared to the natural isolate virus.

In various embodiments, reducing the codon-pair bias can comprise identifying a codon pair in the parent protein-encoding sequence having a codon-pair score that can be reduced, and reducing the codon-pair bias by substituting the codon pair with a codon pair that has a lower codon-pair score. In various embodiments, reducing the codon-pair bias can comprise rearranging the codons of a parent protein-encoding sequence.

In various embodiments, each of the recoded HA protein-encoding sequence and the recoded NA protein-encoding sequence of the attenuated influenza virus can have a codon pair bias less than −0.05, less than −0.1, less than −0.15, less than −0.2, less than −0.25, less than −0.3, less than −0.35, or less than −0.4.

In various embodiments, each of the recoded HA protein-encoding sequence and the recoded NA protein-encoding sequence of the attenuated influenza virus can be recoded by increasing the number of CpG or UpA di-nucleotides compared to the natural isolate virus. In various embodiments, each of the recoded HA protein-encoding sequence and the recoded NA protein-encoding sequence of the attenuated influenza virus can be recoded by increasing at least 5, at least 10, at least 15, at least 20 or at least 25 CpG or UpA di-nucleotides compared to the natural isolate virus.

In various embodiments, one or both of the HA protein-encoding sequence and the NA protein-encoding sequence of the attenuated influenza can be recoded by replacing ten or more codons with synonymous codons that are less frequent in the viral host. In various embodiments, one or both of the HA protein-encoding sequence and the NA protein-encoding sequence of the attenuated influenza can be recoded by replacing ten or more codons with synonymous codons that are less frequent in the influenza virus.

In various embodiments, the HA protein of the live attenuated virus can be encoded by a HA protein encoding sequence comprising SEQ ID NO:1, SEQ ID NO:3, SEQ ID NO:5, or SEQ ID NO:7 or a HA protein encoding sequence selected from Table 1. In various embodiments, the HA protein of the live attenuated virus can be encoded by a HA protein encoding sequence comprising SEQ ID NO: 15, ORF of SEQ ID NO:15, SEQ ID NO:16, ORF of SEQ ID NO:16, SEQ ID NO: 17, or ORF of SEQ ID NO:17.

In various embodiments, the NA protein of the live attenuated virus can be encoded by a NA protein encoding sequence comprising SEQ ID NO:2, SEQ ID NO:4, SEQ ID NO: 6, SEQ ID NO:8, SEQ ID NO:18, or ORF of SEQ ID NO:18 or a NA protein encoding sequence selected from Table 1.

In various embodiments, eliciting the immune response can comprise at least a 50-fold, at least a 100-fold increase in anti-hemagglutinin (HA) IgG titer in the subject after about 21 days after parenteral administration of the composition as compared to day 0 before parenteral administration of the composition.

In various embodiments, eliciting the immune response can comprise about 100-fold to 700-fold increase in anti-hemagglutinin (HA) IgG titer in the subject after about 21 days after parenteral administration of the composition as compared to day 0 before parenteral administration of the composition.

Various embodiments of the invention provide for a live attenuated influenza virus comprising a hemagglutinin (HA) protein encoded by a HA encoding sequence, a neuraminidase (NA) protein encoded by a NA encoding sequence or both, wherein the HA protein encoding sequence comprises SEQ ID NO:1, SEQ ID NO:3, SEQ ID NO:7, SEQ ID NO:15, ORF of SEQ ID NO:15, SEQ ID NO:16, ORF of SEQ ID NO:16, SEQ ID NO:17, or ORF of SEQ ID NO:17 and the NA protein encoding sequence comprises SEQ ID NO:2, SEQ ID NO:4, SEQ ID NO:8, SEQ ID NO: or ORF of SEQ ID NO:18.

In various embodiments, the nucleic acid having SEQ ID NO:1, SEQ ID NO:2, SEQ ID NO:3, SEQ ID NO:4, SEQ ID NO:7, SEQ ID NO:8, SEQ ID NO:18, SEQ ID NO:15, ORF of SEQ ID NO:15, SEQ ID NO:16, ORF of SEQ ID NO:16, SEQ ID NO:17, ORF of SEQ ID NO:17, or ORF of SEQ ID NO:18 or any combination thereof can impart the attenuation compared to its natural isolate form.

Various embodiments of the invention provide for a parenterally administrable composition, comprising: a live attenuated influenza virus in which expression of hemagglutinin (HA) and neuraminidase (NA) is reduced compared to a natural isolate virus, wherein the reduction in expression is a result of recoding the HA protein-encoding sequence and recoding the NA protein-encoding sequence, thereby eliciting the immune response in the subject, or a live attenuated influenza virus in which its hemagglutinin (HA) protein encoding sequence and neuraminidase (NA) protein encoding sequence are recoded, and wherein the amino acid sequence of HA protein or NA protein of the live attenuated influenza virus remains the same, or wherein the amino acid sequence of the HA protein or NA protein of the live attenuated influenza virus comprises up to 20 amino acid substitutions, additions, or deletions compared to the natural isolate virus; and a pharmaceutically acceptable carrier or excipient suitable for administration via parenterally administration.

Various embodiments of the invention provide for a parenterally administrable composition of the invention as described herein, wherein the live attenuated influenza virus can comprise a hemagglutinin (HA) protein encoded by a HA encoding sequence, a neuraminidase (NA) protein encoded by a NA encoding sequence or both, wherein the HA protein encoding sequence comprises SEQ ID NO:1, SEQ ID NO:3, SEQ ID NO:7, SEQ ID NO:15, ORF of SEQ ID NO:15, SEQ ID NO:16, ORF of SEQ ID NO:16, SEQ ID NO:17 or ORF of SEQ ID NO:17 and the NA protein encoding sequence comprises SEQ ID NO:2, SEQ ID NO:4, SEQ ID NO:8, or SEQ ID NO:18.

In various embodiments, the nucleic acid having SEQ ID NO:1, SEQ ID NO:2, SEQ ID NO:3, SEQ ID NO:4, SEQ ID NO:8, SEQ ID NO:7, SEQ ID NO:15, ORF of SEQ ID NO:15, SEQ ID NO:16, ORF of SEQ ID NO:16, SEQ ID NO:17, ORF of SEQ ID NO:17, SEQ ID NO:18 or ORF of SEQ ID NO:18 or any combination thereof can impart the attenuation compared to its natural isolate form.

Various embodiments of the invention provide for a parenterally administrable composition of the present invention as described herein, wherein the pharmaceutically acceptable carrier or excipient suitable for administration via parenteral administration is suitable for administration via intramuscular injection or subcutaneous injection, or both.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts LD50 of A/California/07/2009(H1N1)-Mouse Adapted in CD1 mice, in accordance with various embodiments of the present invention.

FIG. 2 depicts LD50 of A/Aichi/2/1968(H3N2) in CD1 mice in accordance with various embodiments of the present invention.

FIG. 3 depicts Relative Body Weight and Survival of H1N1 IM/SC vaccinated CD1 mice against lethal challenge with A/California/07/2009(H1N1)-Mouse Adapted in accordance with various embodiments of the present invention.

FIG. 4 depicts Relative Body Weight and Survival of H3N2 IM/SC vaccinated CD1 mice against lethal challenge with A/Aichi/2/1968(H3N2) in accordance with various embodiments of the present invention.

FIG. 5 depicts HAI titer of H1N1 IM/SC vaccinated mice in accordance with various embodiments of the present invention.

FIG. 6 depicts HAI titer of H3N2 IM/SC vaccinated mice in accordance with various embodiments of the present invention.

FIG. 7 depicts body weight of the ferrets in the H1N1 study in accordance with various embodiments of the present invention.

FIG. 8 depicts body weight variation percentage of the ferrets in the H1N1 study in accordance with various embodiments of the present invention.

FIG. 9 depicts body temperature of the ferrets in the H1N1 study in accordance with various embodiments of the present invention.

FIG. 10 depicts body temperature variation percentage of ferrets in the H1N1 study in accordance with various embodiments of the present invention.

FIG. 11 depicts HAI Titers in Sera (California strains) from ferrets in the H1N1 study in accordance with various embodiments of the present invention.

FIG. 12 depicts comparison of antibody levels of monkey treated with intramuscular injections of CodaVax-H1N1 at day 0, 21, 35 in accordance with various embodiments of the present invention.

FIG. 13 depicts body weight of ferrets in the H3N2 study in accordance with various embodiments of the present invention.

FIG. 14 depicts body weight variation of ferrets in the H3N2 study in accordance with various embodiments of the present invention.

FIG. 15 depicts body temperature of ferrets in the H3N2 study in accordance with various embodiments of the present invention.

FIG. 16 depicts hemagglutination inhibition in sera from ferrets in the H3N2 study in accordance with various embodiments of the present invention. Top: Hemagglutination Inhibition in Sera H1N1 (California strain); Bottom Hemagglutination Inhibition in Sera H3N2 (Texas strain).

FIG. 17 depicts hemagglutination inhibition in sera from ferrets in the H3N2 study in accordance with various embodiments of the present invention. Top: Hemagglutination Inhibition in Sera H1N1 (Guangdon-Maonan strain); Bottom Hemagglutination Inhibition in Sera H3N2 (Delaware strain).

FIG. 18 depicts viral titer in nasal washes (Day 0, Day 1, Day 2, Day 4 and Day 6) in accordance with various embodiments of the present invention.

FIG. 19 depicts viral titer in nasal washes (Day 8, Day 14, Day 15, Day 16 and D17) in accordance with various embodiments of the present invention.

FIG. 20 depicts viral titer in nasal washes (Day 19, Day 21, Day 23, Day 29 and Day 30) in accordance with various embodiments of the present invention.

FIG. 21 depicts viral titer in sera (Day 0, Day 2, Day 14, Day 28 and Day 30) in accordance with various embodiments of the present invention.

FIG. 22 depicts viral titer in olfactory bulbs (Day 2 and Day 30) in accordance with various embodiments of the present invention.

FIG. 23 depicts viral titer in nasal turbinates (Day 2 and Day 30) in accordance with various embodiments of the present invention.

FIG. 24 depicts viral titer in lungs (Day 2 and Day 30) in accordance with various embodiments of the present invention.

FIG. 25 depicts a comparison of anti-HA2(A/Vietnam/1203/2004)(H5N1) IgG levels of non-human primates vaccinated with intramuscular injections of CodaVax-H1N1 at day 0, 21, 35 in accordance with various embodiments of the present invention.

FIG. 26 depicts HAI titer against A/California/07/2009(H1N1).

FIG. 27 depicts HAI titer against A/Singapore/lNF1 MH-16-0019/2016 (H3N2).

FIG. 28 depicts HAI titer for bivalent vaccination group. The green line (HAI titer of 40) is considered seroconverted.

FIG. 29 depicts the challenge with MAD H1N1 Cal4 WT.

FIG. 30 depicts the challenge with H3N2 Aichi WT.

FIG. 31 depicts both live Cal07 WT (1e+6 PFU) and CodaVax-H1N1 (1e+6 PFU) induced much higher titer of HAI against wild type A/California/07/2009(H1N1) when compared to their inactivated form. UV-inactivation and BPL-inactivation induce similar HAI titer. A boost is required for inactivated viruses to achieve a detectable HAI titer. Live attenuated CodaVax-H1N1 can also be boosted with a more than 2-fold increase in HAI titer to achieve a similar level to Cal07 WT.

FIG. 32 depicts all vaccine candidates, both Cal07 WT and CodaVax-H1N1, Live or UV-, BPL-inactivated viruses, triggered high ELISA titers against H5 HA2. In general, live viruses have a 2- to 8-fold higher ELISA titer when compared to the inactivated viruses. UV-inactivated viruses also induced higher ELISA titer than BPL-inactivated viruses. Due to the variation, they are not statistically significant comparing each other (Two-way ANOVA).

FIG. 33 depicts ELISA titers against influenza M2 were all very low. However, only live Cal07 WT and CodaVax-H1N1 induce quantifiable titers against M2, while UV- or BPL-inactivated influenza viruses failed to generate sufficient M2 binding antibodies. No difference was observed between live Cal07 WT and CodaVax-H1N1 (One-way ANOVA).

FIG. 34 shows individual HAI Titer against A/Singapore/INFIMH16-0019/2016 in African Green Monkeys.

FIG. 35 depicts a schematic of A/CA07/09 (HA-NA)^{Min} Hemagglutinin and Neuraminidase segments.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared.

“Natural isolate” as used herein with reference to influenza virus refers to a virus such as influenza that has been isolated from a host (e.g., human, bird, or any other host) or natural reservoir. The sequence of the natural isolate can be identical or have mutations that arose naturally through the virus' replication cycles as it replicates in and/or transmits between hosts, for example, humans.

“Parent virus” as used herein refer to a reference virus to which a recoded nucleotide sequence is compared for encoding the same or similar amino acid sequence.

“Frequently used codons” or “codon usage bias” as used herein refer to differences in the frequency of occurrence of synonymous codons in coding nucleic acid for a particular species.

“Codon pair bias” as used herein refers to synonymous codon pairs that are used more or less frequently than statistically predicted in a particular species, for example, human, influenza.

“Deoptimized” as used herein with respect to the viruses refer to modified viruses in which their genome, in whole or in part, has synonymous codons and/or codon rearrangements and/or variation of codon pair bias. The substitution of synonymous codons alters various parameters, including for example, codon bias, codon pair bias, density of deoptimized codons and deoptimized codon pairs, RNA secondary structure, CpG dinucleotide content, C+G content, UpA dinucleotide content, translation frameshift sites, translation pause sites, the presence or absence of tissue specific microRNA recognition sequences, or any combination thereof, in the genome.

A “subject” as used herein means any animal or artificially modified animal. Animals include, but are not limited to, humans, non-human primates, cows, horses, sheep, pigs, dogs, cats, rabbits, ferrets, rodents such as mice, rats and guinea pigs, bats, snakes, and birds. Artificially modified animals include, but are not limited to, SCID mice with human immune systems. In a preferred embodiment, the subject is a human.

A “viral host” means any animal or artificially modified animal that a virus can infect. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems. In various embodiments, the viral host is a mammal. In various embodiments, the viral host is a primate. In various embodiments, the viral host is human. Embodiments of birds are domesticated poultry species, including, but not limited to, chickens, turkeys, ducks, and geese.

A “prophylactically effective dose” is any amount of a vaccine or virus composition that, when administered to a subject prone to viral infection or prone to affliction with a virus-associated disorder, induces in the subject an immune response that protects the subject from becoming infected by the virus or afflicted with the disorder. “Protecting” the subject means either reducing the likelihood of the subject's becoming infected with the virus, or lessening the likelihood of the disorder's onset in the subject, by at least two-fold, preferably at least ten-fold, 25-fold, 50-fold, or 100-fold. For example, if a subject has a 1% chance of becoming infected with a virus, a two-fold reduction in the likelihood of the subject becoming infected with the virus would result in the subject having a 0.5% chance of becoming infected with the virus.

As used herein, a “therapeutically effective dose” is any amount of a vaccine or virus composition that, when administered to a subject afflicted with a disorder against which the vaccine is effective, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

Currently approved vaccines are intramuscularly administered inactivated vaccines and an intranasally administered live attenuated vaccine (FLUMIST). The inventors' group previously described in International Patent Publication WO 2014/145290, herein incorporated by reference as though fully set forth, deoptimized live attenuated influenza vaccines that are administered intranasally. There was no prior evidence to show that influenza can replicate following injection. Further, as influenza is a respiratory disease, conventional wisdom with respect to live vaccines is to use a route of administration that provides access to the respiratory system. Thus, live influenza vaccines are given intranasally. Accordingly, live attenuated influenza vaccines are not given parenterally, such as intramuscularly or subcutaneously as it was not known to replicate and it does not provide access for infection to the respiratory system.

Going against conventional wisdom, described herein are methods of parenterally administering various live attenuated influenza viruses, such as intramuscular (IM) injection, subcutaneous (SC) injection, intradermal injection of different influenza vaccines. Specific live attenuated influenza viruses are also provided, which can be administered through conventional routes (e.g., intranasal) and via IM, SC and parenteral.

Described herein, both IM and SC injection of different influenza vaccines of the present invention showed very good results, with high HAI titers after boost. Both IM and SC injection induced high HAI titer in CD1 mice against wildtype virus. Boosting induced greater than 4-fold increase in HAI titer in most mice. IM vaccination yielded a higher HAI titer than SC. Both IM and SC vaccinated mice were protected from lethal challenge with homologous H1 N1 challenge. IM group also provided protection against lethal, heterologous H3N2 virus challenge.

For H1N1 influenza vaccine (CodaVax-H1N1) study in mice, IM injection shows even higher HAI titer against H1N1 WT Cal7 when compared to SC injection at 1.0e+8 PFU, along with more mice with a 4-fold HAI increase upon boost. All CodaVax-H1N1 IM or SC vaccinated CD1 mice were completely protected from lethal challenge with wildtype H1N1 Cal7. For H3N2 Singapore (HA+NA)^{Min} influenza vaccine (where the HA and NA proteins are based on the Singapore strain), both IM and SC injection in mice induced high HAI titer against H3N2 Singapore wildtype virus. IM injection shows higher HAI titer and more mice with a 4-fold increase in HAI after the boost as compared to SC injection. Although almost no HAI titer was seen in IM group against H3N2 Aichii strain, all IM vaccinated mice survived the heterologous H3N2 virus challenge. In conclusion, IM and SC are both good routes for inducing high HAI titer, and elicit protection against wt influenza.

The safety, immunogenicity, and efficacy of the H1N1 Influenza vaccine were also evaluated in ferrets. Ferrets were immunized with H1N1 Influenza Vaccine candidate on Day 0 with following boost immunizations on Day 21. The immunogenicity of the vaccine, with respect to the induction of functional hemagglutinin specific antibodies, was verified by HAI.

Based on the results obtained, slight loss of body weight and increased body temperature, likely caused by anesthesia, were observed in two of the five animals after the first dose, the animals gained progressive body weight and maintained stable body temperature during the study. No clinical signs were observed. At necropsy, slight findings in the lungs including dark discoloration in the lung lobes and mottled lung, possibly related to incomplete exsanguination, were noted. But histological examination will be required for confirmation of the residual blood in the lung or other causes.

Based on functional measurement by HAI, antibodies titers were detected 14 days post prime immunization and enhanced further by boost immunization on Day 21. The antibody titers peaked on Day 35 post immunization. The immunization of CodaVax-H1N1 by IM route is immunogenic and induced the production of functional antibody against A/California/07/2009 (H1N1).

The immunogenicity of the vaccine candidate CodaVax-H1N1 was also evaluated in the African Green monkey model. No abnormal clinical findings or changes in body weight or changes in body temperatures were observed in any of the vaccinated animals throughout the study period. A transient loss of appetite was observed in some animals following primary and secondary vaccination. No remarkable changes in chemistry or hematological parameters were observed in any of the animals during the study period. qRT-PCR analysis for the presence of viral genome in plasma, nasal swabs, and tracheal lavage samples were carried out at Codagenix Inc. All samples were below the lower limit of quantification. The single positive plasma sample by qRT-PCR had no detectable infectious virus via plaque assay. Serum was collected from all animals on Days 0, 21 and 35 and the HAI titer was determined at Codagenix Inc. All animals had HAI serum antibody titers. Serum collected at Day 21 and Day 35 showed about 30-fold and more than 150-fold respectively higher antibody titer as compared to Day 0. Overall, intramuscular vaccination with live attenuated CodaVax-H1N1 vaccine did not cause flu-like symptoms in vaccinated African Green monkeys. The vaccinated animals did not develop viremia or viral shedding in nasal or tracheal secretions. The vaccination also induced HAI antibodies and enhanced titers were observed following booster vaccination on Day 21.

Additionally, the safety, immunogenicity, and efficacy of H3N2 influenza vaccine was also assessed. It was assessed against A/Singapore/INFIMH-16-0019/2016 WT H3N2 challenge in ferret model. Ferrets were immunized with different formulations on Day 0 with or without following boost immunizations on Day 14. One active comparator group was immunized with Fluzone. Immunized animals were challenged with Influenza A/Singapore/INFIMH-16-0019/2016 WT H3N2 on D28. The immunogenicity of the vaccine, with respect to the induction of functional hemagglutinin specific antibodies, was verified by hemagglutinin inhibition assay. Protective efficacy against influenza challenge was assessed by serum viremia (measured by viral titration) and histopathological analysis.

The vaccine did not induce changes in the body weight or body temperature of the ferrets and no clinical signs were observed. At necropsy, slight findings in the lungs, often related to viral infection, were noted. If observations were made 7 to 10 days post infection it could give an indication about viral clearance. However, when assessing lungs at 2 days-post challenge with a relatively large (1 mL) volume of inoculum, it is difficult to draw conclusions about whether the challenge virus was being cleared. The results demonstrated that the attenuated candidate vaccines were not more aggressive for the animals than the active comparator. In consequence, no safety issues were detected.

The data summarized in this report indicate that even though viral titers were not detectable, it appeared that a protection was induced by immunization, as shown by an increase in the functional antibodies titers against A/California/07/2009 (H1N1) and A/Texas/50/2012 (H3N2). In consequence, it appeared that the severity of neutrophilic inflammation in the nasal turbinates in animals from IN H3 Texas/50/2012 Prime Only (Groups 1), IN H3 Texas/50/2012 Prime+Boost (Group 2), and IN Codavax H3 Prime Only (Group 3) were reduced following challenge, as opposed to SC Codavax H3+H1 Prime+Boost (Groups 5) and Mock Control (Group 6) which provoked increased neutrophilic infiltration in nasal turbinates and lungs. Neutrophil infiltration due to vaccine could be related to a decrease in IgA production.

Accordingly, various embodiments of the present are based, at least in part, on these findings.

Various embodiments of the present invention provide for a method of eliciting an immune response in a subject in need thereof, comprising: parenterally administering a composition comprising a live attenuated influenza virus in which expression of hemagglutinin (HA) and neuraminidase (NA) is reduced compared to a natural isolate virus, wherein the reduction in expression is a result of recoding the HA protein-encoding sequence and recoding the NA protein-encoding sequence, thereby eliciting the immune response in the subject.

Various embodiments of the present invention provide for a method of eliciting an immune response in a subject in need thereof, comprising: parenterally administering a composition comprising a live attenuated influenza virus in which its hemagglutinin (HA) protein encoding sequence and neuraminidase (NA) protein encoding sequence are recoded, and wherein the amino acid sequence of HA protein or NA protein of the live attenuated influenza virus remains the same, or wherein the amino acid sequence of the HA protein or NA protein of the live attenuated influenza virus comprises up to 20 amino acid substitutions, additions, or deletions compared to the natural isolate virus, thereby eliciting the immune response in the subject.

In various embodiments, the composition comprises two or more different subtypes of the live attenuated influenza viruses. As such, the composition can be bi-valent, tri-valent, quad-valent, and the like. In one nonlimiting example, the composition comprises a recoded H1N1 subtype and a recoded H3N2 subtype.

In various embodiments, the composition each subtype of the live attenuated influenza virus comprises a single recoded strain. For example, the composition comprises a recoded A/California/07/2009 H1N1 strain and a recoded Singapore H3N2 strain, or the composition comprises a recoded A/California/07/2009 H1N1 strain and a recoded A/Texas/50/2012 H3N2 strain.

In various embodiments, the composition each subtype of the live attenuated influenza virus comprises two or more recoded strains. For example, if the composition comprises two subtypes, the composition can comprise a recoded A/California/07/2009 H1N1 strain, a recoded Singapore H3N2 strain and a recoded A/Texas/50/2012 H3N2 strain.

In various embodiments, the method further comprises identifying the subject in need of eliciting the immune response before parenterally administering the composition comprising the live attenuated influenza virus. Identifying the subject may be made by a health care professional, such as a doctor, nurse, nurse practitioner physician assistant or pharmacist, or be made by the subject himself/herself/themselves. In various embodiments, identifying the subject in need of eliciting the immune response comprises actively identifying the subject in need of an influenza vaccine. In various embodiments, identifying the subject in need of eliciting the immune response comprises actively identifying the subject as desiring an influenza vaccine. In various embodiments, identifying the subject in need of eliciting the immune response does not comprise identifying a subject in need of or desiring a different prophylactic or therapeutic treatment or benefit from the composition comprising the live attenuated influenza virus. In various embodiments, the subject in need of eliciting the immune response is not a subject who is in need of or who desires prophylactic or therapeutic treatment or benefit relating to tumor or malignant tumor.

In various embodiments, parenterally administering comprises injecting. In various embodiments, injecting comprises subcutaneous injection. In various embodiments, injecting comprises intramuscular injection. In various embodiments, injecting comprises intradermal injection.

In various embodiments, the composition used in the method comprises about 10³-10⁹ PFU of the live attenuated influenza virus. In various embodiments, the composition used in the method comprises about 10⁷-10⁸ PFU of the live attenuated influenza virus. In various embodiments, the composition used in the method comprises about 1×10³, 1×10⁴, 1×10⁵, 1×10⁶, 10⁷, 1×10⁸ or 1×10⁹, PFU of the live attenuated influenza virus. In various embodiments, the composition used in the method comprises about 2×10³, 2×10⁴, 2×10⁵ 2×10⁶, 2×10⁷, 2×10⁸ or 2×10⁹, PFU of the live attenuated influenza virus. In various embodiments, the composition used in the method comprises about 5×10³, 5×10⁴, 5×10⁵, 5×10⁶, 5×10⁷, 5×10⁸ or 5×10⁹, PFU of the live attenuated influenza virus. In various embodiments, the composition used in the method comprises about 5×10⁴ PFU of the live attenuated influenza virus. In various embodiments, the composition used in the method comprises about 5×10⁵ PFU of the live attenuated influenza virus. In various embodiments, the composition used in the method comprises about 5×10⁶ PFU of the live attenuated influenza virus. In various embodiments a single dose is parenterally administered.

In various embodiments, the composition used in the method comprises about 0.03-0.3 μg of HA protein. In various embodiments, the composition used in the method comprises about 0.01-0.05 μg of HA protein. In various embodiments, the composition used in the method comprises about 0.05-0.1 μg of HA protein. In various embodiments, the composition used in the method comprises about 0.1-0.15 μg of HA protein. In various embodiments, the composition used in the method comprises about 0.15-0.2 μg of HA protein. In various embodiments, the composition used in the method comprises about 0.2-0.25 μg of HA protein. In various embodiments, the composition used in the method comprises about 0.25-0.3 μg of HA protein.

In various embodiments, parenterally administering the composition comprises parenterally administering a single dose.

In various embodiments, parenterally administering the composition comprises parenterally administering a prime dose and parenterally administering at least one boost dose.

In various embodiments, the prime dose is about 10³-10⁹ PFU of the live attenuated influenza virus. In various embodiments, the prime dose is about 10⁷-10⁸ PFU of the live attenuated influenza virus. In various embodiments, the prime dose is about 1×10³, 1×10⁴, 1×10⁵, 1×10⁶, 10⁷, 1×10⁸ or 1×10⁹, PFU of the live attenuated influenza virus. In various embodiments, the prime dose is about 2×10³, 2×10⁴, 2×10⁵, 2×10⁶, 2×10⁷, 2×10⁸ or 2×10⁹, PFU of the live attenuated influenza virus. In various embodiments, the prime dose is about 5×10³, 5×10⁴, 5×10⁵, 5×10⁶, 5×10⁷, 5×10⁸ or 5×10⁹, PFU of the live attenuated influenza virus.

In various embodiments, the prime dose is about 0.03-0.3 μg of HA protein. In various embodiments, the prime dose is about 0.01-0.05 μg of HA protein. In various embodiments, the prime dose is about 0.05-0.1 μg of HA protein. In various embodiments, the prime dose is about 0.1-0.15 μg of HA protein. In various embodiments, the prime dose is about 0.15-0.2 μg of HA protein. In various embodiments, the prime dose is about 0.2-0.25 μg of HA protein. In various embodiments, the prime dose is about 0.25-0.3 μg of HA protein.

In various embodiments, each of the least one boost dose is about 10³-10⁹ PFU of the live attenuated influenza virus. In various embodiments, each of the least one boost dose is about 10⁷-10⁸ PFU of the live attenuated influenza virus. In various embodiments, each of the least one boost dose is about 1×10³, 1×10⁴, 1×10⁵, 1×10⁶, 10⁷, 1×10⁸ or 1×10⁹, PFU of the live attenuated influenza virus. In various embodiments, each of the least one boost dose is about 2×10³, 2×10⁴, 2×10⁵, 2×10⁶, 2×10⁷, 2×10⁸ or 2×10⁹, PFU of the live attenuated influenza virus. In various embodiments, each of the least one boost dose is about 5×10³, 5×10⁴, 5×10⁵, 5×10⁶, 5×10⁷, 5×10⁸ or 5×10⁹, PFU of the live attenuated influenza virus.

In various embodiments, each of the least one boost dose is about 0.03-0.3 μg of HA protein. In various embodiments, each of the least one boost dose is about 0.01-0.05 μg of HA protein. In various embodiments, each of the least one boost dose is about 0.05-0.1 μg of HA protein. In various embodiments, each of the least one boost dose is about 0.1-0.15 μg of HA protein. In various embodiments, each of the least one boost dose is about 0.15-0.2 μg of HA protein. In various embodiments, each of the least one boost dose is about 0.2-0.25 μg of HA protein. In various embodiments, each of the least one boost dose is about 0.25-0.3 μg of HA protein.

In various embodiments, the prime dose is smaller than the boost done. In other embodiments, the boost dose is smaller than the prime dose.

In various embodiments, parenterally administering the at least one boost dose occurs about 2 weeks or more after parenterally administering the prime dose. In various embodiments, parenterally administering the at least one boost dose occurs about 3 weeks or more after parenterally administering the prime dose. In various embodiments, parenterally administering the at least one boost dose occurs about 4 weeks or more after parenterally administering the prime dose. In various embodiments, parenterally administering the at least one boost dose occurs about 3-4 weeks after parenterally administering the prime dose. In various embodiments, parenterally administering the at least one boost dose occurs about 4-6 weeks after parenterally administering the prime dose. In various embodiments, parenterally administering the at least one boost dose occurs about 8-12 weeks after parenterally administering the prime dose. In various embodiments, parenterally administering the at least one boost dose occurs about 4-6 months after parenterally administering the prime dose. In various embodiments, parenterally administering the at least one boost dose occurs about 7-9 months after parenterally administering the prime dose. In various embodiments, parenterally administering the at least one boost dose occurs about 10-12 months after parenterally administering the prime dose. In various embodiments, parenterally administering the at least one boost dose occurs 12 months or more after parenterally administering the prime dose.

In various embodiments, the least one boost dose comprises a first boost dose and a second boost dose, and the second boost dose is parenterally administered about 2 weeks or more after parenterally administering the first boost dose. In various embodiments, the least one boost dose comprises a first boost dose and a second boost dose, and the second boost dose is parenterally administered about 3 weeks or more after parenterally administering the first boost dose. In various embodiments, the least one boost dose comprises a first boost dose and a second boost dose, and the second boost dose is parenterally administered about 4 weeks or more after parenterally administering the first boost dose. In various embodiments, the least one boost dose comprises a first boost dose and a second boost dose, and the second boost dose is parenterally administered about 3-4 weeks after parenterally administering the first boost dose. In various embodiments, the least one boost dose comprises a first boost dose and a second boost dose, and the second boost dose is parenterally administered about 1-2 months after parenterally administering the first boost dose. In various embodiments, the least one boost dose comprises a first boost dose and a second boost dose, and the second boost dose is parenterally administered about 3-4 months after parenterally administering the first boost dose. In various embodiments, the least one boost dose comprises a first boost dose and a second boost dose, and the second boost dose is parenterally administered about 5-6 months after parenterally administering the first boost dose. In various embodiments, the least one boost dose comprises a first boost dose and a second boost dose, and the second boost dose is parenterally administered about 7-8 months after parenterally administering the first boost dose. In various embodiments, the least one boost dose comprises a first boost dose and a second boost dose, and the second boost dose is parenterally administered about 9-10 months after parenterally administering the first boost dose. In various embodiments, the least one boost dose comprises a first boost dose and a second boost dose, and the second boost dose is parenterally administered about 11-12 months after parenterally administering the first boost dose. In various embodiments, the least one boost dose comprises a first boost dose and a second boost dose, and the second boost dose is parenterally administered about 1-3 months after parenterally administering the first boost dose. In various embodiments, the least one boost dose comprises a first boost dose and a second boost dose, and the second boost dose is parenterally administered about 4-6 months after parenterally administering the first boost dose. In various embodiments, the least one boost dose comprises a first boost dose and a second boost dose, and the second boost dose is parenterally administered about 7-9 months after parenterally administering the first boost dose. In various embodiments, the least one boost dose comprises a first boost dose and a second boost dose, and the second boost dose is parenterally administered about 10-12 months after parenterally administering the first boost dose. In various embodiments, the least one boost dose comprises a first boost dose and a second boost dose, and the second boost dose is parenterally administered about 12-18 months after parenterally administering the first boost dose. In various embodiments, the least one boost dose comprises a first boost dose and a second boost dose, and the second boost dose is parenterally administered 18 months or more after parenterally administering the first boost dose.

In various embodiments, injecting comprises intramuscular injection. In various embodiments, injecting comprises subcutaneous injection. In various embodiments, injecting comprises intradermal injection.

In various embodiments, eliciting the immune response provides protective immunity against an influenza virus of the same subtype. For example, administering an H1N1 attenuated influenza provides protective immunity against other H1N1 influenza viruses.

In various embodiments, eliciting the immune response provides cross protective immunity against a heterologous influenza virus. For example, administering an H1N1 attenuated influenza provides protective immunity against other H3N2 influenza viruses.

In various embodiments, eliciting the immune response comprises at least a 10-fold increase in hemagglutination inhibition (HAI) titer, neuraminidase inhibition (NAI) titer, or both, in the subject about 14 to 35 days after injection of the composition as compared to day 0 before injection with the composition. In various embodiments, eliciting the immune response comprises at least a 20-fold increase in hemagglutination inhibition (HAI) titer, neuraminidase inhibition (NAI) titer, or both, in the subject about 14 to 35 days after injection of the composition as compared to day 0 before injection with the composition. In various embodiments, eliciting the immune response comprises at least a 30-fold increase in hemagglutination inhibition (HAI) titer, neuraminidase inhibition (NAI) titer, or both, in the subject about 14 to 35 days after injection of the composition as compared to day 0 before injection with the composition. In various embodiments, eliciting the immune response comprises at least a 50-fold increase in hemagglutination inhibition (HAI) titer, neuraminidase inhibition (NAI) titer, or both, in the subject about 14 to 35 days after injection of the composition as compared to day 0 before injection with the composition.

In various embodiments, eliciting the immune response comprises a 30-150-fold increase in hemagglutination inhibition (HAI) titer, neuraminidase inhibition (NAI) titer, or both, in the subject about 14 to 35 days after injection of the composition as compared to day 0 before injection with the composition. In various embodiments, eliciting the immune response comprises a 10-30-fold increase in hemagglutination inhibition (HAI) titer, neuraminidase inhibition (NAI) titer, or both, in the subject about 14 to 35 days after injection of the composition as compared to day 0 before injection with the composition. In various embodiments, eliciting the immune response comprises a 40-75-fold increase in hemagglutination inhibition (HAI) titer, neuraminidase inhibition (NAI) titer, or both, in the subject about 14 to 35 days after injection of the composition as compared to day 0 before injection with the composition. In various embodiments, eliciting the immune response comprises a 75-100-fold increase in hemagglutination inhibition (HAI) titer, neuraminidase inhibition (NAI) titer, or both, in the subject about 14 to 35 days after injection of the composition as compared to day 0 before injection with the composition. In various embodiments, eliciting the immune response comprises a 100-150-fold increase in hemagglutination inhibition (HAI) titer, neuraminidase inhibition (NAI) titer, or both, in the subject about 14 to 35 days after injection of the composition as compared to day 0 before injection with the composition. In various embodiments, eliciting the immune response comprises a 150-200-fold increase in hemagglutination inhibition (HAI) titer, neuraminidase inhibition (NAI) titer, or both, in the subject about 14 to 35 days after injection of the composition as compared to day 0 before injection with the composition. In various embodiments, eliciting the immune response comprises a 200-fold or more increase in hemagglutination inhibition (HAI) titer, neuraminidase inhibition (NAI) titer, or both, in the subject about 14 to 35 days after injection of the composition as compared to day 0 before injection with the composition.

In various embodiments, eliciting the immune response comprises at least a 50-fold increase in anti-hemagglutinin (HA) IgG titer in the subject after about 21 days after parenteral administration of the composition as compared to day 0 before parenteral administration of the composition. In various embodiments, eliciting the immune response comprises at least a 75-fold increase in anti-hemagglutinin (HA) IgG titer in the subject after about 21 days after parenteral administration of the composition as compared to day 0 before parenteral administration of the composition. In various embodiments, eliciting the immune response comprises at least a 100-fold increase in anti-hemagglutinin (HA) IgG titer in the subject after about 21 days after parenteral administration of the composition as compared to day 0 before parenteral administration of the composition.

In various embodiments, eliciting the immune response comprises at least a 600-fold increase in anti-hemagglutinin (HA) IgG titer in the subject after about 35 days after parenteral administration of the composition as compared to day 0 before parenteral administration of the composition. In various embodiments, eliciting the immune response comprises at least a 650-fold increase in anti-hemagglutinin (HA) IgG titer in the subject after about 35 days after parenteral administration of the composition as compared to day 0 before parenteral administration of the composition. In various embodiments, eliciting the immune response comprises at least a 700-fold increase in anti-hemagglutinin (HA) IgG titer in the subject after about 35 days after parenteral administration of the composition as compared to day 0 before parenteral administration of the composition.

In various embodiments, eliciting the immune response comprises about 50-fold to 800-fold increase in anti-hemagglutinin (HA) IgG titer in the subject after about 21 days after parenteral administration of the composition as compared to day 0 before parenteral administration of the composition. In various embodiments, eliciting the immune response comprises about 50-fold to 800-fold increase in anti-hemagglutinin (HA) IgG titer in the subject after about 35 days after parenteral administration of the composition as compared to day 0 before parenteral administration of the composition. In various embodiments, eliciting the immune response comprises about 50-fold to 800-fold increase in anti-hemagglutinin (HA) IgG titer in the subject after about 21 days after parenteral administration of the composition as compared to day 0 before parenteral administration of the composition. In various embodiments, eliciting the immune response comprises about 50-fold to 800-fold increase in anti-hemagglutinin (HA) IgG titer in the subject after about 35 days after parenteral administration of the composition as compared to day 0 before parenteral administration of the composition.

In various embodiments, eliciting the immune response comprises about 200-fold to 600-fold increase in anti-hemagglutinin (HA) IgG titer in the subject after about 21 days after parenteral administration of the composition as compared to day 0 before parenteral administration of the composition. In various embodiments, eliciting the immune response comprises about 200-fold to 600-fold increase in anti-hemagglutinin (HA) IgG titer in the subject after about 35 days after parenteral administration of the composition as compared to day 0 before parenteral administration of the composition.

In various embodiments, the expression of other influenza proteins in attenuated influenza virus are not substantially reduced (e.g., influenza proteins other than HA protein or NA protein).

In various embodiments, a continuous segment of its HA protein encoding sequence and NA protein encoding sequence is recoded, wherein the continuous segment is about ⅘ the length of the HA protein encoding sequence and ⅘ the length of the NA protein encoding sequence. In various embodiments, a continuous segment of its HA protein encoding sequence and NA protein encoding sequence is recoded, wherein the continuous segment is about ¾ the length of the HA protein encoding sequence and ¾ the length of the NA protein encoding sequence. In various embodiments, a continuous segment of its HA protein encoding sequence and NA protein encoding sequence is recoded, wherein the continuous segment is about ½ the length of the HA protein encoding sequence and about ½ the length of the NA protein encoding sequence. In various embodiments, a continuous segment of its HA protein encoding sequence and NA protein encoding sequence is recoded, wherein the continuous segment is about ⅓ the length of the HA protein encoding sequence and about ⅓ the length of the NA protein encoding sequence. In various embodiments, a continuous segment of its HA protein encoding sequence and NA protein encoding sequence is recoded, wherein the continuous segment is about ¼ the length of the HA protein encoding sequence and about ¼ the length of the NA protein encoding sequence.

In various embodiments, a continuous segment of its HA protein encoding sequence and NA protein encoding sequence is recoded, wherein the continuous segment is about 20-30% of the length of the HA protein encoding sequence and about 20-30% of the length of the NA protein encoding sequence. In various embodiments, a continuous segment of its HA protein encoding sequence and NA protein encoding sequence is recoded, wherein the continuous segment is about 25-35% of the length of the HA protein encoding sequence and about 25-35% of the length of the NA protein encoding sequence. In various embodiments, a continuous segment of its HA protein encoding sequence and NA protein encoding sequence is recoded, wherein the continuous segment is about 30-40% of the length of the HA protein encoding sequence and about 30-40% of the length of the NA protein encoding sequence. In various embodiments, a continuous segment of its HA protein encoding sequence and NA protein encoding sequence is recoded, wherein the continuous segment is about 35-45% of the length of the HA protein encoding sequence and about 35-45% of the length of the NA protein encoding sequence. In various embodiments, a continuous segment of its HA protein encoding sequence and NA protein encoding sequence is recoded, wherein the continuous segment is about 40-50% of the length of the HA protein encoding sequence and about 40-50% of the length of the NA protein encoding sequence. In various embodiments, a continuous segment of its HA protein encoding sequence and NA protein encoding sequence is recoded, wherein the continuous segment is about 45-55% of the length of the HA protein encoding sequence and about 45-55% of the length of the NA protein encoding sequence. In various embodiments, a continuous segment of its HA protein encoding sequence and NA protein encoding sequence is recoded, wherein the continuous segment is about 50-60% of the length of the HA protein encoding sequence and about 50-60% of the length of the NA protein encoding sequence. In various embodiments, a continuous segment of its HA protein encoding sequence and NA protein encoding sequence is recoded, wherein the continuous segment is about 55-65% of the length of the HA protein encoding sequence and about 55-65% of the length of the NA protein encoding sequence. In various embodiments, a continuous segment of its HA protein encoding sequence and NA protein encoding sequence is recoded, wherein the continuous segment is about 60-70% of the length of the HA protein encoding sequence and about 60-70% of the length of the NA protein encoding sequence. In various embodiments, a continuous segment of its HA protein encoding sequence and NA protein encoding sequence is recoded, wherein the continuous segment is about 65-75% of the length of the HA protein encoding sequence and about 65-75% of the length of the NA protein encoding sequence. In various embodiments, a continuous segment of its HA protein encoding sequence and NA protein encoding sequence is recoded, wherein the continuous segment is about 70-80% of the length of the HA protein encoding sequence and about 70-80% of the length of the NA protein encoding sequence. In various embodiments, a continuous segment of its HA protein encoding sequence and NA protein encoding sequence is recoded, wherein the continuous segment is about 75-85% of the length of the HA protein encoding sequence and about 75-85% of the length of the NA protein encoding sequence.

In various embodiments, a continuous segment of its HA protein encoding sequence and NA protein encoding sequence is recoded, wherein the continuous segment is about 76-80% of the length of the HA protein encoding sequence and about 76-80% of the length of the NA protein encoding sequence. In various embodiments, a continuous segment of its HA protein encoding sequence and NA protein encoding sequence is recoded, wherein the continuous segment is about 78% of the length of the HA protein encoding sequence and about 78% of the length of the NA protein encoding sequence.

In various embodiments, about 10-50% of the continuous segment of the HA protein encoding sequence and about 10-50% of the continuous segment of the NA protein encoding sequence are replaced with synonymous mutations. In various embodiments, about 10-20% of the continuous segment of the HA protein encoding sequence and about 10-20% of the continuous segment of the NA protein encoding sequence are replaced with synonymous mutations. In various embodiments, about 20-30% of the continuous segment of the HA protein encoding sequence and about 20-30% of the continuous segment of the NA protein encoding sequence are replaced with synonymous mutations. In various embodiments, about 22-26% of the continuous segment of the HA protein encoding sequence and about 22-26% of the continuous segment of the NA protein encoding sequence are replaced with synonymous mutations. In various embodiments, about 24% of the continuous segment of the HA protein encoding sequence and about 24% of the continuous segment of the NA protein encoding sequence are replaced with synonymous mutations. In various embodiments, about 30-40% of the continuous segment of the HA protein encoding sequence and about 30-40% of the continuous segment of the NA protein encoding sequence are replaced with synonymous mutations.

In various embodiments, one or both of the HA protein-encoding sequence and the NA protein-encoding sequence on the attenuated influenza virus are recoded by lowering the codon-pair bias of the protein-encoding sequence as compared to the natural isolate virus.

In various embodiments, reducing the codon-pair bias comprises identifying a codon pair in the parent protein-encoding sequence having a codon-pair score that can be reduced, and reducing the codon-pair bias by substituting the codon pair with a codon pair that has a lower codon-pair score.

In various embodiments, reducing the codon-pair bias comprises rearranging the codons of a parent protein-encoding sequence.

In various embodiments, each of the recoded HA protein-encoding sequence and/or the recoded NA protein-encoding sequence of the attenuated influenza virus has a codon pair bias less than −0.05, less than −0.1, less than −0.15, less than −0.2, less than −0.25, less than −0.3, less than −0.35, or less than −0.4. In some embodiments, each of the recoded HA protein-encoding sequence and/or the recoded NA protein-encoding sequence of the attenuated influenza virus has a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In various embodiments, each of the recoded HA protein-encoding sequence and the recoded NA protein-encoding sequence of the attenuated influenza virus is recoded by increasing the number of CpG or UpA di-nucleotides compared to the natural isolate virus.

In various embodiments, each of the recoded HA protein-encoding sequence and the recoded NA protein-encoding sequence of the attenuated influenza virus is recoded by increasing at least 5, at least 10, at least 15, at least 20, at least 25, at least 30, at least 35, at least 40, at least 45, at least 50, at least 55, at least 60, at least 65, at least 70, or at least 75 CpG or UpA di-nucleotides compared to the natural isolate virus.

In various embodiments, one or both of the HA protein-encoding sequence and the NA protein-encoding sequence of the attenuated influenza are recoded by replacing 10 or more codons with synonymous codons that are less frequent in the viral host. In various embodiments, one or both of the HA protein-encoding sequence and the NA protein-encoding sequence of the attenuated influenza are recoded by replacing ten or more codons with synonymous codons that are less frequent in the viral host. In various embodiments, one or both of the HA protein-encoding sequence and the NA protein-encoding sequence of the attenuated influenza are recoded by replacing 20 or more codons with synonymous codons that are less frequent in the viral host. In various embodiments, one or both of the HA protein-encoding sequence and the NA protein-encoding sequence of the attenuated influenza are recoded by replacing 50 or more codons with synonymous codons that are less frequent in the viral host. In various embodiments, one or both of the HA protein-encoding sequence and the NA protein-encoding sequence of the attenuated influenza are recoded by replacing 75 or more codons with synonymous codons that are less frequent in the influenza virus. In various embodiments, one or both of the HA protein-encoding sequence and the NA protein-encoding sequence of the attenuated influenza are recoded by replacing 100 or more codons with synonymous codons that are less frequent in the viral host.

In various embodiments, the HA protein of the live attenuated virus is encoded by a HA protein encoding sequence comprising SEQ ID NO:1 or a HA protein encoding sequence selected from Table 1. In various embodiments, the NA protein of the live attenuated virus is encoded by a NA protein encoding sequence comprising SEQ ID NO:2 or a NA protein encoding sequence selected from Table 1.

In various embodiments, the HA protein of the live attenuated virus is encoded by a HA protein encoding sequence comprising SEQ ID NO:3 and the NA protein of the live attenuated virus is encoded by a NA protein encoding sequence comprising SEQ ID NO:4.

In various embodiments, the HA protein of the live attenuated virus is encoded by a HA protein encoding sequence comprising SEQ ID NO:5 and the NA protein of the live attenuated virus is encoded by a NA protein encoding sequence comprising SEQ ID NO:6.

In various embodiments, the HA protein of the live attenuated virus is encoded by a HA protein encoding sequence comprising SEQ ID NO:7 and the NA protein of the live attenuated virus is encoded by a NA protein encoding sequence comprising SEQ ID NO:8.

In various embodiments, the HA protein of the live attenuated virus is encoded by a HA protein encoding comprising SEQ ID NO:15 or the ORF of SEQ ID NO:15. In various embodiments, the HA protein of the live attenuated virus is encoded by a HA protein encoding comprising SEQ ID NO:15 or the ORF of SEQ ID NO:15 and the NA protein of the live attenuated virus is encoded by a NA protein encoding comprising SEQ ID NO:4.

In various embodiments, the HA protein of the live attenuated virus is encoded by a HA protein encoding sequence comprising SEQ ID NO:16 or the ORF of SEQ ID NO: 16. In various embodiments, the HA protein of the live attenuated virus is encoded by a HA protein encoding sequence comprising SEQ ID NO:16 or the ORF of SEQ ID NO: 16 and the NA protein of the live attenuated virus is encoded by a NA protein encoding comprising SEQ ID NO:4.

In various embodiments, the HA protein of the live attenuated virus is encoded by a HA protein encoding sequence comprising SEQ ID NO:17 and the NA protein of the live attenuated virus is encoded by a NA protein encoding sequence comprising SEQ ID NO:18. In various embodiments, the HA protein of the live attenuated virus is encoded by a HA protein encoding sequence comprising ORF of SEQ ID NO:17 and the NA protein of the live attenuated virus is encoded by a NA protein encoding sequence comprising ORF of SEQ ID NO:18.

In various embodiments, the sequences in the above noted SEQ ID NOs each independently comprises up to 20 amino acid substitutions, additions, or deletions compared to its respective sequence identified in its SEQ ID NO. In various embodiments, the sequences in the above noted SEQ ID NOs each independently comprises up to 10 amino acid substitutions, additions, or deletions compared to its respective sequence identified in its SEQ ID NO. In various embodiments, the sequences in the above noted SEQ ID NOs each independently comprises up to 5 amino acid substitutions, additions, or deletions compared to its respective sequence identified in its SEQ ID NO. In various embodiments, the sequences in the above noted SEQ ID NOs each independently comprises up to 4 amino acid substitutions, additions, or deletions compared to its respective sequence identified in its SEQ ID NO. In various embodiments, the sequences in the above noted SEQ ID NOs each independently comprises up to 3 amino acid substitutions, additions, or deletions compared to its respective sequence identified in its SEQ ID NO. In various embodiments, the sequences in the above noted SEQ ID NOs each independently comprises up to 2 amino acid substitutions, additions, or deletions compared to its respective sequence identified in its SEQ ID NO. In various embodiments, the sequences in the above noted SEQ ID NOs each independently comprises up to 1 amino acid substitution, addition, or deletion compared to its respective sequence identified in its SEQ ID NO.

Various embodiments of the present invention provide for a method of eliciting an immune response in a subject in need thereof, comprising: parenterally administering a composition comprising a live attenuated influenza virus in which expression of hemagglutinin (HA) and neuraminidase (NA) is reduced compared to a natural isolate virus, wherein the reduction in expression is a result of recoding the HA protein-encoding sequence and recoding the NA protein-encoding sequence, thereby eliciting the immune response in the subject.

In various embodiments, the HA protein of the live attenuated influenza virus is encoded by a nucleic acid having at least 99% sequence identity to SEQ ID NOs: 1, 3, 5, 7, 15, 16, 17, open reading frame of 15, open reading frame of 16, or open reading frame of 17, and the NA protein of the live attenuated influenza virus is encoded by a nucleic acid having at least 99% sequence identity to SEQ ID NOs: 2, 4, 6, 8, 18, or open reading frame of 18. In various embodiments, an t, PB2, PB1, PA, NS and NP protein of the live attenuated influenza virus are each encoded by a nucleic acid having at least 99% sequence identity to SEQ ID NOs: 9, 10, 11, 12, 13, and 14, respectively, or having at least 99% sequence identity to ORF of SEQ ID NOs: 43, 40, 39, 41, 44, and 42 respectively.

In various embodiments, the HA protein of the live attenuated influenza virus is encoded by a nucleic acid having at least 99% sequence identity to SEQ ID NOs: 1, 3, 5, 7, 15, 16, 17, open reading frame of 15, open reading frame of 16, or open reading frame of 17, and the NA protein of the live attenuated influenza virus is encoded by a nucleic acid having at least 99% sequence identity to SEQ ID NOs: 2, 4, 6, 8, 18, or open reading frame of 18. In various embodiments, an t, PB2, PB1, PA, NS and NP protein of the live attenuated influenza virus are each encoded by a nucleic acid having at least 99% sequence identity to SEQ ID NOs: 43, 40, 39, 41, 44, and 42 respectively, or having at least 99% sequence identity to ORF of SEQ ID NOs: 43, 40, 39, 41, 44, and 42 respectively.

In various embodiments, the HA gene does not comprise the sequence having SEQ ID NO:5. In various embodiments, the NA gene does not comprise the sequence having SEQ ID NO:6. In various embodiments, the attenuated influenza virus' genome does not comprise a wild-type influenza virus genome. In various embodiments, the attenuated influenza virus' genome does not comprise a natural isolate influenza virus genome.

### Viruses and Compositions

Various embodiments of the present invention provide for a live attenuated influenza virus comprising a hemagglutinin (HA) protein encoded by a HA encoding sequence, a neuraminidase (NA) protein encoded by a NA encoding sequence or both, wherein the HA protein encoding sequence comprises SEQ ID NO:1, SEQ ID NO:3, SEQ ID NO: 7, SEQ ID NO:17, or ORF of SEQ ID NO:17 and the NA protein encoding sequence comprises SEQ ID NO:2, SEQ ID NO:4, SEQ ID NO: 8, SEQ ID NO:18 or ORF of SEQ ID NO:18. In various embodiments, the nucleic acid having the sequence of SEQ ID NO:1, SEQ ID NO:2, SEQ ID NO:3, SEQ ID NO: 4, SEQ ID NO:7, SEQ ID NO:8, SEQ ID NO:17, ORF of SEQ ID NO:17, SEQ ID NO:18 or ORF of SEQ ID NO:18 or any combination thereof imparts the attenuation compared to its natural isolate form.

Various embodiments of the present invention provide for a live attenuated influenza virus comprising a hemagglutinin (HA) protein encoded by a HA encoding sequence, a neuraminidase (NA) protein encoded by a NA encoding sequence or both, wherein the HA protein encoding sequence comprises SEQ ID NO:1 and the NA protein encoding sequence comprises SEQ ID NO:2. In various embodiments, the nucleic acid having sequence of SEQ ID NO:1, SEQ ID NO:2, or both imparts the attenuation compared to its natural isolate form.

Various embodiments of the present invention provide for a live attenuated influenza virus comprising a hemagglutinin (HA) protein encoded by a HA encoding sequence, a neuraminidase (NA) protein encoded by a NA encoding sequence or both, wherein the HA protein encoding sequence comprises SEQ ID NO:3 and the NA protein encoding sequence comprises SEQ ID NO:4. In various embodiments, the nucleic acid having the sequence of SEQ ID NO:3, SEQ ID NO:4, or both imparts the attenuation compared to its natural isolate form.

Various embodiments of the present invention provide for a live attenuated influenza virus comprising a hemagglutinin (HA) protein encoded by a HA encoding sequence, a neuraminidase (NA) protein encoded by a NA encoding sequence or both, wherein the HA protein encoding sequence comprises SEQ ID NO:5 and the NA protein encoding sequence comprises SEQ ID NO:6. In various embodiments, the nucleic acid having the sequence of SEQ ID NO:5, SEQ ID NO:6, or both imparts the attenuation compared to its natural isolate form.

Various embodiments of the present invention provide for a live attenuated influenza virus comprising a hemagglutinin (HA) protein encoded by a HA encoding sequence, a neuraminidase (NA) protein encoded by a NA encoding sequence or both, wherein the HA protein encoding sequence comprises SEQ ID NO:7 and the NA protein encoding sequence comprises SEQ ID NO:8. In various embodiments, the nucleic acid having the sequence of SEQ ID NO:7, SEQ ID NO:8, or both imparts the attenuation compared to its natural isolate form.

Various embodiments of the present invention provide for a live attenuated influenza virus comprising a hemagglutinin (HA) protein encoded by a HA encoding sequence, a neuraminidase (NA) protein encoded by a NA encoding sequence or both, wherein the HA protein encoding sequence comprises SEQ ID NO:17 or ORF of SEQ ID NO:17 and the NA protein encoding sequence comprises SEQ ID NO:18 or ORF of SEQ ID NO:18. In various embodiments, the nucleic acid having the sequence of SEQ ID NO:17, ORF of SEQ ID NO: 17, SEQ ID NO:18, or ORF of SEQ ID NO:18 or a combination thereof imparts the attenuation compared to its natural isolate form.

Various embodiments of the present invention provide for a live attenuated influenza virus comprising a hemagglutinin (HA) protein encoded by a HA encoding sequence, a neuraminidase (NA) protein encoded by a NA encoding sequence or both, wherein the HA protein encoding sequence comprises SEQ ID NO:15 or the ORF of SEQ ID NO:15. In various embodiments, the nucleic acid having the sequence of SEQ ID NO:15, or ORF of SEQ ID NO:15 imparts the attenuation compared to its natural isolate form.

Various embodiments of the present invention provide for a live attenuated influenza virus comprising a hemagglutinin (HA) protein encoded by a HA encoding sequence, a neuraminidase (NA) protein encoded by a NA encoding sequence or both, wherein the HA protein encoding sequence comprises SEQ ID NO:16 or the ORF of SEQ ID NO: 16. In various embodiments, the nucleic acid having the sequence of SEQ ID NO:16, or ORF of SEQ ID NO:16 imparts the attenuation compared to its natural isolate form.

Various embodiments of the present invention provide for a live attenuated influenza virus comprising a hemagglutinin (HA) protein encoded by a HA encoding sequence, a neuraminidase (NA) protein encoded by a NA encoding sequence or both, wherein the HA protein of the live attenuated influenza virus is encoded by a nucleic acid having at least 99% sequence identity to SEQ ID NOs: 1, 3, 5, 7, 15, 16, 17, open reading frame of 15, open reading frame of 16, or open reading frame of 17 and the NA protein of the live attenuated influenza virus is encoded by a nucleic acid having at least 99% sequence identity to SEQ ID NOs: 2, 4, 6, 8, 18 or open reading frame of 18. In various embodiments, an M, PB2, PB1, PA, NS and NP protein of the live attenuated influenza virus are each encoded by a nucleic acid having at least 99% sequence identity to SEQ ID NOs: 9, 10, 11, 12, 13, and 14, respectively. In various embodiments, an M, PB2, PB1, PA, NS and NP protein of the live attenuated influenza virus are each encoded by a nucleic acid having at least 99% sequence identity to SEQ ID NOs: 43, 40, 39, 41, 44, and 42, respectively, or having at least 99% sequence identity to ORF of SEQ ID NOs: 43, 40, 39, 41, 44, and 42 respectively.

In various embodiments, one or more of the protein encoding sequence imparts the attenuation compared to its natural isolate form.

In various embodiments, the HA gene does not comprise the sequence having SEQ ID NO:5. In various embodiments, the NA gene does not comprise the sequence having SEQ ID NO:6. In various embodiments, the attenuated influenza virus' genome does not comprise a wild-type influenza virus genome. In various embodiments, the attenuated influenza virus' genome does not comprise a natural isolate influenza virus genome.

In various embodiments, the live attenuated influenza virus comprises a hemagglutinin (HA) protein encoded by a HA encoding sequence, a neuraminidase (NA) protein encoded by a NA encoding sequence or both, wherein the HA protein encoding sequence comprises a nucleic acid having at least 99% sequence identity to SEQ ID NO:1 and the NA protein encoding sequence comprises a nucleic acid having at least 99% sequence identity to SEQ ID NO:2. In various embodiments, the nucleic acid having the sequence of SEQ ID NO:1, SEQ ID NO:2, or both imparts the attenuation compared to its natural isolate form.

In various embodiments, the live attenuated influenza virus comprises a hemagglutinin (HA) protein encoded by a HA encoding sequence, a neuraminidase (NA) protein encoded by a NA encoding sequence or both, wherein the HA protein encoding sequence comprises a nucleic acid having at least 99% sequence identity to SEQ ID NO:3 and the NA protein encoding sequence comprises a nucleic acid having at least 99% sequence identity to SEQ ID NO:4 In various embodiments, the nucleic acid having at least 99% sequence identity to SEQ ID NO:3, SEQ ID NO:4, or both imparts the attenuation compared to its natural isolate form.

In various embodiments, the live attenuated influenza virus comprises a hemagglutinin (HA) protein encoded by a HA encoding sequence, a neuraminidase (NA) protein encoded by a NA encoding sequence or both, wherein the HA protein encoding sequence comprises a nucleic acid having at least 99% sequence identity to SEQ ID NO:5 and the NA protein encoding sequence comprises a nucleic acid having at least 99% sequence identity to SEQ ID NO:6. In various embodiments, the nucleic acid having at least 99% sequence identity to SEQ ID NO:5, SEQ ID NO:6, or both imparts the attenuation compared to its natural isolate form.

In various embodiments, the live attenuated influenza virus comprises a hemagglutinin (HA) protein encoded by a HA encoding sequence, a neuraminidase (NA) protein encoded by a NA encoding sequence or both, wherein the HA protein encoding sequence comprises a nucleic acid having at least 99% sequence identity to SEQ ID NO:7 and the NA protein encoding sequence comprises a nucleic acid having at least 99% sequence identity to SEQ ID NO:8. In various embodiments, the nucleic acid having at least 99% sequence identity to SEQ ID NO:7, SEQ ID NO:8, or both imparts the attenuation compared to its natural isolate form.

In various embodiments, the live attenuated influenza virus comprises a hemagglutinin (HA) protein encoded by a HA encoding sequence, a neuraminidase (NA) protein encoded by a NA encoding sequence or both, wherein the HA protein encoding sequence comprises a nucleic acid having at least 99% sequence identity to SEQ ID NO:17 or to the ORF of SEQ ID NO:17 and the NA protein encoding sequence comprises a nucleic acid having at least 99% sequence identity to SEQ ID NO:18 or to the ORF of SEQ ID NO:18. In various embodiments, the nucleic acid having at least 99% sequence identity to SEQ ID NO:17, ORF of SEQ ID NO:17, SEQ ID NO:18, or ORF of SEQ ID NO:18, or any combination thereof imparts the attenuation compared to its natural isolate form.

In various embodiments, the live attenuated influenza virus comprises a hemagglutinin (HA) protein encoded by a HA encoding sequence, a neuraminidase (NA) protein encoded by a NA encoding sequence or both, wherein the HA protein encoding sequence comprises a nucleic acid having at least 99% sequence identity to SEQ ID NO:15 or to the ORF of SEQ ID NO:15. In various embodiments, the nucleic acid having at least 99% sequence identity to SEQ ID NO:15, or to the ORF of SEQ ID NO:15 imparts the attenuation compared to its natural isolate form.

In various embodiments, the live attenuated influenza virus comprises a hemagglutinin (HA) protein encoded by a HA encoding sequence, a neuraminidase (NA) protein encoded by a NA encoding sequence or both, wherein the HA protein encoding sequence comprises a nucleic acid having at least 99% sequence identity to SEQ ID NO:16 or to the ORF of SEQ ID NO: 16. In various embodiments, the nucleic acid having at least 99% sequence identity to SEQ ID NO:16, or to ORF of SEQ ID NO:16 imparts the attenuation compared to its natural isolate form.

Various embodiments of the present invention provide for a parenterally administrable composition, comprising: a live attenuated influenza virus in which expression of hemagglutinin (HA) and neuraminidase (NA) is reduced compared to a natural isolate virus, wherein the reduction in expression is a result of recoding the HA protein-encoding sequence and recoding the NA protein-encoding sequence, thereby eliciting the immune response in the subject.

Various embodiments of the present invention provide for a parenterally administrable composition, comprising: a live attenuated influenza virus in which its hemagglutinin (HA) protein encoding sequence and neuraminidase (NA) protein encoding sequence are recoded, and wherein the amino acid sequence of HA protein or NA protein of the live attenuated influenza virus remains the same, or wherein the amino acid sequence of the HA protein or NA protein of the live attenuated influenza virus comprises up to 20 amino acid substitutions, additions, or deletions compared to the natural isolate virus; and a pharmaceutically acceptable carrier or excipient suitable for parenteral administration.

In various embodiments, the live attenuated influenza virus comprises a hemagglutinin (HA) protein encoded by a HA encoding sequence, a neuraminidase (NA) protein encoded by a NA encoding sequence or both, wherein the HA protein encoding sequence comprises SEQ ID NO:1, SEQ ID NO:3, SEQ ID NO: 7, SEQ ID NO:17 or ORF of SEQ ID NO:17, and the NA protein encoding sequence comprises SEQ ID NO:2. In various embodiments, the nucleic acid having the sequence of SEQ ID NO:1, SEQ ID NO:2, SEQ ID NO: 4, SEQ ID NO:8, SEQ ID NO:18, or ORF of SEQ ID NO:18 or any combination thereof imparts the attenuation compared to its natural isolate form.

In various embodiments, the live attenuated influenza virus comprising a hemagglutinin (HA) protein encoded by a HA encoding sequence, a neuraminidase (NA) protein encoded by a NA encoding sequence or both, wherein the HA protein encoding sequence comprises SEQ ID NO:15 or the ORF of SEQ ID NO:15. In various embodiments, the nucleic acid having the sequence of SEQ ID NO:15 or ORF of SEQ ID NO:15 imparts the attenuation compared to its natural isolate form.

In various embodiments, the live attenuated influenza virus comprising a hemagglutinin (HA) protein encoded by a HA encoding sequence, a neuraminidase (NA) protein encoded by a NA encoding sequence or both, wherein the HA protein encoding sequence comprises SEQ ID NO:16 or the ORF of SEQ ID NO: 16. In various embodiments, the nucleic acid having the sequence of SEQ ID NO:16, or ORF of SEQ ID NO:16 imparts the attenuation compared to its natural isolate form.

In various embodiments, the sequences in the above noted SEQ ID NOs each independently comprises up to 20 amino acid substitutions, additions, or deletions compared to its respective sequence identified in its SEQ ID NO. In various embodiments, the sequences in the above noted SEQ ID NOs each independently comprises up to 10 amino acid substitutions, additions, or deletions compared to its respective sequence identified in its SEQ ID NO. In various embodiments, the sequences in the above noted SEQ ID NOs each independently comprises up to 5 amino acid substitutions, additions, or deletions compared to its respective sequence identified in its SEQ ID NO.

Various embodiments of the present invention provide for an parenterally administrable composition, comprising: a live attenuated influenza virus in which expression of hemagglutinin (HA) and neuraminidase (NA) is reduced compared to a natural isolate virus, wherein the reduction in expression is a result of recoding the HA protein-encoding sequence and recoding the NA protein-encoding sequence, thereby eliciting the immune response in the subject, or a live attenuated influenza virus in which its hemagglutinin (HA) protein encoding sequence and neuraminidase (NA) protein encoding sequence are recoded, and the HA protein of the live attenuated influenza virus is encoded by a nucleic acid having at least 99% sequence identity to SEQ ID NOs: 1, 3, 5, 7, 15, 16, 17, open reading frame of 15, open reading frame of 16, or open reading frame of 17 and the NA protein of the live attenuated influenza virus is encoded by a nucleic acid having at least 99% sequence identity to SEQ ID NOs: 2, 4, 6, 8, 18 or open reading frame of 18; and a pharmaceutically acceptable carrier or excipient suitable for parenteral administration. In various embodiments an M, PB2, PB1, PA, NS and NP protein of the influenza virus are each encoded by a nucleic acid having at least 99% sequence identity to SEQ ID NOs: 9, 10, 11, 12, 13, and 14, respectively. In various embodiments, an M, PB2, PB1, PA, NS and NP protein of the live attenuated influenza virus are each encoded by a nucleic acid having at least 99% sequence identity to SEQ ID NOs: 43, 40, 39, 41, 44, and 42, respectively, or having at least 99% sequence identity to ORF of SEQ ID NOs: 43, 40, 39, 41, 44, and 42 respectively.

In various embodiments, the HA gene does not comprise the sequence having SEQ ID NO:5. In various embodiments, the NA gene does not comprise the sequence having SEQ ID NO:6. In various embodiments, the attenuated influenza virus' genome does not comprise a wild-type influenza virus genome.

In various embodiments, the composition comprises a dosage of about 10³-10⁹ PFU of the live attenuated influenza virus. In various embodiments, the composition comprises a dosage of about 10⁷-10⁸ PFU of the live attenuated influenza virus. In various embodiments, the composition comprises a dosage of about 1×10³, 1×10⁴, 1×10⁵, 1×10⁶, 10⁷, 1×10⁸ or 1×10⁹, PFU of the live attenuated influenza virus. In various embodiments, the composition comprises a dosage of about 2×10³, 2×10⁴, 2×10⁵, 2×10⁶, 2×10⁷, 2×10⁸ or 2×10⁹, PFU of the live attenuated influenza virus. In various embodiments, the composition comprises a dosage of about 5×10³, 5×10⁴, 5×10⁵, 5×10⁶, 5×10⁷, 5×10⁸ or 5×10⁹, PFU of the live attenuated influenza virus. In various embodiments, the composition comprises a dosage of about 5×10⁴ PFU of the live attenuated influenza virus. In various embodiments, the composition comprises a dosage of about 5×10⁵ PFU of the live attenuated influenza virus. In various embodiments, the composition comprises a dosage of about 5×10⁶ PFU of the live attenuated influenza virus.

In various embodiments, the composition comprises a dosage of about 0.03-0.3 μg of HA protein. In various embodiments, the composition comprises a dosage of about 0.01-0.05 μg of HA protein. In various embodiments, the composition comprises a dosage of about 0.05-0.1 kg of HA protein. In various embodiments, the composition comprises a dosage of about 0.1-0.15 kg of HA protein. In various embodiments, the composition comprises a dosage of about 0.15-0.2 μg of HA protein. In various embodiments, the composition comprises a dosage of about 0.2-0.25 μg of HA protein. In various embodiments, the comprises a dosage of about 0.25-0.3 μg of HA protein.

In various embodiments, the pharmaceutically acceptable carrier or excipient suitable for administration via injection is suitable for administration via intramuscular injection. In various embodiments, the pharmaceutically acceptable carrier or excipient suitable for administration via injection is suitable for administration via subcutaneous injection. In various embodiments, the pharmaceutically acceptable carrier or excipient suitable for administration via intradermal injection. In various embodiments, the pharmaceutically acceptable carrier or excipient suitable for administration via intravenous administration.

In various embodiments, the method of the present invention utilizes viruses that are highly attenuated by recoding its protein encoding sequences as described herein, and induce immunity against a plurality of influenza types and/or subtypes. Such flu varieties include viruses bearing all possible HA-NA combinations. Currently, there are 16 recognized hemagglutinins and nine neuraminidases, each of which has mutational variants. Examples of type A subtypes include, but are not limited to, H10N7, H10N1, H10N2, H10N3, H10N4, H10N5, H10N6, H10N7, H10N8, H10N9, H11N1, H11N2, H11N3, H11N4, H11N6, H11N8, H11N9, H12N1, H12N2, H12N4, H12N5, H12N6, H12N8, H12N9, H13N2, H13N3, H13N6, H13N9, H14N5, H14N6, H15N2, H15N8, H15N9, H16N3, H1N1, H1N2, H1N3, H1N5, H1N6, H1N8, H1N9, H2N1, H2N2, H2N3, H2N4, H2N5, H2N6, H2N7, H2N8, H2N9, H3N1, H3N2, H3N3, H3N4, H3N5, H3N6, H3N8, H3N9, H4N1, H4N2, H4N3, H4N4, H4N5, H4N6, H4N7, H4N8, H4N9, H5N1, H5N2, H5N3, H5N4, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N7, H7N8, H7N9, H8N2, H8N4, H8N5, H9N1, H9N2, H9N3, H9N4, H9N5, H9N6, H9N7, H9N8, H9N9.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication.

In various embodiments, the pharmaceutical compositions according to the invention may be formulated for delivery via parenteral administration. In various embodiments, the pharmaceutical compositions according to the invention may be formulated for delivery via injection. In various embodiments, the pharmaceutical compositions according to the invention may be formulated for delivery via intramuscular administration. In various embodiments, the pharmaceutical compositions according to the invention may be formulated for delivery via subcutaneous administration. In various embodiments, the pharmaceutical compositions according to the invention may be formulated for delivery via intradermal administration.

TABLE 1
Reduced-Expression Influenza A Virus Genes

| | Recoded Coding Sequence | | |
|---|---|---|---|
| Gene | SEQ ID NO | Recoded Codons | CPB |

Singapore H3N2 Sequences

| | |
|---|---|
| HA | 1 |
| NA | 2 |

H1N1 A/California/07/2009

| | | |
|---|---|---|
| HA | 3 | 49-537 |
| NA | 4 | 38-436 |

H1N1 A/California/07/2009

| | | |
|---|---|---|
| HA | 5 | 49-537 |
| NA | 6 | 38-436 |

H3N2 A/Texas/50/2012

| | |
|---|---|
| HA | 7 |
| NA | 8 |

H1N1 A/California/07/2009

| | |
|---|---|
| HA | 15 |
| HA | 16 |
| HA | 17 |
| NA | 18 |

H10N7 (A/northern shoveler/California/HKWF392sm/2007)(Avian)

| | | |
|---|---|---|
| HA | 1-561 | −0.441 |
| NA | 1-498 | −0.449 |

H1N1(A/New York/3568/2009)(Human)

| | | |
|---|---|---|
| HA | 1-566 | −0.410 |
| NA | 1-469 | −0.456 |

HIN2 (A/New York/211/2003)(Human)

| | | |
|---|---|---|
| HA | 1-565 | −0.421 |
| NA | 1-469 | −0.476 |

H2N2 (A/Albany/22/1957)(Human)

| | | |
|---|---|---|
| HA | 1-562 | −0.422 |
| NA | 1-469 | −0.453 |

H3N2 (A/New York/933/2006)(Human)

| | | |
|---|---|---|
| HA | 1-566 | −0.447 |
| NA | 1-469 | −0.463 |

H5Nl (A/Jiangsu/1/2007)(Human)

| | | |
|---|---|---|
| HA | 1-567 | −0.435 |
| NA | 1-449 | −0.407 |

H7N2 (A/chicken/NJ/294508-12/2004)(Avian)

| | | |
|---|---|---|
| HA | 1-552 | −0.377 |
| NA | 1-453 | −0.491 |

H7N3 (A/Canada/rv504/2004)(Human)

| | | |
|---|---|---|
| HA | 1-567 | −0.405 |
| NA | 1-469 | −0.413 |

H7N7 (A/Netherlands/219/03)(Human)

| | | |
|---|---|---|
| HA | 1-569 | −0.447 |
| NA | 1-471 | −0.423 |

H9N2 (A/Hong Kong/I 073/99)(Human)

| | | |
|---|---|---|
| HA | 1-560 | −0.440 |
| NA | 1-467 | −0.453 |

Sequences

| | |
|---|---|
| Singapore | ATGAAGACTATCATTGCTTTGAGCTACATTCTATGTCTGGTTTTCGCTCAAA |
| H3N2 HA | AAATTCCTGGAAATGACAATAGCACGGCAACGCTGTGCCTTGGGCACCAT |
| SEQ ID | GCAGTACCAAACGGAACGATAGTGAAAACAATCACTAACGATAGAATCG |
| NO: 1 | AAGTGACTAACGCTACTGAGTTAGTGCAAAATAGCTCAATCGGTGAGATA |
| | TGCGATAGCCCACACCAAATATTGGACGGAGAGAATTGTACACTGATAGA |
| | CGCACTGTTAGGCGATCCACAATGCGACGGATTCCAAAATAAAAAATGGG |
| | ACCTATTCGTTGAGCGATCTAAGGCATACTCTAATTGTTATCCATACGACG |
| | TACCCGATTACGCTAGTCTTAGGTCACTAGTTGCGTCAAGCGGAACACTCG |
| | AATTCAAAAACGAATCATTCAATTGGACAGGCGTTACGCAAAACGGAACA |
| | TCTAGCGCATGCATTAGGGGGTCAAGCTCAAGCTTTTTTAGTAGACTGAAT |
| | TGGTTGACACATCTGAATTATACTTATCCCGCTCTTAACGTTACTATGCCTA |
| | ATAAAGAGCAATTCGATAAACTGTATATATGGGGCGTACACCATCCCGGA |
| | ACTGACAAAGACCAGATATTCCTATACGCTCAATCTAGCGGACGGATAAC |
| | CGTTAGTACTAAACGATCACAGCAAGCCGTTATACCGAATATCGGATCTA |
| | GACCTAGAATTAGAGACATACCGTCACGAATATCAATCTATTGGACTATC |
| | GTTAAGCCAGGCGATATACTGTTGATTAATAGTACAGGTAATCTAATCGCA |
| | CCTAGGGGGTATTTCAAAATTAGATCCGGTAAGTCTAGTATTATGAGATCC |
| | GACGCTCCAATCGGTAAGTGTAAAAGCGAATGCATAACACCTAATGGGTC |
| | AATACCGAACGATAAGCCATTCCAAAATGTGAATAGGATTACATACGGAG |
| | CATGCCCTAGATACGTTAAGCATAGTACACTTAAACTCGCAACCGGAATG |
| | CGAAACGTACCCGAAAAACAGACTAGGGGGATATTCGGAGCTATAGCCGG |
| | ATTTATCGAAAACGGATGGGAGGGAATGGTAGACGGATGGTACGGATTTA |
| | GACACCAAAATAGCGAAGGTAGGGGGCAAGCCGCAGACCTTAAAAGTAC |
| | ACAAGCCGCAATCGATCAGATTAACGGAAAGCTTAATAGGTTGATCGGTA |
| | AGACTAACGAAAAATTTCACCAAATCGAAAAAGAGTTTAGCGAAGTCGAG |
| | GGTAGGGTGCAAGACCTTGAGAAATACGTTGAGGATACTAAAATCGATCT |
| | ATGGTCATACAATGCCGAACTGTTGGTTGCGCTTGAGAATCAGCATACAAT |
| | CGATCTAACCGATAGCGAAATGAATAAATTGTTCGAAAAAACTAAAAAAC |
| | AATTGAGAGAGAATGCCGAAGACATGGGTAACGGATGTTTTAAGATATAT |
| | CACAAATGCGATAACGCTTGTATCGGATCAATTAGAAACGAAACATACGA |
| | TCATAACGTATATAGGGACGAAGCACTGAATAATAGATTCCAAATTAAGG |
| | GCGTAGAGTTGAAATCCGGATACAAAGATTGGATATTGTGGATATCCTTTG |
| | CCATATCATGTTTTTTGCTTTGTGTTGCTTTGTTGGGGTTCATCATGTGGGC |
| | CTGCCAAAAGGGCAACATTAGATGCAACATTTGCATTTGA |
| Singapore | ATGAATCCAAATCAAAAGATAATAACGATTGGCTCTGTTTCTCTCACCATT |
| H3N2 NA | TCCACAATATGCTTCTTCATGCAAATTGCCATCCTGATAACTACTGTAACA |
| SEQ ID | TTGCATTTCAAGCAATACGAATTCAACTCACCCCCTAACAATCAGGTTATG |
| NO: 2 | TTGTGCGAACCTACTATAATCGAACGTAATATAACCGAGATAGTGTATCTG |
| | ACTAATACCACAATCGAAAAAGAGATATGCCCTAAACCCGCTGAGTATAG |
| | GAATTGGTCTAAACCGCAATGCGGTATTACCGGTTTCGCACCATTTTCGAA |
| | AGATAACTCCATTAGGTTGTCCGCCGGAGGCGATATATGGGTGACACGCG |
| | AACCATATGTGAGTTGCGATCCCGATAAATGTTATCAATTCGCACTCGGAC |
| | AGGGGACTACACTTAATAACGTTCATAGCAATAATACCGTTAGGGATAGG |
| | ACACCTTATAGAACACTATTGATGAACGAATTGGGAGTGCCATTCCATCTA |
| | GGTACGAAACAGGTGTGTATCGCATGGTCCTCTAGCTCATGTCATGACGGT |
| | AAGGCATGGTTGCACGTATGCATAACCGGAGACGATAAAAACGCTACCGC |
| | TAGTTTCATATATAACGGACGGTTAGTCGATAGCGTAGTGAGTTGGTCTAA |
| | AGACATACTTAGAACACAGGAATCCGAATGCGTATGCATTAACGGTACAT |
| | GTACAGTCGTTATGACTGACGGAAATGCTACGGGaAAGGCCGATACTAAG |
| | ATACTGTTTATCGAAGAGGGAAAGATAGTGCATACTAGTAAGCTATCCGG |
| | ATCCGCTCAACATGTCGAAGAGTGCTCATGTTATCCTAGATATCCCGGAGT |
| | GAGATGCGTTTGTAGGGATAATTGGAAAGGGTCAAATAGACCTATCGTTG |
| | ACATAAACATAAAAGATCATTCAATCGTTAGCTCATACGTTTGTTCCGGAC |
| | TGGTAGGCGATACACCTAGAAAAAACGATAGCTCAAGCTCAAGCCATTGT |
| | CTTAACCCTAATAACGAAGAGGGGGGGCATGGCGTTAAAGGGTGGGCATT |
| | CGACGACGGTAACGACGTATGGATGGGACGGACAATTAACGAAACTAGTA |
| | GATTGGGGTATGAGACATTCAAAGTCGTTGAGGGATGGTCTAACCCTAAA |
| | TCCAAATTGCAAATTAATCGGCAAGTGATAGTCGATAGAGGCGATAGATC |
| | CGGATATTCCGGTATTTTTAGCGTTGAGGGAAAGTCATGTATTAATAGGTG |
| | TTTTTATGTCGAATTGATTAGGGGGCGAAAAGAGGAAACCGAAGTGCTTT |
| | GGACCTCAAACAGTATTGTTGTGTTTTGTGGCACCTCAGGTACATATGGAA |
| | CAGGCTCATGGCCTGATGGGGCGGACCTCAATCTCATGCATATATAA |
| H1N1 HA | ATGAAGGCAATACTAGTAGITCTGCTATATACATTTGCAACCGCAAATGCA |
| A/California/ | GACACATTATGTATAGGTTATCATGCGAACAATTCAACAGACACTGTAGA |
| 07/2009 | CACAGTACTAGAAAAGAATGTAACAGTAACACACTCTGTTAACCTATTGG |
| Min | AGGATAAGCATAACGGTAAGCTATGCAAACTGAGAGGCGTAGCACCATTG |
| SEQ ID | CATCTAGGTAAGTGTAATATAGCCGGATGGATTCTAGGGAATCCCGAATG |
| NO: 3 | CGAATCACTATCAACCGCTAGCTCATGGTCATACATAGTCGAAACACCATC |
| | AAGCGATAACGGTACATGTTATCCCGGAGACTTTATCGATTACGAAGAGC |
| | TTAGAGAGCAATTGTCTAGCGTAAGCTCATTCGAAAGATTCGAAATTTTTC |
| | CGAAAACTAGCTCATGGCCTAATCACGATAGTAATAAAGGCGTAACTGCC |
| | GCATGCCCACACGCCGGAGCTAAATCATTCTATAAGAATCTGATTTGGTTA |
| | GTGAAAAAAGGGAATTCATATCCGAAACTATCTAAATCATACATTAACGA |
| | TAAGGGTAAGGAGGTACTAGTGTTGTGGGGGATACACCATCCATCAACTA |
| | GCGCCGATCAGCAATCATTGTATCAGAACGCAGACGCATACGTATTCGTA |
| | GGGTCTAGTAGATACTCTAAAAAATTTAAACCCGAAATCGCAATTAGACC |
| | GAAAGTGAGAGACCAAGAGGGTAGAATGAATTACTATTGGACACTAGTCG |
| | AACCAGGCGATAAGATTACATTCGAAGCGACAGGGAATCTAGTCGTACCG |
| | AGATACGCATTCGCAATGGAGAGAAACGCCGGATCCGGAATTATTATTAG |
| | CGATACACCCGTACACGATTGCAATACTACATGTCAGACACCAAAAGGCG |
| | CAATTAATACTAGTCTGCCATTTCAGAATATACACCCAATTACAATCGGTA |
| | AGTGTCCAAAATACGTTAAGTCAACTAAGTTGAGACTCGCAACAGGGTTG |
| | AGAAATATACCGTCAATTCAATCTAGGGGGTTGTTCGGAGCAATCGCAGG |
| | GTTTATCGAAGGGGGGTGGACAGGTATGGTTGACGGATGGTACGGATACC |
| | ATCATCAAAACGAACAGGGATCCGGATACGCAGCCGATCTGAAAAGTACA |
| | CAGAACGCTATAGACGAAATTACGAATAAAGTGAATAGCGTAATCGAAAA |
| | AATGAATACGCAATTTACAGCCGTAGGTAAGGAGTTTAATCATCTCGAAA |
| | AAAGGATTGAGAATCTGAATAAAAAAGTCGACGACGGATTCTTAGACATT |
| | TGGACTTATAACGCCGAACTGTTAGTGTTACTCGAAAACGAAAGAACACT |
| | AGACTATCACGATTCAAACGTTAAGAATCTATACGAAAAAGTGAGATCGC |
| | AATTGAAAAATAACGCTAAAGAGATAGGGAATGGGTGTTTCGAATTCTAT |
| | CATAAATGCGATAATACATGTATGGAATCCGTTAAAAACGGAACATACGA |
| | TTACCCTAAGTATAGCGAAGAGGCTAAACTGAATAGGGAAGAGATAGACG |
| | GAGTGAAACTCGAATCAACTAGGATTTATCAGATACTCGCAATTTATAGTA |
| | CGGTTGCCAGTTCATTGGTACTGGTAGTCTCCCTGGGGGCAATCAGTTTCT |
| | GGATGTGCTCTAATGGGTCTCTACAGTGTAGAATATGTATTTAA |
| H1N1 NA | ATGAATCCAAACCAAAAGATAATAACCATTGGTTCGGTCTGTATGACAAT |
| A/California/ | TGGAATGGCTAACTTAATATTACAAATTGGAAACATAATCTCAATATGGAT |
| 07/2009 | TAGCCACTCAATCCAATTGGGGAATCAGAATCAAATCGAAACATGCAATC |
| Min | AATCCGTAATTACATACGAGAATAATACTTGGGTGAATCAGACATACGTT |
| SEQ ID | AACATATCGAATACTAATTTCGCTGCCGGACAATCCGTCGTGAGTGTGAA |
| NO: 4 | ACTAGCCGGTAATAGTAGTCTATGTCCCGTTAGCGGATGGGCTATATACTC |
| | TAAAGACAATAGCGTTAGAATCGGATCTAAAGGCGACGTATTCGTTATAC |
| | GCGAACCATTCATAAGTTGTAGTCCATTAGAGTGTAGGACTTTTTTTCTGA |
| | CACAGGGCGCACTATTGAACGATAAGCATTCTAACGGTACAATCAAAGAT |
| | AGGTCACCATATAGAACACTAATGTCATGTCCGATAGGCGAAGTGCCTAG |
| | TCCATACAATAGTAGATTCGAATCCGTCGCTTGGTCCGCTAGCGCATGCCA |
| | TGACGGTATTAATTGGTTGACAATCGGGATTAGCGGACCCGATAACGGCG |
| | CAGTCGCCGTACTTAAGTATAACGGTATAATTACCGATACTATTAAGAGTT |
| | GGCGAAATAATATATTGCGAACACAGGAATCCGAATGCGCATGCGTTAAC |
| | GGATCATGTTTTACCGTTATGACTGACGGACCATCTAACGGGCAAGCGTCA |
| | TATAAGATTTTTAGAATCGAAAAAGGTAAGATAGTGAAATCCGTCGAAAT |
| | GAACGCTCCTAATTATCATTACGAAGAGTGCTCATGTTATCCCGATTCTAG |
| | CGAAATTACATGCGTATGTAGAGACAATTGGCACGGATCTAATAGACCTT |
| | GGGTGTCATTCAATCAGAATCTAGAGTATCAAATCGGGTATATATGCTCAG |
| | GGATATTCGGAGACAATCCTAGACCTAACGATAAGACAGGGTCATGCGGA |
| | CCAGTGAGTTCTAACGGCGCTAACGGCGTTAAGGGGTTTAGTTTCAAATAC |
| | GGTAACGGCGTATGGATAGGGAGAACTAAGTCAATCTCTAGTAGAAACGG |
| | ATTCGAAATGATATGGGACCCTAACGGATGGACCGGAACCGATAATAATT |
| | TTTCGATTAAACAGGATATCGTAGGGATTAACGAATGGTCAGGGTATAGC |
| | GGATCATTCGTACAGCATCCAGAGTTAACCGGACTCGATTGCATACGACC |
| | ATGTTTTTGGGTCGAACTGATTAGGGGGAGACCGAAAGAGAATACTATAT |
| | GGACTAGCGGGAGCAGCATATCCTTTTGTGGTGTAAACAGTGACACTGTG |
| | GGTTGGTCTTGGCCAGACGGTGCTGAGTTGCCATTTACCATTGACAAGTAA |
| H1N1 HA | atgaaggcaatactagtagttctgctatatacatttgcaaccgcaaatgcagacacattatgtataggttatcatgcgaacaattca |
| A/California/ | acagacactgtagacacagtactagaaaagaatgtaacagtaacacactctgttaacctattggaggataagcataacagtaag |
| 07/2009 | ctatgcaaactgagaggcgtagcaccattgcatctaggtaagtgtaatatagccggatggattctagggaatcccgaatgcgaa |
| M100/V6 | tcactatcaaccgctagctcatggtcatacatagtcgaaacaccatcaagcgataacggtacatgttatcccggagactttatcga |
| SEQ ID | ttacgaagagcttagagagcaattgtctagcgtaagctcattcgaaagattcgaaatttttccgaaaactagctcatggcctaatca |
| NO: 5 | cgatagtagtaaaggcgtaactgccgcatgcccacacgccggagctaaatcattctataagaatctgatttggttagtgaaaaaa |
| | gagaattcatatccgaaactatctaaatcatacattaacgataagggtaaggaggtactagtgttgtgggggatacaccatccatc |
| | aactagcgccgatcagcaatcattgtatcagaacgcagacgcatacgtattcgtagggtctagtagatactctaaaaaatttaaac |
| | ccgaaatcgcaattagaccgaaagtgagaggccaagagggtagaatgaattactattggacactagtcgaaccaggcgataa |
| | gattacattcgaagcgacagggaatctagtcgtaccgagatacgcattcgcaatggagagaaacgccggatccggaattattat |
| | tagcgatacacccgtacacgattgcaatactacgtgtcagacaccaaaaggcgcaattaatactagtctgccatttcagaatatac |
| | acccaattacaatcggtaagtgtccaaaatacgttaagtcaactaagttgagactcgcaacagggttgagaaatacaccgtcaat |
| | tcaatctagggggttgttcggagcaatcgcagggtttatcgaaggggggtggacaggtatggttgacggatggtacggatacc |
| | atcatcaaaacgaacagggatccggatacgcagccgaactgaaaagtacacagaacgctatagacgaaattacgaataaagt |
| | gaatagcgtaatcgaaaaaatgaatacgcaatttacagccgtaggtaaggagtttaatcatctcgaaaaaaggattgagaatctg |
| | aataaaaaagtcgacgacggattcttagacatttggacttataacgccgaaatgttagtgttactcgaaaacgaaagaacactag |
| | actatcacgattcaaacgttaagaatctatacgaaaaagtgagatcgcaattgaaaaataacgctaaagagatagggaatgggt |
| | gtttcgaattctatcataaatgcgataatacatgtatggaatccgttaaaaacggaacatacgattaccctaagtatagcgaagag |
| | gctaaactgaatagggaagagatagacggagtggaacttgaatcaactaggatttatcagatactcgcaatttatagtacggttg |
| | ccagttcattggtactggtagtctccctgggggcaatcagtttctggatgtgctctaatgggtctctacagtgtagaatatgtatttaa |
| H1N1 NA | ATGAATCCAAACCAAAAGATAATAACCATTGGTTCGGTCTGTATGACAAT |
| A/California/ | TGGAATGGCTaacttaatattacaaattggaaacataatctcaatatggattagccactcaatccaattggggaatcaga |
| 07/2009 | atcaaatcgaaacatgcaatcaatccgtaattacatacgagaataatacttgggtgaatcagacatacgttaacatatcgaatacta |
| M100/V6 | atttcactgccggacaatccgtcgtgagtgtgaaactagccggtaatagtagtctatgtcccgttagcggatgggctatatactcta |
| SEQ ID | aagacaatagcgttagaatcggatctaaaggcgacgtattcgttatacgcgaaccattcataagttgtagtccattagagtgtagg |
| NO: 6 | actttttttctgacacagggcgcactattgaacgataagcattctaacggtacaatcaaagataggtcaccatatagaacactaatg |
| | tcatgtccgataggcgaagtgcctagtccatacaatagtagattcgaatccgtcgcttggtccgctagcgcatgccatgacggta |
| | ttaattggttgacaatcgggattagcggacccgataacggcgcagtcgccgtacttaagtataacggtataattaccgatactatt |
| | aagagttggcgaaataatatattgcgaacacaggaatccgaatgcgcatgcgttaacggatcatgttttaccgttatgactgacg |
| | gaccatctaacgggcaagcgtcatataagatttttagaatcgaaaaaggtaagatagtgaaatccgtcgaaatgaacgctcctaa |
| | ttatcattacgaagagtgctcatgttatcccgattctagcgaaattacatgcgtatgtagagacaattggcacggatctaatagacc |
| | ttgggtgtcattcaatcagaatctagagtatcaaatcgggtatatatgctcagggatattcggagacaatcctagacctaacgataa |
| | gacagggtcatgcggaccagtgagttctaacggcgctaacggcgttaaggggtttagtttcaaatacggtaacggcgtatggat |
| | agggagaactaagtcaatctctagtagaaacggattcgaaatgatatgggaccctaacggatggaccggaaccgataataattt |
| | ttcgattaaacaggatatcgtagggattaacgaatggtcagggtatagcggatcattcgtacagcatccagagttaaccggactc |
| | gattgcatacgaccatgtttttgggtcgaactgattagggggagaccgaaagagaatactatatggactagcgggagcagcata |
| | tccttttgtggtgtaaacagtgacactgtgggttggtcttggccagacggtgctgagttgccatttaccattgacaagtaa |
| H3N2 HA | ATGAAGACTATCATTGCTTTGAGCTACATTCTATGTCTGGTTTTCGCTCAAA |
| A/Texas/50/ | AACTTCCTGGAAATGACAATAGCACGGCAACGCTGTGCCTTGGGCACCAT |
| 2012 | GCAGTACCGAACGGAACTATCGTTAAGACTATTACTAACGATAGAATCGA |
| SEQ ID | AGTGACTAACGCTACCGAATTGGTGCAAAATTCTTCTATCGGCGAAATTTG |
| NO: 7 | CGATAGCCCACACCAAATACTTGACGGAGAGAATTGTACACTGATAGACG |
| | CACTGTTAGGCGATCCACAATGCGACGGATTCCAAAATAAAAAATGGGAC |
| | CTATTCGTTGAGCGATCTAAAGCATACTCTAATTGTTATCCATACGACGTA |
| | CCCGATTACGCTAGTCTTAGATCACTGGTTGCGTCAAGCGGAACACTCGAA |
| | TTTAATAACGAATCATTCAATTGGAACGGCGTAACGCAAAACGGAACAAG |
| | TAGCGCATGTATTAGACGGTCTAATAATAGCTTCTTCAGCAGACTGAATTG |
| | GTTGACACATCTGAATTTCAAATATCCCGCTCTTAACGTTACTATGCCTAA |
| | TAACGAACAATTCGATAAACTGTATATATGGGGCGTACACCATCCCGGAA |
| | CTGACAAAGACCAAATCTTTCTATACGCTCAACCTAGCGGTAGGATTACCG |
| | TTTCTACTAAACGATCACAGCAAGCCGTTATACCGAATATCGGATCAAGA |
| | CCTAGAATACGGAATATACCGTCACGAATTAGCATATATTGGACAATCGTT |
| | AAACCAGGAGACATACTGTTAATCAATAGTACAGGGAATCTAATCGCACC |
| | AAGGGGGTATTTCAAAATTAGATCCGGTAAGAGTTCAATTATGAGATCCG |
| | ACGCACCAATCGGCAAGTGTAAATCCGAATGCATAACACCTAACGGATCA |
| | ATCCCAAACGATAAACCGTTTCAGAATGTGAATAGGATTACATACGGCGC |
| | CTGCCCTAGATACGTTAAGCAATCTACACTTAAACTAGCTACCGGTATGCG |
| | AAACGTACCCGAAAAACAGACTAGGGGGATATTCGGAGCGATAGCCGGA |
| | TTCATAGAGAATGGGTGGGAGGGAATGGTAGACGGATGGTACGGATTCAG |
| | ACACCAAAATAGCGAAGGTAGGGGGCAAGCCGCAGACCTTAAATCAACG |
| | CAAGCCGCAATCGATCAGATTAACGGCAAGTTGAATAGGTTGATCGGCAA |
| | GACTAACGAAAAATTTCACCAAATCGAAAAAGAGTTTAGCGAAGTGGAGG |
| | GTAGGATACAGGATCTCGAAAAATACGTTGAGGACACTAAAATCGATCTA |
| | TGGTCATACAATGCCGAACTGTTAGTCGCACTTGAGAATCAGCATACAATC |
| | GATCTAACCGATAGCGAAATGAATAAGTTGTTCGAAAAAACTAAAAAACA |
| | ATTGAGAGAGAACGCTGAGGATATGGGGAATGGGTGTTTTAAGATATACC |
| | ATAAATGCGATAACGCTTGCATAGGGTCAATTAGAAACGGAACATACGAT |
| | CACGACGTATATAGAGACGAAGCACTGAATAATAGATTCCAAATCAAAGG |
| | CGTAGAGCTTAAAAGCGGATATAAGGATTGGATATTGTGGATATCCTTTGC |
| | CATATCATGTTTTTTGCTTTGTGTTGCTTTGTTGGGGTTCATCATGTGGGCC |
| | TGCCAAAAGGGCAACATTAGGTGCAACATTTGCATTTGA |
| H3N2 NA | ATGAATCCAAATCAAAAGATAATAACGATTGGCTCTGTTTCTCTCACCATT |
| A/Texas/50/ | TCCACAATATGCTTCTTCATGCAAATTGCCATCTTGATAACTACTGTAACA |
| 2012 | TTGCATTTCAAACAATACGAGTTCAATAGCCCCCCTAACAATCAGGTTATG |
| SEQ ID | TTGTGCGAACCTACTATAATCGAAAGAAATATTACCGAAATCGTTTATCTG |
| NO: 8 | ACTAACACTACTATCGAAAAAGAAATATGCCCTAAACCCGCTGAGTATAG |
| | GAATTGGTCAAAACCGCAATGCGGAATAACCGGATTCGCTCCATTTAGTA |
| | AGGATAACTCAATTAGGTTGTCCGCCGGAGGAGATATATGGGTTACACGC |
| | GAACCATATGTGAGTTGCGATCCCGATAAGTGTTATCAATTCGCACTAGGT |
| | CAGGGTACGACACTTAATAACGTACATTCCAATAATACCGTACACGATAG |
| | AACCCCTTATAGGACACTATTGATGAACGAACTCGGAGTGCCATTCCATCT |
| | AGGGACTAAGCAAGTGTGTATCGCATGGTCAAGCTCTAGTTGCCATGACG |
| | GAAAGGCATGGTTGCATGTGTGTATTACCGGTGACGATAAAAACGCTACC |
| | GCAAGCTTTATATATAACGGGAGGCTTGTCGATAGCGTAGTGAGTTGGTCT |
| | AAAGAGATACTTAGAACACAGGAATCCGAATGCGTATGCATAAACGGTAC |
| | ATGTACAGTCGTTATGACCGACGGTTCCGCTTCCGGAAAAGCCGATACTA |
| | AGATACTGTTTATCGAAGAGGGGAAAATAGTGCATACAAGTACACTTAGC |
| | GGCTCCGCTCAACATGTCGAAGAGTGTTCATGTTATCCTCGGTATCCCGGA |
| | GTGAGATGCGTTTGTAGGGATAATTGGAAAGGGTCAAATAGACCTATAGT |
| | CGATATTAACATAAAGGACCATTCAATCGTTAGCTCATACGTTTGTTCCGG |
| | ACTGGTAGGCGATACACCTAGAAAAAACGATAGCTCTAGCTCAAGTCATT |
| | GCTTAGACCCTAATAACGAAGAGGGAGGGCATGGCGTTAAGGGATGGGC |
| | ATTCGACGACGGAAACGACGTTTGGATGGGTAGAACGATTAACGAAACAT |
| | CACGATTGGGGTATGAGACATTCAAAGTGATAGAGGGATGGTCTAACCCT |
| | AAATCGAAATTGCAAATTAATAGGCAGGTGATAGTCGATAGAGGCGATAG |
| | ATCCGGATATTCCGGTATTTTTTCCGTTGAGGGCAAATCATGTATTAATCG |
| | GTGTTTTTATGTCGAACTGATTAGGGGTAGGAAAGAGGAAACCGAAGTGT |
| | TGTGGACTTCAAACAGTATTGTTGTGTTTTGTGGCACCTCAGGTACATATG |
| | GAACAGGCTCATGGCCTGATGGGGCGGACCTCAATCTCATGCCTATATAA |
| MP4 | AGCAAAAGCAGGTAGATATTTAAAGATGAGTCTTCTAACCGAGGTCGAAA |
| (SEQ ID | CGTACGTTCTTTCTATCATCCCGTCAGGCCCCCTCAAAGCCGAGATCGCGC |
| NO: 9) | AGAGACTGGAAAGTGTCTTTGCAGGAAAGAACACAGATCTTGAGGCTCTC |
| | ATGGAATGGCTAAAGACAAGACCAATCTTGTCACCTCTGACTAAGGGAAT |
| | TTTAGGATTTGTGTTCACGCTCACCGTGCCCAGTGAGCGAGGACTGCAGCG |
| | TAGACGCTTTGTCCAAAATGCCCTAAATGGGAATGGGGACCCGAACAACA |
| | TGGATAGAGCAGTTAAACTATACAAGAAGCTCAAAAGAGAAATAACGTTC |
| | CATGGGGCCAAGGAGGTGTCACTAAGCTATTCAACTGGTGCACTTGCCAG |
| | TTGCATGGGCCTCATATACAACAGGATGGGAACAGTGACCACAGAAGCTG |
| | CTTTTGGTCTAGTGTGTGCCACTTGTGAACAGATTGCTGATTCACAGCATC |
| | GGTCTCACAGACAGATGGCTACTACCACCAATCCACTAATCAGGCATGAA |
| | AACAGAATGGTGCTGGCTAGCACTACGGCAAAGGCTATGGAACAGATGGC |
| | TGGATCGAGTGAACAGGCAGCGGAGGCCATGGAGGTTGCTAATCAGACTA |
| | GGCAGATGGTACATGCAATGAGAACTATTGGGACTCATCCTAGCTCCAGT |
| | GCTGGTCTGAAAGATGACCTTCTTGAAAATTTGCAGGCCTACCAGAAGCG |
| | AATGGGAGTGCAGATGCAGCGATTCAAGTGATCCTCTCGTCATTGCAGCA |
| | AATATCATTGGGATCTTGCACCTGATATTGTGGATTACTGATCGTCTTTTTT |
| | TCAAATGTATTTATCGTCGCTTTAAATACGGTTTGAAAAGAGGGCCTTCTA |
| | CGGAAGGAGTGCCTGAGTCCATGAGGGAAGAATATCAACAGGAACAGCA |
| | GAGTGCTGTGGATGTTGACGATGGTCATTTTGTCAACATAGAGCTAGAGTA |
| | AAAAACTACCTTGTTTCTACT |
| PB2 P4 | AGCGAAAGCAGGTCAAATATATTCAATATGGAGAGAATAAAAGAACTGA |
| (SEQ ID | GAGATCTAATGTCGCAGTCCCGCACTCGCGAGATACTCACTAAGACCACT |
| NO: 10) | GTGGACCATATGGCCATAATCAAAAAGTACACATCAGGAAGGCAAGAGA |
| | AGAACCCCGCACTCAGAATGAAGTGGATGATGGCAATGAGATACCCAATT |
| | ACAGCAGACAAGAGAATAATGGACATGATTCCAGAGAGGAATGAACAAG |
| | GACAAACCCTCTGGAGCAAAACAAACGATGCTGGATCAGACCGAGTGATG |
| | GTATCACCTCTGGCCGTAACATGGTGGAATAGGAATGGCCCAACAACAAG |
| | TACAGTTCATTACCCTAAGGTATATAAAACTTATTTCGAAAAGGTCGAAAG |
| | GTTGAAACATGGTACCTTCGGCCCTGTCCACTTCAGAAATCAAGTTAAAAT |
| | AAGGAGGAGAGTTGATACAAACCCTGGCCATGCAGATCTCAGTGCCAAGG |
| | AGGCACAGGATGTGATTATGGAAGTTGTTTTCCCAAATGAAGTGGGGGCA |
| | AGAATACTGACATCAGAGTCACAGCTGGCAATAACAAAAGAGAAGAAAG |
| | AAGAGCTCCAGGATTGTAAAATTGCTCCCTTGATGGTGGCGTACATGCTAG |
| | AAAGAGAATTGGTCCGTAAAACAAGGTTTCTCCCAGTAGCCGGCGGAACA |
| | GGCAGTGTTTATATTGAAGTGTTGCACTTAACCCAAGGGACGTGCTGGGA |
| | GCAGATGTACACTCCAGGAGGAGAAGTGAGAAATGATGATGTTGACCAAA |
| | GTTTGATTATCGCTGCTAGAAACATAGTAAGAAGAGCAGCAGTGTCAGCA |
| | GACCCATTAGCATCTCTCTTGGAAATGTGCCACAGCACACAGATTGGAGG |
| | AGTAAGGATGGTGGACATCCTTAGACAGAATCCAACTGAGGAACAAGCCG |
| | TAGACATATGCAAGGCAGCAATAGGGTTGAGGATTAGCTCATCTTTCAGTT |
| | TTGGTGGGTTCACTTTCAAAAGGACAAGCGGATCATCAGTCAAGAAAGAA |
| | GAAGAAGTGCTAACGGGCAACCTCCAAACACTGAAAATAAGAGTACATG |
| | AAGGGTATGAAGAATTCACAATGGTTGGGAGAAGAGCAACAGCTATTCTC |
| | AGAAAGGCAACCAGGAGATTGATCCAGTTGATAGTAAGCGGGAGAGACG |
| | AGCAGTCAATTGCTGAGGCAATAATTGTGGCCATGGTATTCTCACAGGAG |
| | GATTGCATGATCAAGGCAGTTAGGGGCGATCTGAACTTTGTCAATAGGGC |
| | AAACCAGCGACTGAACCCCATGCACCAACTCTTGAGGCATTTCCAAAAAG |
| | ATGCAAAAGTGCTTTTCCAGAACTGGGGAATTGAATCCATCGACAATGTG |
| | ATGGGAATGATCGGAATACTGCCCGACATGACCCCAAGCACGGAGATGTC |
| | GCTGAGAGGGATAAGAGTCAGCAAAATGGGAGTAGATGAATACTCCAGC |
| | ACGGAGAGAGTGGTAGTGAGTATTGACCGATTTTTAAGGGTTAGAGATCA |
| | AAGAGGGAACGTACTATTGTCTCCCGAAGAAGTCAGTGAAACGCAAGGAA |
| | CTGAGAAGTTGACAATAACTTATTCGTCATCAATGATGTGGGAGATCAAT |
| | GGCCCTGAGTCAGTGCTAGTCAACACTTATCAATGGATAATCAGGAACTG |
| | GGAAATTGTGAAAATTCAATGGTCACAAGATCCCACAATGTTATACAACA |
| | AAATGGAATTTGAACCATTTCAGTCTCTTGTCCCTAAGGCAACCAGAAGCC |
| | GGTACAGTGGATTCGTAAGGACACTGTTCCAGCAAATGCGGGATGTGCTT |
| | GGGACATTTGACACTGTCCAAATAATAAAACTTCTCCCCTTTGCTGCTGCC |
| | CCACCAGAACAGAGTAGGATGCAATTTTCCTCATTGACTGTGAATGTGAG |
| | AGGATCAGGGTTGAGGATACTGGTAAGAGGCAATTCTCCAGTATTCAATT |
| | ACAACAAGGCAACCAAACGACTTACAGTTCTTGGAAAGGATGCAGGTGCA |
| | TTGACTGAAGATCCAGATGAAGGCACATCTGGGGTGGAGTCTGCTGTCCT |
| | GAGAGGATTTCTCATTTTGGGCAAAGAAGACAAGAGATATGGCCCAGCAT |
| | TAAGCATCAATGAACTGAGCAATCTTGCAAAAGGAGAGAAGGCTAATGTG |
| | CTAATTGGGCAAGGGGACGTAGTGTTGGTAATGAAACGAAAACGGGACTC |
| | TAGCATACTTACTGACAGCCAGACAGCGACCAAAAGAATTCGGATGGCCA |
| | TCAATTAGTGTCGAATTGTTTAAAAACGACCTTGTTTCTACT |
| PB1 P4 | AGCGAAAGCAGGCAAACCATTTGAATGGATGTCAATCCGACTCTACTTTTC |
| (SEQ ID | CTAAAAATTCCAGCGCAAAATGCCATAAGCACCACATTCCCTTATACTGG |
| NO: 11) | AGATCCTCCATACAGCCATGGAACAGGAACAGGATACACCATGGACACAG |
| | TAAACAGAACACACCAATACTCAGAAAAGGGAAAGTGGACGACAAACAC |
| | AGAGACTGGTGCACCCCAGCTCAACCCGATTGATGGACCACTACCTGAGG |
| | ATAATGAACCAAGTGGGTATGCACAAACAGACTGTGTTCTAGAGGCTATG |
| | GCTTTCCTTGAAGAATCCCACCCAGGAATATTTGAGAATTCATGCCTTGAA |
| | ACAATGGAAGTTGTTCAACAAACAAGGGTAGATAAACTAACTCAAGGTCG |
| | CCAGACTTATGATTGGACATTAAACAGAAATCAACCGGCAGCAACTGCAT |
| | TGGCCAACACCATAGAAGTCTTTAGATCGAATGGCCTAACAGCTAATGAG |
| | TCAGGAAGGCTAATAGATTTCTTAAAGGATGTAATGGAATCAATGAACAA |
| | AGAGGAAATAGAGATAACAACCCACTTTCAAAGAAAAAGGAGAGTAAGA |
| | GACAACATGACCAAGAAGATGGTCACGCAAAGAACAATAGGGAAGAAAA |
| | AACAAAGACTGAATAAGAGAGGCTATCTAATAAGAGCACTGACATTAAAT |
| | ACGATGACCAAAGATGCAGAGAGAGGCAAGTTAAAAAGAAGGGCTATCG |
| | CAACACCTGGGATGCAGATTAGAGGTTTCGTATACTTTGTTGAAACTTTAG |
| | CTAGGAGCATTTGCGAAAAGCTTGAACAGTCTGGGCTCCCAGTAGGGGGC |
| | AATGAAAAGAAGGCCAAACTGGCAAATGTTGTGAGAAAGATGATGACTA |
| | ATTCACAAGACACAGAGATTTCTTTCACAATCACTGGGGACAACACTAAG |
| | TGGAATGAAAATCAAAATCCTCGAATGTTCCTGGCGATGATTACATATATC |
| | ACCAGAAATCAACCCGAGTGGTTCAGAAACATCCTGAGCATGGCACCCAT |
| | AATGTTCTCAAACAAAATGGCAAGACTAGGGAAAGGGTACATGTTCGAGA |
| | GTAAAAGAATGAAGATTCGAACACAAATACCAGCAGAAATGCTAGCAAG |
| | CATTGACCTGAAGTACTTCAATGAATCAACAAAGAAGAAAATTGAGAAAA |
| | TAAGGCCTCTTCTAATAGATGGCACAGCATCACTGAGTCCTGGGATGATG |
| | ATGGGCATGTTCAACATGCTAAGTACGGTCTTGGGAGTCTCGATACTGAAT |
| | CTTGGACAAAAGAAATACACCAAGACAATATACTGGTGGGATGGGCTCCA |
| | ATCATCCGACGATTTTGCTCTCATAGTGAATGCACCAAACCATGAGGGAAT |
| | ACAAGCAGGAGTGGACAGATTCTACAGGACCTGCAAGTTAGTGGGAATCA |
| | ACATGAGCAAAAAGAAGTCCTATATAAATAAGACAGGGACATTTGAATTC |
| | ACAAGCTTTTTTTATCGCTATGGATTTGTGGCTAATTTTAGCATGGAGCTAC |
| | CCAGCTTTGGAGTGTCTGGAGTAAATGAATCAGCTGACATGAGTATTGGA |
| | GTAACAGTGATAAAGAACAACATGATAAACAATGACCTTGGACCTGCAAC |
| | GGCCCAGATGGCTCTTCAATTGTTCATCAAAGACTACAGATACACATATAG |
| | GTGCCATAGGGGAGACACACAAATTCAGACGAGAAGATCATTTGAGTTAA |
| | AGAAGCTGTGGGATCAAACCCAATCAAAAGTAGGGCTATTAGTATCAGAT |
| | GGAGGACCAAACTTATACAATATACGGAATCTTCACATTCCTGAAGTCTGC |
| | TTAAAATGGGAGCTAATGGATGATGATTATCGGGGAAGACTTTGTAATCC |
| | CCTGAATCCCTTTGTCAGTCATAAAGAGATTGATTCTGTAAACAATGCTGT |
| | GGTAATGCCAGCCCATGGTCCAGCCAAAAGCATGGAATATGATGCCGTTG |
| | CAACTACACATTCCTGGATTCCCAAGAGGAATCGTTCTATTCTCAACACAA |
| | GCCAAAGGGGAATTCTTGAGGATGAACAGATGTACCAGAAGTGCTGCAAT |
| | CTATTCGAGAAATTTTTCCCTAGCAGTTCATATAGGAGACCGGTTGGAATT |
| | TCTAGCATGGTGGAGGCCATGGTGTCTAGGGCCCGGATTGATGCCAGGGT |
| | CGACTTCGAGTCTGGACGGATCAAGAAAGAAGAGTTCTCTGAGATCATGA |
| | AGATCTGTTCCACCATTGAAGAACTCAGACGGCAAAAATAATGAATTTAA |
| | CTTGTCCTTCATGAAAAAATGCCTTGTTTCTACT |
| PA P4 | AGCGAAAGCAGGTACTGATCCAAAATGGAAGACTTTGTGCGACAATGCTT |
| (SEQ ID | CAATCCAATGATCGTCGAGCTTGCGGAAAAGGCAATGAAAGAATATGGGG |
| NO: 12) | AAGATCCGAAAATCGAAACTAACAAGTTTGCTGCAATATGCACACATTTG |
| | GAAGTTTGTTTCATGTATTCGGATTTCCATTTCATCGACGAACGGGGTGAA |
| | TCAATAATTGTAGAATCTGGTGACCCGAATGCACTATTGAAGCACCGATTT |
| | GAGATAATTGAAGGAAGAGACCGAATCATGGCCTGGACAGTGGTGAACA |
| | GTATATGTAACACAACAGGGGTAGAGAAGCCTAAATTTCTTCCTGATTTGT |
| | ATGATTACAAAGAGAACCGGTTCATTGAAATTGGAGTAACACGGAGGGAA |
| | GTCCACATATATTACCTAGAGAAAGCCAACAAAATAAAATCTGAGAAGAC |
| | ACACATTCACATCTTTTCATTCACTGGAGAGGAGATGGCCACCAAAGCGG |
| | ACTACACCCTTGACGAAGAGAGCAGGGCAAGAATCAAAACTAGGCTTTTC |
| | ACTATAAGACAAGAAATGGCCAGTAGGAGTCTATGGGATTCCTTTCGTCA |
| | GTCCGAAAGAGGCGAAGAGACAATTGAAGAAAAATTTGAGATTACAGGA |
| | ACTATGCGCAAGCTTGCCGACCAAAGTCTCCCACCGAACTTCCCCAGCCTT |
| | GAAAACTTTAGAGCCTATGTAGATGGATTCGAGCCGAACGGCTGCATTGA |
| | GGGCAAGCTTTCCCAAATGTCAAAAGAAGTGAACGCCAAAATTGAACCAT |
| | TCTTGAGGACGACACCACGCCCCCTCAGATTGCCTGATGGGCCTCTTTGCC |
| | ATCAGCGGTCAAAGTTCCTGCTGATGGATGCTCTGAAATTAAGTATTGAAG |
| | ACCCGAGTCACGAGGGGGAGGGAATACCACTATATGATGCAATCAAATGC |
| | ATGAAGACATTCTTTGGCTGGAAAGAGCCTAACATAGTCAAACCACATGA |
| | GAAAGGCATAAATCCCAATTACCTCATGGCTTGGAAGCAGGTGCTAGCAG |
| | AGCTACAGGACATTGAAAATGAAGAGAAGATCCCAAGGACAAAGAACAT |
| | GAAGAGAACAAGCCAATTGAAGTGGGCACTCGGTGAAAATATGGCACCA |
| | GAAAAAGTAGACTTTGATGACTGCAAAGATGTTGGAGACCTTAAACAGTA |
| | TGACAGTGATGAGCCAGAGCCCAGATCTCTAGCAAGCTGGGTCCAAAATG |
| | AATTCAATAAGGCATGTGAATTGACTGATTCAAGCTGGATAGAACTTGAT |
| | GAAATAGGAGAAGATGTTGCCCCGATTGAACATATCGCAAGCATGAGGAG |
| | GAACTATTTTACAGCAGAAGTGTCCCACTGCAGGGCTACTGAATACATAA |
| | TGAAGGGAGTGTACATAAATACGGCCTTGCTCAATGCATCCTGTGCAGCC |
| | ATGGATGACTTTCAGCTGATCCCAATGATAAGCAAATGTAGGACCAAAGA |
| | AGGAAGACGGAAAACAAACCTGTATGGGTTCATTATAAAAGGAAGGTCTC |
| | ATTTGAGAAATGATACTGATGTGGTGAACTTTGTAAGTATGGAGTTCTCAC |
| | TCACTGACCCGAGACTGGAGCCACACAAATGGGAAAAATACTGTGTTCTT |
| | GAAATAGGAGACATGCTCTTGAGGACTGCGATAGGCCAAGTGTCGAGGCC |
| | CATGTTCCTATATGTGAGAACCAATGGAACCTCCAAGATCAAGATGAAAT |
| | GGGGCATGGAAATGAGGCGCTGCCTTCTTCAGTCTCTTCAGCAGATTGAG |
| | AGCATGATTGAGGCCGAGTCTTCTGTCAAAGAGAAAGACATGACCAAGGA |
| | ATTCTTTGAAAACAAATCGGAAACATGGCCAATCGGAGAGTCACCCAGGG |
| | GAGTGGAGGAAGGCTCTATTGGGAAAGTGTGCAGGACCTTACTGGCAAAA |
| | TCTGTATTCAACAGTCTATATGCGTCTCCACAACTTGAGGGGTTTTCGGCT |
| | GAATCTAGAAAATTGCTTCTCATTGTTCAGGCACTTAGGGACAACCTGGAA |
| | CCTGGAACCTTCGATCTTGGGGGGCTATATGAAGCAATCGAGGAGTGCCT |
| | GATTAATGATCCCTGGGTTTTGCTTAATGCATCTTGGTTCAACTCCTTCCTC |
| | ACACATGCACTGAAGTAGTTGTGGCAATGCTACTATTTGCTATCCATACTG |
| | TCCAAAAAAGTACCTTGTTTCTACT |
| NS P4 | AGCAAAAGCAGGGTGACAAAAACATAATGGACTCCAACACCATGTCAAG |
| (SEQ ID | CTTTCAGGTAGACTGTTTCCTTTGGCATATCCGCAAGCGATTTGCAGACAA |
| NO: 13) | TGGATTGGGTGATGCCCCATTCCTTGATCGGCTCCGCCGAGATCAAAAGTC |
| | CTTAAAAGGAAGAGGCAACACCCTTGGCCTCGATATCGAAACAGCCACTC |
| | TTGTTGGGAAACAAATCGTGGAATGGATCTTGAAAGAGGAATCCAGCGAG |
| | ACACTTAGAATGACAATTGCATCTGTACCTACTTCGCGGTACCTTTCTGAC |
| | ATGACCCTCGAGGAAATGTCACGAGACTGGTTCATGCTCATGCCTAGGCA |
| | AAAGATAATAGGCCCTCTTTGCGTGCGATTGGACCAGGCGATCATGGAAA |
| | AGAACATAGTACTGAAAGCGAACTTCAGTGTAATCTTTAACCGATTAGAG |
| | ACCTTGATACTACTAAGGGCTTTCACTGAGGAGGGAGCAATAGTTGGAGA |
| | AATTTCACCATTACCTTCTCTTCCAGGACATACTTATGAGGATGTCAAAAA |
| | TGCAGTTGGGGTCCTCATCGGAGGACTTGAATGGAATGGTAACACGGTTC |
| | GAGTCTCTGAAAATATACAGAGATTCGCTTGGAGAAACTGTGATGAGAAT |
| | GGGAGACCTTCACTACCTCCAGAGCAGAAATGAAAAGTGGCGAGAGCAAT |
| | TGGGACAGAAATTTGAGGAAATAAGGTGGTTAATTGAAGAAATGCGGCAC |
| | AGATTGAAAGCGACAGAGAATAGTTTCGAACAAATAACATTTATGCAAGC |
| | CTTACAACTACTGCTTGAAGTAGAACAAGAGATAAGAGCTTTCTCGTTTCA |
| | GCTTATTTAATGATAAAAAACACCCTTGTTTCTACT |
| NP P4 | AGCAAAAGCAGGGTAGATAATCACTCAATGAGTGACATCGAAGCCATGGC |
| (SEQ ID | GTCTCAAGGCACCAAACGATCATATGAACAAATGGAGACTGGTGGGGAGC |
| NO: 14) | GCCAGGATGCCACAGAAATCAGAGCATCTGTCGGAAGAATGATTGGTGGA |
| | ATCGGGAGATTCTACATCCAAATGTGCACTGAACTCAAACTCAGTGATTAT |
| | GATGGACGACTAATCCAGAATAGCATAACAATAGAGAGGATGGTGCTTTC |
| | TGCTTTTGATGAGAGAAGAAATAAATACCTAGAAGAGCATCCCAGTGCTG |
| | GGAAGGACCCTAAGAAAACAGGAGGACCCATATATAGAAGAGTAGACGG |
| | AAAGTGGATGAGAGAACTCATCCTTTATGACAAAGAAGAAATAAGGAGA |
| | GTTTGGCGCCAAGCAAACAATGGCGAAGATGCAACAGCAGGTCTTACTCA |
| | TATCATGATTTGGCATTCCAACCTGAATGATGCCACATATCAGAGAACAA |
| | GAGCGCTTGTTCGCACCGGAATGGATCCCAGAATGTGCTCTCTAATGCAA |
| | GGTTCAACACTTCCCAGAAGGTCTGGTGCCGCAGGTGCTGCGGTGAAAGG |
| | AGTTGGAACAATAGCAATGGAGTTAATCAGAATGATCAAACGTGGAATCA |
| | ATGACCGAAATTTCTGGAGGGGTGAAAATGGACGAAGGACAAGGGTTGCT |
| | TATGAAAGAATGTGCAATATCCTCAAAGGAAAATTTCAAACAGCTGCCCA |
| | GAGGGCAATGATGGATCAAGTAAGAGAAAGTCGAAACCCAGGAAACGCT |
| | GAGATTGAAGACCTCATTTTCCTGGCACGGTCAGCACTCATTCTGAGGGGA |
| | TCAGTTGCACATAAATCCTGCCTGCCTGCTTGTGTGTATGGGCTTGCAGTA |
| | GCAAGTGGGCATGACTTTGAAAGGGAAGGGTACTCACTGGTCGGGATAGA |
| | CCCATTCAAATTACTCCAAAACAGCCAAGTGGTCAGCCTGATGAGACCAA |
| | ATGAAAACCCAGCTCACAAGAGTCAATTGGTGTGGATGGCATGCCACTCT |
| | GCTGCATTTGAAGATTTAAGAGTATCAAGTTTCATAAGAGGAAAGAAAGT |
| | GATTCCAAGAGGAAAGCTTTCCACAAGAGGGGTCCAGATTGCTTCAAATG |
| | AGAATGTGGAAACCATGGACTCCAATACCCTGGAACTGAGAAGCAGATAC |
| | TGGGCCATAAGGACCAGGAGTGGAGGAAATACCAATCAACAAAAGGCAT |
| | CCGCAGGCCAGATCAGTGTGCAGCCTACATTCTCAGTGCAGCGGAATCTC |
| | CCTTTTGAAAGAGCAACCGTTATGGCAGCATTCAGCGGGAACAATGAAGG |
| | ACGGACATCCGACATGCGAACAGAAGTTATAAGAATGATGGAAAGTGCA |
| | AAGCCAGAAGATTTGTCCTTCCAGGGGGGGGGAGTCTTCGAGCTCTCGGA |
| | CGAAAAGGCAACGAACCCGATCGTGCCTTCCTTTGACATGAGTAATGAAG |
| | GGTCTTATTTCTTCGGAGACAATGCAGAGGAGTATGACAGTTGAGGAAAA |
| | ATACCCTTGTTTCTACT |
| HA P6 | AGCAAAAGCAGGGGAAAATAAAAGCAACAAAAATGAAGGCAATACTAGT |
| (SEQ ID | AGTTCTGCTATATACATTTGCAACCGCAAATGCAGACACATTATGTATAGG |
| NO: 15) | TTATCATGCGAACAATTCAACAGACACTGTAGACACAGTACTAGAAAAGA |
| | ATGTAACAGTAACACACTCTGTTAACCTATTGGAGGATAAGCATAACGGT |
| | AAGCTATGCAAACTGAGAGGCGTAGCACCATTGCATCTAGGTAAGTGTAA |
| | TATAGCCGGATGGATTCTAGGGAATCCCGAATGCGAATCACTATCAACCG |
| | CTAGCTCATGGTCATACATAGTCGAAACACCATCAAGCGATAACGGTACA |
| | TGTTATCCCGGAGACTTTATCGATTACGAAGAGCTTAGAGAGCAATTGTCT |
| | AGCGTAAGCTCATTCGAAAGATTCGAAATTTTTCCGAAAACTAGCTCATGG |
| | CCTAATCACGATAGTAATAAAGGCGTAACTGCCGCATGCCCACACGCCGG |
| | AGCTAAATCATTCTATAAGAATCTGATTTGGTTAGTGAAAAAAGGGAATT |
| | CATATCCGAAACTATCTAAATCATACATTAACGATAAGGGTAAGGAGGTA |
| | CTAGTGTTGTGGGGGATACACCATCCATCAACTAGCGCCGATCAGCAATC |
| | ATTGTATCAGAACGCAGACGCATACGTATTCGTAGGGTCTAGTAGATACTC |
| | TAAAAAATTTAAACCCGAAATCGCAATTAGACCGAAAGTGAGAGACCAAG |
| | AGGGTAGAATGAATTACTATTGGACACTAGTCGAACCAGGCGATAAGATT |
| | ACATTCGAAGCGACAGGGAATCTAGTCGTACCGAGATACGCATTCGCAAT |
| | GGAGAGAAACGCCGGATCCGGAATTATTATTAGCGATACACCCGTACACG |
| | ATTGCAATACTACATGTCAGACACCAAAAGGCGCAATTAATACTAGTCTG |
| | CCATTTCAGAATATACACCCAATTACAATCGGTAAGTGTCCAAAATACGTT |
| | AAGTCAACTAAGTTGAGACTCGCAACAGGGTTGAGAAATATACCGTCAAT |
| | TCAATCTAGGGGGTTGTTCGGAGCAATCGCAGGGTTTATCGAAGGGGGGT |
| | GGACAGGTATGGTTGACGGATGGTACGGATACCATCATCAAAACGAACAG |
| | GGATCCGGATACGCAGCCGATCTGAAAAGTACACAGAACGYTATAGACGA |
| | AATTACGAATAAAGTGAATAGCGTAATCGAAAAAATGAATACGCAATTTA |
| | CAGCCGTAGGTAAGGAGTTTAATCATCTCGAAAAAAGGATTGAGAATCTG |
| | AATAAAAAAGTCGACGACGGATTCTTAGACATTTGGACTTATAACGCCGA |
| | ACTGTTAGTGTTACTCGAAAACGAAAGAACACTAGACTATCACGATTCAA |
| | ACGTTAAGAATCTATACGAAAAAGTGAGATCGCAATTGAAAAATAACGCT |
| | AAAGAGATAGGGAATGGGTGTTTCGAATTCTATCATAAATGCGATAATAC |
| | ATGTATGGAATCCGTTAAAAACGGAACATACGATTACCCTAAGTATAGCG |
| | AAGAGGCTAAACTGAATAGGGAAGAGATAGACGGAGTGAAACTCGAATC |
| | AACTAGGATTTATCAGATACTCGCAATTTATAGTACGGTTGCCAGTTCATT |
| | GGTACTGGTAGTCTCCCTGGGGGCAATCAGTTTCTGGATGTGCTCTAATGG |
| | GTCTCTACAGTGTAGAATATGTATTTAACATTAGGATTTCAGAAGCATGAG |
| | AAAAACACCCTTGTTTCTACT |
| | Y is C or T. |
| HA P7 | AGCAAAAGCAGGGGAAAATAAAAGCAACAAAAATGAAGGCAATACTAGT |
| (SEQ ID | AGTTCTGCTATATACATTTGCAACCGCAAATGCAGACACATTATGTATAGG |
| NO: 16) | TTATCATGCGAACAATTCAACAGACACTGTAGACACAGTACTAGAAAAGA |
| | ATGTAACAGTAACACACTCTGTTAACCTATTGGAGGATAAGCATAACGGT |
| | AAGCTATGCAAACTGAGAGGCGTAGCACCATTGCATCTAGGTAAGTGTAA |
| | TATAGCCGGATGGATTCTAGGGAATCCCGAATGCGAATCACTATCAACCG |
| | CTAGCTCATGGTCATACATAGTCGAAACACCATCAAGCGATAACGGTACA |
| | TGTTATCCCGGAGACTTTATCGATTACGAAGAGCTTAGAGAGCAATTGTCT |
| | AGCGTAAGCTCATTCGAAAGATTCGAAATTTTTCCGAAAACTAGCTCATGG |
| | CCTAATCACGATAGTAATAAAGGCGTAACTGCCGCATGCCCACACGCCGG |
| | AGCTAAATCATTCTATAAGAATCTGATTTGGTTAGTGAAAAAAGGGAATT |
| | CATATCCGAAACTATCTAAATCATACATTAACGATAAGGGTAAGGAGGTA |
| | CTAGTGTTGTGGGGGATACACCATCCATCAACTAGCGCCGATCAGCAATC |
| | ATTGTATCAGAACGCAGACGCATACGTATTCGTAGGGTCTAGTAGATACTC |
| | TAAAAAATTTAAACCCGAAATCGCAATTAGACCGAAAGTGAGAGACCAAG |
| | AGGGTAGAATGAATTACTATTGGACACTAGTCGAACCAGGCGATAAGATT |
| | ACATTCGAAGCGACAGGGAATCTAGTCGTACCGAGATACGCATTCGCAAT |
| | GGAGAGAAACGCCGGATCCGGAATTATTATTAGCGATACACCCGTACACG |
| | ATTGCAATACTACATGTCAGACACCAAAAGGCGCAATTAATACTAGTCTG |
| | CCATTTCAGAATATACACCCAATTACAATCGGTAAGTGTCCAAAATACGTT |
| | AAGTCAACTAAGTTGAGACTCGCAACAGGGTTGAGAAATATACCGTCAAT |
| | TCAATCTAGGGGGTTGTTCGGAGCAATCGCAGGGTTTATCGAAGGGGGGT |
| | GGACAGGTATGGTTGACGGATGGTACGGATACCATCATCAAAACGAACAG |
| | GGATCCGGATACGCAGCCGAWCTGAAAAGTACACAGAACGYTATAGACG |
| | AAATTACGAATAAAGTGAATAGCGTAATCGAAAAAATGAATACGCAATTT |
| | ACAGCCGTAGGTAAGGAGTTTAATCATCTCGAAAAAAGGATTGAGAATCT |
| | GAATAAAAAAGTCGACGACGGATTCTTAGACATTTGGACTTATAACGCCG |
| | AACTGTTAGTGTTACTCGAAAACGAAAGAACACTAGACTATCACGATTCA |
| | AACGTTAAGAATCTATACGAAAAAGTGAGATCGCAATTGAAAAATAACGC |
| | TAAAGAGATAGGGAATGGGTGTTTCGAATTCTATCATAAATGCGATAATA |
| | CATGTATGGAATCCGTTAAAAACGGAACATACGATTACCCTAAGTATAGC |
| | GAAGAGGCTAAACTGAATAGGGAAGAGATAGACGGAGTGAAACTCGAAT |
| | CAACTAGGATTTATCAGATACTCGCAATTTATAGTACGGTTGCCAGTTCAT |
| | TGGTACTGGTAGTCTCCCTGGGGGCAATCAGTTTCTGGATGTGCTCTAATG |
| | GGTCTCTACAGTGTAGAATATGTATTTAACATTAGGATTTCAGAAGCATGA |
| | GAAAAACACCCTTGTTTCTACT |
| | Y is C or T. W is T or A. |
| A/CA/07/200 | agcaaaagcaggggaaaataaaagcaacaaaaATGAAGGCAATACTAGTAGTTCTGCTATAT |
| 9-HA-Min- | ACATTTGCAACCGCAAATGCAGACACATTATGTATAGGTTATCATGCGAA |
| Vero17P | CAATTCAACAGACACTGTAGACACAaTACTAGAAAAGAATGTAACAGTAA |
| (SEQ ID | CACACTCTGTTAACCTATTGGAGGATAAGCATAACGGTAAGCTATGCAAA |
| NO: 17) | CTGAGAGGCGTAGCACCATTGCATCTAGGTAAGTGTAATATAGCCGGATG |
| | GATTCTAGGGAATCCCGAATGCGAATCACTATCAACCGCTAGCTCATGGTC |
| | ATACATAGTCGAAACACCATCAAGCGATAACGGTACATGTTATCCCGGAG |
| | ACTTTATCGATTACGAAGAGCTTAGAGAGCAATTGTCTAGCGTAAGCTCAT |
| | TCGAAAGATTCGAAATTTTTCCGAAAACTAGCTCATGGCCTgATCACGATA |
| | GTAATAAAGGCGTAACTGCCGCATGCCCACACGCCGGAGCTAAATCATTC |
| | TATgAGAATCTGgTTTGGTTAGTGAAAAAAGGGAATTCATATCCGAAACTA |
| | TCTAAATCATACATTAACGATAAGGGTAAGGAGGTACTAGTGTTGTGGGG |
| | GATACACCATCCATCAACTAGCGCCGATCAGCAATCATTGTATCAGAACG |
| | CAGACGCATACGTATTCGTAGGGTCTAGTAGATACTCTAAAAAATTTAAA |
| | CCCGAAATCGCAATTAGACCGAAAGTGAGAGgCCAAGAGGGTAGAATGAA |
| | TTACTATTGGACACTAGTCGAACCAGGCGATAAGATTACATTCGAAGCGA |
| | CAGGGAATCTAGTCGTACCGAGATACGCATTCGCAATGGAGAGAAACGCC |
| | GGATCCGGAATTATTATTAGCGATACACCCGTACACGATTGCAATACTACA |
| | TGTCAGACACCAgAAGGCGCAATTAATACTAGTCTGCCATTTCAGAATATA |
| | CACCCAATTACAATCGGTAAGTGTCCAAAATACGTTAAGTCAACTAAGTT |
| | GAGACTCGCAACAGGGTTGAGAAATATACCGTCAATTCAATCTAGGGGGT |
| | TGTTCGGAGCAATCGCAGGGTTTATCGAAGGGGGGTGGACAGGTATGGTT |
| | GACGGATGGTACGGATACCATCATCAAAACGAACAtGGATCCGGATACGC |
| | AGCCGAaCTGAAAAGTACACAGAACGCTATAGACGAAATTACGAATAAAG |
| | TGAATAGCGTAATCGAAAAAATGAATACGCAATTTACAGCCGTAGGTAAG |
| | GAGTTTAATCATCTCGAAAAAAGGATTGAGAATCTGAATAAAAAAGTCGA |
| | CGACGGATTCTTAGACATTTGGACTTATAACGCCGAACTGTTAGTGTTACT |
| | CGAAAACGAAAGAACACTAGACTATCACGATTCAAACGTTAAGAATCTAT |
| | ACGAAAAAGTGAGATCGCAATTGAAAAATAACGCTAAAGAGATAGGGAA |
| | TGGGTGTTTCGAATTCTATCATAAATGCGATAATACATGTATGGAATCCGT |
| | TAAAAACGGAACATACGATTACCCTAAGTATAGCGAAGAGGCTAAACTGA |
| | ATAGaGAAGAGATAGACGGAGTGgAACTCGAgTCAACTAGGATTTATCAGA |
| | TACTCGCAATTTATAGTACGGTTGCCAGTTCATTGGTACTGGTAGTCTCCCT |
| | GGGGGCAATCAGTTTCTGGATGTGCTCTAATGGGTCTCTACAGTGTAGAAT |
| | ATGTATTTAAcattaggatttcagaagcatgagaaaaacacccttgtttctact |
| A/CA/07/200 | agcaaaagcaggggaaaataaaagcaacaaaaATGAAGGCAATACTAGTAGTTCTGCTATAT |
| 9-NA-Min- | ACATTTGCAACCGCAAATGCAGACACATTATGTATAGGTTATCATGCGAA |
| Vero17P | CAATTCAACAGACACTGTAGACACAaTACTAGAAAAGAATGTAACAGTAA |
| (SEQ ID | CACACTCTGTTAACCTATTGGAGGATAAGCATAACGGTAAGCTATGCAAA |
| NO: 18) | CTGAGAGGCGTAGCACCATTGCATCTAGGTAAGTGTAATATAGCCGGATG |
| | GATTCTAGGGAATCCCGAATGCGAATCACTATCAACCGCTAGCTCATGGTC |
| | ATACATAGTCGAAACACCATCAAGCGATAACGGTACATGTTATCCCGGAG |
| | ACTTTATCGATTACGAAGAGCTTAGAGAGCAATTGTCTAGCGTAAGCTCAT |
| | TCGAAAGATTCGAAATTTTTCCGAAAACTAGCTCATGGCCTgATCACGATA |
| | GTAATAAAGGCGTAACTGCCGCATGCCCACACGCCGGAGCTAAATCATTC |
| | TATgAGAATCTGgTTTGGTTAGTGAAAAAAGGGAATTCATATCCGAAACTA |
| | TCTAAATCATACATTAACGATAAGGGTAAGGAGGTACTAGTGTTGTGGGG |
| | GATACACCATCCATCAACTAGCGCCGATCAGCAATCATTGTATCAGAACG |
| | CAGACGCATACGTATTCGTAGGGTCTAGTAGATACTCTAAAAAATTTAAA |
| | CCCGAAATCGCAATTAGACCGAAAGTGAGAGgCCAAGAGGGTAGAATGAA |
| | TTACTATTGGACACTAGTCGAACCAGGCGATAAGATTACATTCGAAGCGA |
| | CAGGGAATCTAGTCGTACCGAGATACGCATTCGCAATGGAGAGAAACGCC |
| | GGATCCGGAATTATTATTAGCGATACACCCGTACACGATTGCAATACTACA |
| | TGTCAGACACCAgAAGGCGCAATTAATACTAGTCTGCCATTTCAGAATATA |
| | CACCCAATTACAATCGGTAAGTGTCCAAAATACGTTAAGTCAACTAAGTT |
| | GAGACTCGCAACAGGGTTGAGAAATATACCGTCAATTCAATCTAGGGGGT |
| | TGTTCGGAGCAATCGCAGGGTTTATCGAAGGGGGGTGGACAGGTATGGTT |
| | GACGGATGGTACGGATACCATCATCAAAACGAACAtGGATCCGGATACGC |
| | AGCCGAaCTGAAAAGTACACAGAACGCTATAGACGAAATTACGAATAAAG |
| | TGAATAGCGTAATCGAAAAAATGAATACGCAATTTACAGCCGTAGGTAAG |
| | GAGTTTAATCATCTCGAAAAAAGGATTGAGAATCTGAATAAAAAAGTCGA |
| | CGACGGATTCTTAGACATTTGGACTTATAACGCCGAACTGTTAGTGTTACT |
| | CGAAAACGAAAGAACACTAGACTATCACGATTCAAACGTTAAGAATCTAT |
| | ACGAAAAAGTGAGATCGCAATTGAAAAATAACGCTAAAGAGATAGGGAA |
| | TGGGTGTTTCGAATTCTATCATAAATGCGATAATACATGTATGGAATCCGT |
| | TAAAAACGGAACATACGATTACCCTAAGTATAGCGAAGAGGCTAAACTGA |
| | ATAGaGAAGAGATAGACGGAGTGgAACTCGAgTCAACTAGGATTTATCAGA |
| | TACTCGCAATTTATAGTACGGTTGCCAGTTCATTGGTACTGGTAGTCTCCCT |
| | GGGGGCAATCAGTTTCTGGATGTGCTCTAATGGGTCTCTACAGTGTAGAAT |
| | ATGTATTTAAcattaggatttcagaagcatgagaaaaacacccttgtttctactagcaaaagcaggggtttaaaATG |
| | AATCCAAACCAAAAGATAATAACCATTGGTTCGGTCTGTgTGACAATTGGA |
| | ATGGCTAACTTAATATTACAAATTGGAAACATAATCTCAATATGGATTAGC |
| | CACTCAATCCAATTGGGGAATCAGAATCAAATCGAAACATGCAATCAATC |
| | CGTAATTACATACGAGAATAATACTTGGGTGAATCAGACATACGTTAACA |
| | TATCGAATACTAATTTCaCTGCCGGACcATCCGTCGTGAGTGTGAAACTAGC |
| | CGGTAATAGTAGTCTATGTCCCGTTAGCGGATGGGCTATATACTCTAAAGA |
| | CAATAGCGTTAGAATCGGATCTAAAGGCGACGTATTCGTTATACGCGAAC |
| | CATTCATAAGTTGTAGTCCATTAGAGTGTAGGACTTTTTTTCTGACACAGG |
| | GCGaACTATTGAACGATAAGCATTCTAACGGTACAATCAAAGATAGGTCA |
| | CCATATAGAACACTAATGTCATGTCCGATAGGCGAAGTGCCTAGTCCATA |
| | CAATAGTAaATTCGAATCCGTCGCTTGGTCCGCTAGCGCATGCCATGACGG |
| | TATTAATTGGTTGACAATCGGGATTAGCGGACCCGATAACGGCGCAGTCG |
| | CCGTACTTAAGTATAACGGTATAATTACCGATACTATTAAGAGTTGGCGAA |
| | ATAATATATTGCGAACACAGGAATCCGAATGCGCATGCGTTAACGGATCA |
| | TGTTTTACCGTTATGACTGACGGACCATCTAACGGGCAAGCGTCATATAAG |
| | ATTTTTAGAATCGAAAAAGGTAAGATAGTGAAATCCGTCGAAATGAACGC |
| | TCCTAATTATCATTACGAAGAGTGCTCATGTTATCCCGATTCTAGCGAAAT |
| | TACATGCGTATGTAGAGACAATTGGCACGGATCTAATAGACCTTGGGTGT |
| | CATTCAATCAGAATCTAGAGTATCAAATCGGGTATATATGCTCAGGGATAT |
| | TCGGAGACAATCCTAGACCTAACGATAAGACAGGGTCATGCGGACCAGTG |
| | AGTTCTAACGGCGCTAACGGCGTTAAGGGGTTTAGTTTCAAATACGGTAA |
| | CGGCGTATGGATAGGGAGAACTAAGTCAATCTCTAGTAGAAACGGATTCG |
| | AAATGATATGGGACCCTAACGGATGGACCGGAACCGATAATAATTTTTCG |
| | ATTAAACAGGATATCGTAGGGATTAACGAATGGTCAGGGTATAGCGGATC |
| | ATTCGTACAGCATCCAGAGTTAACCGGACTCGATTGCATACGACCATGTTT |
| | TTGGGTCGAACTGATTAGGGGGAGACCGAAAGAGAATACTATATGGACTA |
| | GCGGGAGCAGCATATCCTTTTGTGGTGTAAACAGTGACACTGTGGGTTGGT |
| | CTTGGCCAGACGGTGCTGAGTTGCCATTTACCATTGACAAGTAAtttgttcaaaaaa |
| | ctccttgtttctact |
| H10N7 HA | atgtataaga tagtgctcgt actcgcacta ttaggcgcag tgcacggact cgacaaaatt 60 |
| SEQ ID | tgcctagggc atcacgcagt gcctaacgga actatcgtta agacacttac taacgaaaaa 120 |
| NO: 19 | gaggaagtga ctaacgctac cgaaacagtc gaatcaaaat cactcgacaa attgtgtatg 180 |
| | aaaagtcgga attataaaga cctaggcaat tgccatccga tagggatggt gatagggact 240 |
| | cccgcttgcg atctgcatct gacagggaca tgggatacac ttatcgaacg ggacaatagt 300 |
| | atagcgtatt gttatccagg cgctacagtg aacgaagagg cacttagaca aaaaattatg 360 |
| | gaatccggcg aaatcgataa gattagtacc ggattcacat acgaatcctc tattaatccc 420 |
| | gcaggaacaa ctaaggcttg tatgcgaaac ggtaagaatt cgttttacgc tgaactgaaa 480 |
| | tggcttgtga gtaaggacaa aggtaggaat ttcccacaaa ctactaatac ttataggaat 540 |
| | accgattcaa ccgaacatct gattatatgg gggatacacc atccaagttc gacacaagag 600 |
| | aaaaacgatc tatacggaac gcaatccctt agcattagcg tagggtctag tacttatcag 660 |
| | aataatttcg taccggtagt gggcgctaga ccgcaagtga acggacaatc cggtagaatc 720 |
| | gatttccatt gggctatggt gcaaccaggc gataacataa cttttagcca taacggcgga 780 |
| | ctgatagcgc ctagtagagt gagtaagctt aagggaaggg ggttggggat acaatccggc 840 |
| | gctagcgtag acaacgattg cgaatcaaaa tgcttttgga aaggggggtc aattaatact 900 |
| | aaattgccat ttcagaatct gtcacctaga acagtgggac aatgccctaa atacgttaat 960 |
| | aagaaaagtc tgttactcgc aaccggtatg cgaaacgtac cagaggtagt gcaaggtagg 1020 |
| | gggctattcg gagcgatagc gggatttatc gaaaacggat gggagggtat ggtcgacgga 1080 |
| | tggtacgggt ttagacacca aaacgcacag ggaaccggac aggcagcaga ctataaatcg 1140 |
| | acacaagccg ctatagacca aattaccggt aagcttaaca gactgatcga aaagactaat 1200 |
| | accgaattcg aatcaatcga atccgaattt agcgaaatcg aacaccaaat cggaaacgta 1260 |
| | attaattgga caaaagactc aattaccgat atatggacat atcaagccga actgttagtc 1320 |
| | gctatggaga atcagcatac aatcgatatg gccgatagcg aaatgcttaa cctttacgaa 1380 |
| | agggtgagaa aacagcttag acaaaacgct gaagaggacg gtaaggggtg tttcgaaata 1440 |
| | taccataaat gcgacgataa ttgtatggag tctatacgga ataacacata cgaccatacg 1500 |
| | caatatagag aggaagcact actgaataga cttaacatta atccggttaa gctatctagc 1560 |
| | ggatataaag acgtgatatt gtggttctca ttcggagcgt catgtttcgt attgctcgca 1620 |
| | gtgattatgg gactcgtatt cttttgcctt aaaaacggta atatgagatg cacaatttgc 1680 |
| | ata 1683 |
| H10N7 NA | atgaatccta accaaaagct attcgcacta agcggagtcg ccatagccct atcaatactg 60 |
| SEQ ID | aatctgttaa tcggaatatc gaacgttggg ttgaacgtta gtttgcacct taaggggtca 120 |
| NO: 20 | tccgaccaag acaaaaattg gacatgtact agcgttacgc aaacaaatac gactttgatc 180 |
| | gaaaatacat acgttaacaa tacgacagtg ataaataaag agaccggaac tactaagcaa 240 |
| | aactatctga tgctgaataa gtcactatgt aaggtcgagg gatgggtggt agtcgctaaa 300 |
| | gacaacgcaa taaggttcgg cgaaagcgaa cagataatcg tgacacgcga accatacgtt 360 |
| | agttgcgatc cgttagggtg taagacatac gcattacacc aagggactac gatacggaat 420 |
| | aaacactcta acggaacgat acacgacaga accgcattta gggggttgat atcgacacct 480 |
| | ctcggatcac ctcccgtagt gagtaatagc gatttcttat gcgtggggtg gtcaagtact 540 |
| | agttgtcacg acggaatcgg acgtatgaca atatgcgtac aggggaataa caataacgca 600 |
| | accgcaacag tgtattacga taggagactg actacaacaa ttaagacttg ggccggtaag 660 |
| | atactgagaa cacaggaaag cgaatgcgtt tgccataacg gtacatgcgt agtgattatg 720 |
| | acagacggat ccgcaagttc gcaagcccat acgaaagtgc tatattttca caaagggctc 780 |
| | gtaatcaaag aggaagccct taagggatcc gctagacata tcgaagagtg tagttgttac 840 |
| | ggacacaata gtaaggttac atgcgtatgt agggacaatt ggcaaggcgc aaatagacca 900 |
| | gtgatagaga tagacatgaa cgctatggag catacgagtc agtatctatg taccggagtg 960 |
| | ttaaccgaca ctagtagacc tagcgataag agtatgggcg attgcaataa tccgataacc 1020 |
| | ggatcacccg gagcaccagg cgttaagggg ttcgggtttc tcgatagcga taatacatgg 1080 |
| | ttaggtagga caatctcacc taggtcaaga tccggattcg aaatgctcaa aatccctaac 1140 |
| | gccggaacag accctaatag taggattacc gaacgacaag agatagtcga caataacaat 1200 |
| | tggtcagggt atagcggatc tttcatagac tattgggacg aatcaagcgt atgttataac 1260 |
| | ccatgtttct atgtcgaact gattaggggg agacccgaag aggccaaata tgtgtggtgg 1320 |
| | actagtaata gtctcgtagc cctatgcgga tcaccgataa gcgtagggtc agggtcattc 1380 |
| | ccagacggag cccaaatcca atattttagt 1410 |
| H1N1 HA | atgaaagcga ttctagtcgt actgctatat acattcgcta ccgctaacgc cgatacacta 60 |
| (A/New | tgcatagggt atcacgctaa taatagtaca gacacagtag acacagtact cgaaaaaaac 120 |
| York/3568/ | gttacggtta cacattccgt taatctgtta gaggataagc ataacggtaa gctatgtaaa 180 |
| 2009) | ctgagaggcg tagcaccatt gcatttgggt aagtgtaata tagccggatg gatactaggt 240 |
| SEQ ID | aatcccgaat gcgaatcact atcaactgca agttcatggt cttatatagt cgaaactagt 300 |
| NO: 21 | tcaagcgata acggtacatg ttatcccgga gactttatcg attacgaaga gttgagagag 360 |
| | caattgtcta gcgtaagctc attcgaaaga ttcgaaattt ttccgaaaac tagttcatgg 420 |
| | cctaatcacg attcaaataa gggggtaaca gccgcatgcc cacacgcagg cgctaagtca 480 |
| | ttctataaaa atctgatatg gctagtgaaa aaagggaatt cttatccgaa actatcaaaa 540 |
| | tcatatatta acgataaggg taaggaggta ctcgtattgt gggggataca ccatccatca 600 |
| | actagcgcag accaacaatc tctgtatcag aatgccgacg catacgtatt cgtagggact 660 |
| | agtaggtact ctaaaaaatt taaacccgaa atcgctatta gaccgaaagt gagagaccag 720 |
| | gagggaagaa tgaattacta ttggacacta gtcgaaccag gcgataagat tacattcgaa 780 |
| | gcgacaggga atctagtggt accgagatac gcattcgcaa tggagagaaa cgccggatcc 840 |
| | ggaattatta ttagcgatac tcccgtacac gattgcaata caacatgtca gacaccaaaa 900 |
| | ggggcaatta atactagcct accatttcag aatatacacc caattacaat cggtaagtgt 960 |
| | ccaaaatacg ttaagtctac gaaacttaga ttggcaacag ggttgagaaa cgtaccatca 1020 |
| | atacagtcta gagggttgtt cggagcaatc gccggattca tagagggggg gtggaccggt 1080 |
| | atggtcgacg gatggtacgg ataccatcat caaaacgaac aggggtccgg atacgcagcc 1140 |
| | gatctgaaat caacacagaa cgcaatcgac gaaattacga ataaagtgaa tagcgtaatc 1200 |
| | gaaaaaatga atactcagtt tacagccgta ggtaaggaat ttaatcatct cgaaaaaaga 1260 |
| | attgagaatc tgaataaaaa ggtagacgac gggtttctag acatttggac atataatgcc 1320 |
| | gaactgttag tgttactcga aaacgaaaga acattagact atcacgattc taacgttaag 1380 |
| | aatctatacg aaaaagtgag atcgcaattg aagaataacg caaaagagat agggaatggg 1440 |
| | tgtttcgaat tctaccataa atgcgataat acatgtatgg aatccgtaaa aaacggtaca 1500 |
| | tacgattatc cgaaatatag cgaagaagca aaactgaata gggaagagat tgacggagtt 1560 |
| | aagttggagt caactaggat ttaccagata ctcgcaattt actctacagt cgcatcaagt 1620 |
| | ctagtgttag tcgttagctt aggcgcaatt agtttttgga tgtgttcaaa cggatcactg 1680 |
| | caatgtagga tttgcata 1698 |
| H1N1 NA | atgaatccta accaaaaaat tataacaatc ggatccgttt gtatgacaat cggtatggct 60 |
| (A/New | aacctaatac tgcaaatcgg taatattata tcgatttgga tctcacatag tatacaattg 120 |
| York/3568/ | ggtaatcaga atcagataga gacatgcaat caatccgtta ttacatacga aaataatact 180 |
| 2009) | tgggttaatc agacatacgt taacatatcg aatactaatt tcgctgccgg acaatccgtc 240 |
| SEQ ID | gttagcgtta agttagccgg taatagttca ctatgccccg ttagcgggtg ggctatatac 300 |
| NO: 22 | tctaaagaca attcgattag aatcggatct aagggcgacg tattcgtaat acgcgaacca 360 |
| | ttcataagtt gtagtccatt agagtgtaga actttttttc taacacaagg cgctctattg 420 |
| | aacgataagc atagtaacgg tacaattaag gatagatcac cttatagaac attgatgtca 480 |
| | tgtcctatcg gcgaagtgcc tagtccatac aatagtagat tcgaatccgt cgcatggtcc 540 |
| | gctagcgcat gtcacgacgg gattaattgg ttgactatag ggattagcgg acccgataac 600 |
| | ggcgcagtcg ctgtgcttaa gtataacggt attattaccg acactataaa gagttggcga 660 |
| | aataacatac tgagaacaca ggaatccgaa tgcgcatgcg taaacggttc atgttttacc 720 |
| | gtaatgactg acggacctag cgacggacaa gcgtcatata agatttttag aatcgaaaaa 780 |
| | ggtaagatag tgaaatctgt cgagatgaac gctccgaatt atcattacga agagtgtagt 840 |
| | tgttatcccg attctagcga aattacatgc gtatgtaggg acaattggca cgggtctaat 900 |
| | cgaccatggg tgtcattcaa tcagaactta gagtatcaga tagggtatat atgctcaggg 960 |
| | atattcggcg ataatcctag accgaacgat aaaaccggat catgcggacc agtgtcatct 1020 |
| | aacggcgcta acggagtgaa agggtttagt ttcaaatacg gtaacggcgt atggatcgga 1080 |
| | cgaactaagt ctatatctag taggaacgga ttcgaaatga tatgggaccc aaacgggtgg 1140 |
| | accggtaccg ataataactt ttcaatcaaa caggacatag tcggaattaa cgaatggtcc 1200 |
| | gggtatagcg gatcattcgt gcaacatcca gagttaaccg gactcgattg cataagacca 1260 |
| | tgtttttggg tcgaattgat tagggggaga ccaaaagaga atactatatg gactagcgga 1320 |
| | tctagtatta gcttttgcgg agtgaatagc gataccgtag ggtggtcatg gccagacgga 1380 |
| | gccgaactac catttacaat cgataag 1407 |
| H1N2 HA | atgaaagtga aactgttaat actgttgtgc acttttaccg ctacatacgc cgatacaatt 60 |
| (A/New | tgcatagggt atcacgctaa taatagtacc gatacagtcg acactgtgtt ggaaaagaac 120 |
| York/211/ | gtaaccgtta cacactccgt taatctgtta gaggattccc ataacggtaa gttgtgtctg 180 |
| 2003) | ttgaaaggga tcgcaccatt gcaattgggt aattgtagcg tagccggatg gatattgggg 240 |
| SEQ ID | aatcccgaat gcgaactatt gattagtaaa gagtcatggt catatatagt cgagacacct 300 |
| NO: 23 | aatcccgaaa acggagcatg ctatcccgga tatttcgccg attacgaaga gcttagagag 360 |
| | caattgtcta gcgtaagctc attcgaaaga ttcgaaattt ttccaaaaga gtcaagttgg 420 |
| | cctaatcata ccgtaacagg cgtatccgca tcatgtagtc ataacggtaa gtcaagcttt 480 |
| | tataagaatc tgttatggtt aaccggtaaa aacggactgt atccaaatct atctaagtca 540 |
| | tacgcaaata ataaagagaa agaggtactg attctatggg gggtgcatca cccacctaat 600 |
| | ataggcgatc aaagaacatt gtatcatacc gaaaacgcat acgtatccgt cgttagctca 660 |
| | cactatagta gaaggtttac acccgaaatt actaagagac ctaaggtaag ggatcaggag 720 |
| | ggtaggatta attattattg gactctactt gaaccaggcg atactatcat attcgaagct 780 |
| | aacggaaatc taatcgcacc atggtacgca ttcgcactat ctagggggtt cggatccggg 840 |
| | attattactt ctaacgctcc aatggacgaa tgcgacgcaa agtgtcagac accacaggga 900 |
| | gcgattaata gttccctacc attccaaaac gtacaccccg ttacaatcgg cgaatgtccg 960 |
| | aaatacgtta gatccgctaa acttagaatg gtgaccggac tgagaaatat accatcaatc 1020 |
| | caatctaggg ggctattcgg agccatagcc ggatttatcg aaggggggtg gacagggatg 1080 |
| | gtcgacggat ggtatgggta tcaccaccaa aacgaacagg gatccggata cgccgccgat 1140 |
| | cagaaatcca cacaaaacgc tattaacgga attacgaata aagtgaatag cgtaatcgaa 1200 |
| | aaaatgaata cacaatttac tgccgtaggt aaggaattca ataagttaga gagaaggatg 1260 |
| | gagaatctga ataaaaaagt cgacgacgga ttcctagaca tatggacata taacgccgaa 1320 |
| | ctgttagtgt tgcttgagaa cgaaaggaca ctagactttc acgattcaaa cgttaaaaat 1380 |
| | ctatacgaaa aagtcaaatc ccaattgaaa aataacgcta aagagatagg gaatgggtgt 1440 |
| | ttcgaattct atcataagtg taataacgaa tgtatggaat ccgttaaaaa cggaacatac 1500 |
| | gattatccaa agtatagcga agagtcaaaa ctgaataggg aaaaaatcga cggagtcaaa 1560 |
| | cttgactcaa tgggggtgta tcagatactc gcaatctata gtacagtcgc atctagccta 1620 |
| | gtactgttag tgagtctggg agcgataagc ttttggatgt gttctaacgg atcactgcaa 1680 |
| | tgtaggatat gcatatga 1698 |
| H1N2 NA | atgaacccta atcaaaaaat aattacaatc ggatccgtta gtctgacaat cgctactata 60 |
| (A/New | tgttttctga ctcagatagc gatactcgtt acaaccgtta cattgcattt caaacaatac 120 |
| York/211/ | gaatgcaatt ccccccctaa caatcaggta atgttgtgcg aacctacaat aatcgaacgg 180 |
| 2003) | aatattaccg agatagtgta tctgactaat acgactatcg aaaaagagat atgcccaaaa 240 |
| SEQ ID | ctagccgaat atcggaattg gtcaaaaccg caatgtaaca taaccggatt cgcaccattt 300 |
| NO: 24 | tcgaaagaca attcgattag gttgtccgcc ggaggcgata tttgggttac acgcgaacct 360 |
| | tatgtgtcat gcgatcccga taaatgctat caattcgcac tcggacaggg gactaccctt 420 |
| | aataacggac attctaacga taccgtacac gatagaactc catatcgaac attgctaatg 480 |
| | aacgagttag gcgtaccatt ccatttgggc actaaacagg tatgtatcgc atggtctagc 540 |
| | tctagttgcc atgacggtaa ggcttggttg catgtgtgcg ttaccggcga cgataagaac 600 |
| | gcaaccgcta gctttatata taacggtagg ttggtcgact caatcgggtc atggtcaaaa 660 |
| | aaaatactta gaacgcaaga gtccgaatgc gtatgcataa acggtacatg caccgtagtg 720 |
| | atgaccgacg gatccgctag cggtaaggcc gatacgaaaa tactgtttat cgaagagggt 780 |
| | aagatagtgc atacgagtct actatccgga tccgctcaac atgtcgaaga gtgttcatgt 840 |
| | tatcctaggt atcccggcgt tagatgcgta tgtagggata attggaaagg gagtaataga 900 |
| | cctatagtcg atattaacgt taaggattat tcaatcgtaa gtagttatgt gtgtagcgga 960 |
| | ctcgtaggcg atacacctag aaaaaacgat agctctagta gctcacattg cctagaccct 1020 |
| | aataacgaag agggggggca tggcgttaag ggatgggcat tcgacgacgg taacgacgtt 1080 |
| | tggatgggta ggactattag cgaaaagctt agatccgggt atgagacatt caaagtgata 1140 |
| | gagggatggt ctaaacctaa ttcaaaactg caaattaata ggcaagtgat agtcgatagg 1200 |
| | gataatagat ccgggtattc cggaattttt agcgttgagg gtaagtcatg tattaatagg 1260 |
| | tgtttttatg tcgagcttat tagggggaga aatcaggaaa ccgaagtgtt gtggacatcc 1320 |
| | aattcaatcg tcgttttttg cggaactagc ggaacatacg gtaccggatc atggcccgac 1380 |
| | ggagccgata ttaaccttat gcctatataa 1410 |
| H2N2 HA | atggcaataa tctatctgat actgttgttt acagccgtta ggggcgatca gatatgcata 60 |
| (A/Albany/ | gggtatcacg ctaataatag taccgaaaaa gtcgatacaa tactcgaaag aaacgtaacc 120 |
| 22/1957) | gttacacacg ctaaagatat actcgaaaag acacataacg gtaagctatg caaacttaac 180 |
| SEQ ID | ggtataccac cacttgagtt aggcgattgc tcaatcgcag gatggttgtt ggggaatccc 240 |
| NO: 25 | gaatgcgata ggctattgag cgtacccgaa tggtcttata ttatggaaaa agagaatcct 300 |
| | agagacggat tgtgttatcc cggatctttt aacgattacg aagagcttaa acatctgcta 360 |
| | tctagcgtta aacatttcga aaaagtgaaa attctgccaa aagataggtg gacacagcat 420 |
| | acgactaccg gaggatctag ggcatgcgcc gttagcggta atccgtcatt ctttagaaat 480 |
| | atggtatggt tgacaaaaaa ggggtctaat tatccagtcg ctaagggatc gtataataat 540 |
| | acaagcggag agcaaatgtt gattatatgg ggagtgcatc accctaacga cgaaaccgaa 600 |
| | caacggacac tgtatcaaaa cgtcggaaca tacgttagcg tcggtacacc aactctgaat 660 |
| | aaaagatcga ctcccgatat cgcaactaga ccaaaagtga acggacaggg ggggagaatg 720 |
| | gagtttagtt ggacactact cgatatgtgg gatacaatta atttcgaatc aaccggtaat 780 |
| | ctgatcgcac ccgaatacgg gtttaagatt agtaaaaggg ggtcatccgg tattatgaaa 840 |
| | accgaaggta cactagggaa ttgcgaaact aagtgtcaga caccactagg ggctattaat 900 |
| | acaacactac catttcataa tgtgcatcca ttgacaatcg gagagtgtcc taagtatgtg 960 |
| | aaatccgaaa aactagtgct tgcaaccgga ctgagaaacg taccgcaaat cgaatccaga 1020 |
| | gggttgttcg gagcaatcgc agggtttatc gaaggggggt ggcagggaat ggtcgacgga 1080 |
| | tggtatgggt atcatcactc taacgatcag ggatccggat acgcagccga taaggagtca 1140 |
| | acccaaaaag cattcgacgg aattactaat aaggtgaata gcgtaatcga aaaaatgaat 1200 |
| | acacaattcg aagccgtcgg taaagagttt tcgaatctcg aaaggagact tgagaatctg 1260 |
| | aataaaaaaa tggaggacgg attcttagac gtatggacat ataatgccga actgttagtc 1320 |
| | cttatggaga acgaacggac actagacttt cacgatagta acgttaagaa tctgtatgac 1380 |
| | aaagtgagaa tgcaattgag agacaatgtg aaagagctag gtaacggatg tttcgaattc 1440 |
| | tatcataaat gcgacgacga gtgtatgaat agcgttaaaa acggtacata tgactatcct 1500 |
| | aagtatgagg aagagtcaaa gcttaataga aacgagatta agggagtgaa actatctagt 1560 |
| | atgggagtgt atcagatact cgcaatatac gctacagtcg ccggatccct atcacttgcg 1620 |
| | attatgatgg ccggaattag cttttggatg tgctctaacg gatcattgca atgtaggatt 1680 |
| | tgcatatga 1689 |
| H2N2 NA | atgaatccta accagaaaat tattactata gggtcagtgt cattgactat cgcaaccgta 60 |
| (A/Albany/ | tgctttatta tgcaaatagc gatactcgca actaccgtaa cattgcattt taaacaacac 120 |
| 22/1957) | gaatgcgata gtcccgctag caatcaggta atgccatgcg aacctattat aatcgaacgg 180 |
| SEQ ID | aatattaccg agatagtgta tcttaacaat actactatcg aaaaagagat atgcccagag 240 |
| NO: 26 | gccgtcgagt atagaaattg gtctaaacct caatgtcaga ttaccggatt cgcaccattc 300 |
| | tctaaagaca attcgattag attgtccgcc ggaggcgata tatgggtgac acgcgaacct 360 |
| | tatgtgtcat gcgatcccgg taagtgttat caattcgcac tcggacaggg gactacactc 420 |
| | gataataaac attctaacgg tacgatacac gataggattc cacataggac actattgatg 480 |
| | aacgagttag gcgtaccgtt tcatctaggc actaaacagg tatgcgttgc gtggtctagc 540 |
| | tcatcatgtc atgacggtaa ggcatggttg catgtgtgcg taaccggcga cgatagaaac 600 |
| | gctaccgcta gttttatata cgacggtagg ctagtcgatt caatcggatc atggtcacag 660 |
| | aatatactta gaacacagga atccgaatgc gtttgtatta acggtacatg tacagtcgtt 720 |
| | atgaccgacg gatccgcatc cggtagggcc gatactagga tactgtttat aaaagagggc 780 |
| | aaaatcgtgc atattagccc acttagcgga tccgcacaac atatcgaaga gtgtagttgc 840 |
| | tatcctaggt atcctgacgt tagatgtatt tgcagagaca attggaaagg gtctaataga 900 |
| | cccgtaatcg atatcaatat ggaggattat tcaatcgata gctcttatgt gtgtagcgga 960 |
| | ttagtcggcg atacacctag aaacgacgat agctctagta attcgaattg tagggaccct 1020 |
| | aataacgaga gaggcaatcc cggcgttaaa gggtgggcat tcgataacgg cgacgacgtt 1080 |
| | tggatggggc gaacaattaa taaggactct agatccgggt atgagacatt caaagtgata 1140 |
| | ggggggtggt ctacacctaa ctcaaaatct caagtgaata ggcaagtgat agtcgacaat 1200 |
| | aacaattggt cagggtatag cggtatattc tcagtcgagg gtaagtcatg tattaataga 1260 |
| | tgtttttacg ttgagttgat tagggggcga ccacaagaga ctagagtgtg gtggactagt 1320 |
| | aatagtatag tcgttttttg cggaactagc ggtacatacg gaaccggatc atggcctgac 1380 |
| | ggagcgaata ttaattttat gccaatctaa 1410 |
| H3N2 HA | atgaaaacaa ttatcgcact gtcatacata ctgtgtctgg tattcgctca aaaattgccc 60 |
| (A/New | ggtaacgaca attcaaccgc tacattgtgc ttagggcatc acgccgtacc gaacggaact 120 |
| York/933/ | atcgttaaga caattactaa cgaccaaatc gaagtgacta acgctacaga gttggtgcaa 180 |
| 2006) | tcctctagta caggcgaaat atgcgattca ccacaccaaa tccttgacgg agagaattgt 240 |
| SEQ ID | acacttatcg acgcactatt aggcgatcca caatgcgacg gatttcagaa taaaaaatgg 300 |
| NO: 27 | gatctattcg ttgagagatc caaagcttat tcaaattgtt atccatacga cgtaccggat 360 |
| | tacgctagcc ttaggtcact cgttgcgtca agcggtactc tcgaattcaa taacgagtca 420 |
| | ttcaattgga ctggcgttac gcaaaacgga actagtagcg catgtaaaag acggtctaat 480 |
| | aatagctttt ttagcagact gaattggttg actcatctga aattcaaata tcccgcactt 540 |
| | aacgttacta tgcctaataa cgaaaaattc gataagctat atatatgggg cgtacaccat 600 |
| | cccggaacgg ataacgatca gatattcttg tacgctcaag ctagcggtag gattaccgtt 660 |
| | agtactaaaa gatcccaaca aaccgtaatt ccgaatatcg gatctagacc tagggtgaga 720 |
| | ratataccgt ctaggattag catatattgg actatcgtta aacccggaga catactgttg 780 |
| | atcaatagta caggcaatct gatcgcacct agggggtatt tcaaaattag atccggtaag 840 |
| | tctagcatta tgagatccga cgcaccaatc ggtaaatgta atagcgaatg cattacacca 900 |
| | aacggatcaa tccctaacga taagccattc caaaacgtaa ataggattac atacggcgca 960 |
| | tgccctagat acgttaaaca gaatacgctt aaacttgcga caggtatgcg aaacgtaccc 1020 |
| | gaaaaacaga ctagggggat attcggcgca atcgccggat ttatcgaaaa cggatgggag 1080 |
| | ggtatggtcg acggatggta cggatttaga catcaaaata gcgaagggat agggcaagcc 1140 |
| | gccgatctga aatcaacgca agccgctatt aatcaaatta acggaaaact gaatagattg 1200 |
| | atcggtaaga ctaacgaaaa atttcaccaa atcgaaaaag agtttagcga agttgaggga 1260 |
| | aggatacaag accttgagaa atacgttgag gatactaaga tcgacctatg gtcatataat 1320 |
| | gccgagttgc tagtcgcact cgagaatcag catacaatcg atctgactga tagcgaaatg 1380 |
| | aataaattgt tcgaaagaac gaaaaaacaa ttgcgcgaaa acgccgaaga catggggaat 1440 |
| | gggtgtttta agatatacca taaatgcgat aacgcatgca tagggtcaat cagaaacgga 1500 |
| | acatacgatc acgacgtata tagagacgaa gcccttaata atagattcca aattaaaggc 1560 |
| | gttgagctta aaagcggata caaagactgg atactgtgga ttagtttcgc aatctcatgc 1620 |
| | tttctattgt gcgttgtgct attggggttc ataatgtggg catgtcagaa agggaatatt 1680 |
| | agatgcaata tttgtatatg a 1701 |
| H3N2 NA | atgaatccta accaaaagat tattacaatc ggatccgtta gccttactat atccacaatt 60 |
| (A/New | tgttttttta tgcaaatagc gatactgata actaccgtta cattgcattt caaacaatac 120 |
| York/933/ | gaattcaatt caccccctaa taatcaggtt atgttgtgcg aacctactat tatcgaacgg 180 |
| 2006) | aatataaccg agatagtgta tctaacgaac actacaatcg aaaaagagat atgccctaag 240 |
| SEQ ID | ctcgcagagt atagaaattg gtcaaaaccc caatgcgata taaccggatt cgcaccattt 300 |
| NO: 28 | agtaaggata atagtattag gttgtccgcc ggaggcgata tatgggttac acgcgaacca 360 |
| | tacgtgtcat gcgatcccga taaatgctat caattcgctc tcggacaggg aacgacattg 420 |
| | aataacgtac attcaaacga taccgtacac gataggacac cttatagaac actattgatg 480 |
| | aacgaactag gcgtaccttt ccatctcgga actaaacagg tttgtatcgc ttggtctagt 540 |
| | agctcatgcc atgacggtaa ggcatggttg catgtgtgcg ttaccggcga cgataaaaac 600 |
| | gcaaccgcta gtttcatata taacggtagg ttagtcgata gcgtagtgag ttggtctaaa 660 |
| | gacatactgc gaacacagga atccgagtgc gtatgcataa acggtacatg taccgtagtg 720 |
| | atgaccgacg gatccgctag cggtaaggcc gatacgaaaa tattgttcat agaggagggt 780 |
| | aagatagtgc atacaagtac actatccgga tccgctcaac atgtcgaaga gtgctcatgt 840 |
| | tatcctagat atcccggcgt tagatgcgta tgtagagaca attggaaagg gtctaataga 900 |
| | ccgatagtcg acattaatat taaaaactat tcaatcgtta gctcatatgt gtgttccgga 960 |
| | ttagtcggcg atacccctag aaaaaccgat agctctagct catcccattg tcttgaccct 1020 |
| | aataacgaag agggggggca tggcgttaag ggatgggcat tcgacgacgg taacgacgtt 1080 |
| | tggatgggac ggacaattag cgaaaaactt agattggggt atgagacttt taaggtaatc 1140 |
| | gaagggtggt ctaatcctaa ttcgaaactg caaattaata ggcaagtgat agtcgatagg 1200 |
| | gggaataggt ccggatatag cggaatcttt tccgttgagg gtaagtcatg tattaatagg 1260 |
| | tgtttttatg tcgaactgat tagggggaga aaagaggaaa ccgaagtgtt atggactagt 1320 |
| | aactcaatcg ttgtgttttg cggtacatcc ggtacttatg gaaccggatc atggccagac 1380 |
| | ggagccgata taaaccttat gccaatttaa 1410 |
| H5N1 HA | atggagaaaa tagtgctact actcgcaatc gttagtctgg ttaagtccga tcagatatgc 60 |
| (A/Jiangsu/ | atagggtatc acgctaacaa tagtaccgaa caggtcgaca ctattatgga aaaaaacgtt 120 |
| 1/2007) | accgttacac acgcacagga catactcgaa aaaacccata acggtaagtt atgcgattta 180 |
| SEQ ID | gacggagtta agccactgat acttagggat tgttcagtcg ccggatggtt gttagggaat 240 |
| NO: 29 | ccaatgtgcg acgaattcat taacgtaccc gaatggtcat acatagtcga aaaagcgaat 300 |
| | cccgctaacg atctatgtta tccagggaat tttaacgatt acgaagagct taagcatcta 360 |
| | ctatctagaa taaaccattt cgaaaagatt cagataatac cgaaatcgag ttggtccgat 420 |
| | cacgaagcgt caagcggagt gagtagcgca tgcccatacc aaggaacacc atcattcttt 480 |
| | agaaacgtcg tttggttgat taaaaaaaat aatacatatc cgactattaa gagatcatat 540 |
| | aataatacaa accaagagaa tctactgata ctatggggga tacaccatag taacgacgca 600 |
| | gccgaacaga ttaagctata tcagaatcca actacataca ttagcgtagg gactagtaca 660 |
| | cttaatcaga gactcgtacc taaaatcgct actagatcga aggtaaacgg acaatccggt 720 |
| | agaatggact ttttttggac tatactgaaa cctaacgacg caattaattt cgaatctaac 780 |
| | ggaaatttta tcgctcccga atacgcatat aagatagtga aagaggggga tagcgcaatt 840 |
| | atgaaatccg aagtcgaata cggaaattgc aatactaagt gtcagacacc aatcggagca 900 |
| | attaactcta gtatgccatt ccataacata catccactta caatcggaga atgccctaaa 960 |
| | tacgttaagt ctaacaaact cgtactcgca accggactta ggaatagtcc acttagagag 1020 |
| | agacgaagaa agagagggtt gttcggagca atcgcagggt tcatagaggg ggggtggcag 1080 |
| | ggtatggtcg acggatggta cgggtatcat cattctaacg aacagggatc cggatacgca 1140 |
| | gccgataaag agagtactca gaaagcaatc gacggagtga cgaataaagt gaattcgata 1200 |
| | atcgataaga tgaatacgca attcgaagcc gtaggtaggg aattcaataa tctcgagaga 1260 |
| | cgaatcgaaa accttaacaa aaaaatggaa gacggattcc tagacgtatg gacttataac 1320 |
| | gccgaactgt tagtgcttat ggagaacgaa agaacccttg actttcacga ttctaacgtt 1380 |
| | aagaatctat acgatagagt gagactgcaa ttgagggata acgctaaaga gttagggaac 1440 |
| | gggtgtttcg aattctatca taaatgcgat aacgaatgta tggagtcagt gagaaacggt 1500 |
| | acatacgact atccgcaata ttccgaagag gctagattga aaagagagga gattagcgga 1560 |
| | gtgaaacttg agtcaatggg gacatatcag atattgtcaa tatactcaac cgtcgctagt 1620 |
| | agtctcgcac tcgcaattat ggtcgccgga ctgtcactat ggatgtgttc aaacggtagt 1680 |
| | ctgcaatgta ggatttgtat ataa 1704 |
| H5N1 NA | atgaatccga atcaaaaaat tataacaata gggtcaatct gtatggtaat cggtatagtg 60 |
| (A/Jiangsu/ | tcacttatgt tacaaatcgg gaatattata tctatttggg tgtcacactc aatccaaacc 120 |
| 1/2007) | ggtaatcaac accaagacga acctatacgg aatgcgaatt tcttaacaga gaatgccgta 180 |
| SEQ ID | gctagcgtta cgttagccgg taatagttca ttgtgtcccg ttagggggtg ggctgtgcat 240 |
| NO: 30 | agtaaggata atagtattag gatagggtct aaaggcgacg tattcgtgat acgcgaacct 300 |
| | tttatctctt gctcacactt agagtgtaga acattttttc tgactcaagg cgcactgtta 360 |
| | aacgataaac actctaacgg tacagttaag gataggtcac cacataggac attgatgtca 420 |
| | tgtcccgtag gcgaagctcc tagtccatat aatagtagat tcgaaagcgt tgcatggtcc 480 |
| | gctagcgctt gtcacgacgg aactagttgg ttgacaatcg ggatatccgg acccgataat 540 |
| | ggcgcagtcg cagtgttgaa gtataatggg attataaccg atactatcaa atcatggaga 600 |
| | aataatatac tgagaacaca ggagtccgaa tgcgcttgcg ttaacggatc atgctttacc 660 |
| | gttatgactg acggaccatc taacgggcaa gctagttata aaattttcaa aatggagaaa 720 |
| | ggtaaggtag tgaaatccgt tgagcttaac gctccaaatt atcattacga agagtgtagt 780 |
| | tgctatccag acgctggcga aattacttgc gtatgtagag acaattggca cggatctaat 840 |
| | agaccatggg ttagctttaa tcagaattta gagtatcaga tagggtatat atgttccgga 900 |
| | gtgttcggcg ataatcctag acctaacgac ggtacagggt catgcgatcc agtgagtcca 960 |
| | aacggcgcat acggaattaa agggtttagc tttaagtatg ggaatggcgt atggatcggt 1020 |
| | aggactaagt ctactaatag tagatccgga ttcgaaatga tatgggaccc taatgggtgg 1080 |
| | actgagactg atagtagttt tagcgtaaaa caggatatag tcgctataac cgattggagc 1140 |
| | gggtatagcg gatcattcgt acagcatccc gaattgactg ggttagactg tattagacca 1200 |
| | tgcttttggg tcgaattgat tagggggaga ccaaaagagt caactatatg gactagcgga 1260 |
| | tctagtatta gtttttgcgg agtgaattcc gataccgtta gttggtcatg gccagacgga 1320 |
| | gctgagttgc catttacaat cgataaatag 1350 |
| H7N2 HA | atgaatatac agatactcgc attcatagct tgcgtactta ccggagctaa aggcgataag 60 |
| (A/chicken/ | atatgtctag ggcatcacgc agtcgcaaac ggaacgaaag tgaatacact tacagagaga 120 |
| NJ/294508- | gggatagagg tcgttaacgc tacagagaca gtcgaaaccg caaatattaa aaaaatttgt 180 |
| 12/2004) | acacaaggaa aacgaccaac cgatctggga caatgcggac tgttagggac actgatagga 240 |
| SEQ ID | ccaccacaat gcgatcaatt ccttgagttt agtagcgatc tgataatcga acgaagagag 300 |
| NO: 31 | ggaactgacg tttgttatcc cggtaagttc actaacgaag agagtcttag acagatactg 360 |
| | agacggtcag ggggaatcgg aaaagagtca atggggttta cgtattctgg gattaggact 420 |
| | aatggcgcaa ctagcgcatg tactagaagc ggatcatcat tctatgccga aatgaaatgg 480 |
| | ttgttgtcga attccgataa cgctgcattc ccacaaatga ctaaatcgta tagaaatcct 540 |
| | aggaataaac ccgcactgat aatatgggga gtgcatcata gcgaatccgt aagtgaacag 600 |
| | actaaattgt acggatcagg taataaactg attaaagtga gatctagtaa gtatcagcaa 660 |
| | tcgtttacac ctaatcccgg agctagacgt atcgatttcc attggctatt gctcgaccct 720 |
| | aacgataccg ttacattcac attcaatggc gcattcatag cgccagatag ggcaagtttt 780 |
| | tttagaggcg aatcaatcgg agtgcaatca gacgcaccac ttgactcaag ttgcggaggg 840 |
| | aattgtttcc atagcggagg gactatagtg agtagtctgc cattccaaaa tattaatcct 900 |
| | agaacagtgg gtaagtgtcc tagatacgtt aaacagaaaa gtctgttact cgcaaccgga 960 |
| | atgcgtaacg tacccgaaaa acctaaaaaa aggggattgt tcggagcgat agccggattc 1020 |
| | atagagaatg gatgggaggg actgattaac ggatggtacg gatttagaca ccaaaacgct 1080 |
| | cagggagagg gaaccgcagc cgattataaa tcgacacaat ctgcaatcga tcagattacc 1140 |
| | ggtaagctta atagattgat tggtaagact aatcagcaat tcggactgat agacaatgag 1200 |
| | tttaacgaag tcgagcaaca gatagggaat gtgattaatt ggacacaaga cgctatgact 1260 |
| | gagatttggt cttataatgc cgaactgcta gtcgctatgg agaatcaaca cacaatcgat 1320 |
| | ctaaccgata gcgaaatgtc aaaattgtat gagagagtga gaaaacagct tagagagaat 1380 |
| | gcagaggaag acggaactgg gtgtttcgag atattccata aatgcgacga tcactgtatg 1440 |
| | gaatctatta gaaataatac atacgatcat acacagtata gaacagagtc acttcaaaat 1500 |
| | cggatacaga tagacccagt taaactatct agcggatata aagacataat actgtggttc 1560 |
| | tcattcggag ctagttgttt tctgttgctc gcaatcgcta tgggacttgt attcatatgt 1620 |
| | attaaaaacg gtaatatgca atgtacaatt tgcatatag 1659 |
| H7N2 NA | atgaatccga atcagaaaat cattactatc ggatccgtta gcttgacaat cgcaaccgta 60 |
| (A/chicken/ | tgttttctta tgcagattgc gatactcgca atgaccgtta cattgcattt tagacaaaac 120 |
| NJ/294508- | gagtgttcta ttagcgctaa ctctcaggtc gtgccatgcg aacctacaac cgaaaaagag 180 |
| 12/2004) | gtttgttcaa acgtagtcga ttataggtca tggtctaaac cgcaatgtca gattaccgga 240 |
| SEQ ID | ttcgcaccat tttcgaaaga caattcgatt agactatccg ccggaggcga tatttggata 300 |
| NO: 32 | actagggaac catacgtgtc atgcgataca agtaagtgtt atcaattcgc actcggccaa 360 |
| | gggactacac tcgataacaa acactctaac ggtacaatac acgataggat tagtcatagg 420 |
| | acactgctta tgaacgagtt aggcgtacca ttccatctgg gaactaaaca ggtatgcata 480 |
| | gcctggtcat ctagttcatg tcacgacggt aaggcatggt tgcacgtatg cgtaaccggc 540 |
| | gacgatagaa acgctaccgc ctcattcata tataacggta tgctagtcga ctcaatcggg 600 |
| | tcatggtcac aaaatatact taggacacag gaatccgaat gcgtatgtat taacggatca 660 |
| | tgtacagtcg ttatgaccga cggatccgct agcggtaagg ccgatacacg gatactgttc 720 |
| | gttaaagagg gtaagatagt gcatattagc ccacttagcg gatccgccca acatatcgaa 780 |
| | gagtgttcat gttatcctag atatccgaac gttaggtgcg tttgtaggga taattggaaa 840 |
| | gggtctaatc gacccgttat cgatattaat atggccgatt atagtatcga tagttcatac 900 |
| | gtttgttccg gattagtcgg cgatactcct agaaacgaag atagttctag ctctagtaat 960 |
| | tgtagagacc caaacgaaga gagagggaat cccggagtga aagggtgggc attcgatagc 1020 |
| | ggtgacgacg tttggatggg taggacaatt agtagggact ctagatccgg gtatgagact 1080 |
| | tttagggtga taggcggatg gacaaccgca aactctaaga gtcagactag tagacaggtg 1140 |
| | atagtcgata ataataattg gtccgggtat agcgggattt ttagcgtcga gcataagtca 1200 |
| | tgtattaatc ggtgttttta tgtcgaattg attagggggc gacctaaaga gactagggtg 1260 |
| | tggtggacta gcaattcgat agtcgttttt tgcggtacta gcggaacata cggaaccgga 1320 |
| | agttggccag acggagcgaa tattaatttt atgcctatat aa 1362 |
| H7N3 HA | atgaatacac agatactcgc attcatagcg tgtatgctta tcggaactaa aggcgataaa 60 |
| (A/Canada/ | atttgcttag ggcatcacgc agtcgctaac ggaactaaag tgaatacgct taccgaacgc 120 |
| rv504/2004) | ggaatagagg tcgtgaacgc taccgagaca gtcgaaacag tcaatataaa aaaaatttgt 180 |
| SEQ ID | acacagggaa aaagaccaac cgatctggga caatgcggac tgttagggac actaatcgga 240 |
| NO: 33 | ccaccacaat gcgatcaatt cctcgaattc gacgctaatc tgataatcga acggagagag 300 |
| | ggaactgacg tatgctatcc cggtaagttt acgaacgaag agtcacttag acagatactt 360 |
| | agggggtcag gggggataga caaagagtct atggggttta catatagcgg aatacggact 420 |
| | aacggagcta caagtgcatg tagacgatcc ggatcatcgt tttacgccga aatgaaatgg 480 |
| | ttgttgtcta atagcgataa cgctgcattc ccacaaatga ctaagtctta taggaatcct 540 |
| | agaaataaac ccgcactgat tatttgggga gtgcatcata gtggatcagc aaccgaacag 600 |
| | actaagttgt acggatcagg taataaactg attacagtcg gatcgagtaa atatcagcaa 660 |
| | tcgttcacac ctagtcccgg agctagaccg caagtgaacg gacaatctgg taggattgac 720 |
| | tttcattggt tgcttctaga cccaaacgat acagtgacat tcacttttaa cggagcattt 780 |
| | atcgcacccg atagggctag tttctttagg ggagagtcac tcggagtgca atcagacgta 840 |
| | ccacttgata gcggatgcga aggcgattgt tttcactcag ggggaactat agtgagtagt 900 |
| | ctgccattcc aaaatattaa tcctagaacc gtcggtaagt gtcctaggta cgttaaacag 960 |
| | actagtctat tgctcgcaac cggaatgcgt aacgtacccg aaaatcctaa acaggcatat 1020 |
| | cagaaacgga tgactagggg gctattcgga gcgattgccg gattcataga gaatgggtgg 1080 |
| | gagggactga tagacggatg gtacgggttc agacaccaaa acgctcaggg agagggaaca 1140 |
| | gccgcagact ataagtctac gcaatcggca atcgatcaga ttaccggtaa gcttaataga 1200 |
| | ctgatagaca aaactaatca gcaattcgaa ctgatagaca acgaatttag tgagatagag 1260 |
| | caacagatag ggaatgtgat aaattggact agagactcaa tgactgaggt atggtcatat 1320 |
| | aacgccgaac tgttggtcgc aatggagaat cagcatacaa tcgatctagc cgatagcgaa 1380 |
| | atgaataaac tttacgaaag ggtgcgaaaa caattgcgag agaatgcgga agaggacgga 1440 |
| | accggatgtt tcgaaatttt ccataaatgc gacgatcaat gtatggaatc gattaggaat 1500 |
| | aatacatacg atcatacaca atatagaacc gaatcacttc agaataggat tcaaatcgat 1560 |
| | cccgttaagt tgagtagcgg atataaagac attatactat ggttctcatt cggagctagt 1620 |
| | tgctttctat tgcttgcgat agctatggga ttggtgttca tatgcataaa aaacggtaat 1680 |
| | atgcgatgta cgatttgcat atag 1704 |
| H7N3 NA | atgaatccta atcagaaaat aattactata ggggtcgtta atactacact atctacaatc 60 |
| (A/Canada/ | gctctactaa tcggagtcgg taatctagtc tttaatacag tgatacacga aaagataggc 120 |
| rv504/2004) | gaccatcaga tagtgacaca tcctacaatt atgacacccg aagtgcctaa ttgtagcgat 180 |
| SEQ ID | acaataatta catataacaa taccgttata aacaatatta caacaacaat tataaccgaa 240 |
| NO: 34 | gccgaacgac cattcaaaag tccactaccc ctatgtccat ttagggggtt ttttccgttt 300 |
| | cataaggata acgctatacg gttaggcgaa aataaagacg taatcgttac tagggagcca 360 |
| | tacgttagtt gcgataacga taattgttgg tcattcgcac tcgctcaagg cgcactgtta 420 |
| | gggactaaac actctaacgg aacaattaaa gacagaacac cttataggtc actgataaga 480 |
| | ttccctatcg gaaccgctcc cgtactaggc aattataaag agatatgcat agcatggtca 540 |
| | agttcgtcat gtttcgacgg taaagagtgg atgcacgtat gtatgaccgg taacgataac 600 |
| | gacgctagcg cacagataat atacggaggg cgaatgacag actcaattaa gagttggcgt 660 |
| | aaagacatac tgagaacaca agagtccgaa tgccaatgca tagacggaac ttgcgtagtc 720 |
| | gccgttacag acggacccgc agctaactcc gctgaccata gagtgtattg gattagggag 780 |
| | ggaaggataa taaagtatga gaacgtgcct aagactaaga tacaacatct tgaagagtgt 840 |
| | tcatgttatg tcgacataga cgtgtattgc atatgtagag acaattggaa agggtctaat 900 |
| | aggccatgga tgagaataaa taacgaaact atactcgaaa ccggatacgt atgttctaag 960 |
| | ttccatagcg atacacctag acccgcagac ccatctatta tgtcatgcga tagcccatct 1020 |
| | aacgttaacg gcggacccgg agtcaaaggg ttcggattca aagccggtaa cgacgtttgg 1080 |
| | ttagggagaa ccgttagtac tagcggtagg tccggattcg aaattataaa ggttacagag 1140 |
| | gggtggataa atagtccgaa tcacgttaag tcaattacac aaacacttgt gtctaataac 1200 |
| | gattggtccg gatatagcgg atcattcata gtcaaagcta aggattgctt tcagccatgt 1260 |
| | ttttacgtcg aactgataag ggggagaccg aataaaaacg acgacgttag ttggactagt 1320 |
| | aattcgatag tgacattttg cggattggac aacgaacccg gatccggtaa ttggcctgac 1380 |
| | ggatcgaata tagggtttat gcctaaataa 1410 |
| H7N7 HA | agtaagagta gggggtataa aatgaataca cagatactcg tattcgcact cgttgcgtca 60 |
| (A/ | ataccgacaa acgccgataa gatttgccta gggcatcacg cagtgtcaaa cggaactaaa 120 |
| Netherlands/ | gtgaatacac ttaccgaaag gggcgttgag gtagtgaacg ctacagagac tgtcgaacgg 180 |
| 219/03) | actaacgtac ctaggatttg tagtaagggt aaaagaacag tcgacctagg gcaatgcgga 240 |
| SEQ ID | ctgttaggca caattaccgg accaccacaa tgcgaccaat ttctcgaatt tagcgctgat 300 |
| NO: 35 | ctgattatcg aacggagaga gggatccgac gtttgttatc ccggtaaatt cgttaacgaa 360 |
| | gaggcactga gacagatact tagagaatcc ggagggatag acaaagagac aatggggttt 420 |
| | acatatagcg gaattagaac taacggaact actagcgcat gtaggagatc cggatctagc 480 |
| | ttttacgccg aaatgaaatg gttactgtca aataccgata acgccgcatt tccgcaaatg 540 |
| | actaagtcat ataagaatac taggaaagac cccgcactga taatttgggg gatacaccat 600 |
| | agcggatcga ctaccgaaca gacaaagcta tacggtagcg ggaataaact gataacagtg 660 |
| | ggatcaagta attaccaaca gtcattcgta ccgagtccag gcgctagacc acaagtgaac 720 |
| | ggacaatccg gacgtataga tttccattgg ttgatactga atccgaacga tacagtgaca 780 |
| | tttagcttta acggcgcatt catagcaccc gatagggcat cattccttag gggtaagagt 840 |
| | atggggatac aaagcgaagt gcaagtcgac gctaattgcg aaggcgattg ttatcatagc 900 |
| | ggggggacta ttattagtaa tctgccattc caaaatatta atagtagggc agtgggaaag 960 |
| | tgtccaaggt acgttaaaca ggaatcactg ttactcgcaa ccggaatgaa aaacgtacca 1020 |
| | gagataccta agagacgaag aagggggttg ttcggcgcta tagccggatt catagagaac 1080 |
| | ggatgggagg gactgataga cggatggtac gggttcagac accaaaacgc tcaaggcgaa 1140 |
| | gggacagccg cagactataa gagtacacaa tccgctatcg atcaaattac cggtaagctt 1200 |
| | aatagactga tcgaaaaaac taatcaacaa ttcgaactaa tcgataacga atttacggaa 1260 |
| | gtcgaaagac agattggcaa tgtgataaat tggactagag actctatgac tgaggtttgg 1320 |
| | tcatataacg ccgaactgtt agtcgcaatg gaaaatcagc atacgataga ccttgccgat 1380 |
| | agcgaaatga ataagctata cgaaagggtg aaacgacaat tgagggaaaa cgccgaagag 1440 |
| | gacggaacag ggtgtttcga aatttttcac aaatgcgacg acgattgtat ggctagtatt 1500 |
| | aggaataata catacgacca tagtaagtat agagaggaag cgatacagaa taggattcaa 1560 |
| | atcgatcccg taaaactgtc tagcggatac aaagacgtta tactgtggtt ctcattcgga 1620 |
| | gcgtcatgtt tcatactgct tgcaatcgct atggggttag tgttcatatg cgttaaaaac 1680 |
| | ggaaatatgc gatgtactat ttgtatttaa 1710 |
| H7N7 NA | atgaatccga accaaaaatt gttcgcatta agcggagtcg caatcgcact aagcgtactg 60 |
| (A/ | aatctgttga tagggataag taacgtaggg ttgaacgtat cactacattt gaaagagaaa 120 |
| Netherlands/ | gggcctaaac aggaagagaa tttgacatgt actacaatta atcagaataa tactaccgta 180 |
| 219/03) | gtcgaaaata catacgttaa caatacaaca attattacta agggaaccga tctgaaaact 240 |
| SEQ ID | ccaagttatc tgttactgaa taaatctcta tgtaacgttg agggatgggt agtgatcgca 300 |
| NO: 36 | aaggataacg ccgttagatt cggcgaaagc gaacagatta tagtgactag agagccatac 360 |
| | gtatcatgcg atccaaccgg atgcaaaatg tacgcattac accaagggac aactattagg 420 |
| | aataaacact ctaacggtac gatacacgat agaaccgcat ttagggggtt gattagtaca 480 |
| | ccactcggta caccaccaac cgtttcgaat agcgacttta tgtgcgtagg gtggtctagt 540 |
| | actacatgtc acgacggaat cgctagaatg acaatttgca tacaggggaa taacgataac 600 |
| | gctaccgcaa ccgtatatta taatagaaga ctaactacta ctattaagac atgggctagg 660 |
| | aatatactga gaacgcaaga atccgaatgc gtttgtcata acggtacatg cgccgtagtg 720 |
| | atgaccgacg gatccgctag ttcgcaagca tatactaagg taatgtattt tcacaaaggg 780 |
| | ttagtagtga aagaggaaga gttgaggggg tccgctagac atattgagga atgctcatgt 840 |
| | tacggacata atcaaaaggt gacatgcgta tgtagagaca attggcaagg cgcaaataga 900 |
| | cccattatcg aaatcgatat gagtacactc gaacatacta gtagatatgt gtgtaccgga 960 |
| | atactaaccg atacgagtag acccggcgat aagtctagcg gagattgctc aaacccaatt 1020 |
| | accggatcac ccggagtgcc aggcgttaag ggattcggat tccttaacgg agacaataca 1080 |
| | tggttaggga gaactattag tcctaggagt aggtccggat tcgaaatgct taagatacct 1140 |
| | aacgccggaa ccgacccaaa tagtaggatt gccgaacgac aagagattgt cgacaataac 1200 |
| | aattggtccg gatatagcgg atcattcata gactattgga acgacaatag cgaatgctat 1260 |
| | aacccatgtt tttacgttga gttgattagg ggtagacccg aagaggcaaa atacgtttgg 1320 |
| | tgggcatcta acagtctaat cgcattatgc ggatcaccat ttcccgtagg tagcggatca 1380 |
| | tttcccgacg gagcccaaat tcaatatttt agttaa 1416 |
| H9N2 HA | atggagacaa ttagtctgat tactatacta ttggtcgtta cagcgtcaaa cgctgacaaa 60 |
| (A/Hong | atatgtatag gccatcaatc cactaattca accgaaacag tcgatacact aaccgaaacg 120 |
| Kong/ | aatgtgccag tgacacacgc taaagagcta ctgcataccg aacataacgg aatgctatgc 180 |
| I073/99) | gctactagcc tagggcatcc actgatactc gatacatgta ctatcgaggg actcgtatac 240 |
| SEQ ID | ggtaatccta gttgcgatct actgttaggc ggtagggaat ggtcatacat agtcgaacga 300 |
| NO: 37 | tcatccgccg taaacggaac atgttatccc ggtaatgtcg agaatctcga agagcttagg 360 |
| | acactattct catccgctag ctcataccaa cgaatacaga tttttcccga tactacatgg 420 |
| | aatgtgacat ataccggaac tagtagggca tgttccggat cattctatag atcaatgaga 480 |
| | tggttgacac aaaaatccgg cttttaccct gtgcaagacg cacaatatac gaataatagg 540 |
| | ggtaaatcta tactattcgt atggggtata catcatccac ctacttatac cgaacagact 600 |
| | aatctgtata ttagaaacga tacaactaca tccgttacaa ccgaagactt gaataggaca 660 |
| | ttcaaacccg taatcggacc tagaccacta gtgaacggat tgcagggtag aatcgattac 720 |
| | tattggtccg tacttaagcc agggcaaaca cttagagtga gatctaacgg taatctaatc 780 |
| | gcaccatggt acggacacgt acttagcgga gggtcacacg gtaggatact taagaccgat 840 |
| | ctgaaagggg ggaattgcgt agtgcaatgc caaaccgaaa aaggcggact gaattcgaca 900 |
| | ctaccattcc ataatattag caaatacgca ttcggaacat gtcctaagta cgttagggtg 960 |
| | aatagtctga aactcgcagt gggattgaga aacgtacccg ctagatcgag tagggggcta 1020 |
| | ttcggcgcaa tcgcagggtt tatcgaaggc ggatggccag gactagttgc cggatggtac 1080 |
| | ggattccaac atagtaacga tcaaggcgta gggatggccg ccgataggga tagcacacaa 1140 |
| | aaagcaatcg ataagattac tagtaaggtt aataatatag tcgataagat gaataagcaa 1200 |
| | tacgaaatta tcgatcacga atttagcgaa gtcgaaacta gactgaatat gataaataat 1260 |
| | aagatagacg atcagataca agacgtatgg gcatataacg ccgaactgtt agtgttgctt 1320 |
| | gagaatcaga agacactcga cgaacacgac gcaaacgtta ataatctgta taataaagtg 1380 |
| | aaaagagcac tagggtctaa cgctatggag gacggtaagg gatgtttcga actatatcat 1440 |
| | aaatgcgacg atcaatgcat ggagacaatt agaaacggta catataatcg gagaaagtat 1500 |
| | agagaggaat ctagactcga aagacagaaa atcgaaggcg ttaaactcga atccgaagga 1560 |
| | acatataaga tactgactat ttatagtaca gtcgctagct cactagtgct tgctatggga 1620 |
| | ttcgccgcat tcttgttttg ggctatgtca aacggatcat gtaggtgtaa tatttgtatt 1680 |
| | taa 1683 |
| H9N2 NA | atgaatccga atcagaaaat aatcgcatta gggtccgttt cgattactat agcgactata 60 |
| (A/Hong | tgcctattga tgcaaatcgc aatactcgca acgactatga cattgcattt taacgaatgc 120 |
| Kong/ | actaatccct ctaataatca ggccgttcca tgcgaaccaa tcataatcga acggaatatt 180 |
| I073/99) | accgagatag tgcatcttaa caatacgact atcgaaaaag agtcatgccc taaggtagcg 240 |
| SEQ ID | gaatataaaa attggtctaa gcctcaatgt cagattaccg gattcgcacc attctctaaa 300 |
| NO: 38 | gataattcaa ttaggcttag cgcaggcgga gatatatggg tgactagaga gccatacgta 360 |
| | agttgcggac tcggtaagtg ttatcaattc gcattaggcc aagggacaac ccttaataat 420 |
| | aagcatagta acggtactat acacgatagg agtccacata ggactcttct tatgaacgag 480 |
| | ttaggcgtac cattccattt agggactaaa caggtttgta tcgcatggtc tagtagttca 540 |
| | tgtcatgacg gtaaggcatg gttgcatgtt tgcgttaccg gcgacgatag aaacgctacc 600 |
| | gcttcaatca tatacgacgg tatgcttacc gattcaatcg gatcatggtc taaaaatata 660 |
| | cttagaaccc aagagtccga atgcgtatgt attaacggta catgtacagt cgttatgaca 720 |
| | gacggatccg ctagcggtag ggccgataca aagatactat tcatacgcga aggtaagata 780 |
| | gtgcatatcg gaccattgtc cggatccgca caacacgttg aggaatgctc atgttatcct 840 |
| | agatatcccg aagtgagatg cgtatgtaga gataattgga aagggtcaaa tagacccgta 900 |
| | ctgtatataa acgttgccga ttatagcgtc gatagttcat atgtgtgtag cggactagtg 960 |
| | ggcgatacac ctagaaacga cgattcatct agtagttcga attgtaggga tcctaataac 1020 |
| | gaaagaggcg gaccaggcgt taaagggtgg gcattcgata acggtaacga cgtttggatg 1080 |
| | gggagaacta ttaaaaaaga ttctagatca gggtatgaga cattcagagt ggtggggggg 1140 |
| | tggactaccg ctaactctaa gtctcaaatt aatagacagg tgatagtcga tagcgataat 1200 |
| | tggtcagggt attccggtat ttttagcgtt gagggtaaga catgtattaa taggtgtttt 1260 |
| | tatgtcgaat tgattagggg gcgaccacaa gagactaggg tttggtggac tagtaattcg 1320 |
| | attatagtgt tttgcggaac tagcggaaca tacggaaccg gatcatggcc agacggagcg 1380 |
| | aatataaatt ttatgtctat ataa 1404 |

Texas 50 H3N2 sequences of additional proteins (utilized in various embodiments of the present invention, such as the Singapore deoptimized live attenuated influenza viral sequence and the Texas deoptimized live attenuated influenza viral sequence.)

>>T50-PB1-
SEQ ID NO: 39
AGCGAAAGCAGGCAAACCATTTGAATGGATGTCAATCCGACTCTA
CTGTTCTTAAAAGTTCCAGCGCAAAATGCCATAAGCACAACATTC
CCTTATACTGGAGATCCTCCATACAGCCATGGAACAGGGACAGGG
TACACTATGGACACAGTCAACAGAACACACCAATATTCAGAGAGG
GGGAAGTGGACGACAAATACAGAAACTGGGGCGCCCCAGCTCAAC
CCAATTGATGGACCACTACCTGAGGATAATGAACCAAGTGGATAT
GCACAAACAGACTGTGTCCTGGAGGCTATGGCCTTCCTTGAAGAA
TCCCACCCAGGTATCTTTGAGAACTCATGCCTTGAAACAATGGAA
GCCGTTCAACAGACAAGGGTGGACAAACTAACCCAAGGTCGCCAG
ACTTATGATTGGACATTAAACAGGAATCAACCGGCAGCAACTGCA
TTAGCCAACACCATAGAAGTCTTTAGATCGAACGGATTAACAGCT
AATGAATCAGGAAGGCTAATAGATTTCCTCAAGGATGTGATGGAA
TCAATGGATAAAGAGGAAATGGAGATAACAACACACTTTCAAAGA
AAAAGGAGAGTAAGGGACAACATGACCAAGAAAATGGTCACACAA
AGAACAATAGGGAAGAAAAAGCAAAGAGTGAATAAGAGAGGCTAC
CTAATAAGAGCTTTGACATTGAACACGATGACCAAAGATGCAGAG
AGAGGCAAATTAAAAAGAAGGGCTATTGCAACACCCGGGATGCAA
ATTAGAGGGTTCGTGTACTTCGTTGAAACTTTAGCTAGAAGCATT
TGCGAAAAGCTTGAACAGTCTGGACTTCCGGTTGGGGGTAATGAA
AAGAAGGCCAAACTGGCAAATGTTGTGAGAAAAATGATGACTAAT
TCACAAGACACAGAGCTTTCTTTCACAATCACTGGGGATAACACT
AAGTGGAATGAAAATCAAAACCCCCGAATGTTTTTGGCGATGATT
ACATACATCACAAAGAATCAACCTGAGTGGTTCAGAAACATCCTG
AGCATCGCACCAATAATGTTCTCAAACAAAATGGCAAGACTGGGA
AAAGGATACATGTTCGAGAGTAAGAGAATGAAGCTCCGGACACAA
ATACCTGCAGAAATGCTAGCAAGCATTGACCTGAAGTATTTCAAT
GAATCAACAAGGAAGAAAATTGAGAAAATAAGGCCTCTTCTAATA
GATGGCACAGCATCATTGAGCCCTGGAATGATGATGGGCATGTTC
AACATGCTAAGTACAGTTTTAGGAGTCTCGATACTGAATCTTGGA
CAAAAGAAATACACCAAGACAACATACTGGTGGGATGGGCTCCAA
TCCTCAGATGATTTTGCCCTCATAGTGAATGCACCAAATCATGAG
GGAATACAAGCAGGAGTGGATAGATTCTACAGGACCTGCAAGTTA
GTGGGAATCAACATGAGCAAAAAGAAGTCCTATATAAATAAAACA
GGGACATTTGAATTCACTAGCTTTTTTTATCGATATGGATTTGTG
GCTAATTTTAGCATGGAGCTGCCAAGTTTTGGAGTGTCTGGAATA
AACGAGTCAGCTGACATGAGCATTGGAGTAACAGTGATAAAGAAC
AACATGATAAACAATGACCTTGGACCAGCAACAGCCCAAATGGCT
CTCCAATTGTTCATCAAAGATTACAGATACACATATCGGTGCCAT
AGAGGAGACACACAAATCCAAACGAGAAGATCATTCGAGATAAAG
AAGCTGTGGGACCAAACCCAATCAAGGACAGGACTATTGGTATCA
GATGGGGGACCAAACTTATACAATATCCGGAATCTTCACATCCCT
GAAGTCTGCTTAAAGTGGGAGCTGATGGATGAGAATTATCGGGGA
AGACTTTGTAATCCCCTGAATCCCTTTGTCAGCCATAAAGAAATT
GAGTCTGTAAACAATGCTGTAGTAATGCCAGCCCATGGTCCGGCC
AAAAGTATGGAATATGATGCCGTTGCAACTACGCACTCCTGGATT
CCCAAAAGGAACCGCTCTATTCTAAACACAAGCCAAAGGGGAATT
CTTGAGGATGAACAGATGTACCAGAAGTGCTGCAACTTGTTCGAG
AAATTTTTCCCTAGTAGTTCATATAGGAGACCGATTGGAATTTCT
AGCATGGTGGAGGCCATGGTGTCTAGGGCCCGGATTGATGCCAGA
ATTGACTTCGAGTCTGGAAGGATTAAGAAGGAAGAGTTCTCTGAG
ATCATGAAGATCTGTTCCACCATTGAAGAACTCAGACGGCAAAAA
TAATGAATTTAGCTTGTCCTTCATGAAAAAATGCCTTGTTTCTACT
>T50-PB2-
SEQ ID NO: 40
AGCAAAAGCAGGTCAATTATATTCAGTATGGAAAGAATAAAAGAA
CTACGGAATCTGATGTCGCAGTCTCGCACTCGCGAGATACTGACA
AAAACCACAGTGGACCATATGGCCATAATTAAGAAGTACACATCA
GGGAGACAGGAAAAGAACCCGTCACTTAGGATGAAATGGATGATG
GCAATGAAATATCCAATCACTGCTGACAAAAGGGTAACAGAAATG
GTTCCGGAGAGAAATGAACAAGGACAAACTCTATGGAGTAAAATG
AGTGATGCTGGATCAGATAGAGTGATGGTATCACCTTTGGCTGTA
ACATGGTGGAATAGGAATGGACCCGTGACAAGTACGGTCCATTAC
CCAAAAGTGTACAAAACTTATTTCGACAAAGTCGAAAGGTTAAAA
CATGGAACCTTTGGCCCTGTCCATTTTAGAAATCAAGTCAAGATA
CGCAGAAGAGTAGACATAAACCCTGGTCATGCAGACCTCAGTGCC
AAAGAGGCACAAGATGTAATTATGGAAGTTGTTTTTCCCAATGAA
GTGGGAGCCAGAATACTAACATCAGAATCACAACTAACAATAACT
AAAGAGAAAAAAGAAGAACTCCGAGATTGCAAAATTTCTCCCTTG
ATGGTCGCATACATGTTAGAGAGAGAACTTGTGCGAAAAACAAGA
TTTCTCCCAGTTGCTGGCGGAACAAGCAGTATATACATTGAAGTT
TTACATTTGACTCAGGGAACGTGTTGGGAACAAATGTACACTCCA
GGTGGAGGAGTGAGGAATGACGATGTTGATCAAAGCCTAATTATT
GCGGCCAGGAACATAGTAAGgAGAGCCGCAGTATCAGCAGATCCA
CTAGCATCTTTATTGGAGATGTGCCACAGCACGCAAATTGGCGGA
ACAAGGATGGTGGACATTCTTAGACAGAACCCTACTGAAGAACAA
GCTGTGGATATATGCAAGGCTGCAATGGGATTGAGAATCAGCTCA
TCCTTCAGCTTTGGTGGCTTTACATTTAAAAGAACAAGCGGGTCA
TCAGTCAAAAAAGAAGAAGAGGTGCTTACAGGCAATCTCCAAACA
TTGAGAATAAGAGTACATGAGGGGTATGAGGAGTTCACAATGGTG
GGGAAAAGAGCAACAGCTATACTAAGAAAAGCAACCAGAAGATTG
GTTCAACTCATAGTGAGTGGAAGAGACGAACAGTCAATAGCCGAA
GCAATAATCGTGGCCATGGTGTTTTCACAAGAAGATTGCATGATA
AAAGCAGTTAGAGGTGACCTGAATTTTGTCAACAGAGCAAATCAG
CGGTTGAACCCCATGCATCAGCTTTTAAGGCATTTTCAGAAAGAT
GCGAAAGTGCTCTTTCAAAATTGGGGAGTTGAACACATCGACAGT
GTGATGGGAATGGTTGGAGTATTACCAGATATGACTCCAAGCACA
GAGATGTCAATGAGAGGAATAAGAGTCAGCAAAATGGGTGTGGAT
GAATACTCCAGTACAGAGAGGGTGGTGGTTAGCATTGATCGGTTT
TTGAGAGTTCGAGACCAACGTGGGAATGTATTATTATCTCCTGAG
GAGGTCAGTGAAACACAGGGAACTGAGAGACTGACAATAACTTAT
TCATCGTCGATGATGTGGGAGATTAACGGTCCTGAGTCGGTTTTG
GTCAATACCTATCAATGGATCATCAGGAATTGGGAAGCTGTCAAA
ATTCAATGGTCTCAGAATCCTGCAATGTTGTACAACAAAATGGAA
TTTGAACCATTTCAATCTTTAGTCCCCAAGGCCATTAGAAGCCAA
TACAGTGGGTTTGTCAGAACTCTATTCCAACAAATGAGAGACGTA
CTTGGGACATTTGACACTGCCCAGATAATAAAGCTTCTCCCTTTT
GCAGCTGCTCCACCGAAGCAAAGCAGAATGCAGTTCTCTTCACTG
ACTGTGAATGTGAGGGGATCAGGGATGAGAATACTTGTAAGGGGC
AATTCTCCTGTATTCAACTACAACAAGACCACTAAAAGGCTAACA
ATTCTCGGAAAAGATGCCGGCACTTTAATTGAAGACCCAGATGAA
AGCACATCCGGAGTGGAGTCCGCCGTCTTGAGAGGGTTCCTCATT
ATAGGTAAGGAAGACAGAAGATACGGACCAGCATTAAGCATCAAT
GAACTGAGTAACCTTGCAAAAGGGGAAAAGGCTAATGTGCTAATC
GGGCAAGGAGACGTGGTGTTGGTAATGAAACGAAAACGGGACTCT
AGCATACTTACTGACAGCCAGACAGCGACCAAAAGAATTCGGATG
GCCATCAATTAATACTGAATAGTTTAAAAACGACCTTGTTTCTAC
T
>T50-PA-
SEQ ID NO: 41
AGCAAAAGCAGGTACTGATTCAAAATGGAAGATTTTGTGCGACAA
TGCTTCAACCCGATGATTGTCGAACTTGCAGAAAAAGCAATGAAA
GAGTATGGGGAGGATCTGAAAATTGAAACCAACAAATTTGCAGCA
ATATGCACTCACTTGGAGGTGTGTTTCATGTATTCAGATTTCCAT
TTCATCAATGAACAAGGCGAATCAATAGTGGTAGAACTTGACGAT
CCAAATGCACTGTTAAAGCACAGATTTGAAATAATCGAGGGGAGA
GACAGAACAATGGCCTGGACAGTAGTAAACAGTATCTGCAACACT
ACTGGAGCTGGAAAACCGAAGTTTCTACCGGATTTGTATGATTAC
AAAGAGAACAGATTCATCGAAATTGGAGTGACAAGGAGAGAAGTC
CACATATATTACCTTGAAAAGGCCAATAAGATTAAATCTGAGAAC
ACACACATTCACATTTTTTCATTCACTGGGGAGGAAATGGCCACA
AAGGCAGACTACACTCTCGACGAGGAAAGCAGGGCTAGGATTAAA
ACCAGGCTGTTTACCATAAGACAAGAAATGGCCAACAGAGGCCTC
TGGGATTCCTTTCGTCAGTCCGAAAGAGGCGAAGAAACAATTGAA
GAAAAATTTGAGATCACAGGAACTATGCGCAGGCTTGCCGACCAA
AGTCTCCCACCGAACTTCTCCTGCCTTGAGAATTTTAGAGCCTAT
GTGGATGGATTCGAACCGAACGGCTGCATTGAGGGCAAGCTTTCT
CAAATGTCCAAAGAAGTGAATGCCCAAATTGAACCTTTTCTGAAG
ACAACACCAAGACCAATCAAACTTCCTAATGGACCTCCTTGTTAT
CAGCGGTCCAAATTCCTCCTGATGGATGCTTTGAAATTGAGCATT
GAAGACCCAAGTCACGAAGGAGAAGGGATCCCATTATATGATGCG
ATCAAGTGCATAAAAACATTCTTTGGATGGAAAGAACCTTATATA
GTCAAACCACACGAAAAGGGAATAAATTCAAATTACCTGCTGTCA
TGGAAGCAAGTACTGTCAGAATTGCAGGACATTGAAAATGAGGAG
AAGATTCCAAGAACTAAAAACATGAAGAAGACGAGTCAACTGAAG
TGGGCTCTTGGTGAAAACATGGCACCAGAGAAGGTAGACTTTGAA
AACTGCAGAGACATAAGCGATTTGAAGCAATATGATAGTGAAGAA
CCTGAATTAAGGTCACTTTCAAGCTGGATACAGAGTGAGTTCAAC
AAGGCCTGTGAGCTAACTGATTCAGTCTGGATAGAGCTCGATGAA
ATTGGAGAGGACGTAGCCCCAATTGAGCACATTGCAAGCATGAGA
AGGAATTATTTCACAGCAGAGGTGTCCCATTGTAGAGCTACTGAA
TACATAATGAAGGGGGTATACATTAACACTGCCCTGCTCAATGCA
TCCTGTGCAGCAATGGACGATTTTCAACTAATTCCCATGATAAGC
AAGTGCAGAACTAAAGAGGGAAGGCGAAAAACCAATTTATATGGA
TTCATCATAAAGGGAAGATCTCATTTGAGGAATGACACAGACGTG
GTAAATTTTGTGAGCATGGAGTTTTCTCTCACAGACCCGAGACTT
GAACCACATAAATGGGAGAAATACTGTGTCCTTGAGATAGGAGAT
ATGTTACTAAGAAGTGCCATAGGCCAAATTTCAAGGCCGATGTTC
TTGTATGTGAGGACAAACGGAACATCAAAGGTCAAAATGAAATGG
GGAATGGAGATGAGACGTTGCCTCCTTCAGTCACTCCAGCAGATC
GAGAGCATGATTGAAGCCGAGTCCTCAGTTAAAGAGAAAGACATG
ACCAAAGAGTTTTTTGAGAATAAATCAGAAGCATGGCCCATTGGA
GAGTCCCCCAAGGGAGTGGAAGAAGGTTCCATTGGGAAAGTCTGT
AGGACTTTATTAGCTAAGTCAGTATTCAATAGCCTGTATGCATCA
CCACAATTGGAAGGATTTTCAGCGGAGTCAAGAAAACTGCTCCTT
GTTGTTCAGGCTCTTAGGGACAACCTCGAACCTGGGACCTTTGAT
CTTGGGGGGCTATATGAAGCAATTGAGGAGTGCCTGATTAATGAT
CCCTGGGTTTTGCTCAATGCGTCTTGGTTCAACTCCTTCCTGACA
CATGCATTAAAATAGTTATGGCAGTGCTACTATTTGTTATCCGTA
CTGTCCAAAAAAGTACCTTGTTTCTACT
>T50-NP-
SEQ ID NO: 42
AGCAAAAGCAGGGTTAATAATCACTCACTGAGTGACATCAAAATC
ATGGCGTCCCAAGGCACCAAACGGTCTTATGAACAGATGGAAACT
GATGGAGATCGCCAGAATGCAACTGAGATTAGGGCATCCGTCGGG
AAGATGATTGATGGAATTGGGAGATTCTACATCCAAATGTGCACT
GAACTTAAACTCAGTGATCATGAAGGGCGGTTGATCCAGAACAGC
TTGACAATAGAGAAAATGGTACTCTCTGCTTTTGATGAAAGAAGG
AATAAATACCTGGAAGAACACCCCAGCGCGGGGAAAGATCCCAAG
AAAACTGGGGGGCCCATATACAGGAGAGTCGATGGGAAATGGATG
AGGGAACTCGTCCTTTATGACAAAGAAGAAATAAGGCGAATCTGG
CGCCAAGCCAACAATGGTGAGGATGCTACATCTGGTCTAACTCAC
ATAATGATTTGGCATTCCAATTTGAATGATGCAACATACCAGAGG
ACAAGAGCTCTTGTTCGAACTGGAATGGATCCCAGAATGTGCTCT
CTGATGCAGGGCTCGACTCTCCCTAGAAGGTCCGGAGCTGCAGGT
GCTGCAGTCAAAGGAATCGGGACAATGGTGATGGAACTGATCAGA
ATGGTCAAACGGGGGATCAATGATCGAAATTTTTGGAGAGGTGAG
AATGGGCGGAAAACAAGAAGTGCTTATGAGAGAATGTGCAACATT
CTTAAAGGAAAATTTCAAACAGCTGCACAAAGAGCAATGGTGGAT
CAAGTTAGAGAAAGTCGGAACCCAGGAAACGCTGAGATCGAAGAT
CTCATATTTTTAGCAAGATCTGCACTGATATTGAGAGGATCAGTT
GCTCACAAATCTTGCCTACCTGCCTGTGCGTATGGACCTGCAGTA
TCCAGTGGGTACGACTTCGAAAAAGAGGGATATTCCTTGGTGGGA
ATAGACCCTTTCAAACTACTTCAAAATAGCCAAATATACAGCTTA
ATCAGACCTAACGAGAATCCAGCACACAAGAGTCAGCTGGTGTGG
ATGGCATGCCATTCTGCTGCATTTGAAGATTTAAGATTGTTAAGC
TTCATCAGAGGGACAAAAGTATCTCCTCGGGGGAAACTGTCAACT
AGAGGAGTACAAATTGCTTCAAATGAGAACATGGATAATATGGGA
TCGAGCACTCTTGAACTGAGAAGCAGGTACTGGGCCATAAGGACC
AGGAGTGGAGGAAACACTAATCAACAGAGGGCCTCCGCAGGCCAA
ACCAGTGTGCAACCTACGTTTTCTGTACAAAGAAACCTCCCATTT
GAAAAGTCAACCATCATGGCAGCATTCACTGGAAATACGGAGGGA
AGAACTTCAGACATGAGGGCAGAAATCATAAGGATGATGGAAGGT
GCAAAACCAGAAGAAGTGTCATTCCGGGGGAGGGGAGTTTTCGAG
CTCTCAGACGAGAAGGCAACGAACCCGATCGTGCCCTCTTTTGAT
ATGAGTAATGAAGGATCTTATTTCTTCGGAGACAATGCAGAAGAG
TACGACAACTAAGGAAAAAATACCCTTGTTTCTACT
>T50-M-
SEQ ID NO: 43
AGCAAAAGCAGGTAGATATTGAAAGATGAGCCTTCTAACCGAGGT
CGAAACGTATGTTCTCTCTATCGTTCCATCAGGCCCCCTCAAAGC
CGAGATCGCGCAGAGACTTGAAGATGTCTTTGCTGGGAAAAACAC
AGATCTTGAGGCTCTCATGGAATGGCTAAAGACAAGACCAATTCT
GTCACCTCTGACTAAGGGGATTTTAGGGTTTGTTTTCACGCTCAC
CGTGCCCAGTGAGCGAGGACTGCAGCGTAGACGCTTTGTCCAAAA
TGCCCTCAATGGGAATGGAGACCCAAATAACATGGACAAAGCAGT
TAAACTGTATAGGAAACTTAAGAGGGAGATAACGTTCCATGGGGC
CAAAGAAATAGCTCTCAGTTATTCTGCTGGTGCACTTGCCAGTTG
CATGGGCCTCATATACAATAGGATGGGGGCTGTAACCACTGAAGT
GGCATTTGGCCTAGTGTGTGCAACATGTGAGCAGATTGCTGATTC
CCAGCACAGGTCTCATAGGCAGATGGTGGCAACAACCAATCCATT
AATAAAACATGAGAACAGAATGGTTTTGGCCAGCACTACAGCTAA
GGCTATGGAGCAAATGGCTGGATCAAGTGAGCAGGCAGCGGAGGC
CATGGAGATTGCTAGTCAGGCCAGGCAGATGGTGCAGGCAATGAG
AGCCATTGGGACTCATCCTAGTTCCAGTACTGGTCTAAGAGATGA
TCTTCTTGAAAATTTGCAGACCTATCAGAAACGAATGGGGGTGCA
GATGCAACGATTCAAGTGACCCGCTTGTTGTTGCCGCGAATATCA
TTGGGATCTTGCACTTGATATTGTGGATTCTTGATCGTCTTTTTT
TCAAATGCGTCTATCGACTCTTCAAACACGGCCTTAAGAGAGGCC
CTTCTACGGAAGGAGTACCTGAGTCTATGAGGGAAGAATATCGAA
AGGAACAGCAGAATGCTGTGGATGCTGACGACAGTCATTTTGTCA
GCATAGAGTTGGAGTAAAAAACTACCTTGTTTCTACT
>T50-NS-
SEQ ID NO: 44
AGCAAAAGCAGGGTGACAAAGACATAATGGATTCCAACACTGTGT
CAAGTTTCCAGGTAGATTGCTTTCTTTGGCATATCCGGAAACAAG
TTGTAGACCAAGAACTGAGTGATGCCCCATTCCTTGATCGGCTTC
GCCGAGATCAGAGGTCCCTAAGGGGAAGAGGCAATACTCTCGGTC
TAGACATCAAAGCAGCCACCCATGTTGGAAAGCAAATTGTAGAAA
AGATTCTGAAAGAAGAATCTGATGAGGCACTTAAAATGACCATGG
TCTCAACACCTGCTTCGCGATACATAACTGACATGACTATTGAGG
AATTGTCAAGAAACTGGTTCATGCTAATGCCCAAGCAGAAAGTGG
AAGGACCTCTTTGCATCAGAATGGACCAGGCAATCATGGAGAAAA
ACATCATGTTAAAAGCGAATTTCAATGTGATTTTTGGCCGACTAG
AGACCATAGTATTACTAAGGGCTTTCACCGAAGAGGGAGCAATTG
TTGGCGAAATCTCACCATTGCCTTCTTTTCCAGGACATACTATTG
AGGATGTCAAAAATGCAATTGGGGTCCTCATCGGAGGACTTGAAT
GGAATGATAACACAGTTCAAGTCTCTAAAAATCTACAGAGATTCG
CTTGGAGAAGCAGTAATGAGAATGGGGGACCTCCACTTACTCCAA
AACAGAAACGGGAAATGGCGAGAACAGCTAGGTCAGAAGTTTGAA
GAGATAAGATGGCTAATTGAAGAAGTGAGACACAGATTAAGAACA
ACCGAAAATAGCTTTGAACAAATAACATTCATGCAAGCATTACAA
CTGCTGTTTGAAGTGGAACAGGAGATAAGAACTTTCTCATTTCAG
CTTATTTAATGATAAAAAACACCCTTGTTTCT

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Influenza Vaccine IM and SC Injection in CD1 Mice

CD1 mice were vaccinated with CodaVax-H1N1 or H3N2 Singapore (HA+NA)^{Min} via IM or SC route. Mice were boosted once with the same virus/dose/route. All mice were challenged 10×LD50 of wildtype virus and the survival were monitored. Sera from all mice were draw pre-vaccination, pre-boost, and pre-challenge. HAI assays were performed to determine HAI titer at different time points.

### Experimental Procedures

Mice: CD1 mice. Cells: Vero (MCB+9). Medium and reagents used: Turkey red blood cells, RDE, OptiPRO, DPBS. Viruses: CodaVax-H1N1, A/California/07/2009(H1N1)-Mouse Adapted, A/California/07/2009(H1N1), H3N2 Singapore (HA+NA)^{Min} A/Aichi/2/1968(H3N2), A/Singapore/lNFIMH-16-0019/2016 (H3N2).

CD1 mouse vaccination and boost

| | | Dose | Injection | Number |
|---|---|---|---|---|
| Group# | Vaccine Strain | (PFU) | Route | of Mice |
| 1 | CodaVax-H1N1 | 1.00E+08 | IM | 10 |
| 2 | CodaVax-HIN1 | 1.00E+08 | SC | 10 |
| 3 | CodaVax-H1N1 | 1.00E+07 | SC | 10 |
| 4 | H3N2 Sing | 1.00E+08 | IM | 10 |
| | (HA + NA)Min | | | |
| 5 | H3N2 Sing | 1.00E+08 | SC | 10 |
| | (HA + NA)Min | | | |
| 6 | Mock Sub-Group-1 | OptiPRO | SC | 5 |
| 7 | Mock Sub-Group-2 | OptiPRO | SC | 5 |

Seven groups of female CD1 mice were vaccinated with different doses of H1N1 or H3N2 vaccines via IM or SC in 50 ul diluted in OptiPRO.

On day −3, pre-vaccination sera were collected for all mice. On day 0, all mice received vaccination without anesthesia. On day 21, pre-boost sera were collected for all mice. On day 22, H1N1 groups (Group #1, #2, #3 and #6) received a boost of the same dose via same injection route as initial vaccination. On day 25, H3N2 groups (Group #4, #5, and #7) received boost of the same dose via same injection route as initial vaccination. On day 37, pre-challenge sera were collected from H1N1 groups (Group #1, #2, #3 and #6). On day 42, pre-challenge sera were collected from H3N2 groups (Group #4, #5, and #7).

### Determine LD50 of H1N1 Call WT and H3N2 Aichi WT in CD1 Mice

To define the challenge dose needed, we first determined the LD50 of WT H1N1 Cal7 and H3N2 Aichi in CD1 mice.

LD50 of A/California/07/2009(H1N1)-Mouse Adapted in CD1 mice

| | | | Number of |
|---|---|---|---|
| Group# | Dose (PFU) | Injection Route | Mice |
| 1 | 1.00E+05 | IN | 10 |
| 2 | 1.00E+04 | IN | 10 |
| 3 | 1.00E+03 | IN | 10 |
| 4 | 1.00E+03 | IN | 10 |
| 5 | Mock | IN | 10 |

LD50 of A/Aichi/2/1968(H3N2) in CD1 mice

| Group# | Dose (PFU) | Injection Route | Number of Mice |
|---|---|---|---|
| 1 | 1.00E+04 | IN | 10 |
| 2 | 1.00E+03 | IN | 10 |
| 3 | 1.00E+02 | IN | 10 |
| 4 | 1.00E+01 | IN | 10 |
| 5 | Mock | IN | 10 |

Body weight and survival were monitored for 14 days.

Challenge CD1 Mice with WT Virus

The intranasal LD5 of mouse adapted H1 N1 Cal7 WI is 2.1e+3 PFU in CD1 mice. The intranasal LD51 of H3N2 Aichi WT in CD1 mice is 1e+2 PFU. All vaccinated mice received 10×LD50 of WT virus (H1 N1 group received 2.1e+4 PFU, H3N2 group received 1.0e+3 PFU).

| Group | | Dose | Vaccination | Number | Challenge Strain |
|---|---|---|---|---|---|
| # | Vaccine Strain | (PFU) | Route | of Mice | 10 × LD50 |
| 1 | CodaVax- | 1.00E+08 | IM | 10 | A/California/07/2009(H1N1)- |
| | H1N1 | | | | Mouse Adapted |
| 2 | CodaVax- | 1.00E+08 | SC | 10 | A/California/07/2009(H1N1)- |
| | H1N1 | | | | Mouse Adapted |
| 3 | CodaVax- | 1.00E+07 | SC | 10 | A/California/07/2009(H1N1)- |
| | H1N1 | | | | Mouse Adapted |
| 4 | H3N2 Sing | 1.00E+08 | IM | 10 | A/Aichi/2/1968(H3N2) |
| | (HA + NA)Min | | | | |
| 5 | H3N2 Sing | 1.00E+08 | SC | 10 | A/Aichi/2/1968(H3N2) |
| | (HA + NA)Min | | | | |
| 6 | Mock-1 | — | SC | 5 | A/California/07/2009(H1N1)- |
| | | | | | Mouse Adapted |
| 7 | Mock-2 | — | SC | 5 | A/Aichi/2/1968(H3N2) |

H1N1 Groups (Group #1, #2, #3, and #6) were challenged on Day 42 post vaccination. H3N2 Groups (Group #4, #5, and #7) were challenged on Day 45 post vaccination.

### Determine HAI Titer

To evaluate the immune response elicited by IM and SC injections of the vaccine strains, HAI assay were performed for sera collected pre-vaccination, pre-boost, and pre-challenge.

### Results

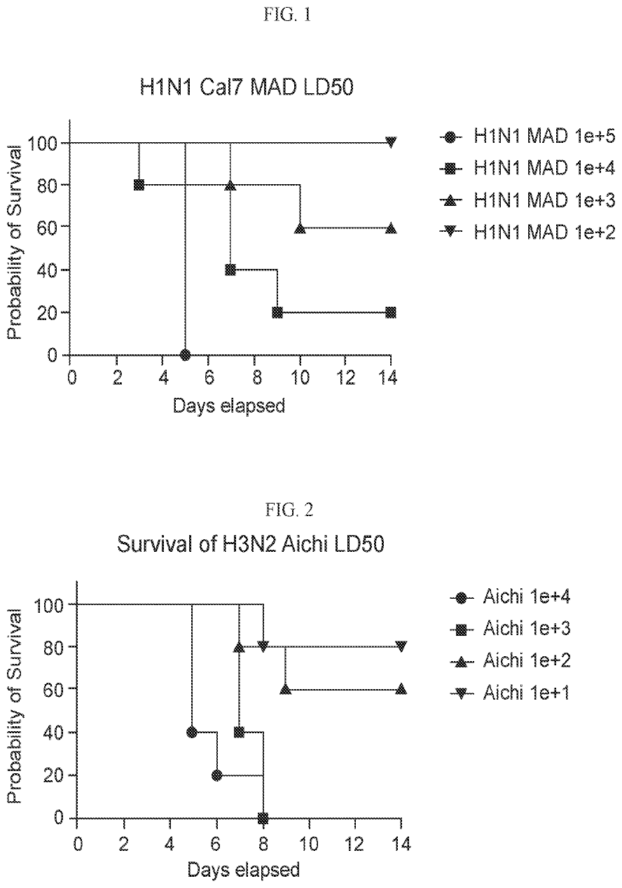

The intranasal LD50 of mouse adapted H1N1 Cal7 WT is 2.1e+3 PFU in CD1 mice. The challenge dose will be 10×LD50=2.1e+4 PFU, diluted in 50 ul OptiPRO. (FIG. 1)

The intranasal LD50 of H3N2 Aichi WT in CD1 mice is 1e+2 PFU. The challenge dose used is 10×LD50=1e+3 PFU, diluted in 50 ul OptiPRO. (FIG. 2)

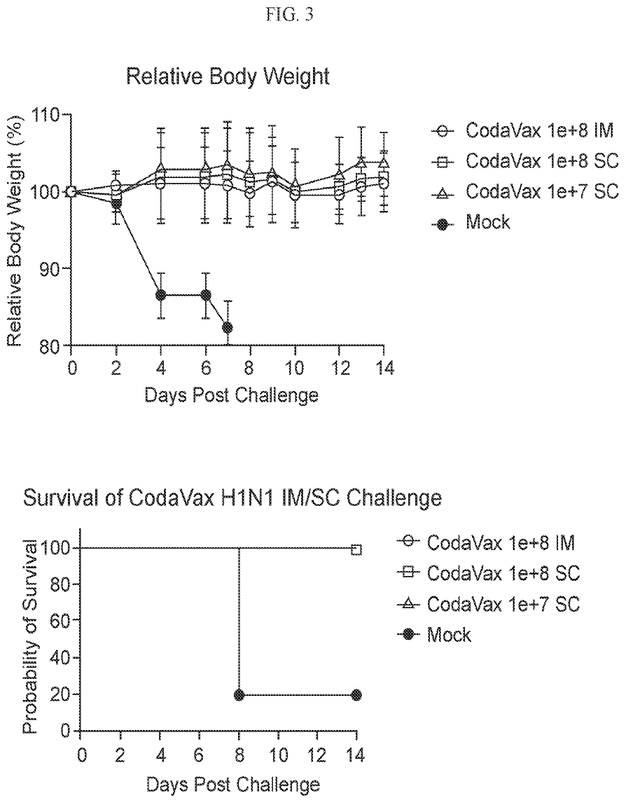

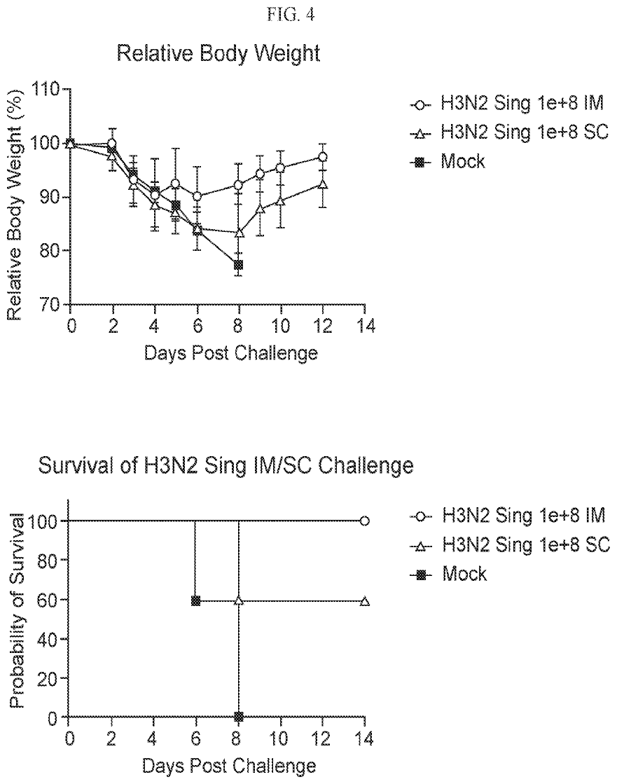

All mice vaccinated with CodaVax-H1N1 either subq or IM were completely protected from challenge with a mouse-adapted wt H1N1 virus. (FIG. 3)

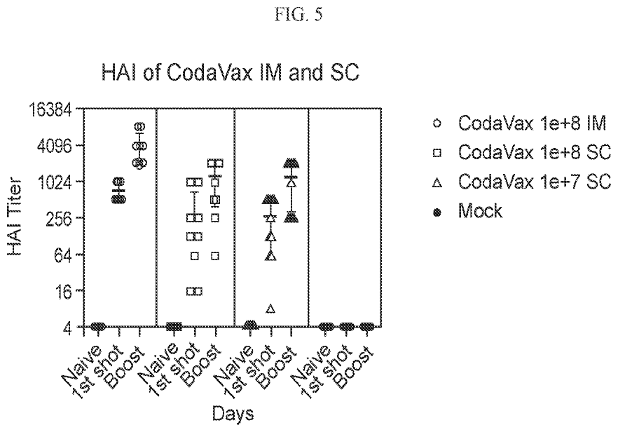

Mice vaccinated IM H3N2 (HA-NA)^{Min} were protected from wt challenge, mice vaccinated subq were partially protected. (FIG. 4)

Number of mice showing 4-fold or more increase in HAI between 1st shot and boost:
1e+8 IM Group: 9 out of 10
1 e+8 SC Group: 8 out of 10
1e+7 SC Group: 7 out of 10

### (FIG. 5)

Number of mice showing a 4-fold or more increase in HAI between 1st shot and boost:
IM Group: 8 out of 10
   SC Group: 6 out of 10

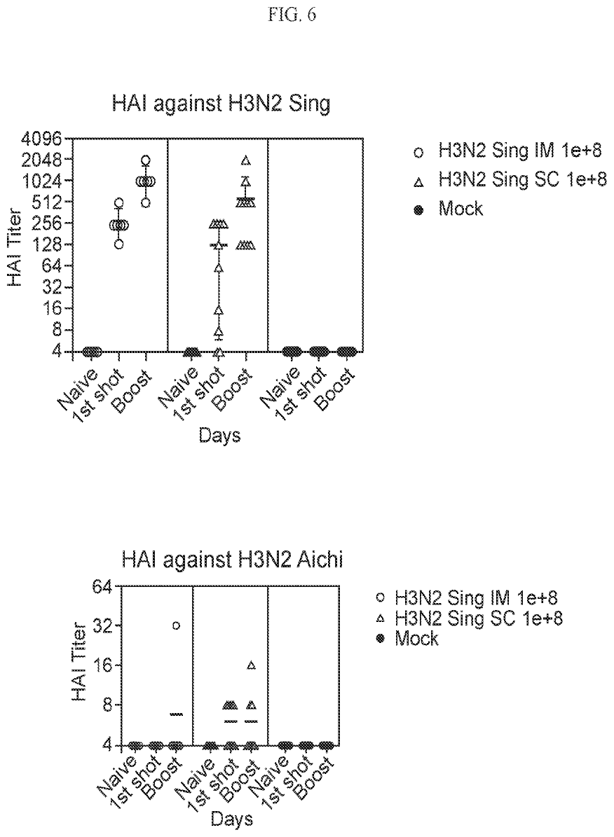

### (FIG. 6)

### Example 2

### H1N1 Influenza Immunogenicity Pilot Study in Ferrets

In this study, CodaVax-H1N1 (CA07-HA/NA-Min) was administered intramuscularly to ferrets in order to evaluate CodaVax-H1N1 immunogenicity in ferrets.

Group Attribution

| | | Adminis- | | | | |
|---|---|---|---|---|---|---|
| | Num- | tration | | | | Eutha- |
| Groups | ber | Route | Candidate | Prime | Boost | nasia |
| 1 | 5 | IM | CodaVax-H1N1 | D 0 | D 21 | D 42 |
| | | | (1 × 10⁸ PFU) | | | |

Animals were pre-screened by HAI assay for the presence of A/California/07/2009 (H1N1) and A/Texas/50/2012 (H3N2) antibodies and needed to be found negative to be included in the study.

### Administrations

IM immunization was performed in 250 μL with 108 PFU of CodaVax H1N1 (CA07-HA/NA-Min) on Day 0 and Day 21.

### Sample Collections

Blood collections were performed on all animals on D0, D2, D4, D6, D14, D21, D23, D25, D27, D35 and D42. The samples were centrifuged at 3200×g for 8 minutes at room temperature (RT) at least 30 minutes after collection. Serum samples were transferred and aliquoted as 5×100 μL/vial in microtubes. The aliquots and remain of serum were stored at −80° C. until use and transportation. All collected blood volumes were per Canadian Council on Animal Care (CCAC) guidelines.

Nasal washes were collected in L15 medium without phenol red on D0, D2, D4, D6, D23, D25 and D27. 4 aliquots of 600 μL per time point were prepared by transferring 540 μL nasal washes into tubes containing 60 μL of bovine serum albumin solution (BSA) solution (20% BSA in L15 medium) and stored at −80° C. until transportation.

### Analysis

HAI against H1N1 CA/07/2009 and H3N2 A/Texas/50/2012 was performed on serum samples.

### Necropsy

Animals were sacrificed on D42. Gross necropsy was performed at euthanasia and findings, if any, were recorded.

### Test System

A total of 5 ferrets (Mustela putorius furo) were obtained from flu free colony at Marshall BioResources and acclimated for 13 days prior to study initiation. Animals had already been identified by supplier with a subcutaneous implant. All animals were pre-screened by HAI and were negative for A/California/07/2009 (H1N1) and A/Texas/50/2012 (H3N2).

For immunization, candidate was performed intramuscularly on Day 0 and Day 21. Group Attribution describes administration route, immunogen type and delivered dose. All animals were observed on the day of immunization and the following 2 days or else weekly for body weight, temperature and clinical signs.

Blood collection were performed on all animals on D0, D2, D4, D6, D14, D21, D23, D25, D27, D35 and D42. Nasal washes were collected on D0, D2, D4, D6, D23, D25 and D27.

### In Vivo Procedures

For immunization, on Day 0 and Day 21, animals were administered intramuscularly with 250 μL of candidate preparations, using disposable Insulin Syringes with 28G needles (BD, Cat. 329424).

Nasal washes were performed with 5 mL sterile syringes (BD, Cat. 309646) to delicately instill 5 mL of L15 medium (Gibco, Cat. 21083027) into nostrils of conscious ferrets. The expulsed liquid was collected into a 100 mm petri dish (Falcon, Cat. 351029) and transferred into a 5 mL collection tube (Corning, Cat. 430656). 540 μL of nasal washes were transferred into tubes containing 60 μL of 20% BSA solution in L15 medium and stored at −80° C. until shipping.

Partial whole blood (D0, D2, D4, D6, D14, D21, D23, D25, D27, D35) collected for serum was obtained via tail vein puncture, using single-use sterile 3 ml syringes with 23GX 1″ needle (Terumo, Cat. SS-03L2325) and 1.1 mL Z-GEL microtubes (Sarstedt, Cat. 41.1378.005). On terminal time point D42, animals were anesthetized by isoflurane, whole blood was collected via abdominal aorta using a Butterfly Safety-Lok collection set with a 21G×12″ needle (BD, Cat. 367281) and collected in 8.5 mL vacutainer tubes (BD, Cat. 367988). After blood collection, tubes were centrifuged for 8 minutes at 3200×g after at least 30 minutes of resting at RT, using a Sorvall Legend RT centrifuge or an Eppendorf 5417 centrifuge. Serum samples were transferred and aliquoted in microtubes and stored at −80° C. After euthanasia, gross necropsy was performed.

### Hemagglutination Inhibition Assay

HAI assay was performed against 2 strains of Influenza: A/California/07/2009(H1N1) and A/Texas/50/2012(H3N2).

To avoid unspecific hemagglutination, samples were treated with a solution of 2% receptor destroying enzyme (RDE) (Cholera Filtrate, Sigma, Cat. C8772) overnight. The reaction was stopped with 1.5% sodium citrate (Millipore Sigma, 1064480500) solution for incubation for 30 minutes and final treatment with 5% chicken RBCs suspension in 0.2% BSA (Sigma, A7030) for one hour at 4° C. After treatment, samples were centrifugated at 1000×g for 10 minutes at 4° C. and the supernatants were transferred for evaluation.

Sample treatment evaluation was performed by hemagglutination (0.5% chicken RBCs) for the assay in V bottom plate (Greiner Bio-One, Cat. 651901). No hemagglutination should be observed after the 45 minutes incubation at RT to qualify treatment as passed.

Virus titration and back titration were performed before the assay to confirm proper virus working dilution (8HA/50 μL) to be used for HAI assay. Viral titers obtained by the last dilution showing hemagglutination corresponded to the HA content.

For HAI assay, positive control reference sera and RBCs alone were added to meet the acceptance criteria. The assay was performed on 2-fold serial dilutions of samples and positive controls in the presence of titrated virus (8 HA/50 μL). Plates were read for hemagglutination after at least 45 minutes of 0.5% RBCs incubation at RT. All samples and positive controls were performed in duplicates. To be compliant with the acceptance criteria, both replicates for a same sample should not be different by more than one dilution.

### Body Weight, Body Temperature and Clinical Signs

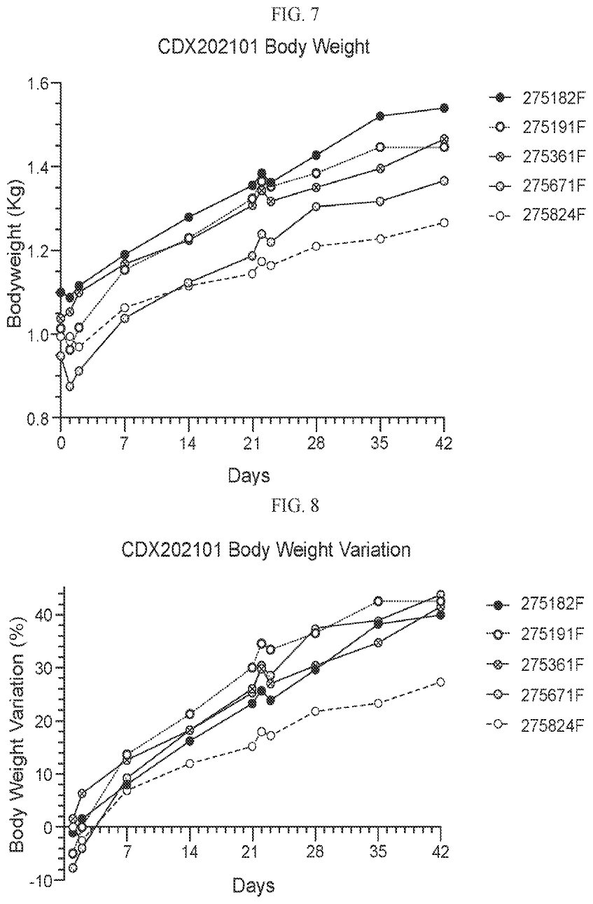

Body weight, body temperature and clinical signs were recorded individually from Day 0 to Day 2, Day 7, Day 14, Day 21 to Day 23, Day 28, Day 35 and Day 42. Body weight measurements showed that all animals experienced progressive weight gain (FIG. 7, FIG. 8; Table 2 and Table 3). It is noteworthy that three out of five animals (275182F, 275191F and 275671F) showed a slight decrease of body weight day 1 after first immunization, which could be due to anesthesia on Day 0. And these ferrets recovered their initial weight and gained weight afterwards,

TABLE 2
Body Weight (kg)

| | Days | | | | | | | | | | |
|---|---|---|---|---|---|---|---|---|---|---|---|
| ID | 0 | 1 | 2 | 7 | 14 | 21 | 22 | 23 | 28 | 35 | 42 |
| 275182F | 1.100 | 1.088 | 1.115 | 1.19 | 1.279 | 1.355 | 1.384 | 1.363 | 1.427 | 1.521 | 1.54 |
| 275191F | 1.015 | 0.963 | 1.016 | 1.153 | 1.231 | 1.322 | 1.366 | 1.354 | 1.385 | 1.448 | 1.448 |
| 275361F | 1.036 | 1.052 | 1.1 | 1.167 | 1.226 | 1.307 | 1.345 | 1.316 | 1.351 | 1.396 | 1.467 |
| 275671F | 0.949 | 0.875 | 0.911 | 1.038 | 1.121 | 1.187 | 1.238 | 1.22 | 1.305 | 1.319 | 1.366 |
| 275824F | 0.994 | 0.995 | 0.969 | 1.063 | 1.114 | 1.144 | 1.173 | 1.165 | 1.21 | 1.228 | 1.266 |

TABLE 3
Body Weight Variation (%)

| | Days | | | | | | | | | |
|---|---|---|---|---|---|---|---|---|---|---|
| ID | 1 | 2 | 7 | 14 | 21 | 22 | 23 | 28 | 35 | 42 |
| 275182F | −1.1 | 1.4 | 8.2 | 16.3 | 23.2 | 25.8 | 23.9 | 29.7 | 38.3 | 40 |
| 275191F | −5.1 | 0.1 | 13.6 | 21.3 | 30.2 | 34.6 | 33.4 | 36.5 | 42.7 | 42.7 |
| 275361F | 1.5 | 6.2 | 12.6 | 18.3 | 26.2 | 29.8 | 27 | 30.4 | 34.7 | 41.6 |
| 275671F | −7.8 | −4 | 9.4 | 18.1 | 25.1 | 30.5 | 28.6 | 37.5 | 39 | 43.9 |
| 275824F | 0.1 | −2.5 | 6.9 | 12.1 | 15.1 | 18 | 17.2 | 21.7 | 23.5 | 27.4 |

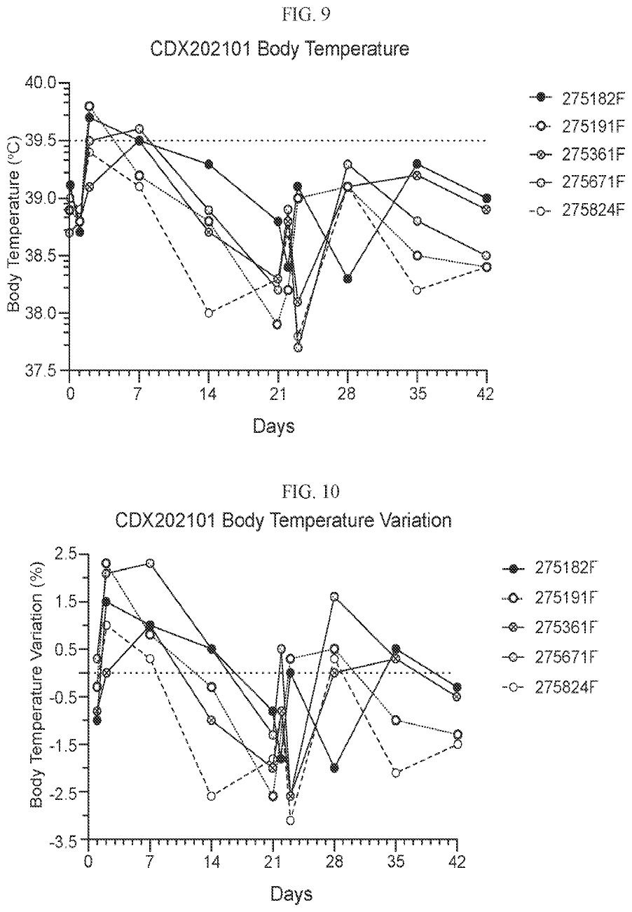

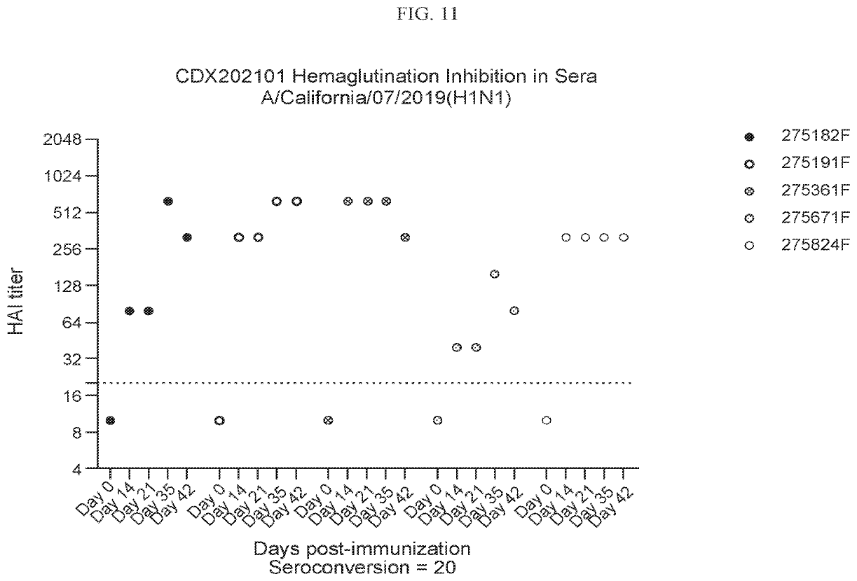

Corresponding to body weight change, animals (275182F, 275191F) had slightly increased temperature (39.7 and 39.8; >39.5 C) after the first immunization (FIG. 9, Table 4; FIG. 10, Table 5). Except that, all animals showed a similar body temperature pattern and no abnormal body temperature was noted throughout the study.

TABLE 4
Body Temperature (° C.)

| | Days | | | | | | | | | | |
|---|---|---|---|---|---|---|---|---|---|---|---|
| ID | 0 | 1 | 2 | 7 | 14 | 21 | 22 | 23 | 28 | 35 | 42 |
| 275182F | 39.1 | 38.7 | 39.7 | 39.5 | 39.3 | 38.8 | 38.4 | 39.1 | 38.3 | 39.3 | 39 |
| 275191F | 38.9 | 38.8 | 39.8 | 39.2 | 38.8 | 37.9 | 38.2 | 39 | 39.1 | 38.5 | 38.4 |
| 275361F | 39.1 | 38.8 | 39.1 | 39.5 | 38.7 | 38.3 | 38.8 | 38.1 | 39.1 | 39.2 | 38.9 |
| 275671F | 38.7 | 38.8 | 39.5 | 39.6 | 38.9 | 38.2 | 38.9 | 37.7 | 39.3 | 38.8 | 38.5 |
| 275824F | 39 | 38.9 | 39.4 | 39.1 | 38 | 38.3 | 38.7 | 37.8 | 39.1 | 38.2 | 38.4 |

TABLE 5
Body Temperature Variation (%)

| | Days | | | | | | | | | |
|---|---|---|---|---|---|---|---|---|---|---|
| ID | 1 | 2 | 7 | 14 | 21 | 22 | 23 | 28 | 35 | 42 |
| 275182F | −1 | 1.5 | 1 | 0.5 | −0.8 | −1.8 | 0 | −2 | 0.5 | −0.3 |
| 275191F | −0.3 | 2.3 | 0.8 | −0.3 | −2.6 | −1.8 | 0.3 | 0.5 | −1 | −1.3 |
| 275361F | −0.8 | 0 | 1 | −1 | −2 | −0.8 | −2.6 | 0 | 0.3 | −0.5 |
| 275671F | 0.3 | 2.1 | 2.3 | 0.5 | −1.3 | 0.5 | −2.6 | 1.6 | 0.3 | −0.5 |
| 275824F | −0.3 | 1 | 0.3 | −2.6 | −1.8 | −0.8 | −3.1 | 0.3 | −2.1 | −1.5 |

No abnormal findings of clinical signs were observed. Only a slight swelling at the injection site followed second immunization was observed.

### Gross Necropsy

At terminal Day 42, some abnormal findings including dark discoloration in the lung lobes and mottled lung, which have a surface having colored spots or blotches, giving an irregular marble appearance, were observed.

### Functional Antibody Measurement by HAI

Results from HAI against A/California/07/2009(H1N1) and A/Texas/50/2012(H3N2) were displayed in FIG. 11. All five animals that received CodaVax-H1N1 immunization gained a functional antibody response against the A/California/07/2009(H1N1) virus from 14 days post immunization. Boost immunization on Day 21 enhanced the seroconversion, as indicated by increased functional antibody titer in three out five animals on Day 35 and Day 42 compared to Day 21. All animals did not show a functional antibody response against A/Texas/50/2012(H3N2).

These results confirmed that CodaVax vaccine candidate induced functional antibodies against H1N1 strain while no induction of functional antibodies against H3N2 strain.

### Example 3

### Detection and Quantification of Plaque Forming Units/mL of CodaVax-H1N1 in Ferret Serum and Nasal Lavage Samples from Example 2

An immunogenicity study using CodaVax-H1N1 in 5 male ferrets (Mustela putorius furo) at least 16 weeks of age was performed. The animals were dosed by the intramuscular route with 1×10⁸ PFU CodaVax-H1N1 at day 0 and boosted at day 21.

275 cryovials of ferret sera collected at day 0, day 2, day 4, day 6, day 14, day 21, day 23, day 25, day 27, day 35 and day 42 (5 aliquots of each time point taken from 5 ferrets) and 175 cryovials of ferret nasal lavage samples collected at day 0, day 2, day 4, day 6, day 23, day 25, and day 27 (5 aliquots of each time point taken from 5 ferrets) were transferred from Nexelis (Laval, Canada) to Codagenix Inc (Farmingdale, NY) on 7/20/21. The samples were received at Codagenix Inc. on 7/21/21 and stored at −80±10° C. until the time of the test.

55 cryovials of ferret serum and 35 vials of ferret nasal lavages from each animal, each time point were thawed and then sorted according to pre-recorded 12-well plate templates prior to the Test.

A plaque assay on MDCK.2 cells was used to determine the presence of CodaVax-H1N1 in serum or nasal lavages of ferrets vaccinated with 1×10⁸ PFU of CodaVax-H1N1. The personnel conducting this experiment were blinded to the identity of the groups.

### Number of Plaques Detected in Nasal Lavages

No virus plaques were detected in any ferret nasal lavage sample at a 1:5 dilution, which suggests that the viral load in all nasal lavages was below the lower level of quantitation (10 PFU/mL).

### Number of Plaques Detected in Serum

During the first plaque assay using a 1:5 dilution of ferret serum, most serum sample wells displayed a degraded MDCK cell monolayer at the conclusion of the assay. This made the viral load unable to be detected as no plaques could be formed. The cause of this apparent cytotoxic effect by the test sera on the MDCK cells is not known, but appears to be unrelated to the treatment of the animals with the CodaVax-H1N1 vaccine candidate, as it was also observed in several d0 serum samples (prior to exposure of the animals to CodaVax-H1N1). For detection of potential live vaccine virus, the serum samples could not be heat inactivated prior to the test. As such, heat-sensitive mediators of cytotoxicity may have been present in the test sera.

The test was repeated with a higher dilution of serum (1:50 dilution) to reduce potential immune effects. The second plaque assay had an intact monolayer for each sample with no virus plaques detected. This suggests that the viral load in all ferret serum samples was below the lower level of quantitation of this set of assays (100 PFU/mL).

### Conclusions

No virus plaques were detected in any ferret nasal lavage sample at the 1:5 dilution. Thus, the virus load in the ferret nasal lavages is beneath the lower limit of quantitation (10 PFU/ml). Upon diluting out any potential cytotoxic serum components of ferret serum through a 1:50 dilution, no viral plaques detected in any of the serum samples. Therefore, the virus load in the ferret serum is beneath the lower limit of quantitation (100 PFU/mL).

### Example 4

### Vaccine Study of CodaVax-H1N1 in African Green Monkeys

The objective of this non-GLP study was to evaluate the immunogenicity of the vaccine candidate CodaVax-H1N1 in the African Green Monkey model. Six African Green Monkeys (three males and three females) were anesthetized and vaccinated with 1×10⁸ PFU CodaVax-H1N1 (0.5 mL/animal) via the intramuscular route on Study Days 0 and 21. Clinical observations, body weights, food consumption, and body temperatures were recorded for all animals over the course of the study. Additionally, blood samples, processed for serum and PBMCs, as well as nasal and tracheal lavage samples were collected at various time points during the study. The samples were assessed for various clinical and hematological parameters.

No abnormal clinical findings or changes in body weight or changes in body temperatures were observed in any of the vaccinated animals throughout the study period. A transient loss of appetite was observed in some animals following primary and secondary vaccination. No remarkable changes in chemistry or hematological parameters were observed in any of the animals during the study period. qRT-PCR analysis for the presence of viral genome in plasma, nasal swabs, and tracheal lavage samples was carried out at Codagenix Inc. All samples were below the lower limit of quantification. The single positive plasma sample by qRT-PCR had no detectable infectious virus via plaque assay. Serum was collected from all animals on Days 0, 21 and 35 and the HAI titer was determined at Codagenix Inc. All animals had HAI serum antibody titers. Serum collected at Day 21 and Day 35 showed about 30-fold and more than 150-fold, respectively, higher antibody titer as compared to Day 0. Overall, intramuscular vaccination with live-attenuated CodaVax-H1N1 vaccine did not cause flu-like symptoms in vaccinated African Green Monkeys. The vaccinated animals did not develop viremia or viral shedding in nasal or tracheal secretions. The vaccination also induced HAI antibodies and enhanced titers were observed following booster vaccination on Day 21.

TABLE 6
HAI titer of monkey serum samples against rA/CA/07/09 H1N1 at
different time post vaccination with live attenuated CodaVax-H1N1.

| Animal | | HAI titer | | |
|---|---|---|---|---|
| ID | Sex | day 0 | day 21 | day 35 |
| 16733 | Male | <5 | 80 | 320/640 |
| 16734 | Male | <5 | 320 | 1280 |
| 16735 | Male | 5 | 160 | 1280 |
| Titer average | | <5/5 | 186.67 | 1013.33 |
| STDEV | | | 122.20 | 461.88 |
| 16745 | Female | <5 | 80 | 320/640 |
| 16746 | Female | <5 | 320 | 1280 |
| 16747 | Female | 5 | 80 | 640 |

| | | | |
|---|---|---|---|
| Titer average | <5/5 | 160.00 | 800.00 |
| STDEV | | 138.56 | 423.32 |

### Test System

Species and Strain: Nonhuman primate, African Green Monkey (Clorocebus aethiops)
Supplier: Approved Vendor
Quarantine: All animals were quarantined in the ABSL-2 facility prior to study initiation. The animals were pre-screened by the Sponsor and were negative for H1N1 and H3N2 antibodies.
Age on Study Day 0: 2-4 years
Weight on Study Day 0: 3.5-4.1 kg
Number on Study (Sex): 3 (male) and 3 (female)

During the study phases, the animals were single-housed in stainless steel cages, in an environmentally monitored and ventilated room maintained per Standard Operating Procedure (SOP). Housing and animal care conformed to the guidelines of the U.S. Department of Agriculture (Animal Welfare Act; Public Law 99-198), the Guide for the Care and Use of Laboratory Animals.

Animals were uniquely identified with a tattoo. They were fed twice per day with Envigo 2050C feed. Water was available ad libitum and provided from the Birmingham public water supply during the quarantine and study periods via water bottles or automatic watering system. Each animal was provided consumable enrichment as well as one or more enrichment devices or toys in the cage per Southern Research SOP.
Vaccine Name: CodaVax-H1N1
Lot Number: 1-031321-1
Manufacturer: Codagenix
Special Handling: The test article is a live-attenuated virus vaccine and was handled under BSL-2/ABSL-2 conditions.
Characterization: Characterization of test material was performed by the Sponsor.
Route: Vaccination material was administered intramuscularly.
Stability and Storage: Vaccine material was stored at ≤−70° C. until administered. Material was stored on wet ice during transfer and vaccination. All residual vaccine was discarded following administration.

### Study Design

Six African Green Monkeys (AGMs) were placed into a single study group as outlined.

Group Assignment

| Group | Vaccine | Vaccine Schedule | Route, Dosage |
|---|---|---|---|
| 1 (n = 6) | CodaVax-H1N1 | Day 0, Day 21 | IM, 1 × 10⁸ PFU/animal |

### In Vivo Test Procedures

In vivo test procedures are outlined below and summarized in Table 7. Animals were anesthetized intramuscularly with Ketamine HCl (10-30 mg/kg) for the following procedures:
Vaccination
Detailed clinical observations
Temperature and body weight collections
Nasal swab collection
Tracheal lavage
Blood collection

### Vaccination

On Study Days 0 and 21, all animals were anesthetized and vaccinated with 1×10⁸ PFU CodaVax-H1N1 (0.5 mL/animal) via the intramuscular (M) route.

### Clinical Observations

The animals were observed twice daily throughout the quarantine and study period for signs of morbidity and mortality. They were assessed for moribundity using the following criteria:
Weight loss of greater than 20% of normal weight (prior to challenge)
Respiratory distress
Appetite loss
Acute dehydration
Neurological deficits
Severe lethargy

### Blood Collection and Processing

Blood samples were collected from all anesthetized animals into EDTA tubes or serum separator tubes (SST) at the time points and volumes indicated in Table 7. Blood collected in appropriate tubes was processed following Southern Research SOPs or Sponsor recommendations, aliquoted into appropriate vials, stored at ≤−70° C., and shipped to the Sponsor at the end of the study. PBMCs were isolated from blood collected in EDTA tubes, stored at ≤−70° C. for at least 12 hours, moved to liquid nitrogen, and then shipped on liquid nitrogen to the Sponsor.

### Body Weight and Temperature

Rectal temperature and body weights were recorded for all animals, as outlined in Table 7.

### Food Consumption

Food consumption was qualitatively assessed daily on Days 0-7 and Days 21-28.

### Clinical Chemistry

Blood was collected as outlined in Table 7 and processed for clinical chemistry. The following parameters were analyzed: Albumin/Globulin Ration (A/G Ratio), Albumin (Alb), Alkaline Phosphatase (ALP), Alanine Aminotransferase (ALT), Aspartate Aminotransferase (AST), Blood Urea Nitrogen (BUN), Calcium (Ca), Chloride (Cl), Cholesterol (Chol), Creatinine (Crea), Globulin (Globulin), Glucose (Gluc), Potassium (K), Sodium (Na), Sodium/Potassium Ratio (Na/K Ratio), Total Bilirubin (TBIL), and Total Protein (TP).

### Hematology

Blood was collected as outlined in Table 7 and processed for complete blood cell counts. The following parameters were analyzed: white blood cell count (WBC), neutrophil count (Neut), neutrophil percent (Neut %), lymphocyte count (Lymph), lymphocyte percent (Lymph %), monocyte count (Mono), monocyte percent (Mono %), eosinophil count (Eosin), eosinophil percent (Eosin %), basophil count (Baso), basophil percent (Baso %), red blood cell count (RBC), hemoglobin (HGB), hematocrit (HCT), mean corpuscular volume (MCV), mean corpuscular hemoglobin (MCH), mean corpuscular hemoglobin concentration (MCHC), red blood cell count distribution width (RDW), platelet count (PLT), and mean platelet volume (MPV).

### Nasal Swab Collection

Nasal swabs were collected from anesthetized animals as outlined in Table 7. The samples were collected by gently swabbing the inside of the nasal cavity with a sterile swab. Following collection, the tip of the swabs was placed immediately into a sterile, individually labeled tube containing ice cold 0.5 mL of L15 media. The tubes were then stored at ≤−70° C. and shipped on dry ice to the sponsor at the end of the study.

### Tracheal Lavage Collection

Tracheal lavage samples were collected at the time points indicated in Table 7. The animals were anesthetized and a tube was inserted into the trachea. Once the end of the tube was situated approximately at the mid-point of the trachea, a syringe containing up to 5 mL of L15 media was attached to the tube and the medium was slowly instilled into the trachea. Once the instillation was complete, negative pressure was immediately applied via the same syringe to collect as much of the L15 media as possible. Samples were snap frozen immediately following collection, stored at ≤−70° C., and shipped on dry ice to the Sponsor at the end of the study.

TABLE 7
Key In Vivo Procedures

| Study Day | −2 | 0 | 1 | 2 | 4 | 7 | 14 | 21 | 23 | 28 | 35 |
|---|---|---|---|---|---|---|---|---|---|---|---|
| Vaccination | | X | | | | | | X | | | |
| Clinical Signs and Food | | Daily from Days 0-7 | | | | | | Daily from Days 21-28 | | | |
| Consumption | | | | | | | | | | | |
| Body Weights and | | X | | X | X | X | | X | X | | X |
| Temperatures | | | | | | | | | | | |
| Tracheal Lavage* | | X | | X | X | X | | X | X | | |
| Nasal Swabs* | | X | | X | X | X | | X | X | | |
| Blood for PBMCs (isolate | 3 | | | | | 3 | 3 | | | 3 | 3 |
| and cryopreserve)* | | | | | | | | | | | |
| EDTA (mL) | | | | | | | | | | | |
| Plasma (shedding)* | | | | 2 | 1 | 1 | | | | | |
| EDTA (mL) | | | | | | | | | | | |
| Blood: Serum for HAI* | | 1 | | | | | | 1 | | | 1 |
| SST Tube (mL) | | | | | | | | | | | |
| Blood: CBC | | 1 | | 1 | | | | | 1 | | |
| EDTA Tube (mL) | | | | | | | | | | | |
| Blood: Chemistry | | 1 | | 1 | | | | | 1 | | |
| Total Blood Volume (mL) | 3 | 3 | | 4 | 1 | 4 | 3 | 1 | 2 | 3 | 4 |
| End of study | | | | | | | | | | | X |
| (no euthanasia) | | | | | | | | | | | |

*Samples shipped to Sponsor for analysis.

### Clinical Observations and Food Consumption

No unusual clinical findings were reported for any of the animals during the study period. Animal food consumption was qualitatively assessed daily on Days 0-7 and Days 21-28. Two females (#16746 and #16747) had reduced food intake on Day 5 following initial vaccination. Animal #16747 did not eat on Day 5. Both animals returned to normal food intake by Day 7 post initial vaccination. Similarly, reduced food intake was noticed on Day 22 and Day 24 following booster vaccination on Day 21. Two females (#16746 and #16747) did not consume food on Day 24. All animals regained appetite by Day 25 and normal food consumption was recorded till the end of study period.

### Blood Analysis: Clinical Chemistry and Hematology

Clinical chemistry and hematology parameters were measured on days indicated in Table 7. No remarkable changes in chemistry or hematological parameters were observed in any of the animals in the study.

### Body Weights

Body weights were recorded on days indicated in Table 7. Overall, the animals maintained body weight over the period of study.

### Body Temperatures

There were no remarkable changes in body temperature over the course of study.

qRT-PCR Analysis of Plasma, Nasal Swab, and Tracheal Lavage Samples

Quantification of plaque forming unit equivalents per milliliter (PFUeq/mL) using qRT-PCR was performed on the plasma, nasal swab, and tracheal lavage samples collected on days outlined in Table 7.

### Hemagglutination Inhibition Assay

Serum samples, collected on days outlined in Table 7, were tested using the Hemagglutination Inhibition (HAI) Assay.

### Results, Discussion and Conclusions

To evaluate the immunogenicity of live attenuated CodaVax-H1N1, serum samples from 6 monkeys treated with CodaVax-H1N1 were tested using HAI assay against wild type rA/CA/07/09 H1N1. Serum (from both male and female subjects) collected at day 0 showed HAI titer<5 or 5. The geometric mean HAI titer of male or female animals was 160.00 and 126.99 on day 21, and 923.04 and 732.62 on day 35, respectively.

Hemagglutination-Inhibition (HAI) serum antibody titer

| | Geometric Mean | Average | STDEV | |
|---|---|---|---|---|
| Day 0 | Male | <5 | <5 | |
| | Female | <5 | | |
| Day 21 | Male | 160 | 143.50 | 23.34 |
| | Female | 126.99 | | |
| Day 25 | Male | 923.04 | 827.83 | 134.65 |
| | Female | 732.62 | | |

Serum antibodies in monkey were assessed using HAI to determine immunogenicity of CodaVax-H1N1 following intramuscular administration. The HAI assay is a principal method for quantifying the relative concentration of viruses, bacteria, or antibodies.

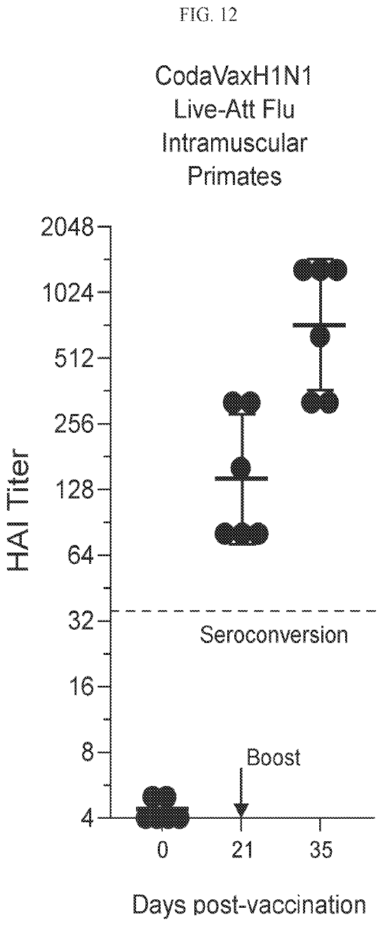

All animals seroconverted. Serum collected at day 21 and day 35 showed about 30-fold and more than 150-fold respectively higher antibody titer as compared to day 0 (FIG. 12). Boosting was beneficial in bringing antibody titers up.

### Example 5

### H3N2 Influenza Vaccine Safety and Efficacy in Ferrets

Ferrets are exquisitely susceptible to infection with human influenza viruses and are widely believed to be an indispensable model for the study of respiratory viruses in general, and influenza viruses in particular. Moreover, the ferret model has tools available which are useful to evaluate the impact of immunomodulatory treatments on normal immune responses. In this study, various candidates were administrated intranasally/subcutaneously/intramuscularly to ferret in order to evaluate their effect on the ferret raised against the candidates and pathogenesis response triggered by influenza infection.

Animals were pre-screened by Hemagglutination Inhibition Assay for the presence of A/California/07/2009 (H1N1) and A/Texas/50/2012 (H3N2) antibodies and needed to be found negative to be included in the study.

Group Attribution

| | | Administration | | | | |
|---|---|---|---|---|---|---|
| Groups | Number | Route | Candidate | Prime | Boost | Euthanasia |
| 1 | 6 | IN | A/Texas/50/2012 | 10⁶ | N/A | D 2/D 30 |
| | | | (H3N2) | | | |
| | | | Control | | | |
| 2 | 6 | IN | A/Texas/50/2012 | 10⁶ | 10⁶ | D 2/D 30 |
| | | | (H3N2) | | | |
| | | | Control | | | |
| 3 | 6 | IN | Codavax H3N2 | 10⁶ | N/A | D 2/D 30 |
| 4 | 6 | SC | Codavax H3N2 | 10⁷ | 10⁷ | D 2/D 30 |
| 5 | 6 | SC | Codavax H3N2 + | 10⁷ + 2 × 10⁷ | 10⁷ + 2 × 10⁷ | D 2/D 30 |
| | | | Codavax H1N1 | | | |
| 6 | 6 | IN | Mock Control | N/A | N/A | D 2/D 30 |
| 7 | 3 | IM | Fluzone | 15 ug/strain | 15 ug/strain | D 30 |

### Candidate Treatment

Intranasal, Subcutaneous or Intramuscular prime immunization was performed in 250 μL, 125 uL per nostril, with 10⁶ pfu of A/Texas/50/2012 (H3N2) (Group 1 and Group 2), 10⁶ pfu of CodaVax-H3N2 (Group 3), 10⁷ pfu of CodaVax-H3N2 (Groups 4 and Group 5), 2×10⁷ pfu of CodaVax-H1N1 (Group 5), virus diluent (Group 6) or Fluzone (Group 7) on Day 0.

Intranasal, Subcutaneous or Intramuscular boost immunization was performed in 250 μL, 125 uL per nostril, with 10⁶ pfu of H3N2 Texas/50/2012 (Group 2), 10⁷ pfu of CodaVax-H3N2 (Groups 4 and Group 5), 2×10⁷ pfu of CodaVax-H1N1 (Group 5) or Fluzone (Group 7) on Day 14.

### Viral Infection

All animals were infected with 10⁶ pfu of A/Singapore/INFIMH-16-0019/2016 (H3N2) in a volume of 1 mL via intranasal route on Day 28.

### Serum Collection

Blood collection were performed on all animals on Day 0, on half of the animals from Group 1 to Group 6 on Day 2 and on remaining animals on Day 14, Day 28 and Day 30.

The samples were centrifuged at 3200×g for 8 minutes at room temperature (RT) at least 30 minutes after collection. Serum samples were transferred and aliquoted in microtubes and stored at −80° C. until use.

### Nasal Washes Collection

Nasal washes were done with 5 mL of PBS. Nasal washes were collected from all animals on Day 0 to Day 2 and from remaining animals on Day 4, Day 6, Day 8, Day 14 to Day 17, Day 19, Day 21, Day 23, Day 29 and Day 30. Nasal washes were vortexed and 540 μL were transferred into tubes containing 60 μL of BSA solution (20%) and stored at −80° C. until viral titration.

### Nasal Turbinates, Olfactory Bulbs and Lungs Collection:

Half of the animals from Group 1 to Group 6 were sacrificed on Day 2 and the remaining animals were sacrificed on Day 30. After euthanasia, lungs (right cranial lobe, right middle lobe, accessory lobe, right caudal lobe), nasal turbinates (right half) and olfactory bulbs (right half) were collected aseptically, weighted and placed in 3 mL of DMEM/Medium 199 with 0.1% FBS in a Precellys tube at 4° C. Lungs, nasal turbinate and olfactory bulbs in Precellys tubes were disrupted with two 20 seconds cycles at 5000 rpm with 5 seconds pause between cycles. Tissue homogenates were frozen at −80° C. until viral titration. Lungs (left cranial lobe and left caudal lobe), nasal turbinates (left half) and olfactory bulbs (left half) were collected and fixed in 10% formalin for histopathology analysis.

### Analysis

Viral titers were determined using TCID50 on nasal washes, serum, nasal turbinates, olfactory bulbs and lungs. Histopathology analysis was performed on lungs, olfactory bulbs, and nasal turbinates by a Board-Accredited pathologist. HAI against H1N1 CA/07/2009 and H3N2 A/Texas/50/2012 (Turkey RBCs) was performed on serum.

### Necropsy

Gross necropsy was performed at euthanasia and findings, if any, were recorded.

### In Vivo Procedures

For immunization, animals were administered intranasally with 250 μL (125 μL per nostril) of candidate preparations (Group 1 to Group 3, Day 0; Group 2, Day 14) or PBS (Group 6, Day 0 and Day 14) with a micropipette. On Day 0 and Day 14, candidate preparations (250 μL, Group 4 to Group 5) or Fluzone (125 μL, Group 7) were administered either by subcutaneous or by intramuscular route, using 25G disposable needles (Terumo, Cat. SS-01T2516) or disposable 0.5 ml prefilled syringes provided by Sanofi.

For viral infection, all animals were administered intranasally with 1 mL (500 μL per nostril) of viral preparation with a micropipette.

Partial whole blood (Day 0) collected for serum were obtained via tail vein puncture, using single-use sterile 1 ml 25G ⅝″ syringes (Terumo, Cat. SS-01T2516) and 1.1 mL Z-GEL microtubes (Sarstedt, Cat. 41.1378.005). Partial whole blood (Day 14 and Day 28) collected for serum were obtained via tail vein puncture, using single-use sterile 3 ml 22G syringes (Terumo, Cat. SS-03L2225) and 3.5 mL vacutainer tubes (BD, Cat. 367983). Tubes were centrifuged (8 minutes at 3200×g) after at least 30 minutes of resting at room temperature, using a Sorvall Legend RT centrifuge or an Eppendorf 5417 centrifuge. Serum samples were transferred and aliquoted in microtubes and stored at −80° C. until use.

Nasal washes were collected using 5 mL sterile syringes (BD, Cat. 309646) to delicately instill 5 mL of D-PBS (Wisent, Cat. 311-010-LL)/0.1% BSA (Sigma, Cat. A7030) solution into nostrils of awaken ferrets. The expulsed liquid was collected into a petri dish and transferred into a 5 mL collection tube (Corning, Cat. 430656). Washes were vortexed and 540 μL were transferred into tubes containing 60 μL of BSA solution (20%) and stored at −80° C. until use.

On terminal time point D30, animals were anesthetized by isoflurane, whole blood was collected via abdominal aorta using a Butterfly Safety-Lok collection set with a 23G needle (BD, Cat. 367296) and collected in 8.5 mL vacutainer tubes (BD, Cat. 367988). After euthanasia, gross necropsy was performed prior to organ collection, lung (around 1 cm³ of right cranial lobe, right middle lobe, accessory lobe, right caudal lobe), nasal turbinates (half) and olfactory bulbs (half) were collected aseptically, weighted and placed in a pre-weighted 7 mL Precellys tube containing 3 mL of in DMEM/Medium 199 with 0.1% FBS at 4° C. Lungs, nasal turbinates and olfactory bulbs in Precellys tubes were weighed, homogenized and frozen for viral titration. Lungs (left cranial lobe and left caudal lobe), nasal turbinates (other half) and olfactory bulbs (other half) were collected and fixed in 10% formalin for histopathological analysis.

### Influenza Viral Load Estimations (TCID50)

Tissue homogenates were cleared of tissue fragments with a 5 min centrifugation at 5000 rpm at 4° C. Then 1 mL of supernatant was transferred to a microcentrifuge tube and centrifuge twice at 14000×g for 5 min at 4° C. Supernatant of tissue homogenates were filter-sterilized (5 minutes at 1000×g at 4° C.), using Spin-X tubes (Corning, Cat. 8160). Nasal washes samples were also filter-sterilized (5 minutes at 1000×g at 4° C.), using Spin-X tubes (Corning, Cat. 8160). The start dilution was 1/3 for tissue samples, 1/2 for nasal washes and serum samples. Ten-fold dilutions of samples were made in titration medium ((49% DMEM (Gibco, Cat. 11965-084) and 49% Medium 199 (Gibco, Cat. 11150-059), supplemented with 0.1% of FBS (Gibco, Cat. 26140-079), L-glutamine 2 mM (Gibco, Cat. 35050-061), 0.1% Gentamicin (Gibco, Cat. 15750-060) and 1/400 TrypLE Select (10×) Enzyme (Thermo Fisher, A1217701)) in sterile microtiter polypropylene tubes.

For the TCID50 titer determination, the assay was performed in octoplicates. MDCK-SIAT1 cells were trypsinized and resuspended at 2.4×10⁵ cells/mL in titration medium. 50 μL of titration media were added to each well except for the negative control wells where 100 μL of titration media were added (no sample). 50 μL of sample's serial-dilutions were added to the appropriate wells (octuplicates) of 96-well plates and 2.4×104 MDCK cells (100 μL) were added to all wells. Samples, in a total volume of 200 μL, were incubated for 4 days at 33° C., 5% CO₂ to allow viral replication.

TCID50 titer was evaluated by hemagglutination, which was achieved by mixing 50 μL of viral growth supernatants with 50 μL of 0.5% turkey red blood cells (RBCs) suspension in V-bottom 96-well plates. Plates were incubated 1 hour at RT and hemagglutination was read.

### Hemagglutination Inhibition Assay

HAI assay was performed against 2 strains of Influenza: A/California/07/2009(H1N1) and A/Texas/50/2012(H3N2).

To avoid unspecific hemagglutination, samples were treated with a solution of 2% receptor destroying enzyme (RDE) (Cholera Filtrate, Sigma, Cat. C8772) overnight. The reaction was stopped with 1.5% sodium citrate (Sigma, S4641) solution for incubation for 30 minutes and final treatment with 5% turkey RBCs suspension in 0.2% bovine serum albumin solution (BSA) (Sigma, A7030) for one hour at 4° C. After treatment, samples were centrifugated at 1000×g for 10 minutes at 4° C. and the supernatants were transferred for evaluation.

Sample treatment evaluation was performed by hemagglutination (0.5% turkey RBCs) for the assay in V bottom plate (Greiner Bio-One, Cat. 651901). No hemagglutination should be observed after the 45 minutes incubation at RT to qualify treatment as passed.

Virus titration and back titration were performed before the assay to confirm proper virus working dilution (8HA/50 L) to be used for HAI assay. Viral titers obtained by the last dilution showing hemagglutination corresponded to the HA content.

For HAI assay, positive control reference sera and RBCs alone were added to meet the acceptance criteria. The assay was performed on 2-fold serial dilutions of samples and positive controls in the presence of titrated virus (8 HA/50 μL). Plates were read for hemagglutination after at least 45 minutes of 0.5% RBCs incubation at RT. All samples and positive controls were performed in duplicates. To be compliant with the acceptance criteria, both replicates for a same sample should not be different by more than one dilution.

Statistical analyses were performed using Prism 8 software (GraphPad Software, Inc.). Following Shapiro-Wilk normality test and Skewness and Kurtosis range evaluation, statistical significance was assessed using a one-way ANOVA or Kruskall-Wallis test when data could not present a normal distribution. Post-hoc multiple comparisons tests used were Tukey for the ANOVA or Dunn's for the Kruskall-Wallis. P Value <0.05 was considered significant. *P<0.05, **P<0.01, ***P<0.001, and ****P<0.0001. Calculated error on presented results is 95% confidence interval.

### Body Weight, Body Temperature and Clinical Signs

Body weight, body temperature and clinical signs were recorded individually on Day 0 to Day 2, Day 4, Day 6, Day 8, Day 14 to Day 17, Day 19, Day 21, Day 28 to Day 30.

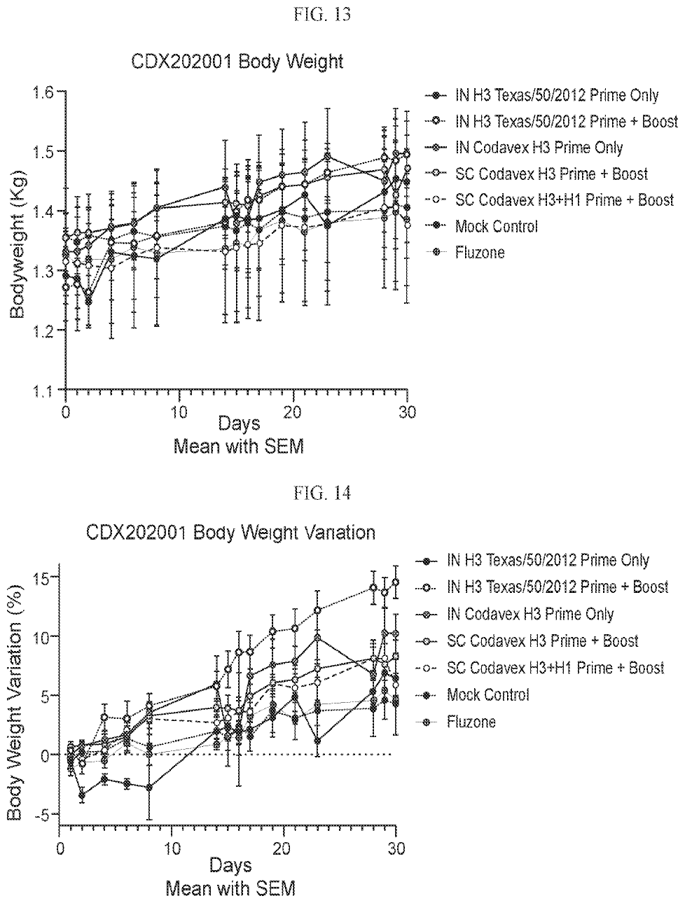

Body weight measurements showed that all animals experienced progressive weight gain (FIG. 13, Table 8). Treatment did not significantly diminished body weight gain compared to Mock control group (Group 6) (FIG. 14, Table 9). It is noteworthy that animals received H3 Texas/50/2012 Prime Only (Group 1) showed a slight decrease of body weight compared to the other groups after immunization. However, ferrets recovered their initial weight and gained weight afterwards. Finally, challenge with A/Singapore/INFIMH-16-0019/2016 WT H3N2 virus did not induce weight loss during the three days of infection.

TABLE 8
Body Weight

| | | Days | | | | | | | | | | | | | | | |
|---|---|---|---|---|---|---|---|---|---|---|---|---|---|---|---|---|---|
| Groups | ID | 0 | 1 | 2 | 4 | 6 | 8 | 14 | 15 | 16 | 17 | 19 | 21 | 23 | 28 | 29 | 30 |
| 1 | 240800F | 1.222 | 1.200 | 1.195 | | | | | | | | | | | | | |
| IN H3 | 241466F | 1.287 | 1.293 | 1.222 | | | | | | | | | | | | | |
| Texas/50/201 | 241849F | 1.169 | 1.173 | 1.158 | | | | | | | | | | | | | |
| 2 Prime Only | 242365F | 1.220 | 1.200 | 1.364 | 1.183 | 1.186 | 1.192 | 1.228 | 1.234 | 1.219 | 1.232 | 1.227 | 1.250 | 1.202 | 1.259 | 1.270 | 1.263 |
| | 242730F | 1.375 | 1.370 | 1.310 | 1.352 | 1.353 | 1.398 | 1.415 | 1.405 | 1.415 | 1.412 | 1.439 | 1.464 | 1.413 | 1.456 | 1.478 | 1.468 |
| | 243370F | 1.481 | 1.484 | 1.433 | 1.457 | 1.436 | 1.357 | 1.515 | 1.535 | 1.523 | 1.521 | 1.542 | 1.585 | 1.512 | 1.582 | 1.814 | 1.615 |
| 2 | 242080F | 1.434 | 1.418 | 1.433 | | | | | | | | | | | | | |
| IN H3 | 242390F | 1.024 | 1.014 | 1.019 | | | | | | | | | | | | | |
| Texas/50/201 | 242993F | 1.260 | 1.258 | 1.207 | | | | | | | | | | | | | |
| 2 Prime + | 243299F | 1.287 | 1.313 | 1.292 | 1.343 | 1.351 | 1.366 | 1.394 | 1.417 | 1.438 | 1.434 | 1.450 | 1.458 | 1.482 | 1.502 | 1.494 | 1.510 |
| Boost | 243353F | 1.380 | 1.385 | 1.388 | 1.416 | 1.414 | 1.410 | 1.418 | 1.449 | 1.476 | 1.469 | 1.504 | 1.508 | 1.523 | 1.543 | 1.539 | 1.542 |
| | 243477F | 1.289 | 1.273 | 1.242 | 1.281 | 1.272 | 1.300 | 1.329 | 1.331 | 1.341 | 1.352 | 1.369 | 1.369 | 1.388 | 1.422 | 1.419 | 1.432 |
| 3 | 242055F | 1.393 | 1.410 | 1.405 | | | | | | | | | | | | | |
| IN Codavax | 242250F | 1.315 | 1.295 | 1.339 | | | | | | | | | | | | | |
| H3 Prime | 243451F | 1.213 | 1.220 | 1.216 | | | | | | | | | | | | | |
| Only | 241946F | 1.280 | 1.263 | 1.279 | 1.381 | 1.308 | 1.315 | 1.343 | 1.284 | 1.331 | 1.353 | 1.370 | 1.372 | 1.394 | 1.355 | 1.410 | 1.405 |
| | 242152F | 1.344 | 1.345 | 1.342 | 1.358 | 1.328 | 1.378 | 1.377 | 1.316 | 1.251 | 1.389 | 1.398 | 1.393 | 1.433 | 1.374 | 1.434 | 1.445 |
| | 243311F | 1.446 | 1.456 | 1.470 | 1.483 | 1.504 | 1.524 | 1.597 | 1.539 | 1.580 | 1.604 | 1.614 | 1.631 | 1.650 | 1.622 | 1.547 | 1.837 |
| 4 | 242624F | 1.337 | 1.349 | 1.353 | | | | | | | | | | | | | |
| SC Codavax | 243094F | 1.502 | 1.510 | 1.511 | | | | | | | | | | | | | |
| H3 Prime + | 243302F | 1.216 | 1.208 | 1.213 | | | | | | | | | | | | | |
| Boost | 241105F | 1.423 | 1.449 | 1.440 | 1.446 | 1.483 | 1.455 | 1.498 | 1.504 | 1.497 | 1.510 | 1.517 | 1.531 | 1.536 | 1.539 | | 1.538 |
| | 241831F | 1.347 | 1.340 | 1.339 | 1.330 | 1.380 | 1.381 | 1.282 | 1.373 | 1.376 | 1.387 | 1.415 | 1.398 | 1.425 | 1.432 | 1.417 | 1.428 |
| | 243566F | 1.308 | 1.328 | 1.329 | 1.337 | 1.316 | 1.346 | 1.358 | 1.360 | 1.354 | 1.381 | 1.380 | 1.404 | 1.411 | 1.434 | 1.437 | 1.449 |
| 5 | 240818F | 1.225 | 1.251 | 1.249 | | | | | | | | | | | | | |
| SC Codavax | 241822F | 1.353 | 1.350 | 1.358 | | | | | | | | | | | | | |
| H3 + H1 | 242187F | 1.420 | 1.401 | 1.398 | | | | | | | | | | | | | |
| Prime + | 242411F | 1.279 | 1.278 | 1.276 | 1.303 | 1.306 | 1.317 | 1.324 | 1.329 | 1.348 | 1.345 | 1.358 | 1.363 | 1.353 | 1.378 | 1.394 | 1.361 |
| Boost | 242721F | 1.497 | 1.482 | 1.473 | 1.504 | 1.544 | 1.578 | 1.544 | 1.562 | 1.559 | 1.569 | 1.605 | 1.602 | 1.619 | 1.652 | 1.648 | 1.608 |
| | 243591F | 1.114 | 1.108 | 1.105 | 1.101 | 1.125 | 1.122 | 1.129 | 1.125 | 1.126 | 1.325 | 1.163 | 1.150 | 1.151 | 1.186 | 1.174 | 1.158 |
| 6 | 241971F | 1.354 | 1.332 | 1.363 | | | | | | | | | | | | | |
| Mock | 242748F | 1.246 | 1.256 | 1.245 | | | | | | | | | | | | | |
| Control | 242845F | 1.500 | 1.501 | 1.507 | | | | | | | | | | | | | |
| | 243248F | 1.427 | 1.435 | 1.452 | 1.472 | 1.476 | 1.481 | 1.492 | 1.479 | 1.527 | 1.484 | 1.543 | 1.521 | 1.554 | 1.549 | 1.539 | 1.562 |
| | 243558F | 1.361 | 1.325 | 1.340 | 1.345 | 1.369 | 1.350 | 1.359 | 1.358 | 1.343 | 1.368 | 1.378 | 1.371 | 1.379 | 1.387 | 1.404 | 1.388 |
| | 243582F | 1.251 | 1.243 | 1.245 | 1.237 | 1.252 | 1.238 | 1.270 | 1.264 | 1.268 | 1.251 | 1.272 | 1.274 | 1.262 | 1.266 | 1.286 | 1.268 |
| 7 | 242381F | 1.107 | 1.092 | 1.103 | 1.105 | 1.122 | 1.097 | 1.120 | 1.123 | 1.136 | 1.145 | 1.143 | 1.133 | 1.155 | 1.155 | 1.169 | 1.187 |
| Fluzone | 242616F | 1.404 | 1.374 | 1.389 | 1.398 | 1.419 | 1.392 | 1.403 | 1.417 | 1.412 | 1.451 | 1.481 | 1.452 | 1.458 | 1.485 | 1.476 | 1.481 |
| | 243574F | 1.468 | 1.468 | 1.462 | 1.455 | 1.475 | 1.495 | 1.490 | 1.495 | 1.483 | 1.509 | 1.525 | 1.511 | 1.530 | 1.523 | 1.550 | 1.529 |

D 29 data for animal 241105R (1.151 kg) was removed due to suspected recording error.

TABLE 9
Body Weight Variation (%)

| | Days | | | | | | | | |
|---|---|---|---|---|---|---|---|---|---|
| Groups | ID | 1 | 2 | 4 | 6 | 8 | 14 | 15 | 16 |
| 1 | 240800F | −1.800 | −2.209 | | | | | | |
| IN H3 | 241466F | 0.488 | −5.051 | | | | | | |
| Texas/50/201 | 241849F | 0.342 | −1.198 | | | | | | |
| 2 Prime Only | 242365F | −1.639 | −4.590 | −3.033 | −2.787 | −2.295 | 0.658 | 1.148 | −0.052 |
| | 242730F | −0.384 | −4.727 | −1.573 | −1.500 | 1.673 | 2.909 | 2.182 | 2.908 |
| | 243370F | 0.203 | −2.903 | −1.621 | −3.038 | −7.698 | 2.296 | 3.646 | 2.836 |
| 2 | 242080F | −1.118 | −0.070 | | | | | | |
| IN H3 | 242390F | −0.977 | −0.488 | | | | | | |
| Texas/50/201 | 242993F | −0.159 | −4.206 | | | | | | |
| 2 Prime + | 243299F | 2.020 | 0.389 | 4.351 | 4.973 | 6.138 | 8.314 | 10.101 | 11.733 |
| Boost | 243353F | 1.838 | 2.059 | 4.118 | 3.971 | 3.876 | 4.265 | 6.544 | 8.529 |
| | 243477F | 0.315 | −2.128 | 0.946 | 0.238 | 2.443 | 4.725 | 4.886 | 5.674 |
| 3 | 242055F | 1.220 | 0.561 | | | | | | |
| IN Codavax | 242250F | −1.521 | 1.825 | | | | | | |
| H3 Prime | 243451F | 0.577 | 0.247 | | | | | | |
| Only | 241946F | −1.328 | −0.078 | 0.078 | 2.188 | 2.734 | 4.922 | 0.313 | 3.984 |
| | 242152F | 0.074 | −0.149 | 0.142 | −1.190 | 2.530 | 2.455 | −2.083 | −8.920 |
| | 243311F | 0.692 | 1.660 | 2.558 | 4.011 | 5.394 | 10.443 | 6.432 | 9.267 |
| 4 | 242624F | 0.898 | 1.197 | | | | | | |
| SC Codavax | 243094F | 0.533 | 0.599 | | | | | | |
| H3 Prime + | 243302F | −0.658 | −0.247 | | | | | | |
| Boost | 241105F | 1.827 | 1.827 | 1.616 | 2.811 | 4.357 | 5.271 | 5.682 | 5.200 |
| | 241831F | −0.520 | −0.520 | −1.262 | 0.965 | 2.524 | 2.595 | 1.930 | 2.153 |
| | 243566F | 1.685 | 1.761 | 2.374 | 0.766 | 3.083 | 3.982 | 4.135 | 3.675 |
| 5 | 240818F | 2.122 | 1.959 | | | | | | |
| SC Codavax | 241822F | −0.222 | 0.222 | | | | | | |
| H3 + H1 | 242187F | −1.338 | −1.549 | | | | | | |
| Prime + | 242411F | −0.078 | −0.235 | 1.876 | 2.111 | 2.971 | 3.518 | 3.909 | 5.395 |
| Boost | 242721F | −1.002 | −1.603 | 0.468 | 3.140 | 5.277 | 3.140 | 4.342 | 4.142 |
| | 243591F | −0.539 | −1.267 | −1.167 | 0.987 | 0.718 | 1.346 | 0.987 | 1.077 |
| 6 | 241971F | −1.625 | 0.665 | | | | | | |
| Mock | 242748F | 0.884 | 0.000 | | | | | | |
| Control | 242845F | 0.067 | 0.467 | | | | | | |
| | 243248F | 0.561 | 1.752 | 3.153 | 3.434 | 3.754 | 4.555 | 3.644 | 7.008 |
| | 243558F | −2.645 | −1.543 | −1.176 | 0.588 | −0.808 | −0.147 | −0.220 | −1.323 |
| | 243582F | −0.799 | −0.480 | −1.119 | 0.080 | −1.039 | 1.519 | 1.039 | 1.439 |
| 7 | 242381F | −1.355 | −0.361 | −0.181 | 1.355 | −0.903 | 1.174 | 1.445 | 2.620 |
| Fluzone | 242616F | −2.137 | −1.068 | −0.427 | 1.068 | −0.855 | −0.071 | 0.926 | 0.570 |
| | 243574F | 0.000 | −0.409 | −0.886 | 0.477 | 1.839 | 1.499 | 1.839 | 1.022 |

| | Days | | | | | | | |
|---|---|---|---|---|---|---|---|---|
| Groups | ID | 17 | 19 | 21 | 23 | 28 | 29 | 30 |
| 1 | 240800F | | | | | | | |
| IN H3 | 241466F | | | | | | | |
| Texas/50/201 | 241849F | | | | | | | |
| 2 Prime Only | 242365F | 0.984 | 0.574 | 2.459 | −1.475 | 3.197 | 4.098 | 3.525 |
| | 242730F | 2.691 | 4.655 | 6.473 | 2.764 | 5.891 | 7.491 | 6.764 |
| | 243370F | 2.701 | 4.119 | 5.672 | 2.093 | 6.820 | 8.980 | 9.048 |
| 2 | 242080F | | | | | | | |
| IN H3 | 242390F | | | | | | | |
| Texas/50/201 | 242993F | | | | | | | |
| 2 Prime + | 243299F | 11.422 | 12.665 | 13.287 | 15.152 | 16.706 | 16.084 | 17.327 |
| Boost | 243353F | 8.015 | 10.588 | 10.882 | 11.985 | 13.456 | 13.162 | 13.382 |
| | 243477F | 6.541 | 7.850 | 7.880 | 9.377 | 12.057 | 11.820 | 12.924 |
| 3 | 242055F | | | | | | | |
| IN Codavax | 242250F | | | | | | | |
| H3 Prime | 243451F | | | | | | | |
| Only | 241946F | 5.703 | 7.031 | 7.188 | 8.906 | 5.859 | 10.156 | 9.800 |
| | 242152F | 3.348 | 4.018 | 3.648 | 6.622 | 2.232 | 8.696 | 7.600 |
| | 243311F | 10.927 | 11.618 | 12.794 | 14.108 | 12.172 | 13.900 | 13.200 |
| 4 | 242624F | | | | | | | |
| SC Codavax | 243094F | | | | | | | |
| H3 Prime + | 243302F | | | | | | | |
| Boost | 241105F | 6.114 | 6.606 | 7.590 | 7.941 | 8.152 | | 7.900 |
| | 241831F | 2.970 | 5.048 | 3.785 | 5.791 | 6.310 | 5.197 | 6.000 |
| | 243566F | 5.743 | 6.432 | 7.504 | 8.040 | 9.851 | 10.031 | 10.900 |
| 5 | 240818F | | | | | | | |
| SC Codavax | 241822F | | | | | | | |
| H3 + H1 | 242187F | | | | | | | |
| Prime + | 242411F | 5.395 | 6.177 | 6.568 | 6.568 | 7.740 | 7.740 | 6.400 |
| Boost | 242721F | 4.810 | 7.214 | 7.014 | 8.150 | 10.354 | 10.354 | 7.300 |
| | 243591F | 0.539 | 4.399 | 3.232 | 3.321 | 6.463 | 8.463 | 3.900 |
| 6 | 241971F | | | | | | | |
| Mock | 242748F | | | | | | | |
| Control | 242845F | | | | | | | |
| | 243248F | 3.994 | 8.129 | 8.587 | 8.900 | 8.549 | 7.849 | 9.450 |
| | 243558F | 0.514 | 1.249 | 0.735 | 1.323 | 1.910 | 3.159 | 1.984 |
| | 243582F | 0.000 | 1.678 | 1.839 | 0.879 | 1.199 | 2.798 | 1.359 |
| 7 | 242381F | 3.704 | 3.252 | 2.349 | 4.336 | 4.336 | 5.601 | 5.420 |
| Fluzone | 242616F | 3.348 | 5.484 | 3.419 | 3.846 | 5.789 | 5.128 | 4.060 |
| | 243574F | 2.793 | 3.883 | 2.929 | 4.223 | 3.747 | 5.586 | 4.155 |

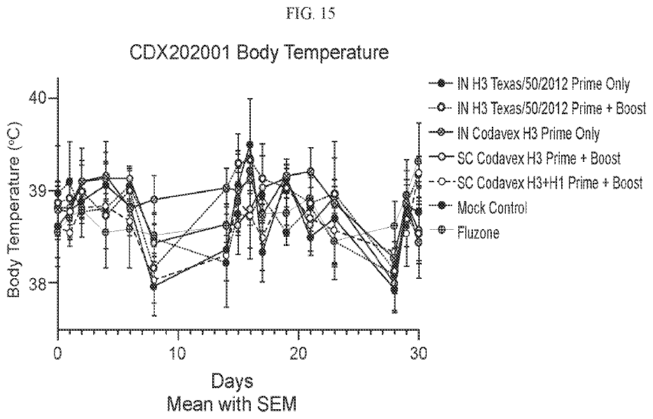

No abnormal body temperature was noted in this study. All groups showed a similar body temperature pattern throughout the study (FIG. 15, Table 10). The monitored clinical signs included decreased activity, sneezing, dehydration and erected fur, but no abnormal findings were observed.

TABLE 10
Body Temperature (° C.)

| | | Days | | | | | | | | | | | | | | | |
|---|---|---|---|---|---|---|---|---|---|---|---|---|---|---|---|---|---|
| Groups | ID | 0 | 1 | 2 | 4 | 6 | 8 | 14 | 15 | 16 | 17 | 19 | 21 | 23 | 28 | 29 | 30 |
| 1 | 240800F | 38.4 | 38.9 | 38.5 | | | | | | | | | | | | | |
| IN H3 | 241466F | 39.3 | 38.4 | 39.1 | | | | | | | | | | | | | |
| Texas/50/201 | 241849F | 39.0 | 39.3 | 38.7 | | | | | | | | | | | | | |
| 2 Prime Only | 242365F | 38.3 | 38.7 | 39.0 | 39.3 | 38.8 | 38.1 | 38.4 | 38.5 | 39.0 | 38.1 | 39.1 | 38.3 | 38.1 | 38.2 | 38.7 | 38.5 |
| | 242730F | 38.2 | 38.8 | 38.9 | 35.8 | 38.8 | 38.2 | 38.2 | 39.5 | 40.0 | 38.2 | 39.3 | 38.5 | 38.6 | 37.9 | 39.1 | 39.1 |
| | 243370F | 38.5 | 36.8 | 39.1 | 39.1 | 38.8 | 37.6 | 38.5 | 39.1 | 39.5 | 38.7 | 39.0 | 38.7 | 39.4 | 37.7 | 38.7 | 38.8 |
| 2 | 242080F | 38.8 | 38.9 | 39.3 | | | | | | | | | | | | | |
| IN H3 | 242390F | 38.9 | 38.9 | 39.3 | | | | | | | | | | | | | |
| Texas/50/201 | 242993F | 38.5 | 38.7 | 38.8 | | | | | | | | | | | | | |
| 2 Prime + | 243299F | 39.2 | 39.1 | 38.8 | 38.9 | 39.3 | 38.3 | 39.1 | 39.4 | 39.5 | 39.4 | 39.1 | 39.2 | 39.1 | 38.0 | 38.8 | 38.9 |
| Boost | 243353F | 38.9 | 38.7 | 38.9 | 38.6 | 38.3 | 38.3 | 39.2 | 39.2 | 39.2 | 38.7 | 39.0 | 38.8 | 38.5 | 38.1 | 38.7 | 38.4 |
| | 243477F | 38.9 | 39.2 | 39.1 | 35.7 | 38.8 | 37.9 | 38.8 | 39.3 | 39.3 | 39.3 | 39.0 | 38.6 | 39.0 | 38.3 | 38.6 | 38.3 |
| 3 | 242055F | 38.7 | 38.8 | 39.5 | 39.2 | 38.6 | 38.8 | 39.2 | 39.3 | 39.3 | 38.8 | 39.1 | 39.4 | 39.3 | 38.7 | 38.9 | 38.7 |
| IN Codavax | 242250F | 38.8 | 38.9 | 39.3 | | | | | | | | | | | | | |
| H3 Prime | 243451F | 38.5 | 38.9 | 39.1 | 38.9 | 38.9 | 38.7 | 38.8 | 38.5 | 38.7 | 35.4 | 39.1 | 38.8 | 38.1 | 38.1 | 38.8 | 38.0 |
| Only | 241946F | 38.8 | 38.6 | 38.4 | | | | | | | | | | | | | |
| | 242152F | 39.1 | 38.8 | 39.2 | 39.4 | 38.3 | 39.2 | 39.1 | 39.2 | 39.4 | 39.0 | 39.3 | 39.3 | 38.2 | 38.0 | 39.1 | 38.6 |
| | 243311F | 38.8 | 38.8 | 39.0 | | | | | | | | | | | | | |
| 4 | 242624F | 38.8 | 38.6 | 39.2 | 39.2 | 39.0 | 38.6 | 38.8 | 38.5 | 38.7 | 39.0 | 39.3 | 38.8 | 39.0 | 38.3 | 38.5 | 38.7 |
| SC Codavax | 243094F | 38.5 | 39.0 | 38.9 | 39.5 | 39.2 | 38.5 | 38.9 | 39.0 | 39.0 | 39.4 | 39.2 | 38.8 | 39.0 | 38.0 | 39.4 | 38.8 |
| H3 Prime + | 243302F | 39.2 | 39.1 | 39.5 | | | | | | | | | | | | | |
| Boost | 241105F | 38.5 | 38.5 | 39.1 | | | | | | | | | | | | | |
| | 241831F | 39.1 | 39.2 | 39.0 | | | | | | | | | | | | | |
| | 243566F | 38.8 | 38.5 | 38.9 | 35.7 | 39.2 | 38.2 | 38.2 | 38.4 | 38.7 | 38.7 | 35.9 | 38.3 | 38.5 | 37.7 | 38.7 | 38.2 |
| 5 | 240818F | 38.9 | 38.8 | 38.7 | | | | | | | | | | | | | |
| SC Codavax | 241822F | 38.3 | 38.4 | 38.9 | | | | | | | | | | | | | |
| H3 + H1 | 242187F | 38.8 | 38.5 | 38.7 | | | | | | | | | | | | | |
| Prime + | 242411F | 38.9 | 38.5 | 35.8 | 38.8 | 38.8 | 38.0 | 38.8 | 39.1 | 39.1 | 38.4 | 39.2 | 38.7 | 38.7 | 38.2 | 38.2 | 39.1 |
| Boost | 242721F | 38.0 | 38.4 | 39.0 | 39.1 | 38.7 | 38.3 | 38.4 | 38.9 | 38.4 | 38.2 | 39.1 | 38.9 | 38.5 | 38.4 | 38.9 | 39.3 |
| | 243591F | 38.6 | 38.5 | 38.8 | 35.8 | 38.5 | 37.8 | 37.7 | 38.7 | 38.7 | 38.9 | 35.8 | 38.5 | 38.5 | 38.4 | 38.5 | 39.2 |
| 6 | 241971F | 38.8 | 38.7 | 38.6 | | | | | | | | | | | | | |
| Mock | 242748F | 38.9 | 39.1 | 35.1 | | | | | | | | | | | | | |
| Control | 242845F | 38.6 | 38.9 | 39.0 | | | | | | | | | | | | | |
| | 243248F | 38.8 | 39.1 | 39.0 | 39.1 | 39.2 | 38.3 | 38.4 | 38.9 | 38.2 | 39.0 | 38.6 | 38.6 | 38.3 | 38.1 | 39.0 | 39.0 |
| | 243558F | 38.8 | 39.0 | 38.5 | 39.1 | 39.1 | 38.8 | 38.3 | 38.5 | 38.9 | 39.0 | 38.6 | 38.9 | 38.8 | 38.2 | 38.8 | 39.3 |
| | 243582F | 40.0 | 39.9 | 38.4 | 35.3 | 38.9 | 38.4 | 38.0 | 38.6 | 39.1 | 38.9 | 38.5 | 39.0 | 38.3 | 38.0 | 38.8 | 39.3 |
| 7 | 242381F | 38.6 | 38.7 | 38.8 | 35.1 | 38.1 | 38.6 | 38.7 | 39.1 | 39.2 | 38.6 | 39.1 | 39.1 | 38.2 | 38.8 | 39.2 | 39.1 |
| Fluzone | 242616F | 38.6 | 38.8 | 38.5 | 38.8 | 38.8 | 38.7 | 38.7 | 39.1 | 39.4 | 38.7 | 38.8 | 38.7 | 38.7 | 38.9 | 39.0 | 39.8 |
| | 243574F | 38.5 | 38.7 | 38.8 | 38.8 | 38.9 | 38.3 | 38.5 | 38.5 | 39.1 | 39.0 | 38.4 | 39.0 | 38.5 | 38.4 | 38.7 | 39.1 |

### Gross Necropsy

At terminal Day 30, no abnormal findings related to infection or treatment were observed on the examined organs except lung with multiple area dark from several animals were observed.

### Functional Antibody Measurement by HAI

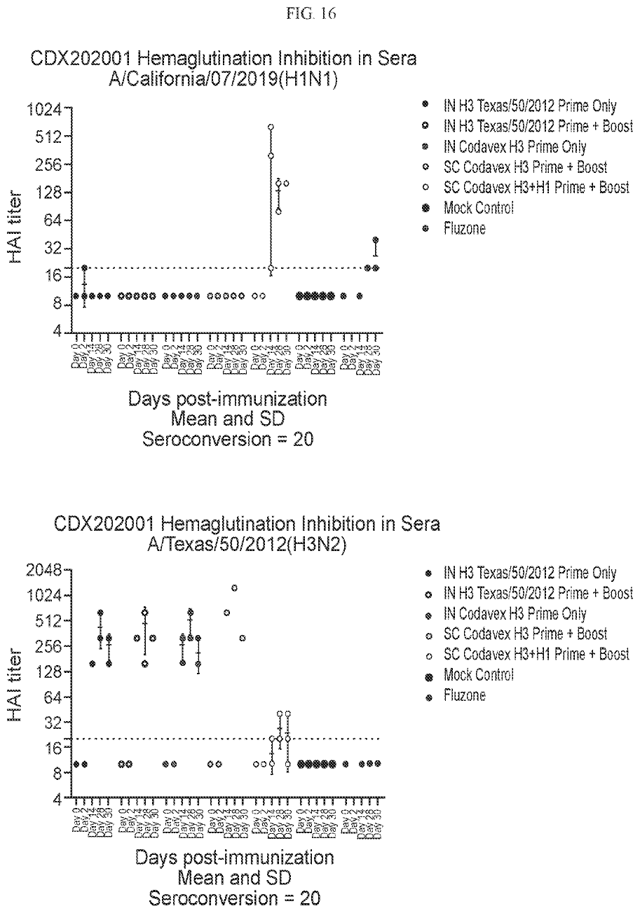

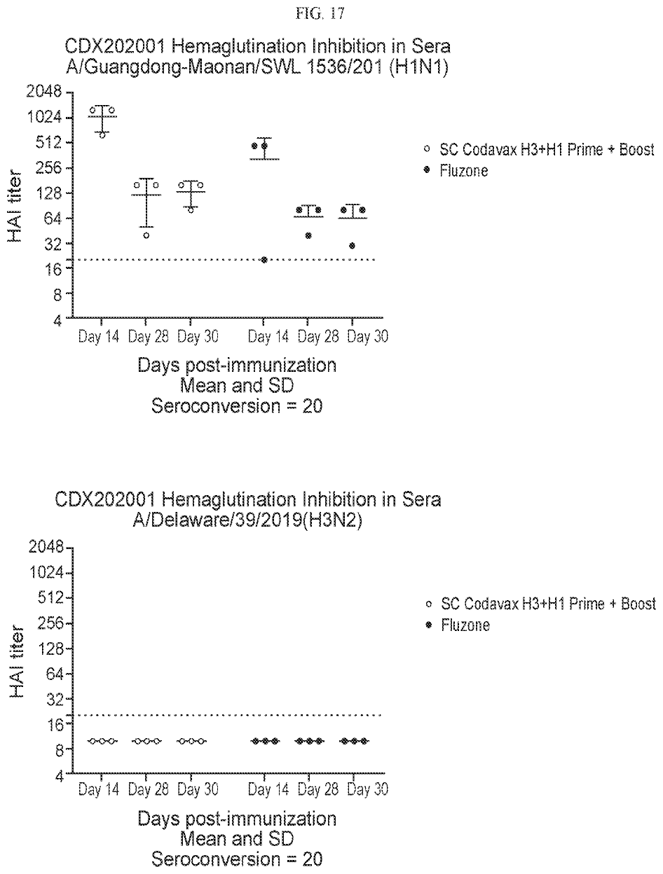

Results from HAI against A/California/07/2009(H1N1) and A/Texas/50/2012(H3N2) were displayed in FIG. 16, Table 11.

TABLE 11
Detailed HAI titers in sera (California and Texas strains)

| | | A/California/07/2019(H1N1) | | | | | A/Texas/50/2012(H3N2) | | | | |
|---|---|---|---|---|---|---|---|---|---|---|---|
| | | Days | | | | | | | | | |
| Groups | ID | 0 | 2 | 14 | 28 | 30 | 0 | 2 | 14 | 28 | 30 |
| 1 | 240800F | <20 | <20 | | | | <20 | <20 | | | |
| IN H3 | 241466F | <20 | 20 | | | | <20 | <20 | | | |
| Texas/50/20 | 241849F | <20 | <20 | | | | <20 | <20 | | | |
| 12 Prime | 242365F | <20 | | <20 | <20 | <20 | <20 | | 160 | 320 | 160 |
| Only | 242730F | <20 | | <20 | <20 | <20 | <20 | | 160 | 320 | 320 |
| | 243370F | <20 | | <20 | <20 | <20 | <20 | | 160 | 640 | 320 |
| 2 | 242080F | <20 | <20 | | | | <20 | <20 | | | |
| IN H3 | 242390F | <20 | <20 | | | | <20 | <20 | | | |
| Texas/50/20 | 242993F | <20 | <20 | | | | <20 | <20 | | | |
| 12 Prime + | 243299F | <20 | | <20 | <20 | <20 | <20 | | 320 | 640 | 320 |
| Boost | 243353F | <20 | | <20 | <20 | <20 | <20 | | 320 | 640 | 320 |
| | 243477F | <20 | | <20 | <20 | <20 | <20 | | 320 | 160 | 320 |
| 3 | 242055F | <20 | <20 | | | | <20 | <20 | | | |
| IN Codavax | 242250F | <20 | <20 | | | | <20 | <20 | | | |
| H3 Prime | 243451F | <20 | <20 | | | | <20 | <20 | | | |
| Only | 241946F | <20 | | <20 | <20 | <20 | <20 | | 160 | 640 | 160 |
| | 242152F | <20 | | <20 | <20 | <20 | <20 | | 320 | 320 | 160 |
| | 243311F | <20 | | <20 | <20 | <20 | <20 | | 320 | 640 | 320 |
| 4 | 242624F | <20 | <20 | | | | <20 | <20 | | | |
| SC Codavax | 243094F | <20 | <20 | | | | <20 | <20 | | | |
| H3 Prime + | 243302F | <20 | <20 | | | | <20 | <20 | | | |
| Boost | 241105F | <20 | | <20 | <20 | <20 | <20 | | 640 | 1280 | 320 |
| | 241831F | <20 | | <20 | <20 | <20 | <20 | | 640 | 1280 | 320 |
| | 243566F | <20 | | <20 | <20 | <20 | <20 | | 640 | 1280 | 320 |
| 5 | 240818F | <20 | <20 | | | | <20 | <20 | | | |
| SC Codavax | 241822F | <20 | <20 | | | | <20 | <20 | | | |
| H3 + H1 | 242187F | <20 | <20 | | | | <20 | <20 | | | |
| Prime + | 242411F | <20 | | 320 | 160 | 160 | <20 | | 20 | 40 | 20 |
| Boost | 242721F | <20 | | 640 | 160 | 160 | <20 | | <20 | 20 | 40 |
| | 243591F | <20 | | 20 | 80 | 160 | <20 | | <20 | 20 | <20 |
| 6 | 241971F | <20 | <20 | | | | <20 | <20 | | | |
| Mock | 242748F | <20 | <20 | | | | <20 | <20 | | | |
| Control | 242845F | <20 | <20 | | | | <20 | <20 | | | |
| | 243248F | <20 | | <20 | <20 | <20 | <20 | | <20 | <20 | <20 |
| | 243558F | <20 | | <20 | <20 | <20 | <20 | | <20 | <20 | <20 |
| | 243582F | <20 | | <20 | <20 | <20 | <20 | | <20 | <20 | <20 |
| 7 | 242381F | <20 | | <20 | 20 | 20 | <20 | | <20 | <20 | <20 |
| Fluzone | 242616F | <20 | | <20 | 20 | 20 | <20 | | <20 | <20 | <20 |
| | 243574F | <20 | | <20 | 20 | 40 | <20 | | <20 | <20 | <20 |

Two out of three animals that received SC Codavax H3+H1 Prime+Boost (Group 5) showed a functional antibody response against the A/California/07/2009(H1N1) virus at 14 days post immunization in tested and all three animals had titers on Day 28 and Day 30.

The data showed that both IN immunization and SC immunization allowed seroconversion. All animals that received H3N2 vaccination (Group 1 to Group 4) seroconverted 14 days post immunization for tested A/Texas/50/2012 (H3N2) strain. SC Codavax H3+H1 Prime+Boost (Group 5) had weaker HAI titers which were close to seroconversion level compared to SC Codavax H3 Prime+Boost (Group 4) when testing for H3N2.

Animals received Fluzone (Group 7) started to have low level of functional antibodies 28 days after immunization against A/California/07/2009(H1N1) but not A/Texas/50/2012(H3N2) strain. As Fluzone contains hemagglutinin of A/Guangdong-Maonan/SW1536/2019(H1N1) pdm09-like strain and A/Hongkong/2671/2019(H3N2) like strain, additional HAI were performed against A/Delaware/39/2019 (H3N2) (A/Hong Kong/45/2019 (H3N2)-like virus) and A/Guangdong-Maonan/SWL1536/2019 (H1N1) (Table 12). Both SC Codavax H3+H1 Prime+Boost (Group 5) and Fluzone (Group 7) showed functional antibodies 14 days post immunization in tested A/Guangdong-Maonan/SWL 1536/2019 (H1N1) but not in A/Delaware/39/2019 (H3N2) strain. These results confirmed that Fluzone induced lower functional antibodies against H3 than H1 strain, even when tested with other strains.

TABLE 12
Detailed HAI titers in sera (Guangdong and Delaware Strains)

| | | A/Guangdong- | | | | | |
|---|---|---|---|---|---|---|---|
| | | Maonan/SWL | | | A/Delaware/39/2019 | | |
| | | 1536/201 (H1N1) | | | (H3N2) | | |
| | | Days | | | | | |
| Groups | ID | 14 | 28 | 30 | 14 | 28 | 30 |
| 5 | 242411F | 1280 | 160 | 160 | 10 | 10 | 10 |
| SC Codavax | 242721F | 1280 | 160 | 160 | 10 | 10 | 10 |
| H3 + H1 | 243591F | 640 | 40 | 80 | 10 | 10 | 10 |
| Prime + Boost | | | | | | | |
| 7 | 242381F | 480 | 40 | 30 | 10 | 10 | 10 |
| Fluzone | 242616F | 480 | 80 | 80 | 10 | 10 | 10 |
| | 243574F | 20 | 80 | 80 | 10 | 10 | 10 |

### Viral Load Estimation by TCID50

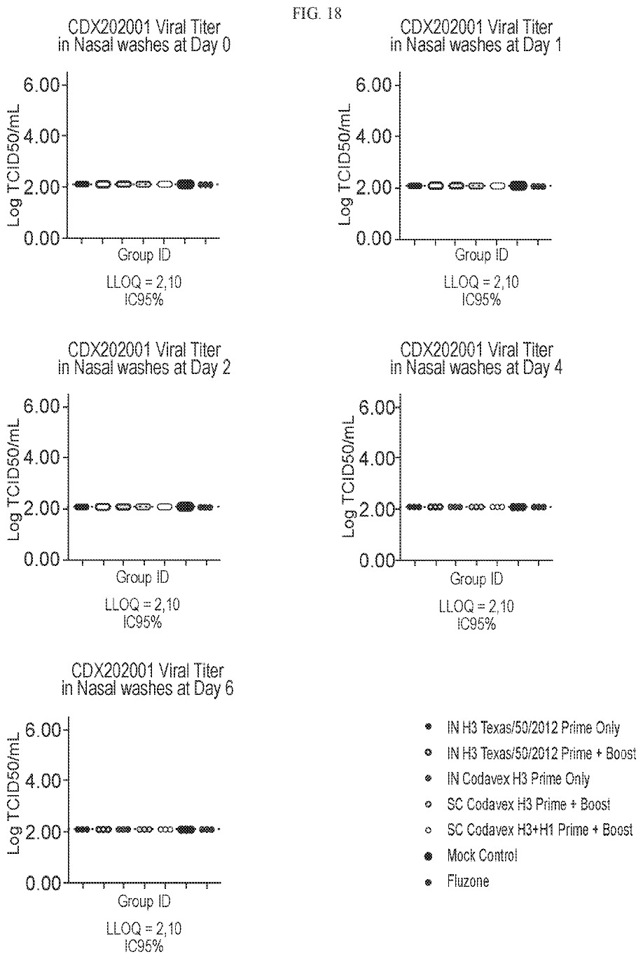

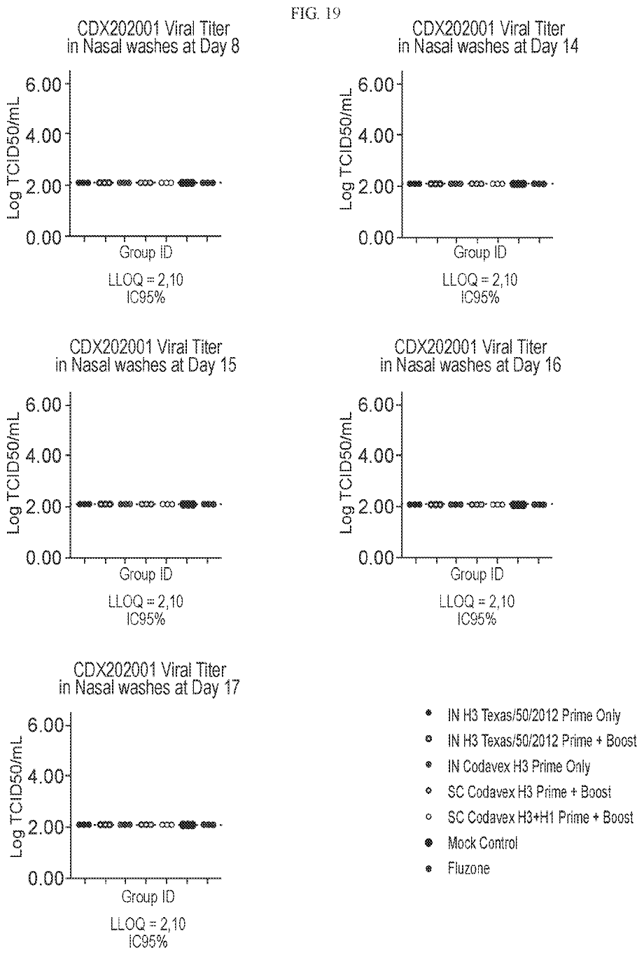

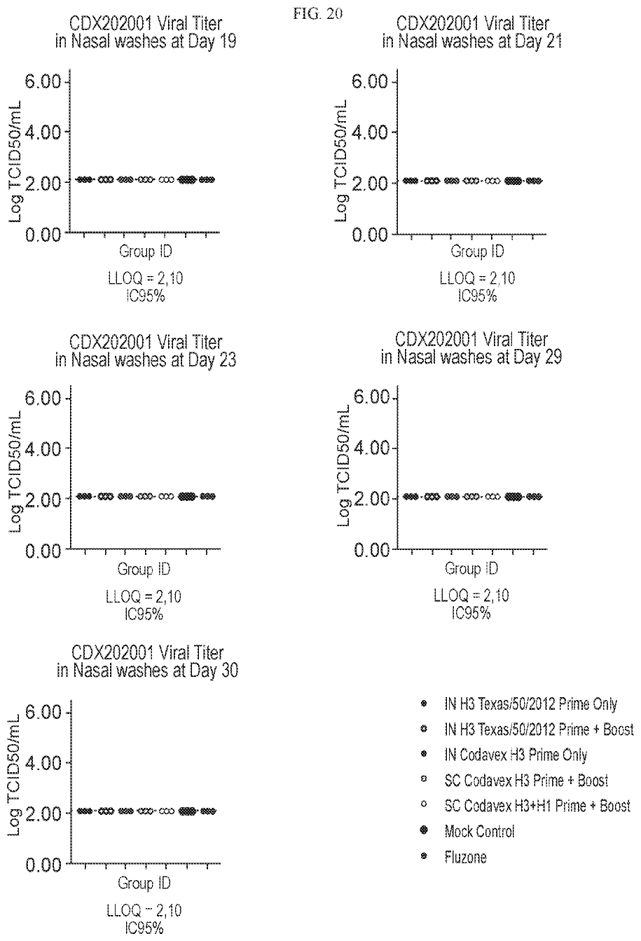

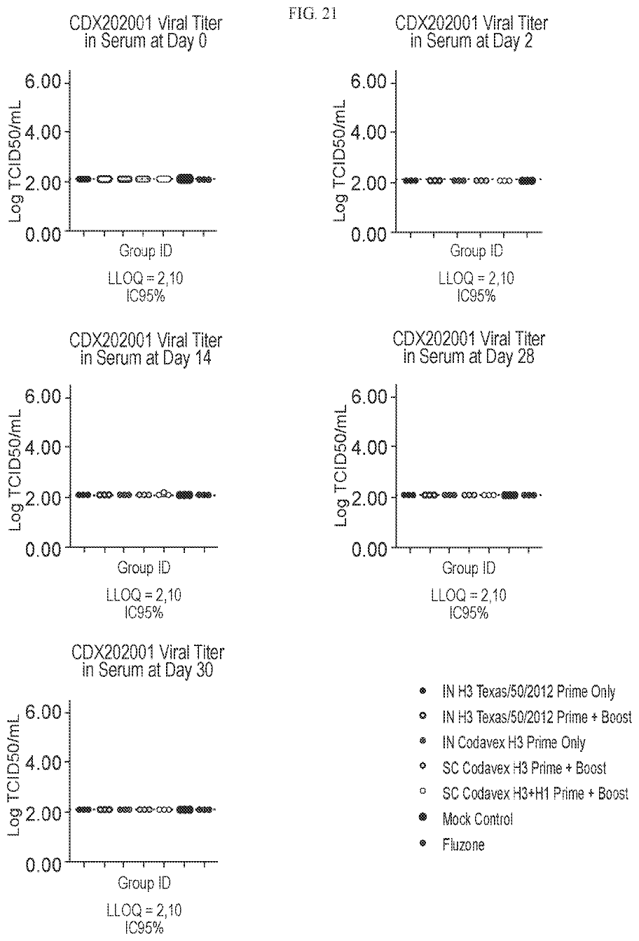

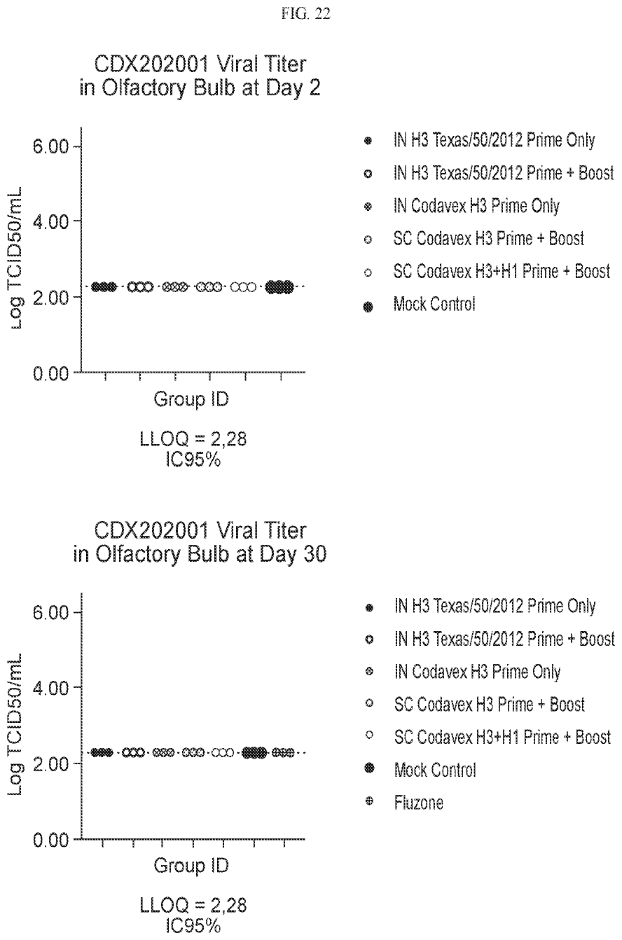

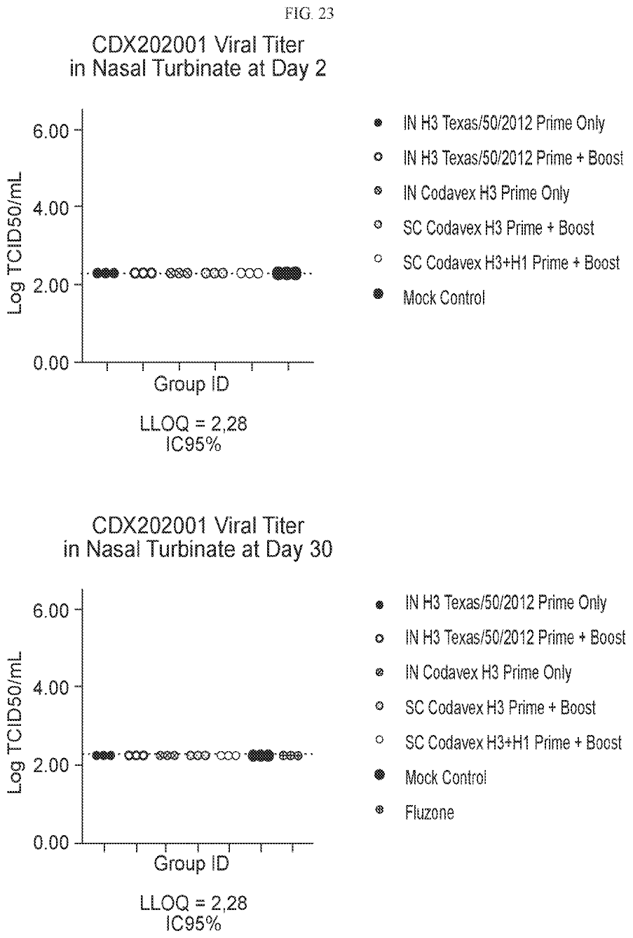

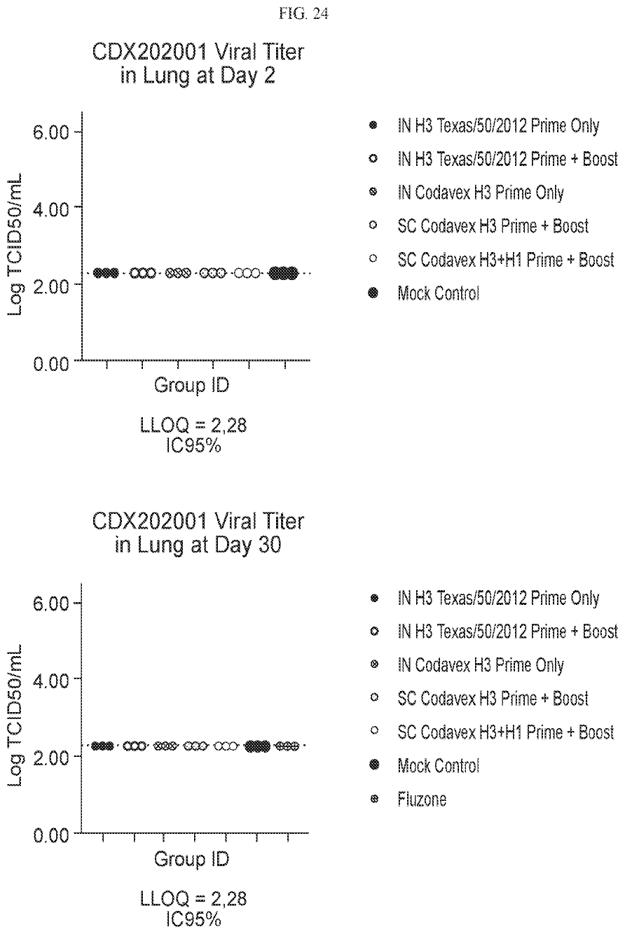

The results from the viral titration in lungs, serum, nasal turbinates, olfactory bulbs, and nasal washes are presented in FIG. 18 to FIG. 24. Influenza virus quantitation by TCID50 showed all the animals had a viral titer below limit of quantification. No virus could be detected at any time point in any of the groups. The virus dose given for the challenge was based on viral titer received from the sponsor. The virus seemed to be cleared from the animal.

Histopathological Evaluation of Nasal Turbinate, Olfactory Bulb and Lung Tissues from Ferrets

Nasal turbinate, olfactory bulbs and lung samples were collected from half of the animals from Group 1 to Group 6 on Day 2 and the remaining animals on Day 30 for histopathology assay.

For animals sacrificed on Day 2, neutrophilic inflammation was observed in nasal turbinates but not other tissues in all groups. The severity of these changes was moderate to marked in animals from IN H3 Texas/50/2012 Prime Only (Groups 1), IN H3 Texas/50/2012 Prime+Boost (Group 2), and IN Codavax H3 Prime Only (Group 3); minimal to mild in SC Codavax H3 Prime+Boost (Group 4) and minimal in SC Codavax H3+H1 Prime+Boost (Groups 5) and Mock Control (Group 6).

At terminal euthanasia (Day 30), the neutrophilic inflammation of the nasal turbinates was still present. But the severity in IN H3 Texas/50/2012 Prime Only (Groups 1), IN H3 Texas/50/2012 Prime+Boost (Group 2), and IN Codavax H3 Prime Only (Group 3) decreased to minimal, mild, and minimal to mild respectively whereas the severity in SC Codavax H3+H1 Prime+Boost (Groups 5) and Mock Control (Group 6) increased to moderate to marked. In addition, one animal from IN H3 Texas/50/2012 Prime Only (Groups 1) developed minimal, two animals from SC Codavax H3+H1 Prime+Boost (Groups 5) and Mock Control (Group 6) developed mild to moderate and one animal from Fluzone (Group 7) developed mild neutrophilic inflammation in the lungs.

### Example 6

### Determination of Serum IgG Antibody Titers Against HA2(A/Vietnam/1203/2004)(H5N1) Via Enzyme-Linked Immunosorbent Assay (ELISA) in Non-Human Primates Vaccinated Intramuscularly (IM) with Live Attenuated Influenza Vaccine, CodaVax-H1N1

Three male and three female seronegative African green monkeys were vaccinated on days 0 and 21 with live attenuated CodaVax-H1N1, (intramuscular injection; 1×10⁸ PFU/ml, lot 1-031321-1) as part of Southern Research study number 16115.01. Serum was collected from all animals on days 0, day 21 and day 35 and the anti-HA2(A/Vietnam/1203/2004)(H5N1) IgG titer was determined using an ELISA assay.

IgG ELISA. Ninety-six well plates were coated with HA2(A/Vietnam/1203/2004)(H5N1) (Immune Technology Corp. cat #: IT-003-0058p) at 6 ng/well in 50 ng/ml BSA/0.05M Carbonate/Bicarbonate Buffer pH 9.6 overnight at 4° C. Two-fold dilutions of purified IgG standard in range of 100-0.1 ng/ml was included in each plate. Plates were blocked with 10% goat serum in PBS for 2 hours at 37° C., washed four times with washing buffer (0.1% Tween 20 in PBS) then incubated with serially diluted monkey serum (1:50 starting dilution and two folds thereafter) in 10% goat serum/0.05% Tween-20 in PBS and incubated 1 hour at 37° C. After the incubation with monkey serum, plates were washed four times with washing buffer then incubated with 1:10,000 horseradish peroxidase (HRP) conjugated, affinity purified goat anti-Monkey IgG (H & L) (Fitzgerald; Cat #43C-CB1603) for 1 hour at 37° C. After the incubation, the plates were washed four times with washing buffer and Thermo Scientific OPD (o-phenylenediamine dihydrochloride) (Lot #UF2687722) was added for colorimetric reaction. Following 10 minutes of incubation in the dark at 25° C., the reaction was stopped by adding 100 ml per well 2.5M sulfuric acid solution and the resultant absorbance was read on a microplate reader at 490 nm. IgG levels among different groups were calculated using a standard curve. The standard curve was plotted as the OD₄₉₀ of each standard solution (X) versus the respective concentration of the standard solution (Y). The IgG (ng/ml) concentration was extrapolated by using the standard curve.

To assess the ability of Live Attenuated Influenza Vaccine, CodaVax-H1N1 to elicit antibodies (Abs) against the stalk domain of hemagglutinin of H5N1 influenza, levels of IgG against HA2(A/Vietnam/1203/2004)(H5N1) in control monkey sera and vaccinated monkey sera were determined using an ELISA assay. The reciprocal of the dilution at which the intensity of o-phenylenediamine dihydrochloride (OPD) colorimetric reaction reached three times above the background (no serum) was recorded for each animal. Relative anti-HA2(A/Vietnam/1203/2004)(H5N1) IgG titers were reported using the following formula:

$$anti - HA 2 ( A / Vietnam / 1203 / 2004 ) ( H 5 N 1 ) IgG Titer = \\ {log}_{10} ( 1 / Ave Dilution to Reach 3 X Above Background Color Intensity )$$

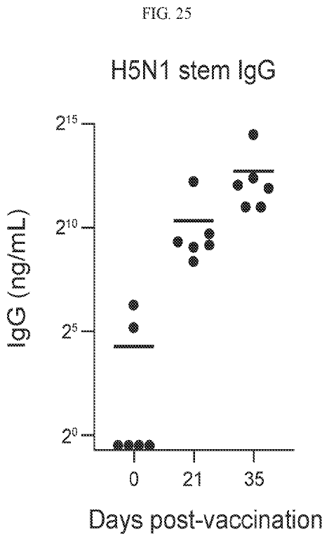

A comparison of the geometric mean of IgG levels against HA2(A/Vietnam/1203/2004)(H5N1) of post-inoculation of non-human primates sera can be found in Table 13 and FIG. 25.

Serum antibodies titer against HA2(A/Vietnam/1203/2004)(H5N1) in non-human primates following intramuscular administration with CodaVax-H1N1 were assessed using ELISA. Serum collected on days 21 and 35 showed in average about 100-fold and about 700-fold respectively higher antibody titer as compared to baseline titers (day 0) (Table 13, FIG. 25). Boosting was beneficial in bringing antibody titers up

TABLE 13
Geometric mean of serum anti-HA2(A/Vietnam/1203/2004)(H5N1) IgG
Titers (ng/ml) of Day 0, 21 and 35 post Coda Vax-H1N1 vaccination of
Non-human Primates

| | GEOMEAN | |
|---|---|---|
| | Males | Females |
| Day 0 | <0.8 | 13.106061 |
| Day 21 | 1099.7703 | 582.96969 |
| Day 35 | 7763.0929 | 2573.9539 |

### Example 7

CD1 mice were vaccinated with CodaVax-H1N1 or CodaVax-H3N2 (Singapore [HA+NA]^{Min}) via IM or IN route. Mice were boosted once with the same virus/dose/route Sera from all mice were drawn pre-vaccination (Day −4), pre-boost (Day 20), and post-boost (Day 37). HAI assays were performed to determine HAI titer at different time points. On Day 45, mice were challenged with Mouse Adapted H1N1 Cal4 WT at 10×LD50 or H3N2 Aichi 10×LD50 via IN route. 3 days after challenge, lungs were taken and viral replication were determined by plaque assay.

### Experimental Procedures:

### Mice

CD1 mice (female for grouped housing, 7 weeks old when first vaccinated)

### Cells

Vero (MCB master cell bank+9)

### Medium and Reagents Used:

Turkey red blood cells
RDE
OptiPRO
DPBS

### Viruses:

CodaVax-H1N1 (Lot: 1-102120-1-10,11,12, 1e+9 PFU/ml stock, L15/25% Sucrose)
A/California/07/2009(H1N1) (Lot: 6-011421-5, OptiPRO)
H3N2 Singapore (HA+NA)^{Min} (Lot: 1-021621-18,19,20, 2e+9 PFU/ml stock, L15/7% Sucrose)
A/Aichi/2/1968(H3N2) (Lot: 1-112420-1-7, L15/CDLC)
A/Singapore/INF1MH-16-0019/2016 (H3N2) (Lot: 1-101920-4-3, L15/25% Sucrose) A/California/04/2009(H1N1)-Mouse Adapted (MAD) (Lot E2842/101)

| | CD1 mouse vaccination and boost groups | | | |
|---|---|---|---|---|
| | | | Injection | Number |
| Group | Vaccine Strain | Dose (PU) | Route | of Mice |
| 1 | CodaVax-H1N1 | 1.00E+07 | IM | 10 |
| 2 | CodaVax-H1N1 | 1.00E+06 | IM | 10 |
| 3 | CodaVax-H3N2 | 1.00E+07 | IM | 10 |
| 4 | CodaVax-H1N1 | 1.00E+07 | IN | 5 |
| 5 | CodaVax-H1N1 | 1.00E+06 | IN | 5 |
| 6 | CodaVax-H3N2 | 1.00E+07 | IN | 5 |
| 7 | CodaVax-H3N2 | 1.00E+06 | IN | 5 |
| 8 | Bivalent (mixed) | 1.00E+07 (each) | IM | 10 |
| | CodaVax-H1N1 + | | | |
| | CodaVax-H3N2 | | | |
| 9 | Mock | OptiPRO | IM | 10 |

Nine groups of female CD1 mice were vaccinated with different doses of H1N1 or H3N2 vaccines or both via IM or IN in 50 uL diluted in OptiPRO. For IM route, viruses were administered to the rear thigh, without anesthesia.

On day −4, pre-vaccination 200-400 ul sera were collected for all mice from the submandibular vein.

On day 0, all mice received vaccination.

On day 20, pre-boost 200-400 ul sera were collected for all mice from the submandibular vein.

On day 22, all mice received a boost via same vaccination route as primary vaccination. On day 37, post-boost 200-400 ul sera were collected for all mice via submandibular vein.

On day 45, mice were challenged with 10×LD50 of wildtype virus (mouse adapted H1N1 or H3N2 Aichi)

### Determine HA Titer

To evaluate the immune response elicited by IM and IN vaccination of the vaccine strains, HAI assays were performed for sera collected pre-vaccination, pre-boost, and post-boost. Briefly, sample serum was treated with RDE and inactivated at 56° C. for 30-40 minutes. Four HA units of virus were added to serial 2-fold dilutions of sera, starting with a 1:10 dilution. Plates were incubated at room temperature for 1 h on a rocking platform, then 50 ul/well of 0.5% of turkey red blood cells (RBC) were added. Negative control wells had only RBC and no virus or sample serum. The plates were incubated at room temperature for 1 h or until the negative RBC control formed a clear button of RBC on the bottom of the well.

### Challenge

| | | Dose | Injection | |
|---|---|---|---|---|
| Group | Vaccine Strain | (PU) | Route | Challenge Virus |
| 1 | CodaVax-H1N1 | 1.00E+07 | IM | MAD H1N1 Cal4 WT |
| 2 | CodaVax-H1N1 | 1.00E+06 | IM | MAD H1N1 Cal4 WT |
| 3 | CodaVax-H3N2 | 1.00E+07 | IM | H3N2 Aichi WT |
| 4 | CodaVax-H1N1 | 1.00E+07 | IN | MAD H1N1 Cal4 WT |
| 5 | CodaVax-H1N1 | 1.00E+06 | IN | H3N2 Aichi WT |
| 6 | CodaVax-H3N2 | 1.00E+07 | IN | H3N2 Aichi WT |
| 7 | CodaVax-H3N2 | 1.00E+06 | IN | H3N2 Aichi WT |
| 8 | Bivalent (mixed) | 1.00E+07 | IM | MAD H1N1 Cal4 WT |
| | CodaVax-H1N1 + | (each) | | (4 mice) |
| | CodaVax-H3N2 | | | H3N2 Aichi WT |
| | | | | (5 mice) |
| 9 | Mock | OptiPRO | IM | MAD H1N1 Cal4 WT |
| | | | | (4 mice) |
| | | | | H3N2 Aichi WT |
| | | | | (5 mice) |

On day 45 post vaccination, mice were received 10×LD50 challenge virus (WT). 3 days post challenge (Day 48 post vaccination), mice were euthanized and their lungs were harvested for plaque assay to determine viral lung replication.

### Results

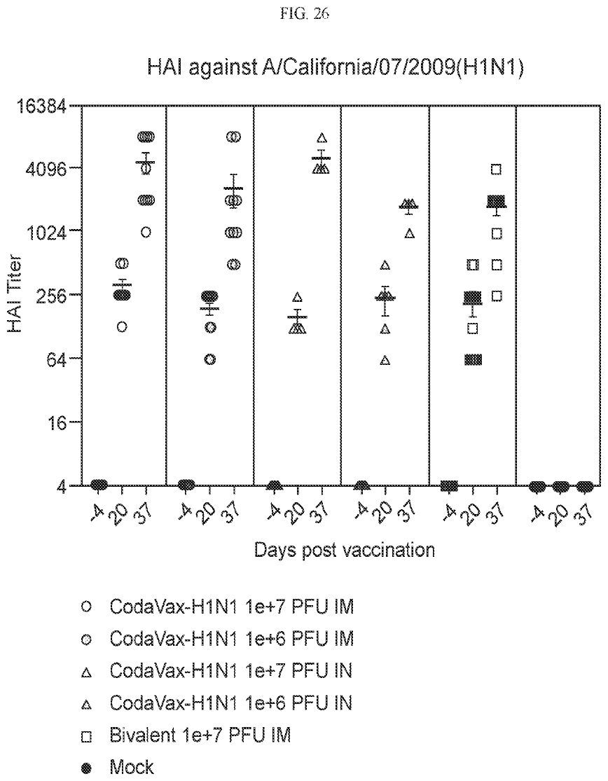

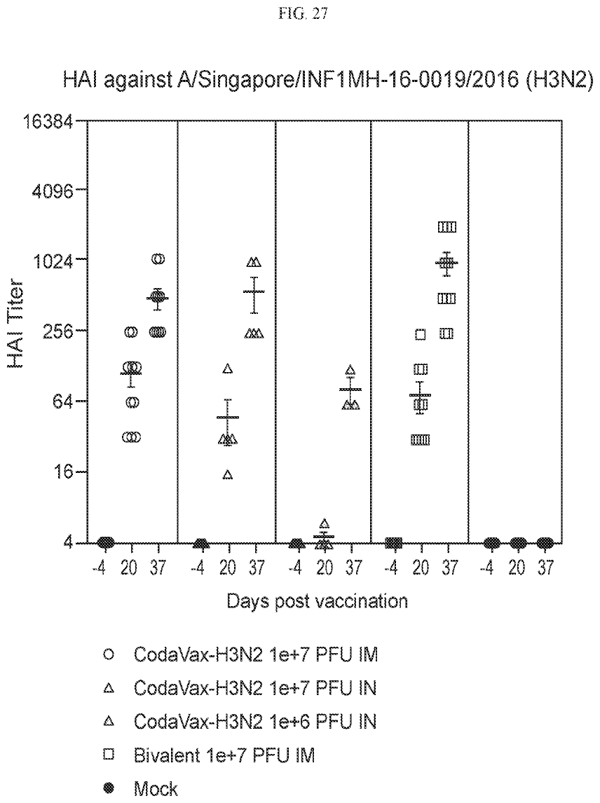

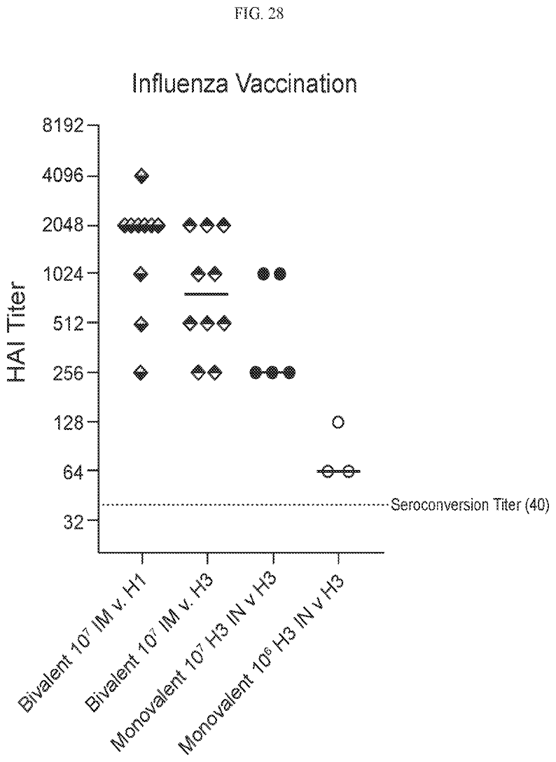

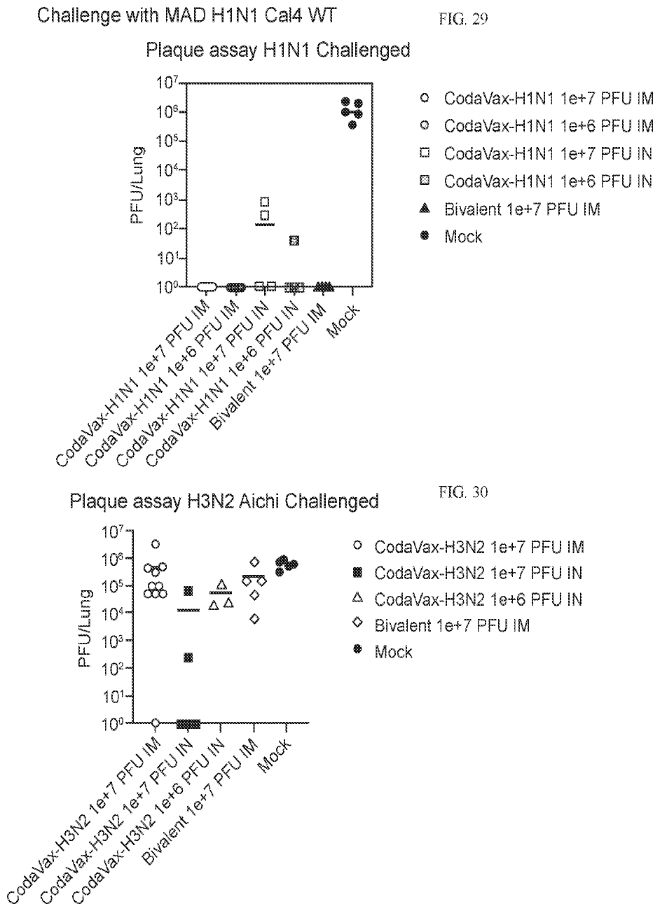

Results are in part depicted in FIGS. 26-30.

Number of mice showing a 4-fold or more increase in HAI against H1 N1 Cal7-WT between D20 (pre-boost) and Day 37 (post-boost):
CodaVax-H1N1 1e+7 PFU IM: 10/10
CodaVax-H1N1 1e+6 PFU IM: 10/10
CodaVax-H1N1 1e+7 PFU IN: 4/4
CodaVax-H1N1 1e+6 PFU IN: 4/4
Bivalent 1e30 7 PFU IM: 10/10

Number of mice showing a 4-fold or more increase in HAI against H3N2 Sing-WT between D20 (pre-boost) and Day 37 (post-boost):
CodaVax-H3N2 1e30 7 PFU IM: 7/10
CodaVax-H3N2 1 e+7 PFU IN: 5/5
CodaVax-H3N2 1e30 6 PFU IN: 3/3
Bivalent 1e30 7 PFU IM: 9/10

Bivalent v H1 is the HAI titer against H1 N1 CA/07/09 where the deoptimized H1N1+deoptimized H3N2 are mixed and injected IM.

Bivalent v H3 is the HAI titer against H3N2 Singapore when CodaVax-H1N1 and CodaVax-H3N2 are mixed and injected IM.

### Discussion

Both IM monovalent and bivalent vaccination of different influenza vaccine candidates showed very good results, with high HAI titers after boost.

For CodaVax-H1N1, IM injection shows very similar HAI titer against H1N1 WT Cal7 when compared to IN inoculation at both 1e+7 and 1e+6 PFU doses. All mice in all IM CodaVax-H1N1 groups showed a 4-fold increase in HAI upon boost. Mice that received 1e+7 PFU CodaVax-H1N1 IM demonstrated a slightly higher HAI titer than 1e+6 PFU group.

For H3N2 Singapore vaccine, CodaVax-H3N2, both IM and IN inoculation at 1e+7 PFU induced high HAI titer against H3N2 Singapore wildtype virus. Interestingly, IN vaccinated mice that received the lower dose of CodaVax-H3N2 (1e+6 PFU) showed almost no HAI titer (less than 8) following primary vaccination, but the HAI titer increased following boosting.

The most interesting results come from bivalent vaccination, in which 10 CD-1 mice received 1e+7 PFU of CodaVax-H1N1 and 1e+7 PFU of CodaVax-H3N2 (mixed in OptiPRO) via IM injection. The HAI titer in mice from this group showed very high HAI titer against both H1N1 Cal7-WT and H3N2 Sing-WT. No significant interference between the two vaccines was observed. Interestingly, bivalent IM injection of 1e+7 PFU (each) induced higher HAI titer against H3N2 Sing-WT than monovalent CodaVax-H3N2 IN vaccination at 1e+7 PFU, and significantly higher HAI titer than monovalent CodaVax-H3N2 IN vaccination with 1e+6 PFU.

In challenge experiments, CodaVax-H1N1 IM vaccination completely protected mice from lethal challenge of homologous MAD H1N1 Cal4 WT, with no virus detected in lung homogenates 3 days after challenge. In contrast, CodaVax-H3N2 IM vaccination did not protect mice from heterologous challenge with H3N2 Aichi.

In conclusion, IM vaccination with CodaVax-H1N1 and CodaVax-H3N2 is a route for inducing high HAI titer. Bivalent IM injection of these two vaccine candidates induced high HAI titer against both WT viruses, with no significant interference when compared to monovalent injection.

### Example 8

### Immunogenicity of Live Attenuated Influenza and It's Inactivated Form in CD1 Mice

Live wildtype influenza virus A/California/07/09 (Cal07-WT) and live attenuated vaccine virus CodaVax-H1N1 were inactivated by either UV light exposure or beta-Propiolactone (BPL). Details on the inactivation can be found in report CY17.

The live viruses as well as the inactivated viruses from CY17 were injected in CD-1 mice to test their immunogenicity as potential vaccine candidates. The group designations can be found in Table 14. In general, group of CD-1 mice received 50 ul inoculum intramuscularly, with 1e+6 PFU of viruses (diluted five-fold further from the CY17 experiment).

TABLE 14
Experimental Groups

| Group | Animal | |
|---|---|---|
| Number | Numbers | Inoculum |
| 1 | 8 | Cal7 WT Live (1e6 PFU) |
| 2 | 8 | Cal7 UV Inactivated (1e6 PFU) |
| 3 | 8 | Cal7 WT BPL Inactivated (1e6 PFU) |
| 4 | 9 | CodaVax-H1N1 Live (1e6 PFU) |
| 5 | 9 | CodaVax-H1N1 UV Inactivated (1e6 PFU) |
| 6 | 9 | CodaVax-H1N1 BPL Inactivated (1e6 PFU) |
| 7 | 9 | Mock (PBS) |

Sera was collected from the mice 2 days pre-vaccination, day 20 post-vaccination (pre-boost), and day 34 post-vaccination. The sera samples were then analyzed for hemagglutination inhibition titers (for homologous protection) and ELISA against H5 HA2 and M2 for heterologous immunity.

### Material and Methods

### Mice:

60 CD1 Mice (Charles River Laboratories; 4-6 weeks of age)

### Viruses:

CodaLytic H1N1 (P7): E2940/17B. ID: 1-092721-1 #046, 1e+9 PFU/ml;
Cal07-WT (P6): E2646/47. ID: 1-090919-1-1, 2e+10 PFU/ml;
A/CA/07/09-wt H1N1rec. ID 3-110218-5 (1-6)
   Medium and reagents used:
beta-Propiolactone (Thermo Scientific, Cat #B23197.06, Lot #S23H051)
Influenza Diluent: 20 mM Tris PH 7.6, 100 mM NaCl, 7.5% Sucrose. Lot: 6-02142022-1
HA2 (A/Vietnam/1203/2004) H5N1: Immune Technology Corp. Cat IT-003-0058p)
M2e-A/Cal07/09: Lot: U1159HB210-1/PE4063
OPD: Thermo Scientific Lot UF2687722

### Equipment:

Benchmark UV-Clade (Cat #B1450-IC)
Pipettes—p20, p200, p1000 and tips
96-well tissue culture plates, round bottom
PECTRAmax 250 (Molecular Devices) and software SoftMax Pro, version 4.8

### Preparation of Viruses

Cal07-WT and CodaVax-H1N1 were diluted to 1e8 PFU/mL in PBS and aliquoted into test samples of approximately 500 uL. These samples were inactivated via UV light or BPL at different concentrations and time.

For this assay, the viruses were further diluted five-fold in PBS for a final concentration of 1e6 PFU/mL of each inactivated virus.

### Hemagglutination Inhibition (HA) Assay

To evaluate the immune response elicited by live and inactivated vaccine strains, HAI assays were performed on sera collected pre-vaccination, pre-boost, and post-boost against A/California/07/09 (H1N1). Serum was treated with Receptor Destroying Enzyme (RDE) II (Denka Seiken, Cat #370013, Lot #589121, Exp. 2020-12) and inactivated at 56° C. for 30-40 minutes. Four hemagglutination units (HA) of virus were added to serial 2-fold dilutions of sera, starting with a 1:10 dilution. Plates were incubated at room temperature for 1 h on a rocking platform, then 50 ul/well of 0.5% of turkey red blood cells (RBC) were added. Negative control wells had only RBC and no virus or sample serum. The plates were incubated at room temperature for 1 h or until the negative RBC control formed a clear button of RBC on the bottom of the well.

The HAI Titer was read as the highest dilution of serum which inhibits hemagglutination.

### Enzyme-Linked Immunosorbent Assays (ELISAs)

96-well plates were coated with different antigen as listed in material and methods in 50 ng/ml BSA/0.05 M carbonate/bicarbonate buffer pH 9.6 overnight at 4 C. 2-fold dilution of purified IgG standard in range of 100-0.1 ng/ml was included in each plate. Plates were blocked with PBS+10% goat serum for 2 hours at 37 C, followed by washing 4 times with PBS+0.1% Tween 20. Mouse serum were serial diluted in PBS/0.05% Tween-20/10% Goat Serum and added to plates. After incubation, plates were washed 4 times with PBS+0.1% Tween-20, and then incubated with 1:10,000 HRP conjugated affinity pure goat anti-mouse IgG for 1 hour at 37 C. The plates were than washed and developed with OPD. Following 10 min of incubate in the dark, the reaction was stopped by adding 100 ul per well 2.5 M sulfuric acid solution and the read on a microplate reader at 490 nm. IgG levels among groups were calculated using a standard curve.

### Results & Discussion

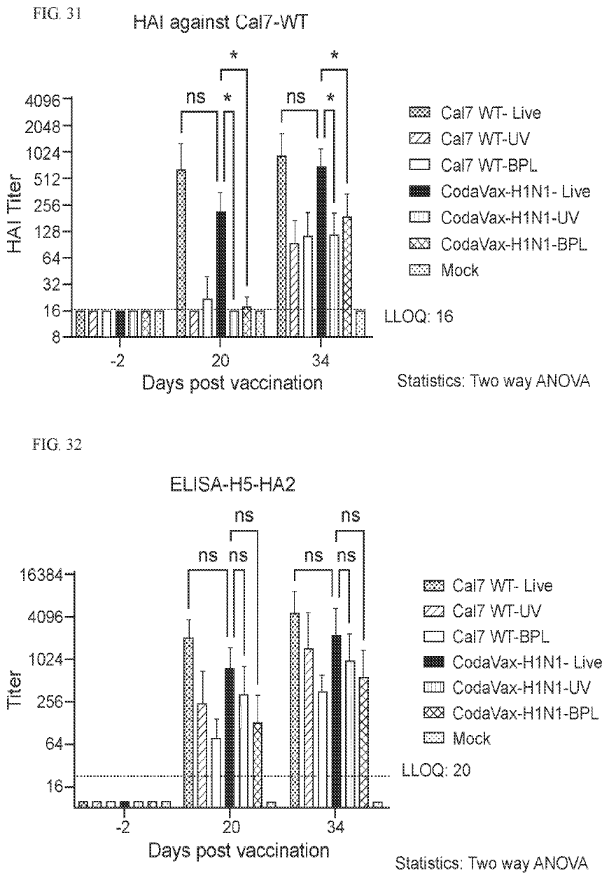

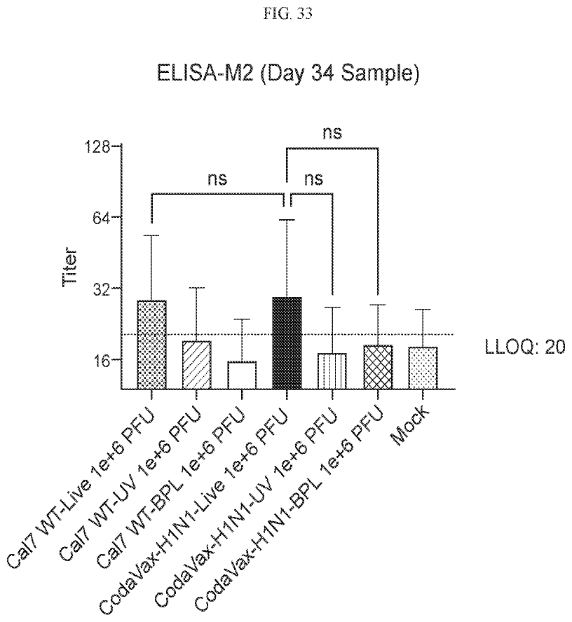

Results are in part depicted in FIGS. 31-33.

HAI assays were performed to evaluate HAI titer for different vaccine candidates against wild type A/California/07/2009(H1N1) viruses.

Although UV-inactivation preserved more HA units compared to BPL-inactivation both methods behave very similar in HAI titer against wild type A/California/07/2009(H1N1). However, the inactivation methods may play a role in cross protection, as UV-inactivated viruses trigger higher binding IgG antibodies against H5 HA2 when compared to BPL-inactivated viruses. As expected, both live Cal07 WT and CodaVax-H1N1 induce higher HAI titer than its inactivated form, indicating virus replication, even via intramuscular injection, offer better humoral immunity than inactivated viruses with the exact same amount of virions or protein compositions. Surprisingly, our CodaVax-H1N1 upon boost achieved the same level of HAI titer as Cal07-WT virus.

In addition, live viruses induced better heterologous protection when compared to inactivated viruses. ELISA showed more H5 HA2 binding IgG were induced by both live Cal07 WT and CodaVax-H1N1 than inactivated viruses. Nonetheless, all candidates induced high level of H5 HA2 binding IgG ELISA titer.

On the contrary, M2 ELISA titers are consistently low or undetectable for all candidates tested. Interestingly, although none of the inactivated viruses induce detectable M2 ELISA titer, both live Cal07 WT and CodaVax-H1N1 have an ELISA titer against M2 around 32, which may offer a marginal advantage against inactivated format.

### Example 9

6 AGMs (N=3 per group). Group 1 received 10⁸ PFU CodaVax-H3N2+neg. control vaccine (non-influenza) by I.M. route on Day 0 and Day 21 Group 2 received the neg. Control Vaccine (non-influenza) by I.M. route on Day 0 and Day 21.

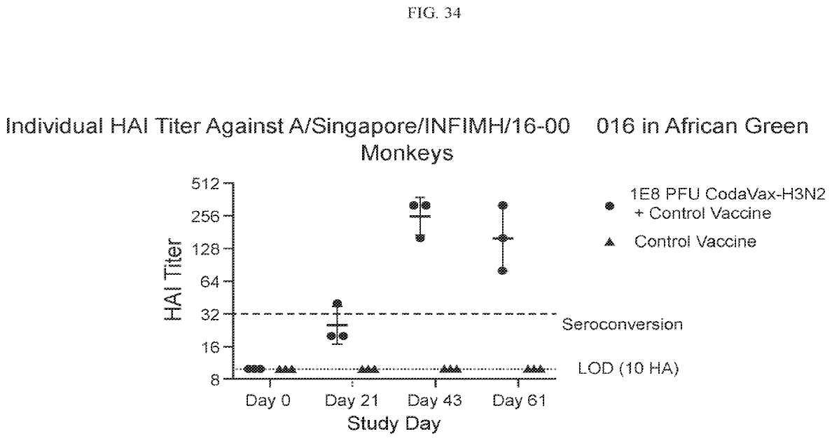

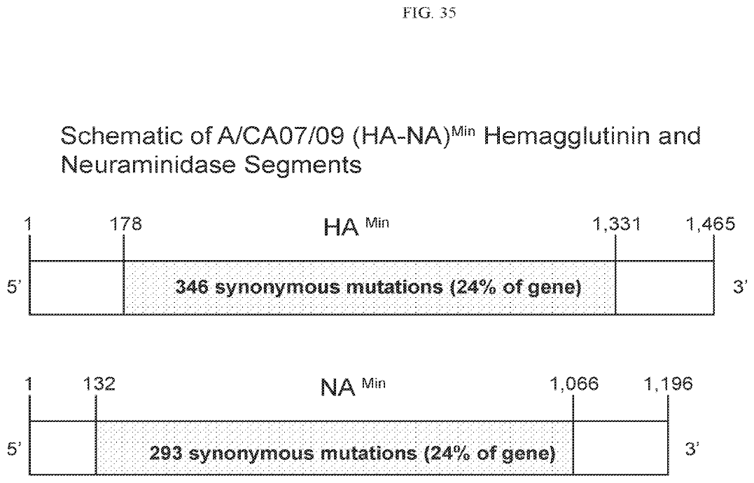

Results are shown in FIG. 34.

### Example 10

While there are inclusion and exclusion criteria, as well as prohibited medications and therapies, noted for this example/study, it is not to be construed as limitations to the claimed invention unless specifically and expressly provided for in the claims. Indeed, subjects excluded from this study and medications and therapies prohibited by this study are encompassed by embodiments of the present invention, unless specifically and expressly excluded in the claims.

### Design

Described herein is a randomized, double-blind, placebo- and active-controlled, clinical trial to evaluate the safety and immunogenicity of CodaVax-H1N1 in adults aged 18 to 49 years. Participants will be screened within 28 days of randomization (Day 1), and eligible participants will be enrolled into 1 of 3 sequential cohorts and randomized in a 3:1:1 ratio within each cohort to receive a single IM dose of CodaVax-H1N1, placebo (normal saline), or licensed injectable seasonal influenza vaccine (Flucelvax Quad) as shown in Table 15.

TABLE 15

| Cohort | CodaVax-H1N1 | Placebo | Fucelvax Quad |
|---|---|---|---|
| 5 × 10⁴ PFU | | | |
| Sentinel group | 2 | 1 | 0 |
| Rest of cohort | 7 | 2 | 3 |
| 5 × 10⁵ PFU | 9 | 3 | 3 |
| 5 × 10⁶ PFU | 9 | 3 | 3 |
| Total | 27 | 9 | 9 |

The SRC will review blinded safety data for all participants through Day 8 before enrolment of rest of the cohort after sentinel group in the low-dose cohort and before escalation to the next dose cohort.

Participants will record reactogenicity (local events, systemic events, and temperature) in a daily diary for 7 days after the dose. Each participant will be contacted by telephone on the day after dosing for safety assessment and review of the diary data. Participants will return to the clinic on Days 4, 8, 29, 91, and 181 for safety and immunogenicity assessments.

All AEs and concomitant medications will be recorded from signing of the ICF to 28 days postdose. Thereafter to the end of the study, only MAAEs, NCIs, SAEs, immunosuppressive medications, blood products, and vaccines will be recorded. A complete physical examination will be performed at Screening, and targeted and symptom-driven physical examinations will be performed predose on Day 1, 2 hours postdose, and at each postdose visit through Day 91. Vital signs will be measured at the same time points. An ECG will be performed at Screening and on Day 29.

A serum sample will be collected predose and on Days 29, 91, and 181 for measurement of HAI and neutralizing antibody titres.

A whole blood sample will be collected predose and on Day 8 and PBMCs isolated for measurement of T-cell response.

If a participant experiences acute symptoms compatible with viral respiratory infection, nasopharyngeal swab samples will be collected for a rapid influenza diagnostic test and
respiratory virus PCR assay panel (including SARS-CoV-2) as indicated for symptomatic respiratory infection and will perform the rapid influenza diagnostic test.

The primary analysis of study data will be conducted after all participants complete the Day 29 visit. The final analysis will be conducted at the end of the study.

### Inclusion Criteria

Participants who meet all the following criteria may be included in the study:
1. Aged 18 to 49 years, inclusive, on the day of signing the ICF
2. Body mass index (BMI)≥18 and ≤35 kg/m²
3. In good health with no history, or current evidence, of clinically significant medical conditions with particular reference to, but not restricted to, thromboembolic disorders, coronary heart disease, chronic obstructive lung disease, diabetes mellitus (gestational diabetes is allowed if treatment was not required postpartum and serum glucose is currently within the normal range) and no clinically significant test abnormalities that will interfere with participant safety, as defined by medical history, physical examination, vital signs, ECG, and laboratory tests as determined by the Investigator
4. Negative SARS-CoV-2 test predose on Day 1
5. For all women, negative pregnancy test
6. Agreement to comply with the following conditions to prevent the spread of genetically modified organisms (GMOs):
   a. Hygiene measures intended to prevent interpersonal transmission of IP must be implemented, including but not limited to frequent handwashing with soap or hand disinfectant, respiratory hygiene, and cough etiquette for 7 days postdose.
   b. No donation of blood, tissue, or organs for 7 days postdose
   c. No contact with or care for severely immunosuppressed persons who require a protective environment for 7 days postdose
   d. All tissues and materials used to collect respiratory secretions for 7 days postdose will be sealed in a primary container and placed within a secondary container so that it is not accessible to children or animals until it is returned to the study site for disposal.
7. Agreement to practice a highly effective, double method of contraception through 28 days after the IP dose
8. Agreement to no blood donation ≥470 mL for 90 days after the IP dose
9. Willingness to participate and comply with all aspects of the study through the entire study period, including all visits and sample collection
10. Provision of written informed consent

### Exclusion Criteria

Participants who meet any of the following criteria will be excluded from the study:
1. Pregnant or lactating women or women who plan to become pregnant through Day 29
2. Inadequate venous access for repeated phlebotomy
3. History of autoimmune or demyelinating illness
4. History of Bell's palsy
5. History of postinfectious or postvaccine neurological sequelae, including Guillain-Barré syndrome
6. History of severe reaction to vaccination
7. History of anaphylaxis or angioedema
8. History of bleeding disorder (e.g., factor deficiency, coagulopathy, or platelet disorder requiring special precautions) or on anticoagulant therapy
9. History of seizures (other than childhood febrile seizures), dementia or progressive neurological disease
10. Known allergy to any of the ingredients in the study IPs
11. Known or suspected malignancy, except for nonmelanoma skin cancers and other early stage surgically excised malignancies that the Investigator considers exceedingly unlikely to recur
12. Immunodeficiency, including the use of systemic corticosteroids (>10 mg/day of prednisone or equivalent), alkylating drugs, antimetabolites, radiation, immune-modulating biologics, or other immunomodulating therapies within 90 days before Day 1 or plan to use any of these during the study (topical steroid creams are allowed)
13. Any other chronic medical condition that is not stable or required medication changes within 30 days before Day 1
14. Receipt of any licensed or investigational influenza vaccine within 6 months before Day 1 or planned receipt through Day 29
15. Receipt of any live vaccine within 30 days before Day 1
16. Receipt of blood transfusion or blood product within 90 days before Day 1 or planned receipt through the end of the study
17. Receipt of an IP within 30 days or 5 half-lives (whichever is longer) before Day 1
18. Receipt of cosmetic fillers in the face within 30 days before Day 1
19. Blood donation of ≥470 mL within 90 days before Day 1
20. Planned hospitalization or surgical procedure through Day 29
21. Positive result for HIV, hepatitis B virus, hepatitis C virus, or active hepatitis A virus infection
22. Screening laboratory values outside of normal range (>Grade 1) (Grade 2 bilirubin is acceptable in participants with Gilbert's syndrome and normal transaminases)
23. Systolic blood pressure >160 mmHg or diastolic blood pressure >90 mmHg) at Screening or on Day 1
24. Tattoo, skin reaction, or other condition at the injection site that would interfere with assessment of local reactogenicity
25. Any medical, psychiatric, or social condition or occupational or other responsibility that, in the judgment of the Investigator, would interfere with or serve as a contraindication to protocol adherence, assessment of safety (including reactogenicity), or a participant's ability to provide informed consent
26. Employee or immediate relative of employee of Codagenix, vendors, or research sites associated with the study

### CodaVax-H1N1

CodaVax-H1N1 is an intranasal, live, attenuated vaccine based on the wild-type influenza strain A/California/07/2009 and created using synthetic attenuated virus engineering (SAVE), a synthetic biology-based method to deoptimize viral genes for slowed translation in the human host cell [Coleman 2008]. The attenuated virus, A/CA07/09 (HA+NA)^{Min}, is a 100% match in all 8 segments to the wild-type strain; however, its HA gene segment, encoding a protein which mediates attachment and penetration of the virus, and its neuraminidase segment, encoding a protein that mediates release of virus from the infected cell, have been synthesized with over 600 silent mutations to introduce codons and codon pairs disfavored by mammalian cells. (FIG. 34).

The final product is a pale pink- to pink-colored liquid in Leibovitz's L-15 medium for IM administration. Codavax-H1N1 is supplied in 2 mL cryovials each containing 1.1 mL of virus suspension at a nominal concentration of 10⁷ PFU/mL (range: 10⁶ to 10⁸ FFU/mL). A single dose of 0.5 mL will be administered IM in the deltoid by qualified investigational site staff.

CodaVax-H1N1 is stored at ≤−70° C. and thawed to room temperature before dose preparation. Further information on preparation and administration of CodaVax-H1N1/placebo is provided in the pharmacy manual.

### Diluent

A commercially available buffered saline solution will be supplied by the investigational site, stored and handled in compliance with manufacturer's instructions, and used as the diluent to prepare the 5×10⁴ and 5×10⁵ nominal doses of CodaVax-H1N1.
to CodaVax-H1N1.

### Flucelvax Quad

Commercially available Flucelvax Quad (Seqirus Pty Ltd, Parkville VIC, Australia) will be supplied by the investigational site, stored, and administered in compliance with the manufacturer's instructions (Appendix 4).

### Labelling

All IPs will be labelled in accordance with applicable regulatory guidelines.

### Storage

All IPs will be stored in a secure place under appropriate storage conditions as stated in the pharmacy manual/product label.

### Blinding and Unblinding

Investigators, participants, and all study staff with direct participant contact will be blinded to assignment to CodaVax-H1N1, placebo, or Flucelvax Quad. A designated unblinded pharmacist (or other qualified staff member) at the site will prepare the blinded syringe for dispensing to the site staff who will administer the IP. The syringe will be masked with translucent tape to obscure minor differences in appearance of vaccine, placebo, and Flucelvax Quad. If possible, the staff member administering the vaccine will not be part of the blinded study team.

Laboratory results (e.g., immunogenicity data) may be unblinding, and access will be limited to unblinded laboratory staff. Investigator site staff who have contact with participants or assess clinical data will have no access to potentially unblinding laboratory results.

Unblinding of vaccine assignment is discouraged. In the event of a medical emergency, for which the identity of the assignment is critical to the care of a participant, the Investigator or designee will contact the Contract Research Organisation (CRO) Medical Monitor to discuss. In the event that unblinding is deemed necessary, a designated unblinded study team member will provide the assignment to the Medical Monitor who will provide the information to the Investigator.

Sponsor personnel will have no access to any individual treatment assignment until after the primary analysis, except as needed to satisfy requirements for prompt reporting of SAEs to Regulatory Authorities. Investigators and investigational site staff (other than unblinded pharmacy and laboratory staff) will remain blinded to individual treatment assignments until database lock for final analysis.

### Randomization and Timing of Doses

Participants will be enrolled into 1 of 3 sequential cohorts and randomized in a 3:1:1 ratio within each cohort to receive a single IM dose of CodaVax-H1N1, placebo (normal saline), or Flucelvax Quad as shown in Table 15.

The SRC (composed of Investigator, Sponsor Medical Monitor, CRO Medical Monitor) will review blinded safety data for all participants through Day 8 before enrolment of rest of the cohort after sentinel group in the low-dose cohort and before escalation to the next dose cohort. If any of the following events occur at any point during the study, then further administration of CodaVax-H1N1/placebo/Flucelvax Quad will be stopped:
A Grade 4 AE or SAE that is considered to be at least possibly related to the IP
Two Grade 3 or clinically significant AEs within the same system organ class that are considered to be at least possibly related to the IP
Bell's palsy of any grade

The SRC will review all available data including the data of the qualifying event(s) and determine if it is appropriate to recommence dosing (after approval of a substantial amendment by the Regulatory Authority and the Ethics Committee [if applicable]) or to terminate the study. In any event, all participants will be followed-up through at least Day 29 and in accordance with Section 9.8.

### Prior and Concomitant Medication and Therapies

### Prohibited Prior Medications and Therapies

Participants who received any of the following medications or therapies may not be enrolled:
Licensed or investigational influenza vaccine within 6 months before Day 1
Systemic corticosteroids (>10 mg/day of prednisone or equivalent), alkylating drugs, antimetabolites, radiation, immune-modulating biologics, or other immunosuppressive therapies within 90 days before Day 1 (topical steroid creams are allowed)
Blood transfusion or blood product within 90 days before Day 1
Any IP within 30 days or 5 half-lives (whichever is longer) before Day 1
Any live vaccine within 30 days before Day 1
Cosmetic fillers in the face within 30 days before Day 1

### Prohibited Concomitant Medications and Therapies

Participants may be administered an approved COVID-19 vaccine and/or influenza vaccine as recommended by local public health authorities for their age and risk group after Day 29. Although adults do not generally receive 2 doses of influenza vaccine in the same season, there is no contraindication to this, and adults may receive a second influenza vaccine in the same year if travelling from Australia to the northern hemisphere during the northern hemisphere influenza season. In addition, Flucelvax Quad is approved as a 2-dose series in children in the US, and in the study cited for the FDA Prescribing Information, the rates of solicited local and systemic adverse reactions were generally lower in children (N=762) after the second dose than after the first dose.

The following medications and vaccines are prohibited during the study:
Systemic corticosteroids (>10 mg/day of prednisone or equivalent), alkylating drugs, antimetabolites, radiation, immune-modulating biologics, or other immunosuppressive therapies at any time during the study (topical steroid creams are allowed)
Blood transfusion or blood product
Any IP other than CodaVax-H1N1/placebo/Flucelvax Quad
Cosmetic fillers in the face through Day 91

Any participant who receives one of these medications or vaccines in the prohibited period should remain in the study but will be excluded from the Per-protocol Population.

### Serum Samples (Immunogenicity)

A serum sample will be collected for measurement of HAI and neutralizing antibody titres at the time points specified in the Schedule of Events. Samples collected on Days 1 and 29 will also be tested by ELISA for H5 stem antibody titre; a larger serum sample will be collected on Days 1 and 29 to support this additional assay. Collection, processing, labelling, storage, and shipping instructions for these samples are provided in the laboratory manual.

### Whole Blood Samples (Isolation of PBMCs)

Whole blood samples will be collected and processed to isolate PBMCs for measurement of T-cell response at the time points specified in the Schedule of Events. Collection, processing, labelling, storage, and shipping instructions for these serum samples are provided in the laboratory manual.

### Respiratory Virus Assay Samples

Nasopharyngeal swab samples will be collected for a rapid influenza diagnostic test and respiratory virus PCR assay panel (including SARS-Cov-2) from any participant who experiences acute symptoms compatible with viral respiratory infection (moderate or severe through Day 29, of any severity thereafter) (Section 8.4). Instructions for processing, labelling, storage, and shipping for these samples are provided in the laboratory manual for the study.

Any sample positive for influenza A will be retained for analysis to determine if it is wild-type virus or vaccine virus strain.

### Dosing Period

If the participant experiences acute moderate or severe symptoms compatible with viral respiratory infection, an Unscheduled Visit/Telephone Contact (Section 8.4) should be performed.

### Day 1

If the participant has experienced any of the following symptoms, the Day 1 visit should be delayed:
Symptoms of upper respiratory infection or fever (subjective or objective) or malaise within 3 days before Day 1
Any symptoms or signs on Day 1 that could inhibit the proper administration of the IP or interpretation of solicited AEs in the participant diary (e.g., temperature >38° C., sunburn in the injection site area, muscle soreness)

Otherwise, the following procedures will be performed before administration of IP:
Concomitant medications recording
AE assessment
Targeted and symptom-driven physical examination
Vital signs
Alcohol breath test
Urine pregnancy test (for all women)
Urine drug screen
SARS-CoV-2 test
Eligibility criteria check

Upon determination that a participant meets all eligibility criteria, the following samples will be collected:
Serum for immunogenicity testing
Whole blood sample for isolation of PBMCs

After sample collection, the participant will be randomized to vaccine assignment as described in Section 6.6. IP will be administered (as described in Section 6.1).

The participant will remain at the investigational site and be observed for at least 2 hours postdose. The following procedures will be performed at least 2 hours after IP administration:
Targeted and symptom-driven physical examination
Vital signs
Concomitant medications recording
AE assessment

Before discharge, the participant will be given a diary and thermometer and instructed on completion of the diary. Site staff will instruct the participant to record temperature, local events, and systemic events at the same time today and each day for the next 7 days (through Day 7). The participant will also be instructed that, if any event is ongoing for more than 7 days after the dose, to follow the event until resolution and report the stop date.

Before discharge, site staff will remind participants to complete the diary and to contact the investigational site if they experience acute symptoms compatible with respiratory infection.

### Day 8 (±1 Day)

The following procedures will be performed:
Review of diary data, collection of diary
Targeted and symptom-driven physical examination
Vitals signs
Concomitant medications recording
AE assessment
Sample collection:
   Safety laboratory tests
   Whole blood sample for isolation of PBMCs

Site staff will remind participants to contact the site if they experience acute symptoms compatible with respiratory infection.

### Day 29 (±3 Days)

The following procedures will be performed:
Concomitant medications recording
AE assessment
Targeted and symptom-driven physical examination
ECG
Vital signs
Sample collection
   Serum pregnancy test (for all women)
   Serum for immunogenicity testing

Site staff will remind the participant to contact the investigational site if they experience acute symptoms compatible with respiratory infection, are hospitalized, or visit the doctor for a new illness.

### Follow-Up Period

If the participant experiences symptoms compatible with viral respiratory infection of any severity, an Unscheduled Visit/Telephone Contact should be performed.

### Day 91 (±7 Days)

The following procedures will be performed:
Recording of immunosuppressive medications, blood products, and vaccines
Assessment of MAAEs, NCIs, SAEs
targeted and symptom-driven physical examination
Vitals signs
Sample collection
   Serum for immunogenicity testing

### Day 181 (14 Days)

The following procedures will be performed:
Recording of immunosuppressive medications, blood products, and vaccines
Assessment of MAAEs, NCIs, SAEs
Sample collection
   Serum for immunogenicity testing

### Safety Analyses

### Primary Endpoints (Reactogenicity and Adverse Events)

The number (percentage) of participants with local and systemic reactogenicity events for 7 days postdose will be summarized by group. Reactogenicity events will also be summarized by seventy.

The number (percentage) of participants with AEs (including MAAEs, NCIs, and SAEs) from Day 1 to Day 29 will be summarized for each MedDRA system organ class and preferred term and by group. The number (percentage) of participants with MAAEs, with NCIs, and with SAEs from Day 1 to Day 181 will be summarized in a similar fashion. The number (percentage) of participants with AEs by severity and by relationship to IP will also be summarized.

### Other Safety Data

Summary statistics for continuous parameters (clinical laboratory results and vital signs) will be presented by group as follows: baseline, postdose, and change from baseline to postdose assessment.

Shift tables which cross-tabulate the baseline and postdose safety laboratory values of each participant by severity grade will be prepared.

Summaries of the number and percentage of participants with normal, abnormal not clinically significant, and abnormal clinically significant ECG interpretations will be presented.

Physical examination data will be listed.

### Immunogenicity Analyses

### Secondary Endpoint (Humoral Immunogenicity)

The primary variable of interest for assessment of humoral immune response to CodaVax-H1N1 is HAI titre against A/California/07/2009 and the current seasonal influenza vaccine H1N1 and H3N2 strains. The following measures and their 95% CIs will be summarized by group:
GMT at baseline and on Days 29, 91, and 181
GMFR on Days 29, 91, and 181
Responder (≥4×baseline value) rates on Days 29, 91, and 181

### Exploratory Endpoints

### Neutralizing Antibody

Neutralizing antibody titres against A/California/07/2009 and the current seasonal influenza vaccine H1N1 and H3N2 strains will be summarized by the following measures and their 95% CIs by group:
GMT at baseline and on Days 29, 91, and 181
GMFR on Days 29, 91, and 181
Responder (≥2×baseline value) rates on Days 29, 91, and 181

### Cellular Immunogenicity

Cellular responses will be summarized by the following measures and their 95% CIs by group:
Geometric mean SFU/10⁶ PBMCs at baseline and on Day 8
SFU GMFR on Day 8
Responder (≥2×baseline value) rates on Day 8

### Immunogenicity to Conserved Hemagglutinin Stem Antigens

H5 stem antibody titre will be summarized by the following measures and their 95% CIs by group:
GMT at baseline and on Day 29
GMFR on Day 29
Responder (≥2× baseline value) rates on Day 29

### Impact of Baseline Influenza Immunity on Immunogenicity of CodaVax-H1N1

Subgroup analysis of selected immunogenicity endpoints will be performed by low and high baseline neutralizing antibody titre against A/California/07/2009 and baseline ELISpot score.

### Respiratory Illness Incidence Analysis

Rapid influenza diagnostic test and PCR assay results from symptomatic participants, results of genetic analysis of any influenza A isolates, and associated symptoms will be listed.

### Sample Size and Power

The sample size for this study was selected as adequate and reasonable for a review of the safety and immunogenicity profile of IM CodaVax-H1N1 in adults. Given 27 participants receiving CodaVax-H1N1, the study will have at least 80% probability of detecting at least 1 AE that occurs at a rate of 5.8%. If no SAEs are observed among the 27 participants who receive CodaVax-H1N1, an approximation to the 1-sided upper bound of the 95% CI of the SAE occurrence rate would be 10.5%.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of eliciting an immune response in a subject in need thereof, comprising:
parenterally administering a composition comprising a live attenuated influenza virus in which expression of hemagglutinin (HA) and neuraminidase (NA) is reduced compared to a natural isolate virus, wherein the reduction in expression is a result of recoding the HA protein-encoding sequence and recoding the NA protein-encoding sequence, thereby eliciting the immune response in the subject,
wherein one or both of the HA protein-encoding sequence and the NA protein-encoding sequence on the attenuated influenza virus are recoded by lowering the codon-pair bias of the protein-encoding sequence as compared to the natural isolate virus.

2. A method of eliciting an immune response in a subject in need thereof, comprising:
parenterally administering a composition comprising a live attenuated influenza virus in which its hemagglutinin (HA) protein encoding sequence and neuraminidase (NA) protein encoding sequence are recoded compared to its natural isolate virus,
wherein one or both of the HA protein-encoding sequence and the NA protein-encoding sequence on the attenuated influenza virus are recoded by lowering the codon-pair bias of the protein-encoding sequence as compared to the natural isolate virus, and
wherein the amino acid sequence of HA protein or NA protein of the live attenuated influenza virus remains the same compared to its natural isolate virus, or
wherein the amino acid sequence of the HA protein or NA protein of the live attenuated influenza virus comprises up to 20 amino acid substitutions, additions, or deletions compared to the natural isolate virus,
thereby eliciting the immune response in the subject.

3. The method of claim 2, wherein the composition comprises two or more different subtypes of the live attenuated influenza viruses.

4. The method of claim 3, wherein two or more different subtypes of the live attenuated influenza viruses comprises a H1N1 subtype and a H3N2 subtype.

5. The method of claim 2, wherein the composition comprises about 10³-10⁹ PFU of the live attenuated influenza virus.

6. (canceled)

7. The method of claim 2, wherein parenterally administering the composition comprises parenterally administering a single dose.

8. The method of claim 2, wherein parenterally administering the composition comprises parenterally administering a prime dose and parenterally administering at least one boost dose.

9. The method of claim 8, wherein
the prime dose is about 10³-10⁹ PFU of the live attenuated influenza virus, and each of the least one boost dose is about 10³-10⁹ PFU of the live attenuated influenza virus, OR
the prime dose is about 10⁷-10⁸ PFU of the live attenuated influenza virus and each of the least one boost dose is about 10⁷-10⁸ PFU of the live attenuated influenza virus.

10. (canceled)

11. (canceled)

12. (canceled)

13. (canceled)

14. (canceled)

15. (canceled)

16. (canceled)

17. The method of claim 2, wherein parenterally administering comprises intramuscular injection or subcutaneous injection.

18. (canceled)

19. (canceled)

20. (canceled)

21. The method of claim 2, wherein
eliciting the immune response comprises at least a 10-fold increase in hemagglutination inhibition (HAI) titer, neuraminidase inhibition (NAI) titer, or both, in the subject about 14 to 35 days after parenteral administration of the composition as compared to day 0 before parenteral administration with the composition.

22. The method of claim 2, wherein eliciting the immune response comprises a 30-150-fold increase in hemagglutination inhibition (HAI) titer, neuraminidase inhibition (NAI) titer, or both, in the subject about 14 to 35 days after parenteral administration of the composition as compared to day 0 before parenteral administration with the composition.

23. The method of claim 2, wherein the expression of other influenza proteins in attenuated influenza virus are not substantially reduced.

24. (canceled)

25. (canceled)

26. (canceled)

27. The method of claim 2, wherein each of the recoded HA protein-encoding sequence and the recoded NA protein-encoding sequence of the attenuated influenza virus have a codon pair bias less than −0.05, less than −0.1, less than −0.15, less than −0.2, less than −0.25, less than −0.3, less than −0.35, or less than −0.4.

28. (canceled)

29. (canceled)

30. (canceled)

31. (canceled)

32. The method of claim 2, wherein the HA protein of the live attenuated virus is encoded by a HA protein encoding sequence comprising SEQ ID NO:1, SEQ ID NO:3, SEQ ID NO:5, or SEQ ID NO:7 or a HA protein encoding sequence selected from Table 1.

33. The method of claim 2, wherein the HA protein of the live attenuated virus is encoded by a HA protein encoding sequence comprising SEQ ID NO: 15, ORF of SEQ ID NO:15, SEQ ID NO:16, ORF of SEQ ID NO:16, SEQ ID NO: 17, or ORF of SEQ ID NO:17.

34. The method of claim 2, wherein the NA protein of the live attenuated virus is encoded by a NA protein encoding sequence comprising SEQ ID NO:2, SEQ ID NO:4, SEQ ID NO: 6, SEQ ID NO:8, SEQ ID NO:18, ORF of SEQ ID NO:18 or a NA protein encoding sequence selected from Table 1.

35. The method of claim 2, wherein eliciting the immune response comprises at least a 50-fold increase in anti-hemagglutinin (HA) IgG titer in the subject after about 21 days after parenteral administration of the composition as compared to day 0 before parenteral administration of the composition.

36. The method of claim 2, wherein eliciting the immune response comprises about 100-fold to 700-fold increase in anti-hemagglutinin (HA) IgG titer in the subject after about 21 days after parenteral administration of the composition as compared to day 0 before parenteral administration of the composition.

37. A live attenuated influenza virus comprising a hemagglutinin (HA) protein encoded by a HA encoding sequence, a neuraminidase (NA) protein encoded by a NA encoding sequence or both, wherein the HA protein encoding sequence comprises SEQ ID NO:1, SEQ ID NO:3, SEQ ID NO:7, SEQ ID NO:15, ORF of SEQ ID NO:15, SEQ ID NO:16, ORF of SEQ ID NO:16, SEQ ID NO:17, or ORF of SEQ ID NO:17 and the NA protein encoding sequence comprises SEQ ID NO:2, SEQ ID NO:4, SEQ ID NO:8, SEQ ID NO:18 or ORF of SEQ ID NO:18.

38. The live attenuated influenza of claim 37, wherein the nucleic acid having SEQ ID NO:1, SEQ ID NO:2, SEQ ID NO:3, SEQ ID NO:4, SEQ ID NO:7, SEQ ID NO:8, SEQ ID NO: 18, SEQ ID NO:15, ORF of SEQ ID NO:15, SEQ ID NO:16, ORF of SEQ ID NO:16, SEQ ID NO:17, ORF of SEQ ID NO:17, or ORF of SEQ ID NO:18 or any combination thereof imparts the attenuation compared to its natural isolate form.

39. A parenterally administrable composition, comprising:
a live attenuated influenza virus in which expression of hemagglutinin (HA) and neuraminidase (NA) is reduced compared to a natural isolate virus, wherein the reduction in expression is a result of recoding the HA protein-encoding sequence and recoding the NA protein-encoding sequence, thereby eliciting the immune response in the subject, or
a live attenuated influenza virus in which its hemagglutinin (HA) protein encoding sequence and neuraminidase (NA) protein encoding sequence are recoded, and
wherein the amino acid sequence of HA protein or NA protein of the live attenuated influenza virus remains the same, or wherein the amino acid sequence of the HA protein or NA protein of the live attenuated influenza virus comprises up to 20 amino acid substitutions, additions, or deletions compared to the natural isolate virus; and
a pharmaceutically acceptable carrier or excipient suitable for administration via parenterally administration.

40. An parenterally administrable composition of claim 39, wherein the live attenuated influenza virus comprises a hemagglutinin (HA) protein encoded by a HA encoding sequence, a neuraminidase (NA) protein encoded by a NA encoding sequence or both, wherein the HA protein encoding sequence comprises SEQ ID NO:1, SEQ ID NO:3, SEQ ID NO:7, SEQ ID NO:15, ORF of SEQ ID NO:15, SEQ ID NO:16, ORF of SEQ ID NO:16, SEQ ID NO: 17 or ORF of SEQ ID NO:17 and the NA protein encoding sequence comprises SEQ ID NO:2, SEQ ID NO:4, SEQ ID NO:8, SEQ ID NO:18, or ORF of SEQ ID NO:18.

41. The parenterally administrable composition of claim 40, wherein the nucleic acid having SEQ ID NO:1, SEQ ID NO:2, SEQ ID NO:3, SEQ ID NO:4, SEQ ID NO:8, SEQ ID NO:7, SEQ ID NO:15, ORF of SEQ ID NO:15, SEQ ID NO:16, ORF of SEQ ID NO:16, SEQ ID NO:17, ORF of SEQ ID NO:17, SEQ ID NO:18 or ORF of SEQ ID NO:18 or any combination thereof imparts the attenuation compared to its natural isolate form.

42. (canceled)
